(19) United States
(12) Patent Application Publication
(10) Pub. No.: US 2024/0024294 A1
(43) Pub. Date: Jan. 25, 2024

(54) COMPOSITIONS AND METHODS FOR THE TREATMENT OR PREVENTION OF PRETERM LABOR

(71) Applicant: XOMA (US) LLC, Emeryville, CA (US)

(72) Inventor: Jean-Pierre GOTTELAND, Geneva (CH)

(21) Appl. No.: 18/037,007

(22) PCT Filed: Nov. 16, 2021
(86) PCT No.: PCT/EP2021/081794

§ 371 Date: May 15, 2023

Related U.S. Application Data
(60) Provisional application No. 63/114,233, filed on Nov. 16, 2020.

(51) Int. Cl. A61K 31/426; A61K 38/095; A61P 15/06

(52) U.S. Cl. CPC A61K 31/426; A61K 38/095; A61P 15/06

(57) ABSTRACT

The disclosure provides compositions and methods for delaying the onset of delivery in a pregnant subject, such as a pregnant human subject, that is undergoing or at risk of undergoing preterm labor at a gestational age of from about 24 weeks to about 34 weeks. Using the compositions and methods described herein, such subjects may be administered atosiban in combination with a prostaglandin F2α (PGF2α) antagonist. Exemplary PGF2α receptor antagonists that may be used for the treatment or prevention of preterm labor as described herein include 1,3-thiazolidine-2-carboxamide compounds, such as (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof (e.g., (3S)-3-({[(2S)-3-(biphenyl-4-ylsulfonyl)-1,3-thiazolidin-2-yl]carbonyl}-amino)-3-(4-fluorophenyl)propyl L-valinate hydrochloride). The compositions and methods described herein provide various clinical benefits. Delivery at an early gestational age is a leading cause of perinatal mortality. By delaying labor in pregnant human patients at an early gestational age, the compositions and methods of the disclosure provide unborn infants with additional time to develop vital organs and tissue systems, thereby significantly improving the likelihood of survival following delivery. The compositions and methods described herein may be used to delay the onset of delivery by one or more hours, days, or weeks, for example, so as to enable pregnant subjects to reach a gestational age at which parturition is substantially safer.

### FIELD OF THE INVENTION

The invention relates to the field of therapeutic treatment of pregnant subjects, such as pregnant human subjects undergoing or at risk of undergoing preterm labor at the early gestational stage.

### BACKGROUND OF THE INVENTION

Preterm delivery represents a prevalent cause of perinatal mortality in the developed world and occurs in approximately 7% to 10% of all deliveries (Berkowitz et al. Epidemiol. Rev. 15:414-443 (1993)). Severe morbidity, especially respiratory distress syndrome, intraventricular hemorrhage, bronchopulmonary dysplasia, and necrotizing enterocolitis, are far more common in preterm than in term infants. Long-term impairments, such as cerebral palsy, visual impairment, and hearing loss, are also more common in preterm infants. At present, preterm birth remains a leading cause of infant mortality and morbidity in the United States, where, despite the significant improvements in obstetrical medicine, the infant mortality rate is higher than in many other industrialized nations, causing costs exceeding $5 billion per year for neonatal intensive care of low birth-weight babies. The actual costs associated with this care are even higher when taking into consideration the healthcare provision of preterm childbirth-related ailments, such as respiratory distress syndrome, heart conditions, cerebral palsy, epilepsy, and severe learning disabilities.

Fundamentally, term and preterm labor are similar processes in that they share a common physiological endpoint characterized by uterine contractions, cervical dilatation, and activation of the fetal membranes. The differences lie in the gestational age at which these processes occur and the mechanisms by which they are activated. Term labor is thought to result from physiological activation of the terminal pathway, whereas preterm labor is a pathological condition characterized by multiple etiologies in which one or more components of this pathway are aberrantly activated.

There remains a need for combinations of agents that can be used to treat or prevent preterm labor, as well as dosing regimens for the use of such agents at levels that are therapeutically effective and physiologically safe.

### SUMMARY OF THE INVENTION

The present disclosure relates to compositions and methods for treating or preventing preterm labor in a patient, such as a mammalian patient, and particularly a human female patient. Using the compositions and methods described herein, a patient undergoing or at risk of undergoing premature parturition, such as a subject being prepared for cesarean delivery, may be administered a prostaglandin F2α (PGF2α) receptor antagonist in combination with an oxytocin receptor antagonist, such as atosiban, so as to delay the onset of delivery, for instance, by a matter of hours, days, or weeks. The compositions and methods described herein can additionally be used to ameliorate one or more symptoms of preterm labor, such as to diminish the frequency of uterine contractions, slow or halt vaginal bleeding, and/or to suppress the rupture of uterine membranes. The PGF2α receptor antagonist may be, for example, a 1,3-thiazolidine-2-carboxamide compound described herein, such as (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof, such as (3S)-3-({[(2S)-3-(biphenyl-4-ylsulfonyl)-1,3-thiazolidin-2-yl]carbonyl}-amino)-3-(4-fluorophenyl)propyl L-valinate hydrochloride, or another 1,3-thiazolidine-2-carboxamide that gives rise to 3-([1,1′-biphenyl]-4-ylsulfonyl)-N-[1-(4-fluorophenyl)-3-hydroxypropyl]-1,3-thiazolidine-2-carboxamide in vivo.

The compositions and methods of the present disclosure provide a variety of important therapeutic benefits. Using the compositions and methods described herein, a patient, such as a pregnant human female patient, undergoing or at risk of undergoing preterm labor may be administered a PGF2α receptor antagonist, such as a 1,3-thiazolidine-2-carboxamide compound described herein, in combination with atosiban. The administration of these agents to the patient can prolong the patient's pregnancy, providing the unborn infant with additional time to develop vital organs and tissue systems. This activity is particularly valuable for patients characterized by an early gestational age, such as a gestational age of from about 24 weeks to about 34 weeks (e.g., a gestational age of from about 28 and 0/7 weeks to about 33 and 6/7 weeks, such as a gestational age of about 28 and 0/7 weeks, 28 and 1/7 weeks, 28 and 2/7 weeks, 28 and 3/7 weeks, 28 and 4/7 weeks, 28 and 5/7 weeks, 28 and 6/7 weeks, 29 and 0/7 weeks, 29 and 1/7 weeks, 29 and 2/7 weeks, 29 and 3/7 weeks, 29 and 4/7 weeks, 29 and 5/7 weeks, 29 and 6/7 weeks, 30 and 0/7 weeks, 30 and 1/7 weeks, 30 and 2/7 weeks, 30 and 3/7 weeks, 30 and 4/7 weeks, 30 and 5/7 weeks, 30 and 6/7 weeks, 31 and 0/7 weeks, 31 and 1/7 weeks, 31 and 2/7 weeks, 31 and 3/7 weeks, 31 and 4/7 weeks, 31 and 5/7 weeks, 31 and 6/7 weeks, 32 and 0/7 weeks, 32 and 1/7 weeks, 32 and 2/7 weeks, 32 and 3/7 weeks, 32 and 4/7 weeks, 32 and 5/7 weeks, 32 and 6/7 weeks, 33 and 0/7 weeks, 33 and 1/7 weeks, 33 and 2/7 weeks, 33 and 3/7 weeks, 33 and 4/7 weeks, 33 and 5/7 weeks, or 33 and 6/7 weeks). At such young gestational ages, unborn infants are likely to have respiratory systems and nervous systems, among other critical organ networks, that have not sufficiently matured. Without being limited by mechanism, by providing patients that are undergoing or at risk of undergoing parturition prior to a full term with a PGF2α receptor antagonist (e.g., (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof, such as (3S)-3-({[(2S)-3-(biphenyl-4-ylsulfonyl)-1,3-thiazolidin-2-yl]carbonyl}-amino)-3-(4-fluorophenyl)propyl L-valinate hydrochloride, or another 1,3-thiazolidine-2-carboxamide that gives rise to 3-([1,1′-biphenyl]-4-ylsulfonyl)-N-[1-(4-fluorophenyl)-3-hydroxypropyl]-1,3-thiazolidine-2-carboxamide in vivo) in combination with atosiban according to a dosing schedule described herein, pregnancy can be extended for a time sufficient to enable unborn infants to achieve critical developments in lung and neuronal growth. This maturation, in turn, can effectuate a substantial increase in the likelihood of neonatal survival.

To obtain these advantageous treatment outcomes, a patient may be administered therapeutically effective amounts of the PGF2α receptor antagonist and atosiban, for example, either concurrently or at different times. The patient may receive multiple, continuous doses of the PGF2α receptor antagonist and/or the atosiban. The patient may receive the PGF2α receptor antagonist and the atosiban on the same or different dosing schedules. For instance, each time the patient receives a dose of one of these agents, the patient may or may not receive a dose of the second agent. The patient may receive the PGF2α receptor antagonist, for example, one or more times per day, such as once or twice daily, and the atosiban may be administered to the subject, for example, one or more times every 24 hours, every 36 hours, every 48 hours, every 60 hours, every 72 hours, every 84 hours, every 96 hours, or more, such as in the form of a single, discrete bolus administration or in the form of a continuous administration, such as a continuous intravenous infusion. The combined administration of atosiban and the PGF2α receptor antagonist may occur one or more times per day, week, or month, as described herein, and may continue, for example, up until the patient undergoes delivery or until a full gestational term has been reached.

In a first aspect, the disclosure features a method of delaying the onset of delivery in a pregnant subject, such as a pregnant human subject, by administering to the subject therapeutically effective amounts of atosiban and a PGF2α receptor antagonist, such as a compound represented by formula (I)

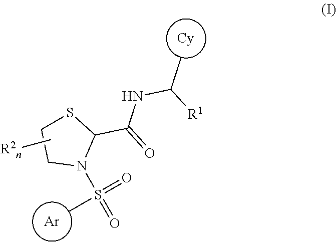

wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof.

In accordance with this aspect, the compound may be administered to the subject in an amount of from about 250 mg to about 2,500 mg per dose.

For example, the compound may be administered to the subject in an amount of from about 250 mg to about 750 mg per dose, such as an amount of from about 255 mg to about 745 mg, from about 260 mg to about 740 mg, from about 265 mg to about 735 mg, from about 270 mg to about 730 mg, from about 275 mg to about 725 mg, from about 280 mg to about 720 mg, from about 285 mg to about 715 mg, from about 290 mg to about 710 mg, from about 295 mg to about 705 mg, from about 300 mg to about 700 mg, from about 305 mg to about 695 mg, from about 310 mg to about 690 mg, from about 315 mg to about 685 mg, from about 320 mg to about 680 mg, from about 325 mg to about 675 mg, from about 330 mg to about 670 mg, from about 335 mg to about 665 mg, from about 340 mg to about 660 mg, from about 345 mg to about 655 mg, from about 350 mg to about 650 mg, from about 355 mg to about 645 mg, from about 360 mg to about 640 mg, from about 365 mg to about 635 mg, from about 370 mg to about 630 mg, from about 375 mg to about 625 mg, from about 380 mg to about 620 mg, from about 385 mg to about 615 mg, from about 390 mg to about 610 mg, from about 395 mg to about 605 mg, from about 400 mg to about 600 mg, from about 405 mg to about 595 mg, from about 410 mg to about 590 mg, from about 415 mg to about 585 mg, from about 420 mg to about 580 mg, from about 425 mg to about 575 mg, from about 430 mg to about 570 mg, from about 435 mg to about 565 mg, from about 440 mg to about 560 mg, from about 445 mg to about 555 mg, from about 450 mg to about 550 mg, from about 455 mg to about 545 mg, from about 460 mg to about 540 mg, from about 465 mg to about 535 mg, from about 470 mg to about 530 mg, from about 475 mg to about 525 mg, from about 480 mg to about 520 mg, from about 485 mg to about 515 mg, from about 490 mg to about 510 mg, or from about 495 mg to about 505 mg per dose. In some embodiments, the compound is administered to the subject in an amount of about 500 mg per dose.

In some embodiments, the compound is administered to the subject in an amount of from about 250 mg to about 2,000 mg per dose, such as an amount of from about 255 mg to about 1,990 mg, from about 260 mg to about 1,980 mg, from about 265 mg to about 1,970 mg, from about 270 mg to about 1,960 mg, from about 275 mg to about 1,950 mg, from about 280 mg to about 1,940 mg, from about 285 mg to about 1,930 mg, from about 290 mg to about 1,920 mg, from about 295 mg to about 1,910 mg, from about 300 mg to about 1,900 mg, from about 305 mg to about 1,890 mg, from about 310 mg to about 1,880 mg, from about 315 mg to about 1,870 mg, from about 320 mg to about 1,860 mg, from about 325 mg to about 1,850 mg, from about 330 mg to about 1,840 mg, from about 335 mg to about 1,830 mg, from about 340 mg to about 1,820 mg, from about 345 mg to about 1,810 mg, from about 350 mg to about 1,800 mg, from about 355 mg to about 1,790 mg, from about 360 mg to about 1,780 mg, from about 365 mg to about 1,770 mg, from about 370 mg to about 1,760 mg, from about 375 mg to about 1,750 mg, from about 380 mg to about 1,740 mg, from about 385 mg to about 1,730 mg, from about 390 mg to about 1,720 mg, from about 395 mg to about 1,710 mg, from about 400 mg to about 1,700 mg, from about 405 mg to about 1,690 mg, from about 410 mg to about 1,680 mg, from about 415 mg to about 1,670 mg, from about 420 mg to about 1,660 mg, from about 425 mg to about 1,650 mg, from about 430 mg to about 1,640 mg, from about 435 mg to about 1,630 mg, from about 440 mg to about 1,620 mg, from about 445 mg to about 1,610 mg, from about 450 mg to about 1,600 mg, from about 455 mg to about 1,590 mg, from about 460 mg to about 1,580 mg, from about 465 mg to about 1,570 mg, from about 470 mg to about 1,560 mg, from about 475 mg to about 1,550 mg, from about 480 mg to about 1,540 mg, from about 485 mg to about 1,530 mg, from about 490 mg to about 1,520 mg, from about 495 mg to about 1,510 mg, from about 500 mg to about 1,500 mg, from about 505 mg to about 1,495 mg, from about 510 mg to about 1,490 mg, from about 515 mg to about 1,485 mg, from about 520 mg to about 1,480 mg, from about 525 mg to about 1,475 mg, from about 530 mg to about 1,470 mg, from about 535 mg to about 1,465 mg, from about 540 mg to about 1,460 mg, from about 545 mg to about 1,455 mg, from about 550 mg to about 1,450 mg, from about 555 mg to about 1,445 mg, from about 560 mg to about 1,440 mg, from about 565 mg to about 1,435 mg, from about 570 mg to about 1,430 mg, from about 575 mg to about 1,425 mg, from about 580 mg to about 1,420 mg, from about 585 mg to about 1,415 mg, from about 590 mg to about 1,410 mg, from about 595 mg t about 1,405 mg, from about 600 mg to about 1,400 mg, from about 605 mg to about 1,395 mg, from about 610 mg to about 1,390 mg, from about 615 mg to about 1,385 mg, from about 620 mg to about 1,380 mg, from about 625 mg to about 1,375 mg, from about 630 mg to about 1,370 mg, from about 635 mg to about 1,365 mg, from about 640 mg to about 1,360 mg, from about 645 mg to about 1,355 mg, from about 650 mg to about 1,350 mg, from about 655 mg to about 1,345 mg, from about 660 mg to about 1,340 mg, from about 665 mg to about 1,335 mg, from about 670 mg to about 1,330 mg, from about 675 mg to about 1,325 mg, from about 680 mg to about 1,320 mg, from about 685 mg to about 1,315 mg, from about 690 mg to about 1,310 mg, from about 695 mg to about 1,305 mg, from about 700 mg to about 1,300 mg, from about 705 mg to about 1,295 mg, from about 710 mg to about 1,290 mg, from about 715 mg to about 1,285 mg, from about 720 mg to about 1,280 mg, from about 725 mg to about 1,275 mg, from about 730 mg to about 1,270 mg, from about 735 mg to about 1,265 mg, from about 740 mg to about 1,260 mg, from about 745 mg to about 1,255 mg, from about 750 mg to about 1,250 mg, from about 755 mg to about 1,245 mg, from about 760 mg to about 1,240 mg, from about 765 mg to about 1,235 mg, from about 770 mg to about 1,230 mg, from about 775 mg to about 1,225 mg, from about 780 mg to about 1,220 mg, from about 785 mg to about 1,215 mg, from about 790 mg to about 1,210 mg, from about 795 mg to about 1,205 mg, from about 800 mg to about 1,200 mg, from about 805 mg to about 1,195 mg, from about 810 mg to about 1,190 mg, from about 815 mg to about 1,185 mg, from about 820 mg to about 1,180 mg, from about 825 mg to about 1,175 mg, from about 830 mg to about 1,170 mg, from about 835 mg to about 1,165 mg, from about 840 mg to about 1,160 mg, from about 845 mg to about 1,155 mg, from about 850 mg to about 1,150 mg, from about 855 mg to about 1,145 mg, from about 860 mg to about 1,140 mg, from about 865 mg to about 1,135 mg, from about 870 mg to about 1,130 mg, from about 875 mg to about 1,125 mg, from about 880 mg to about 1,120 mg, from about 885 mg to about 1,115 mg, from about 890 mg to about 1,110 mg, from about 895 mg to about 1,105 mg, from about 900 mg to about 1,000 mg, from about 905 mg to about 1,095 mg, from about 910 mg to about 1,090 mg, from about 915 mg to about 1,085 mg, from about 920 mg to about 1,080 mg, from about 925 mg to about 1,075 mg, from about 930 mg to about 1,070 mg, from about 935 mg to about 1,065 mg, from about 940 mg to about 1,060 mg, from about 945 mg to about 1,055 mg, from about 950 mg to about 1,050 mg, from about 955 mg to about 1,045 mg, from about 960 mg to about 1,040 mg, from about 965 mg to about 1,035 mg, from about 970 mg to about 1,030 mg, from about 975 mg to about 1,025 mg, from about 980 mg to about 1,020 mg, from about 985 mg to about 1,015 mg, from about 990 mg to about 1,010 mg, or from about 995 mg to about 1,005 mg per dose. In some embodiments, the compound is administered to the subject in an amount of about 1,000 mg per dose.

In some embodiments, the compound is administered to the subject in an amount of from about 1,500 mg to about 2,500 mg per dose, such as an amount of from about 1,505 mg to about 2,495 mg, from about 1,510 mg to about 2,490 mg, from about 1,515 mg to about 2,485 mg, from about 1,520 mg to about 2,480 mg, from about 1,525 mg to about 2,475 mg, from about 1,530 mg to about 2,470 mg, from about 1,535 mg to about 2,465 mg, from about 1,540 mg to about 2,460 mg, from about 1,545 mg to about 2,455 mg, from about 1,550 mg to about 2,450 mg, from about 1,555 mg to about 2,445 mg, from about 1,560 mg to about 2,440 mg, from about 1,565 mg to about 2,435 mg, from about 1,570 mg to about 2,430 mg, from about 1,575 mg to about 2,425 mg, from about 1,580 mg to about 2,420 mg, from about 1,585 mg to about 2,415 mg, from about 1,590 mg to about 2,410 mg, from about 1,595 mg to about 2,405 mg, from about 1,600 mg to about 2,400 mg, from about 1,605 mg to about 2,395 mg, from about 1,610 mg to about 2,390 mg, from about 1,615 mg to about 2,385 mg, from about 1,620 mg to about 2,380 mg, from about 1,625 mg to about 2,375 mg, from about 1,630 mg to about 2,370 mg, from about 1,635 mg to about 2,365 mg, from about 1,640 mg to about 2,360 mg, from about 1,645 mg to about 2,355 mg, from about 1,650 mg to about 2,350 mg, from about 1,655 mg to about 2,345 mg, from about 1,660 mg to about 2,340 mg, from about 1,665 mg to about 2,335 mg, from about 1,670 mg to about 2,330 mg, from about 1,675 mg to about 2,325 mg, from about 1,680 mg to about 2,320 mg, from about 1,685 mg to about 2,315 mg, from about 1,690 mg to about 2,310 mg, from about 1,695 mg to about 2,305 mg, from about 1,700 mg to about 2,300 mg, from about 1,705 mg to about 2,295 mg, from about 1,710 mg to about 2,290 mg, from about 1,715 mg to about 2,285 mg, from about 1,720 mg to about 2,280 mg, from about 1,725 mg to about 2,275 mg, from about 1,730 mg to about 2,270 mg, from about 1,735 mg to about 2,265 mg, from about 1,740 mg to about 2,260 mg, from about 1,745 mg to about 2,255 mg, from about 1,750 mg to about 2,250 mg, from about 1,755 mg to about 2,245 mg, from about 1,760 mg to about 2,240 mg, from about 1,765 mg to about 2,235 mg, from about 1,770 mg to about 2,230 mg, from about 1,775 mg to about 2,225 mg, from about 1,780 mg to about 2,220 mg, from about 1,785 mg to about 2,215 mg, from about 1,790 mg to about 2,210 mg, from about 1,795 mg to about 2,205 mg, from about 1,800 mg to about 2,200 mg, from about 1,805 mg to about 2,195 mg, from about 1,810 mg to about 2,190 mg, from about 1,815 mg to about 2,185 mg, from about 1,820 mg to about 2,180 mg, from about 1,825 mg to about 2,175 mg, from about 1,830 mg to about 2,170 mg, from about 1,835 mg to about 2,165 mg, from about 1,840 mg to about 2,160 mg, from about 1,845 mg to about 2,155 mg, from about 1,850 mg to about 2,150 mg, from about 1,855 mg to about 2,145 mg, from about 1,860 mg to about 2,140 mg, from about 1,865 mg to about 2,135 mg, from about 1,870 mg to about 2,130 mg, from about 1,875 mg to about 2,125 mg, from about 1,880 mg to about 2,120 mg, from about 1,885 mg to about 2,115 mg, from about 1,890 mg to about 2,110 mg, from about 1,895 mg to about 2,105 mg, from about 1,900 mg to about 2,100 mg, from about 1,905 mg to about 2,095 mg, from about 1,910 mg to about 2,090 mg, from about 1,915 mg to about 2,085 mg, from about 1,920 mg to about 2,080 mg, from about 1,925 mg to about 2,075 mg, from about 1,930 mg to about 2,070 mg, from about 1,935 mg to about 2,065 mg, from about 1,940 mg to about 2,060 mg, from about 1,945 mg to about 2,055 mg, from about 1,950 mg to about 2,050 mg, from about 1,955 mg to about 2,045 mg, from about 1,960 mg to about 2,040 mg, from about 1,965 mg to about 2,035 mg, from about 1,970 mg to about 2,030 mg, from about 1,975 mg to about 2,025 mg, from about 1,980 mg to about 2,020 mg, from about 1,985 mg to about 2,015 mg, from about 1,990 mg to about 2,010 mg, or from about 1,995 mg to about 2,005 mg per dose. In some embodiments, the compound is administered to the subject in an amount of about 2,000 mg per dose.

For example, in some embodiments, the compound is administered to the subject in an amount of about 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, 1,150 mg, 1,155 mg, 1,160 mg, 1,165 mg, 1,170 mg, 1,175 mg, 1,180 mg, 1,185 mg, 1,190 mg, 1,195 mg, 1,200 mg, 1,205 mg, 1,210 mg, 1,215 mg, 1,220 mg, 1,225 mg, 1,230 mg, 1,235 mg, 1,240 mg, 1,245 mg, 1,250 mg, 1,255 mg, 1,260 mg, 1,265 mg, 1,270 mg, 1,275 mg, 1,280 mg, 1,285 mg, 1,290 mg, 1,295 mg, 1,300 mg, 1,305 mg, 1,310 mg, 1,315 mg, 1,320 mg, 1,325 mg, 1,330 mg, 1,335 mg, 1,340 mg, 1,345 mg, 1,350 mg, 1,355 mg, 1,360 mg, 1,365 mg, 1,370 mg, 1,375 mg, 1,380 mg, 1,385 mg, 1,390 mg, 1,395 mg, 1,400 mg, 1,405 mg, 1,410 mg, 1,415 mg, 1,420 mg, 1,425 mg, 1,430 mg, 1,435 mg, 1,440 mg, 1,445 mg, 1,450 mg, 1,455 mg, 1,460 mg, 1,465 mg, 1,470 mg, 1,475 mg, 1,480 mg, 1,485 mg, 1,490 mg, 1,495 mg, 1,500 mg, 1,505 mg, 1,510 mg, 1,515 mg, 1,520 mg, 1,525 mg, 1,530 mg, 1,535 mg, 1,540 mg, 1,545 mg, 1,550 mg, 1,555 mg, 1,560 mg, 1,565 mg, 1,570 mg, 1,575 mg, 1,580 mg, 1,585 mg, 1,590 mg, 1,595 mg, 1,600 mg, 1,605 mg, 1,610 mg, 1,615 mg, 1,620 mg, 1,625 mg, 1,630 mg, 1,635 mg, 1,640 mg, 1,645 mg, 1,650 mg, 1,655 mg, 1,660 mg, 1,665 mg, 1,670 mg, 1,675 mg, 1,680 mg, 1,685 mg, 1,690 mg, 1,695 mg, 1,700 mg, 1,705 mg, 1,710 mg, 1,715 mg, 1,720 mg, 1,725 mg, 1,730 mg, 1,735 mg, 1,740 mg, 1,745 mg, 1,750 mg, 1,755 mg, 1,760 mg, 1,765 mg, 1,770 mg, 1,775 mg, 1,780 mg, 1,785 mg, 1,790 mg, 1,795 mg, 1,800 mg, 1,805 mg, 1,810 mg, 1,815 mg, 1,820 mg, 1,825 mg, 1,830 mg, 1,835 mg, 1,840 mg, 1,845 mg, 1,850 mg, 1,855 mg, 1,860 mg, 1,865 mg, 1,870 mg, 1,875 mg, 1,880 mg, 1,885 mg, 1,890 mg, 1,895 mg, 1,900 mg, 1,905 mg, 1,910 mg, 1,915 mg, 1,920 mg, 1,925 mg, 1,930 mg, 1,935 mg, 1,940 mg, 1,945 mg, 1,950 mg, 1,955 mg, 1,960 mg, 1,965 mg, 1,970 mg, 1,975 mg, 1,980 mg, 1,985 mg, 1,990 mg, 1,995 mg, 2,000 mg, 2,005 mg, 2,010 mg, 2,015 mg, 2,020 mg, 2,025 mg, 2,030 mg, 2,035 mg, 2,040 mg, 2,045 mg, 2,050 mg, 2,055 mg, 2,060 mg, 2,065 mg, 2,070 mg, 2,075 mg, 2,080 mg, 2,085 mg, 2,090 mg, 2,095 mg, 2,100 mg, 2,105 mg, 2,110 mg, 2,115 mg, 2,120 mg, 2,125 mg, 2,130 mg, 2,135 mg, 2,140 mg, 2,145 mg, 2,150 mg, 2,155 mg, 2,160 mg, 2,165 mg, 2,170 mg, 2,175 mg, 2,180 mg, 2,185 mg, 2,190 mg, 2,195 mg, 2,200 mg, 2,205 mg, 2,210 mg, 2,215 mg, 2,220 mg, 2,225 mg, 2,230 mg, 2,235 mg, 2,240 mg, 2,245 mg, 2,250 mg, 2,255 mg, 2,260 mg, 2,265 mg, 2,270 mg, 2,275 mg, 2,280 mg, 2,285 mg, 2,290 mg, 2,295 mg, 2,300 mg, 2,305 mg, 2,310 mg, 2,315 mg, 2,320 mg, 2,325 mg, 2,330 mg, 2,335 mg, 2,340 mg, 2,345 mg, 2,350 mg, 2,355 mg, 2,360 mg, 2,365 mg, 2,370 mg, 2,375 mg, 2,380 mg, 2,385 mg, 2,390 mg, 2,395 mg, 2,400 mg, 2,405 mg, 2,410 mg, 2,415 mg, 2,420 mg, 2,425 mg, 2,430 mg, 2,435 mg, 2,440 mg, 2,445 mg, 2,450 mg, 2,455 mg, 2,460 mg, 2,465 mg, 2,470 mg, 2,475 mg, 2,480 mg, 2,485 mg, 2,490 mg, 2,495 mg, or 2,500 mg per dose.

In another aspect, the disclosure features a method of treating or preventing preterm labor in a pregnant subject, such as a pregnant human subject, by administering to the subject therapeutically effective amounts of atosiban and a PGF2α receptor antagonist, such as a compound represented by formula (I)

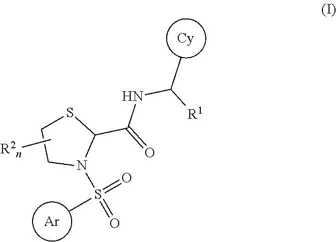

wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof.

In accordance with the foregoing aspect, the compound may be administered to the subject in an amount of from about 250 mg to about 2,500 mg per dose.

For example, the compound may be administered to the subject in an amount of from about 250 mg to about 750 mg per dose, such as an amount of from about 255 mg to about 745 mg, from about 260 mg to about 740 mg, from about 265 mg to about 735 mg, from about 270 mg to about 730 mg, from about 275 mg to about 725 mg, from about 280 mg to about 720 mg, from about 285 mg to about 715 mg, from about 290 mg to about 710 mg, from about 295 mg to about 705 mg, from about 300 mg to about 700 mg, from about 305 mg to about 695 mg, from about 310 mg to about 690 mg, from about 315 mg to about 685 mg, from about 320 mg to about 680 mg, from about 325 mg to about 675 mg, from about 330 mg to about 670 mg, from about 335 mg to about 665 mg, from about 340 mg to about 660 mg, from about 345 mg to about 655 mg, from about 350 mg to about 650 mg, from about 355 mg to about 645 mg, from about 360 mg to about 640 mg, from about 365 mg to about 635 mg, from about 370 mg to about 630 mg, from about 375 mg to about 625 mg, from about 380 mg to about 620 mg, from about 385 mg to about 615 mg, from about 390 mg to about 610 mg, from about 395 mg to about 605 mg, from about 400 mg to about 600 mg, from about 405 mg to about 595 mg, from about 410 mg to about 590 mg, from about 415 mg to about 585 mg, from about 420 mg to about 580 mg, from about 425 mg to about 575 mg, from about 430 mg to about 570 mg, from about 435 mg to about 565 mg, from about 440 mg to about 560 mg, from about 445 mg to about 555 mg, from about 450 mg to about 550 mg, from about 455 mg to about 545 mg, from about 460 mg to about 540 mg, from about 465 mg to about 535 mg, from about 470 mg to about 530 mg, from about 475 mg to about 525 mg, from about 480 mg to about 520 mg, from about 485 mg to about 515 mg, from about 490 mg to about 510 mg, or from about 495 mg to about 505 mg per dose. In some embodiments, the compound is administered to the subject in an amount of about 500 mg per dose.

In some embodiments, the compound is administered to the subject in an amount of from about 250 mg to about 2,000 mg per dose, such as an amount of from about 255 mg to about 1,990 mg, from about 260 mg to about 1,980 mg, from about 265 mg to about 1,970 mg, from about 270 mg to about 1,960 mg, from about 275 mg to about 1,950 mg, from about 280 mg to about 1,940 mg, from about 285 mg to about 1,930 mg, from about 290 mg to about 1,920 mg, from about 295 mg to about 1,910 mg, from about 300 mg to about 1,900 mg, from about 305 mg to about 1,890 mg, from about 310 mg to about 1,880 mg, from about 315 mg to about 1,870 mg, from about 320 mg to about 1,860 mg, from about 325 mg to about 1,850 mg, from about 330 mg to about 1,840 mg, from about 335 mg to about 1,830 mg, from about 340 mg to about 1,820 mg, from about 345 mg to about 1,810 mg, from about 350 mg to about 1,800 mg, from about 355 mg to about 1,790 mg, from about 360 mg to about 1,780 mg, from about 365 mg to about 1,770 mg, from about 370 mg to about 1,760 mg, from about 375 mg to about 1,750 mg, from about 380 mg to about 1,740 mg, from about 385 mg to about 1,730 mg, from about 390 mg to about 1,720 mg, from about 395 mg to about 1,710 mg, from about 400 mg to about 1,700 mg, from about 405 mg to about 1,690 mg, from about 410 mg to about 1,680 mg, from about 415 mg to about 1,670 mg, from about 420 mg to about 1,660 mg, from about 425 mg to about 1,650 mg, from about 430 mg to about 1,640 mg, from about 435 mg to about 1,630 mg, from about 440 mg to about 1,620 mg, from about 445 mg to about 1,610 mg, from about 450 mg to about 1,600 mg, from about 455 mg to about 1,590 mg, from about 460 mg to about 1,580 mg, from about 465 mg to about 1,570 mg, from about 470 mg to about 1,560 mg, from about 475 mg to about 1,550 mg, from about 480 mg to about 1,540 mg, from about 485 mg to about 1,530 mg, from about 490 mg to about 1,520 mg, from about 495 mg to about 1,510 mg, from about 500 mg to about 1,500 mg, from about 505 mg to about 1,495 mg, from about 510 mg to about 1,490 mg, from about 515 mg to about 1,485 mg, from about 520 mg to about 1,480 mg, from about 525 mg to about 1,475 mg, from about 530 mg to about 1,470 mg, from about 535 mg to about 1,465 mg, from about 540 mg to about 1,460 mg, from about 545 mg to about 1,455 mg, from about 550 mg to about 1,450 mg, from about 555 mg to about 1,445 mg, from about 560 mg to about 1,440 mg, from about 565 mg to about 1,435 mg, from about 570 mg to about 1,430 mg, from about 575 mg to about 1,425 mg, from about 580 mg to about 1,420 mg, from about 585 mg to about 1,415 mg, from about 590 mg to about 1,410 mg, from about 595 mg t about 1,405 mg, from about 600 mg to about 1,400 mg, from about 605 mg to about 1,395 mg, from about 610 mg to about 1,390 mg, from about 615 mg to about 1,385 mg, from about 620 mg to about 1,380 mg, from about 625 mg to about 1,375 mg, from about 630 mg to about 1,370 mg, from about 635 mg to about 1,365 mg, from about 640 mg to about 1,360 mg, from about 645 mg to about 1,355 mg, from about 650 mg to about 1,350 mg, from about 655 mg to about 1,345 mg, from about 660 mg to about 1,340 mg, from about 665 mg to about 1,335 mg, from about 670 mg to about 1,330 mg, from about 675 mg to about 1,325 mg, from about 680 mg to about 1,320 mg, from about 685 mg to about 1,315 mg, from about 690 mg to about 1,310 mg, from about 695 mg to about 1,305 mg, from about 700 mg to about 1,300 mg, from about 705 mg to about 1,295 mg, from about 710 mg to about 1,290 mg, from about 715 mg to about 1,285 mg, from about 720 mg to about 1,280 mg, from about 725 mg to about 1,275 mg, from about 730 mg to about 1,270 mg, from about 735 mg to about 1,265 mg, from about 740 mg to about 1,260 mg, from about 745 mg to about 1,255 mg, from about 750 mg to about 1,250 mg, from about 755 mg to about 1,245 mg, from about 760 mg to about 1,240 mg, from about 765 mg to about 1,235 mg, from about 770 mg to about 1,230 mg, from about 775 mg to about 1,225 mg, from about 780 mg to about 1,220 mg, from about 785 mg to about 1,215 mg, from about 790 mg to about 1,210 mg, from about 795 mg to about 1,205 mg, from about 800 mg to about 1,200 mg, from about 805 mg to about 1,195 mg, from about 810 mg to about 1,190 mg, from about 815 mg to about 1,185 mg, from about 820 mg to about 1,180 mg, from about 825 mg to about 1,175 mg, from about 830 mg to about 1,170 mg, from about 835 mg to about 1,165 mg, from about 840 mg to about 1,160 mg, from about 845 mg to about 1,155 mg, from about 850 mg to about 1,150 mg, from about 855 mg to about 1,145 mg, from about 860 mg to about 1,140 mg, from about 865 mg to about 1,135 mg, from about 870 mg to about 1,130 mg, from about 875 mg to about 1,125 mg, from about 880 mg to about 1,120 mg, from about 885 mg to about 1,115 mg, from about 890 mg to about 1,110 mg, from about 895 mg to about 1,105 mg, from about 900 mg to about 1,000 mg, from about 905 mg to about 1,095 mg, from about 910 mg to about 1,090 mg, from about 915 mg to about 1,085 mg, from about 920 mg to about 1,080 mg, from about 925 mg to about 1,075 mg, from about 930 mg to about 1,070 mg, from about 935 mg to about 1,065 mg, from about 940 mg to about 1,060 mg, from about 945 mg to about 1,055 mg, from about 950 mg to about 1,050 mg, from about 955 mg to about 1,045 mg, from about 960 mg to about 1,040 mg, from about 965 mg to about 1,035 mg, from about 970 mg to about 1,030 mg, from about 975 mg to about 1,025 mg, from about 980 mg to about 1,020 mg, from about 985 mg to about 1,015 mg, from about 990 mg to about 1,010 mg, or from about 995 mg to about 1,005 mg per dose. In some embodiments, the compound is administered to the subject in an amount of about 1,000 mg per dose.

In some embodiments, the compound is administered to the subject in an amount of from about 1,500 mg to about 2,500 mg per dose, such as an amount of from about 1,505 mg to about 2,495 mg, from about 1,510 mg to about 2,490 mg, from about 1,515 mg to about 2,485 mg, from about 1,520 mg to about 2,480 mg, from about 1,525 mg to about 2,475 mg, from about 1,530 mg to about 2,470 mg, from about 1,535 mg to about 2,465 mg, from about 1,540 mg to about 2,460 mg, from about 1,545 mg to about 2,455 mg, from about 1,550 mg to about 2,450 mg, from about 1,555 mg to about 2,445 mg, from about 1,560 mg to about 2,440 mg, from about 1,565 mg to about 2,435 mg, from about 1,570 mg to about 2,430 mg, from about 1,575 mg to about 2,425 mg, from about 1,580 mg to about 2,420 mg, from about 1,585 mg to about 2,415 mg, from about 1,590 mg to about 2,410 mg, from about 1,595 mg to about 2,405 mg, from about 1,600 mg to about 2,400 mg, from about 1,605 mg to about 2,395 mg, from about 1,610 mg to about 2,390 mg, from about 1,615 mg to about 2,385 mg, from about 1,620 mg to about 2,380 mg, from about 1,625 mg to about 2,375 mg, from about 1,630 mg to about 2,370 mg, from about 1,635 mg to about 2,365 mg, from about 1,640 mg to about 2,360 mg, from about 1,645 mg to about 2,355 mg, from about 1,650 mg to about 2,350 mg, from about 1,655 mg to about 2,345 mg, from about 1,660 mg to about 2,340 mg, from about 1,665 mg to about 2,335 mg, from about 1,670 mg to about 2,330 mg, from about 1,675 mg to about 2,325 mg, from about 1,680 mg to about 2,320 mg, from about 1,685 mg to about 2,315 mg, from about 1,690 mg to about 2,310 mg, from about 1,695 mg to about 2,305 mg, from about 1,700 mg to about 2,300 mg, from about 1,705 mg to about 2,295 mg, from about 1,710 mg to about 2,290 mg, from about 1,715 mg to about 2,285 mg, from about 1,720 mg to about 2,280 mg, from about 1,725 mg to about 2,275 mg, from about 1,730 mg to about 2,270 mg, from about 1,735 mg to about 2,265 mg, from about 1,740 mg to about 2,260 mg, from about 1,745 mg to about 2,255 mg, from about 1,750 mg to about 2,250 mg, from about 1,755 mg to about 2,245 mg, from about 1,760 mg to about 2,240 mg, from about 1,765 mg to about 2,235 mg, from about 1,770 mg to about 2,230 mg, from about 1,775 mg to about 2,225 mg, from about 1,780 mg to about 2,220 mg, from about 1,785 mg to about 2,215 mg, from about 1,790 mg to about 2,210 mg, from about 1,795 mg to about 2,205 mg, from about 1,800 mg to about 2,200 mg, from about 1,805 mg to about 2,195 mg, from about 1,810 mg to about 2,190 mg, from about 1,815 mg to about 2,185 mg, from about 1,820 mg to about 2,180 mg, from about 1,825 mg to about 2,175 mg, from about 1,830 mg to about 2,170 mg, from about 1,835 mg to about 2,165 mg, from about 1,840 mg to about 2,160 mg, from about 1,845 mg to about 2,155 mg, from about 1,850 mg to about 2,150 mg, from about 1,855 mg to about 2,145 mg, from about 1,860 mg to about 2,140 mg, from about 1,865 mg to about 2,135 mg, from about 1,870 mg to about 2,130 mg, from about 1,875 mg to about 2,125 mg, from about 1,880 mg to about 2,120 mg, from about 1,885 mg to about 2,115 mg, from about 1,890 mg to about 2,110 mg, from about 1,895 mg to about 2,105 mg, from about 1,900 mg to about 2,100 mg, from about 1,905 mg to about 2,095 mg, from about 1,910 mg to about 2,090 mg, from about 1,915 mg to about 2,085 mg, from about 1,920 mg to about 2,080 mg, from about 1,925 mg to about 2,075 mg, from about 1,930 mg to about 2,070 mg, from about 1,935 mg to about 2,065 mg, from about 1,940 mg to about 2,060 mg, from about 1,945 mg to about 2,055 mg, from about 1,950 mg to about 2,050 mg, from about 1,955 mg to about 2,045 mg, from about 1,960 mg to about 2,040 mg, from about 1,965 mg to about 2,035 mg, from about 1,970 mg to about 2,030 mg, from about 1,975 mg to about 2,025 mg, from about 1,980 mg to about 2,020 mg, from about 1,985 mg to about 2,015 mg, from about 1,990 mg to about 2,010 mg, or from about 1,995 mg to about 2,005 mg per dose. In some embodiments, the compound is administered to the subject in an amount of about 2,000 mg per dose.

For example, in some embodiments, the compound is administered to the subject in an amount of about 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, 1,150 mg, 1,155 mg, 1,160 mg, 1,165 mg, 1,170 mg, 1,175 mg, 1,180 mg, 1,185 mg, 1,190 mg, 1,195 mg, 1,200 mg, 1,205 mg, 1,210 mg, 1,215 mg, 1,220 mg, 1,225 mg, 1,230 mg, 1,235 mg, 1,240 mg, 1,245 mg, 1,250 mg, 1,255 mg, 1,260 mg, 1,265 mg, 1,270 mg, 1,275 mg, 1,280 mg, 1,285 mg, 1,290 mg, 1,295 mg, 1,300 mg, 1,305 mg, 1,310 mg, 1,315 mg, 1,320 mg, 1,325 mg, 1,330 mg, 1,335 mg, 1,340 mg, 1,345 mg, 1,350 mg, 1,355 mg, 1,360 mg, 1,365 mg, 1,370 mg, 1,375 mg, 1,380 mg, 1,385 mg, 1,390 mg, 1,395 mg, 1,400 mg, 1,405 mg, 1,410 mg, 1,415 mg, 1,420 mg, 1,425 mg, 1,430 mg, 1,435 mg, 1,440 mg, 1,445 mg, 1,450 mg, 1,455 mg, 1,460 mg, 1,465 mg, 1,470 mg, 1,475 mg, 1,480 mg, 1,485 mg, 1,490 mg, 1,495 mg, 1,500 mg, 1,505 mg, 1,510 mg, 1,515 mg, 1,520 mg, 1,525 mg, 1,530 mg, 1,535 mg, 1,540 mg, 1,545 mg, 1,550 mg, 1,555 mg, 1,560 mg, 1,565 mg, 1,570 mg, 1,575 mg, 1,580 mg, 1,585 mg, 1,590 mg, 1,595 mg, 1,600 mg, 1,605 mg, 1,610 mg, 1,615 mg, 1,620 mg, 1,625 mg, 1,630 mg, 1,635 mg, 1,640 mg, 1,645 mg, 1,650 mg, 1,655 mg, 1,660 mg, 1,665 mg, 1,670 mg, 1,675 mg, 1,680 mg, 1,685 mg, 1,690 mg, 1,695 mg, 1,700 mg, 1,705 mg, 1,710 mg, 1,715 mg, 1,720 mg, 1,725 mg, 1,730 mg, 1,735 mg, 1,740 mg, 1,745 mg, 1,750 mg, 1,755 mg, 1,760 mg, 1,765 mg, 1,770 mg, 1,775 mg, 1,780 mg, 1,785 mg, 1,790 mg, 1,795 mg, 1,800 mg, 1,805 mg, 1,810 mg, 1,815 mg, 1,820 mg, 1,825 mg, 1,830 mg, 1,835 mg, 1,840 mg, 1,845 mg, 1,850 mg, 1,855 mg, 1,860 mg, 1,865 mg, 1,870 mg, 1,875 mg, 1,880 mg, 1,885 mg, 1,890 mg, 1,895 mg, 1,900 mg, 1,905 mg, 1,910 mg, 1,915 mg, 1,920 mg, 1,925 mg, 1,930 mg, 1,935 mg, 1,940 mg, 1,945 mg, 1,950 mg, 1,955 mg, 1,960 mg, 1,965 mg, 1,970 mg, 1,975 mg, 1,980 mg, 1,985 mg, 1,990 mg, 1,995 mg, 2,000 mg, 2,005 mg, 2,010 mg, 2,015 mg, 2,020 mg, 2,025 mg, 2,030 mg, 2,035 mg, 2,040 mg, 2,045 mg, 2,050 mg, 2,055 mg, 2,060 mg, 2,065 mg, 2,070 mg, 2,075 mg, 2,080 mg, 2,085 mg, 2,090 mg, 2,095 mg, 2,100 mg, 2,105 mg, 2,110 mg, 2,115 mg, 2,120 mg, 2,125 mg, 2,130 mg, 2,135 mg, 2,140 mg, 2,145 mg, 2,150 mg, 2,155 mg, 2,160 mg, 2,165 mg, 2,170 mg, 2,175 mg, 2,180 mg, 2,185 mg, 2,190 mg, 2,195 mg, 2,200 mg, 2,205 mg, 2,210 mg, 2,215 mg, 2,220 mg, 2,225 mg, 2,230 mg, 2,235 mg, 2,240 mg, 2,245 mg, 2,250 mg, 2,255 mg, 2,260 mg, 2,265 mg, 2,270 mg, 2,275 mg, 2,280 mg, 2,285 mg, 2,290 mg, 2,295 mg, 2,300 mg, 2,305 mg, 2,310 mg, 2,315 mg, 2,320 mg, 2,325 mg, 2,330 mg, 2,335 mg, 2,340 mg, 2,345 mg, 2,350 mg, 2,355 mg, 2,360 mg, 2,365 mg, 2,370 mg, 2,375 mg, 2,380 mg, 2,385 mg, 2,390 mg, 2,395 mg, 2,400 mg, 2,405 mg, 2,410 mg, 2,415 mg, 2,420 mg, 2,425 mg, 2,430 mg, 2,435 mg, 2,440 mg, 2,445 mg, 2,450 mg, 2,455 mg, 2,460 mg, 2,465 mg, 2,470 mg, 2,475 mg, 2,480 mg, 2,485 mg, 2,490 mg, 2,495 mg, or 2,500 mg per dose.

In a further aspect, the disclosure features a method of preventing labor prior to cesarean delivery in a pregnant subject, such as a pregnant human subject, by administering to the subject therapeutically effective amounts of atosiban and a PGF2α receptor antagonist, such as a compound represented by formula (I)

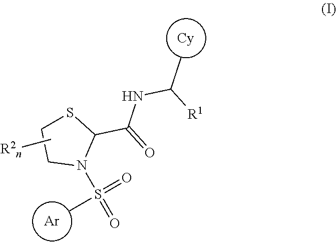

wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof.

In accordance with the foregoing aspect, the compound may be administered to the subject in an amount of from about 250 mg to about 2,500 mg per dose.

For example, the compound may be administered to the subject in an amount of from about 250 mg to about 750 mg per dose, such as an amount of from about 255 mg to about 745 mg, from about 260 mg to about 740 mg, from about 265 mg to about 735 mg, from about 270 mg to about 730 mg, from about 275 mg to about 725 mg, from about 280 mg to about 720 mg, from about 285 mg to about 715 mg, from about 290 mg to about 710 mg, from about 295 mg to about 705 mg, from about 300 mg to about 700 mg, from about 305 mg to about 695 mg, from about 310 mg to about 690 mg, from about 315 mg to about 685 mg, from about 320 mg to about 680 mg, from about 325 mg to about 675 mg, from about 330 mg to about 670 mg, from about 335 mg to about 665 mg, from about 340 mg to about 660 mg, from about 345 mg to about 655 mg, from about 350 mg to about 650 mg, from about 355 mg to about 645 mg, from about 360 mg to about 640 mg, from about 365 mg to about 635 mg, from about 370 mg to about 630 mg, from about 375 mg to about 625 mg, from about 380 mg to about 620 mg, from about 385 mg to about 615 mg, from about 390 mg to about 610 mg, from about 395 mg to about 605 mg, from about 400 mg to about 600 mg, from about 405 mg to about 595 mg, from about 410 mg to about 590 mg, from about 415 mg to about 585 mg, from about 420 mg to about 580 mg, from about 425 mg to about 575 mg, from about 430 mg to about 570 mg, from about 435 mg to about 565 mg, from about 440 mg to about 560 mg, from about 445 mg to about 555 mg, from about 450 mg to about 550 mg, from about 455 mg to about 545 mg, from about 460 mg to about 540 mg, from about 465 mg to about 535 mg, from about 470 mg to about 530 mg, from about 475 mg to about 525 mg, from about 480 mg to about 520 mg, from about 485 mg to about 515 mg, from about 490 mg to about 510 mg, or from about 495 mg to about 505 mg per dose. In some embodiments, the compound is administered to the subject in an amount of about 500 mg per dose.

In some embodiments, the compound is administered to the subject in an amount of from about 250 mg to about 2,000 mg per dose, such as an amount of from about 255 mg to about 1,990 mg, from about 260 mg to about 1,980 mg, from about 265 mg to about 1,970 mg, from about 270 mg to about 1,960 mg, from about 275 mg to about 1,950 mg, from about 280 mg to about 1,940 mg, from about 285 mg to about 1,930 mg, from about 290 mg to about 1,920 mg, from about 295 mg to about 1,910 mg, from about 300 mg to about 1,900 mg, from about 305 mg to about 1,890 mg, from about 310 mg to about 1,880 mg, from about 315 mg to about 1,870 mg, from about 320 mg to about 1,860 mg, from about 325 mg to about 1,850 mg, from about 330 mg to about 1,840 mg, from about 335 mg to about 1,830 mg, from about 340 mg to about 1,820 mg, from about 345 mg to about 1,810 mg, from about 350 mg to about 1,800 mg, from about 355 mg to about 1,790 mg, from about 360 mg to about 1,780 mg, from about 365 mg to about 1,770 mg, from about 370 mg to about 1,760 mg, from about 375 mg to about 1,750 mg, from about 380 mg to about 1,740 mg, from about 385 mg to about 1,730 mg, from about 390 mg to about 1,720 mg, from about 395 mg to about 1,710 mg, from about 400 mg to about 1,700 mg, from about 405 mg to about 1,690 mg, from about 410 mg to about 1,680 mg, from about 415 mg to about 1,670 mg, from about 420 mg to about 1,660 mg, from about 425 mg to about 1,650 mg, from about 430 mg to about 1,640 mg, from about 435 mg to about 1,630 mg, from about 440 mg to about 1,620 mg, from about 445 mg to about 1,610 mg, from about 450 mg to about 1,600 mg, from about 455 mg to about 1,590 mg, from about 460 mg to about 1,580 mg, from about 465 mg to about 1,570 mg, from about 470 mg to about 1,560 mg, from about 475 mg to about 1,550 mg, from about 480 mg to about 1,540 mg, from about 485 mg to about 1,530 mg, from about 490 mg to about 1,520 mg, from about 495 mg to about 1,510 mg, from about 500 mg to about 1,500 mg, from about 505 mg to about 1,495 mg, from about 510 mg to about 1,490 mg, from about 515 mg to about 1,485 mg, from about 520 mg to about 1,480 mg, from about 525 mg to about 1,475 mg, from about 530 mg to about 1,470 mg, from about 535 mg to about 1,465 mg, from about 540 mg to about 1,460 mg, from about 545 mg to about 1,455 mg, from about 550 mg to about 1,450 mg, from about 555 mg to about 1,445 mg, from about 560 mg to about 1,440 mg, from about 565 mg to about 1,435 mg, from about 570 mg to about 1,430 mg, from about 575 mg to about 1,425 mg, from about 580 mg to about 1,420 mg, from about 585 mg to about 1,415 mg, from about 590 mg to about 1,410 mg, from about 595 mg t about 1,405 mg, from about 600 mg to about 1,400 mg, from about 605 mg to about 1,395 mg, from about 610 mg to about 1,390 mg, from about 615 mg to about 1,385 mg, from about 620 mg to about 1,380 mg, from about 625 mg to about 1,375 mg, from about 630 mg to about 1,370 mg, from about 635 mg to about 1,365 mg, from about 640 mg to about 1,360 mg, from about 645 mg to about 1,355 mg, from about 650 mg to about 1,350 mg, from about 655 mg to about 1,345 mg, from about 660 mg to about 1,340 mg, from about 665 mg to about 1,335 mg, from about 670 mg to about 1,330 mg, from about 675 mg to about 1,325 mg, from about 680 mg to about 1,320 mg, from about 685 mg to about 1,315 mg, from about 690 mg to about 1,310 mg, from about 695 mg to about 1,305 mg, from about 700 mg to about 1,300 mg, from about 705 mg to about 1,295 mg, from about 710 mg to about 1,290 mg, from about 715 mg to about 1,285 mg, from about 720 mg to about 1,280 mg, from about 725 mg to about 1,275 mg, from about 730 mg to about 1,270 mg, from about 735 mg to about 1,265 mg, from about 740 mg to about 1,260 mg, from about 745 mg to about 1,255 mg, from about 750 mg to about 1,250 mg, from about 755 mg to about 1,245 mg, from about 760 mg to about 1,240 mg, from about 765 mg to about 1,235 mg, from about 770 mg to about 1,230 mg, from about 775 mg to about 1,225 mg, from about 780 mg to about 1,220 mg, from about 785 mg to about 1,215 mg, from about 790 mg to about 1,210 mg, from about 795 mg to about 1,205 mg, from about 800 mg to about 1,200 mg, from about 805 mg to about 1,195 mg, from about 810 mg to about 1,190 mg, from about 815 mg to about 1,185 mg, from about 820 mg to about 1,180 mg, from about 825 mg to about 1,175 mg, from about 830 mg to about 1,170 mg, from about 835 mg to about 1,165 mg, from about 840 mg to about 1,160 mg, from about 845 mg to about 1,155 mg, from about 850 mg to about 1,150 mg, from about 855 mg to about 1,145 mg, from about 860 mg to about 1,140 mg, from about 865 mg to about 1,135 mg, from about 870 mg to about 1,130 mg, from about 875 mg to about 1,125 mg, from about 880 mg to about 1,120 mg, from about 885 mg to about 1,115 mg, from about 890 mg to about 1,110 mg, from about 895 mg to about 1,105 mg, from about 900 mg to about 1,000 mg, from about 905 mg to about 1,095 mg, from about 910 mg to about 1,090 mg, from about 915 mg to about 1,085 mg, from about 920 mg to about 1,080 mg, from about 925 mg to about 1,075 mg, from about 930 mg to about 1,070 mg, from about 935 mg to about 1,065 mg, from about 940 mg to about 1,060 mg, from about 945 mg to about 1,055 mg, from about 950 mg to about 1,050 mg, from about 955 mg to about 1,045 mg, from about 960 mg to about 1,040 mg, from about 965 mg to about 1,035 mg, from about 970 mg to about 1,030 mg, from about 975 mg to about 1,025 mg, from about 980 mg to about 1,020 mg, from about 985 mg to about 1,015 mg, from about 990 mg to about 1,010 mg, or from about 995 mg to about 1,005 mg per dose. In some embodiments, the compound is administered to the subject in an amount of about 1,000 mg per dose.

In some embodiments, the compound is administered to the subject in an amount of from about 1,500 mg to about 2,500 mg per dose, such as an amount of from about 1,505 mg to about 2,495 mg, from about 1,510 mg to about 2,490 mg, from about 1,515 mg to about 2,485 mg, from about 1,520 mg to about 2,480 mg, from about 1,525 mg to about 2,475 mg, from about 1,530 mg to about 2,470 mg, from about 1,535 mg to about 2,465 mg, from about 1,540 mg to about 2,460 mg, from about 1,545 mg to about 2,455 mg, from about 1,550 mg to about 2,450 mg, from about 1,555 mg to about 2,445 mg, from about 1,560 mg to about 2,440 mg, from about 1,565 mg to about 2,435 mg, from about 1,570 mg to about 2,430 mg, from about 1,575 mg to about 2,425 mg, from about 1,580 mg to about 2,420 mg, from about 1,585 mg to about 2,415 mg, from about 1,590 mg to about 2,410 mg, from about 1,595 mg to about 2,405 mg, from about 1,600 mg to about 2,400 mg, from about 1,605 mg to about 2,395 mg, from about 1,610 mg to about 2,390 mg, from about 1,615 mg to about 2,385 mg, from about 1,620 mg to about 2,380 mg, from about 1,625 mg to about 2,375 mg, from about 1,630 mg to about 2,370 mg, from about 1,635 mg to about 2,365 mg, from about 1,640 mg to about 2,360 mg, from about 1,645 mg to about 2,355 mg, from about 1,650 mg to about 2,350 mg, from about 1,655 mg to about 2,345 mg, from about 1,660 mg to about 2,340 mg, from about 1,665 mg to about 2,335 mg, from about 1,670 mg to about 2,330 mg, from about 1,675 mg to about 2,325 mg, from about 1,680 mg to about 2,320 mg, from about 1,685 mg to about 2,315 mg, from about 1,690 mg to about 2,310 mg, from about 1,695 mg to about 2,305 mg, from about 1,700 mg to about 2,300 mg, from about 1,705 mg to about 2,295 mg, from about 1,710 mg to about 2,290 mg, from about 1,715 mg to about 2,285 mg, from about 1,720 mg to about 2,280 mg, from about 1,725 mg to about 2,275 mg, from about 1,730 mg to about 2,270 mg, from about 1,735 mg to about 2,265 mg, from about 1,740 mg to about 2,260 mg, from about 1,745 mg to about 2,255 mg, from about 1,750 mg to about 2,250 mg, from about 1,755 mg to about 2,245 mg, from about 1,760 mg to about 2,240 mg, from about 1,765 mg to about 2,235 mg, from about 1,770 mg to about 2,230 mg, from about 1,775 mg to about 2,225 mg, from about 1,780 mg to about 2,220 mg, from about 1,785 mg to about 2,215 mg, from about 1,790 mg to about 2,210 mg, from about 1,795 mg to about 2,205 mg, from about 1,800 mg to about 2,200 mg, from about 1,805 mg to about 2,195 mg, from about 1,810 mg to about 2,190 mg, from about 1,815 mg to about 2,185 mg, from about 1,820 mg to about 2,180 mg, from about 1,825 mg to about 2,175 mg, from about 1,830 mg to about 2,170 mg, from about 1,835 mg to about 2,165 mg, from about 1,840 mg to about 2,160 mg, from about 1,845 mg to about 2,155 mg, from about 1,850 mg to about 2,150 mg, from about 1,855 mg to about 2,145 mg, from about 1,860 mg to about 2,140 mg, from about 1,865 mg to about 2,135 mg, from about 1,870 mg to about 2,130 mg, from about 1,875 mg to about 2,125 mg, from about 1,880 mg to about 2,120 mg, from about 1,885 mg to about 2,115 mg, from about 1,890 mg to about 2,110 mg, from about 1,895 mg to about 2,105 mg, from about 1,900 mg to about 2,100 mg, from about 1,905 mg to about 2,095 mg, from about 1,910 mg to about 2,090 mg, from about 1,915 mg to about 2,085 mg, from about 1,920 mg to about 2,080 mg, from about 1,925 mg to about 2,075 mg, from about 1,930 mg to about 2,070 mg, from about 1,935 mg to about 2,065 mg, from about 1,940 mg to about 2,060 mg, from about 1,945 mg to about 2,055 mg, from about 1,950 mg to about 2,050 mg, from about 1,955 mg to about 2,045 mg, from about 1,960 mg to about 2,040 mg, from about 1,965 mg to about 2,035 mg, from about 1,970 mg to about 2,030 mg, from about 1,975 mg to about 2,025 mg, from about 1,980 mg to about 2,020 mg, from about 1,985 mg to about 2,015 mg, from about 1,990 mg to about 2,010 mg, or from about 1,995 mg to about 2,005 mg per dose. In some embodiments, the compound is administered to the subject in an amount of about 2,000 mg per dose.

For example, in some embodiments, the compound is administered to the subject in an amount of about 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, 1,150 mg, 1,155 mg, 1,160 mg, 1,165 mg, 1,170 mg, 1,175 mg, 1,180 mg, 1,185 mg, 1,190 mg, 1,195 mg, 1,200 mg, 1,205 mg, 1,210 mg, 1,215 mg, 1,220 mg, 1,225 mg, 1,230 mg, 1,235 mg, 1,240 mg, 1,245 mg, 1,250 mg, 1,255 mg, 1,260 mg, 1,265 mg, 1,270 mg, 1,275 mg, 1,280 mg, 1,285 mg, 1,290 mg, 1,295 mg, 1,300 mg, 1,305 mg, 1,310 mg, 1,315 mg, 1,320 mg, 1,325 mg, 1,330 mg, 1,335 mg, 1,340 mg, 1,345 mg, 1,350 mg, 1,355 mg, 1,360 mg, 1,365 mg, 1,370 mg, 1,375 mg, 1,380 mg, 1,385 mg, 1,390 mg, 1,395 mg, 1,400 mg, 1,405 mg, 1,410 mg, 1,415 mg, 1,420 mg, 1,425 mg, 1,430 mg, 1,435 mg, 1,440 mg, 1,445 mg, 1,450 mg, 1,455 mg, 1,460 mg, 1,465 mg, 1,470 mg, 1,475 mg, 1,480 mg, 1,485 mg, 1,490 mg, 1,495 mg, 1,500 mg, 1,505 mg, 1,510 mg, 1,515 mg, 1,520 mg, 1,525 mg, 1,530 mg, 1,535 mg, 1,540 mg, 1,545 mg, 1,550 mg, 1,555 mg, 1,560 mg, 1,565 mg, 1,570 mg, 1,575 mg, 1,580 mg, 1,585 mg, 1,590 mg, 1,595 mg, 1,600 mg, 1,605 mg, 1,610 mg, 1,615 mg, 1,620 mg, 1,625 mg, 1,630 mg, 1,635 mg, 1,640 mg, 1,645 mg, 1,650 mg, 1,655 mg, 1,660 mg, 1,665 mg, 1,670 mg, 1,675 mg, 1,680 mg, 1,685 mg, 1,690 mg, 1,695 mg, 1,700 mg, 1,705 mg, 1,710 mg, 1,715 mg, 1,720 mg, 1,725 mg, 1,730 mg, 1,735 mg, 1,740 mg, 1,745 mg, 1,750 mg, 1,755 mg, 1,760 mg, 1,765 mg, 1,770 mg, 1,775 mg, 1,780 mg, 1,785 mg, 1,790 mg, 1,795 mg, 1,800 mg, 1,805 mg, 1,810 mg, 1,815 mg, 1,820 mg, 1,825 mg, 1,830 mg, 1,835 mg, 1,840 mg, 1,845 mg, 1,850 mg, 1,855 mg, 1,860 mg, 1,865 mg, 1,870 mg, 1,875 mg, 1,880 mg, 1,885 mg, 1,890 mg, 1,895 mg, 1,900 mg, 1,905 mg, 1,910 mg, 1,915 mg, 1,920 mg, 1,925 mg, 1,930 mg, 1,935 mg, 1,940 mg, 1,945 mg, 1,950 mg, 1,955 mg, 1,960 mg, 1,965 mg, 1,970 mg, 1,975 mg, 1,980 mg, 1,985 mg, 1,990 mg, 1,995 mg, 2,000 mg, 2,005 mg, 2,010 mg, 2,015 mg, 2,020 mg, 2,025 mg, 2,030 mg, 2,035 mg, 2,040 mg, 2,045 mg, 2,050 mg, 2,055 mg, 2,060 mg, 2,065 mg, 2,070 mg, 2,075 mg, 2,080 mg, 2,085 mg, 2,090 mg, 2,095 mg, 2,100 mg, 2,105 mg, 2,110 mg, 2,115 mg, 2,120 mg, 2,125 mg, 2,130 mg, 2,135 mg, 2,140 mg, 2,145 mg, 2,150 mg, 2,155 mg, 2,160 mg, 2,165 mg, 2,170 mg, 2,175 mg, 2,180 mg, 2,185 mg, 2,190 mg, 2,195 mg, 2,200 mg, 2,205 mg, 2,210 mg, 2,215 mg, 2,220 mg, 2,225 mg, 2,230 mg, 2,235 mg, 2,240 mg, 2,245 mg, 2,250 mg, 2,255 mg, 2,260 mg, 2,265 mg, 2,270 mg, 2,275 mg, 2,280 mg, 2,285 mg, 2,290 mg, 2,295 mg, 2,300 mg, 2,305 mg, 2,310 mg, 2,315 mg, 2,320 mg, 2,325 mg, 2,330 mg, 2,335 mg, 2,340 mg, 2,345 mg, 2,350 mg, 2,355 mg, 2,360 mg, 2,365 mg, 2,370 mg, 2,375 mg, 2,380 mg, 2,385 mg, 2,390 mg, 2,395 mg, 2,400 mg, 2,405 mg, 2,410 mg, 2,415 mg, 2,420 mg, 2,425 mg, 2,430 mg, 2,435 mg, 2,440 mg, 2,445 mg, 2,450 mg, 2,455 mg, 2,460 mg, 2,465 mg, 2,470 mg, 2,475 mg, 2,480 mg, 2,485 mg, 2,490 mg, 2,495 mg, or 2,500 mg per dose.

In an additional aspect, the disclosure features a method of reducing the frequency of, peak amplitude of, duration of, and/or work done by, uterine contractions in a pregnant subject, such as a pregnant human subject, by administering to the subject therapeutically effective amounts of atosiban and a PGF2α receptor antagonist, such as a compound represented by formula (I)

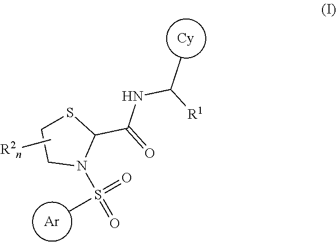

wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof.

In accordance with the foregoing aspect, the compound may be administered to the subject in an amount of from about 250 mg to about 2,500 mg per dose.

For example, the compound may be administered to the subject in an amount of from about 250 mg to about 750 mg per dose, such as an amount of from about 255 mg to about 745 mg, from about 260 mg to about 740 mg, from about 265 mg to about 735 mg, from about 270 mg to about 730 mg, from about 275 mg to about 725 mg, from about 280 mg to about 720 mg, from about 285 mg to about 715 mg, from about 290 mg to about 710 mg, from about 295 mg to about 705 mg, from about 300 mg to about 700 mg, from about 305 mg to about 695 mg, from about 310 mg to about 690 mg, from about 315 mg to about 685 mg, from about 320 mg to about 680 mg, from about 325 mg to about 675 mg, from about 330 mg to about 670 mg, from about 335 mg to about 665 mg, from about 340 mg to about 660 mg, from about 345 mg to about 655 mg, from about 350 mg to about 650 mg, from about 355 mg to about 645 mg, from about 360 mg to about 640 mg, from about 365 mg to about 635 mg, from about 370 mg to about 630 mg, from about 375 mg to about 625 mg, from about 380 mg to about 620 mg, from about 385 mg to about 615 mg, from about 390 mg to about 610 mg, from about 395 mg to about 605 mg, from about 400 mg to about 600 mg, from about 405 mg to about 595 mg, from about 410 mg to about 590 mg, from about 415 mg to about 585 mg, from about 420 mg to about 580 mg, from about 425 mg to about 575 mg, from about 430 mg to about 570 mg, from about 435 mg to about 565 mg, from about 440 mg to about 560 mg, from about 445 mg to about 555 mg, from about 450 mg to about 550 mg, from about 455 mg to about 545 mg, from about 460 mg to about 540 mg, from about 465 mg to about 535 mg, from about 470 mg to about 530 mg, from about 475 mg to about 525 mg, from about 480 mg to about 520 mg, from about 485 mg to about 515 mg, from about 490 mg to about 510 mg, or from about 495 mg to about 505 mg per dose. In some embodiments, the compound is administered to the subject in an amount of about 500 mg per dose.

In some embodiments, the compound is administered to the subject in an amount of from about 250 mg to about 2,000 mg per dose, such as an amount of from about 255 mg to about 1,990 mg, from about 260 mg to about 1,980 mg, from about 265 mg to about 1,970 mg, from about 270 mg to about 1,960 mg, from about 275 mg to about 1,950 mg, from about 280 mg to about 1,940 mg, from about 285 mg to about 1,930 mg, from about 290 mg to about 1,920 mg, from about 295 mg to about 1,910 mg, from about 300 mg to about 1,900 mg, from about 305 mg to about 1,890 mg, from about 310 mg to about 1,880 mg, from about 315 mg to about 1,870 mg, from about 320 mg to about 1,860 mg, from about 325 mg to about 1,850 mg, from about 330 mg to about 1,840 mg, from about 335 mg to about 1,830 mg, from about 340 mg to about 1,820 mg, from about 345 mg to about 1,810 mg, from about 350 mg to about 1,800 mg, from about 355 mg to about 1,790 mg, from about 360 mg to about 1,780 mg, from about 365 mg to about 1,770 mg, from about 370 mg to about 1,760 mg, from about 375 mg to about 1,750 mg, from about 380 mg to about 1,740 mg, from about 385 mg to about 1,730 mg, from about 390 mg to about 1,720 mg, from about 395 mg to about 1,710 mg, from about 400 mg to about 1,700 mg, from about 405 mg to about 1,690 mg, from about 410 mg to about 1,680 mg, from about 415 mg to about 1,670 mg, from about 420 mg to about 1,660 mg, from about 425 mg to about 1,650 mg, from about 430 mg to about 1,640 mg, from about 435 mg to about 1,630 mg, from about 440 mg to about 1,620 mg, from about 445 mg to about 1,610 mg, from about 450 mg to about 1,600 mg, from about 455 mg to about 1,590 mg, from about 460 mg to about 1,580 mg, from about 465 mg to about 1,570 mg, from about 470 mg to about 1,560 mg, from about 475 mg to about 1,550 mg, from about 480 mg to about 1,540 mg, from about 485 mg to about 1,530 mg, from about 490 mg to about 1,520 mg, from about 495 mg to about 1,510 mg, from about 500 mg to about 1,500 mg, from about 505 mg to about 1,495 mg, from about 510 mg to about 1,490 mg, from about 515 mg to about 1,485 mg, from about 520 mg to about 1,480 mg, from about 525 mg to about 1,475 mg, from about 530 mg to about 1,470 mg, from about 535 mg to about 1,465 mg, from about 540 mg to about 1,460 mg, from about 545 mg to about 1,455 mg, from about 550 mg to about 1,450 mg, from about 555 mg to about 1,445 mg, from about 560 mg to about 1,440 mg, from about 565 mg to about 1,435 mg, from about 570 mg to about 1,430 mg, from about 575 mg to about 1,425 mg, from about 580 mg to about 1,420 mg, from about 585 mg to about 1,415 mg, from about 590 mg to about 1,410 mg, from about 595 mg t about 1,405 mg, from about 600 mg to about 1,400 mg, from about 605 mg to about 1,395 mg, from about 610 mg to about 1,390 mg, from about 615 mg to about 1,385 mg, from about 620 mg to about 1,380 mg, from about 625 mg to about 1,375 mg, from about 630 mg to about 1,370 mg, from about 635 mg to about 1,365 mg, from about 640 mg to about 1,360 mg, from about 645 mg to about 1,355 mg, from about 650 mg to about 1,350 mg, from about 655 mg to about 1,345 mg, from about 660 mg to about 1,340 mg, from about 665 mg to about 1,335 mg, from about 670 mg to about 1,330 mg, from about 675 mg to about 1,325 mg, from about 680 mg to about 1,320 mg, from about 685 mg to about 1,315 mg, from about 690 mg to about 1,310 mg, from about 695 mg to about 1,305 mg, from about 700 mg to about 1,300 mg, from about 705 mg to about 1,295 mg, from about 710 mg to about 1,290 mg, from about 715 mg to about 1,285 mg, from about 720 mg to about 1,280 mg, from about 725 mg to about 1,275 mg, from about 730 mg to about 1,270 mg, from about 735 mg to about 1,265 mg, from about 740 mg to about 1,260 mg, from about 745 mg to about 1,255 mg, from about 750 mg to about 1,250 mg, from about 755 mg to about 1,245 mg, from about 760 mg to about 1,240 mg, from about 765 mg to about 1,235 mg, from about 770 mg to about 1,230 mg, from about 775 mg to about 1,225 mg, from about 780 mg to about 1,220 mg, from about 785 mg to about 1,215 mg, from about 790 mg to about 1,210 mg, from about 795 mg to about 1,205 mg, from about 800 mg to about 1,200 mg, from about 805 mg to about 1,195 mg, from about 810 mg to about 1,190 mg, from about 815 mg to about 1,185 mg, from about 820 mg to about 1,180 mg, from about 825 mg to about 1,175 mg, from about 830 mg to about 1,170 mg, from about 835 mg to about 1,165 mg, from about 840 mg to about 1,160 mg, from about 845 mg to about 1,155 mg, from about 850 mg to about 1,150 mg, from about 855 mg to about 1,145 mg, from about 860 mg to about 1,140 mg, from about 865 mg to about 1,135 mg, from about 870 mg to about 1,130 mg, from about 875 mg to about 1,125 mg, from about 880 mg to about 1,120 mg, from about 885 mg to about 1,115 mg, from about 890 mg to about 1,110 mg, from about 895 mg to about 1,105 mg, from about 900 mg to about 1,000 mg, from about 905 mg to about 1,095 mg, from about 910 mg to about 1,090 mg, from about 915 mg to about 1,085 mg, from about 920 mg to about 1,080 mg, from about 925 mg to about 1,075 mg, from about 930 mg to about 1,070 mg, from about 935 mg to about 1,065 mg, from about 940 mg to about 1,060 mg, from about 945 mg to about 1,055 mg, from about 950 mg to about 1,050 mg, from about 955 mg to about 1,045 mg, from about 960 mg to about 1,040 mg, from about 965 mg to about 1,035 mg, from about 970 mg to about 1,030 mg, from about 975 mg to about 1,025 mg, from about 980 mg to about 1,020 mg, from about 985 mg to about 1,015 mg, from about 990 mg to about 1,010 mg, or from about 995 mg to about 1,005 mg per dose. In some embodiments, the compound is administered to the subject in an amount of about 1,000 mg per dose.

In some embodiments, the compound is administered to the subject in an amount of from about 1,500 mg to about 2,500 mg per dose, such as an amount of from about 1,505 mg to about 2,495 mg, from about 1,510 mg to about 2,490 mg, from about 1,515 mg to about 2,485 mg, from about 1,520 mg to about 2,480 mg, from about 1,525 mg to about 2,475 mg, from about 1,530 mg to about 2,470 mg, from about 1,535 mg to about 2,465 mg, from about 1,540 mg to about 2,460 mg, from about 1,545 mg to about 2,455 mg, from about 1,550 mg to about 2,450 mg, from about 1,555 mg to about 2,445 mg, from about 1,560 mg to about 2,440 mg, from about 1,565 mg to about 2,435 mg, from about 1,570 mg to about 2,430 mg, from about 1,575 mg to about 2,425 mg, from about 1,580 mg to about 2,420 mg, from about 1,585 mg to about 2,415 mg, from about 1,590 mg to about 2,410 mg, from about 1,595 mg to about 2,405 mg, from about 1,600 mg to about 2,400 mg, from about 1,605 mg to about 2,395 mg, from about 1,610 mg to about 2,390 mg, from about 1,615 mg to about 2,385 mg, from about 1,620 mg to about 2,380 mg, from about 1,625 mg to about 2,375 mg, from about 1,630 mg to about 2,370 mg, from about 1,635 mg to about 2,365 mg, from about 1,640 mg to about 2,360 mg, from about 1,645 mg to about 2,355 mg, from about 1,650 mg to about 2,350 mg, from about 1,655 mg to about 2,345 mg, from about 1,660 mg to about 2,340 mg, from about 1,665 mg to about 2,335 mg, from about 1,670 mg to about 2,330 mg, from about 1,675 mg to about 2,325 mg, from about 1,680 mg to about 2,320 mg, from about 1,685 mg to about 2,315 mg, from about 1,690 mg to about 2,310 mg, from about 1,695 mg to about 2,305 mg, from about 1,700 mg to about 2,300 mg, from about 1,705 mg to about 2,295 mg, from about 1,710 mg to about 2,290 mg, from about 1,715 mg to about 2,285 mg, from about 1,720 mg to about 2,280 mg, from about 1,725 mg to about 2,275 mg, from about 1,730 mg to about 2,270 mg, from about 1,735 mg to about 2,265 mg, from about 1,740 mg to about 2,260 mg, from about 1,745 mg to about 2,255 mg, from about 1,750 mg to about 2,250 mg, from about 1,755 mg to about 2,245 mg, from about 1,760 mg to about 2,240 mg, from about 1,765 mg to about 2,235 mg, from about 1,770 mg to about 2,230 mg, from about 1,775 mg to about 2,225 mg, from about 1,780 mg to about 2,220 mg, from about 1,785 mg to about 2,215 mg, from about 1,790 mg to about 2,210 mg, from about 1,795 mg to about 2,205 mg, from about 1,800 mg to about 2,200 mg, from about 1,805 mg to about 2,195 mg, from about 1,810 mg to about 2,190 mg, from about 1,815 mg to about 2,185 mg, from about 1,820 mg to about 2,180 mg, from about 1,825 mg to about 2,175 mg, from about 1,830 mg to about 2,170 mg, from about 1,835 mg to about 2,165 mg, from about 1,840 mg to about 2,160 mg, from about 1,845 mg to about 2,155 mg, from about 1,850 mg to about 2,150 mg, from about 1,855 mg to about 2,145 mg, from about 1,860 mg to about 2,140 mg, from about 1,865 mg to about 2,135 mg, from about 1,870 mg to about 2,130 mg, from about 1,875 mg to about 2,125 mg, from about 1,880 mg to about 2,120 mg, from about 1,885 mg to about 2,115 mg, from about 1,890 mg to about 2,110 mg, from about 1,895 mg to about 2,105 mg, from about 1,900 mg to about 2,100 mg, from about 1,905 mg to about 2,095 mg, from about 1,910 mg to about 2,090 mg, from about 1,915 mg to about 2,085 mg, from about 1,920 mg to about 2,080 mg, from about 1,925 mg to about 2,075 mg, from about 1,930 mg to about 2,070 mg, from about 1,935 mg to about 2,065 mg, from about 1,940 mg to about 2,060 mg, from about 1,945 mg to about 2,055 mg, from about 1,950 mg to about 2,050 mg, from about 1,955 mg to about 2,045 mg, from about 1,960 mg to about 2,040 mg, from about 1,965 mg to about 2,035 mg, from about 1,970 mg to about 2,030 mg, from about 1,975 mg to about 2,025 mg, from about 1,980 mg to about 2,020 mg, from about 1,985 mg to about 2,015 mg, from about 1,990 mg to about 2,010 mg, or from about 1,995 mg to about 2,005 mg per dose. In some embodiments, the compound is administered to the subject in an amount of about 2,000 mg per dose.

For example, in some embodiments, the compound is administered to the subject in an amount of about 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, 1,150 mg, 1,155 mg, 1,160 mg, 1,165 mg, 1,170 mg, 1,175 mg, 1,180 mg, 1,185 mg, 1,190 mg, 1,195 mg, 1,200 mg, 1,205 mg, 1,210 mg, 1,215 mg, 1,220 mg, 1,225 mg, 1,230 mg, 1,235 mg, 1,240 mg, 1,245 mg, 1,250 mg, 1,255 mg, 1,260 mg, 1,265 mg, 1,270 mg, 1,275 mg, 1,280 mg, 1,285 mg, 1,290 mg, 1,295 mg, 1,300 mg, 1,305 mg, 1,310 mg, 1,315 mg, 1,320 mg, 1,325 mg, 1,330 mg, 1,335 mg, 1,340 mg, 1,345 mg, 1,350 mg, 1,355 mg, 1,360 mg, 1,365 mg, 1,370 mg, 1,375 mg, 1,380 mg, 1,385 mg, 1,390 mg, 1,395 mg, 1,400 mg, 1,405 mg, 1,410 mg, 1,415 mg, 1,420 mg, 1,425 mg, 1,430 mg, 1,435 mg, 1,440 mg, 1,445 mg, 1,450 mg, 1,455 mg, 1,460 mg, 1,465 mg, 1,470 mg, 1,475 mg, 1,480 mg, 1,485 mg, 1,490 mg, 1,495 mg, 1,500 mg, 1,505 mg, 1,510 mg, 1,515 mg, 1,520 mg, 1,525 mg, 1,530 mg, 1,535 mg, 1,540 mg, 1,545 mg, 1,550 mg, 1,555 mg, 1,560 mg, 1,565 mg, 1,570 mg, 1,575 mg, 1,580 mg, 1,585 mg, 1,590 mg, 1,595 mg, 1,600 mg, 1,605 mg, 1,610 mg, 1,615 mg, 1,620 mg, 1,625 mg, 1,630 mg, 1,635 mg, 1,640 mg, 1,645 mg, 1,650 mg, 1,655 mg, 1,660 mg, 1,665 mg, 1,670 mg, 1,675 mg, 1,680 mg, 1,685 mg, 1,690 mg, 1,695 mg, 1,700 mg, 1,705 mg, 1,710 mg, 1,715 mg, 1,720 mg, 1,725 mg, 1,730 mg, 1,735 mg, 1,740 mg, 1,745 mg, 1,750 mg, 1,755 mg, 1,760 mg, 1,765 mg, 1,770 mg, 1,775 mg, 1,780 mg, 1,785 mg, 1,790 mg, 1,795 mg, 1,800 mg, 1,805 mg, 1,810 mg, 1,815 mg, 1,820 mg, 1,825 mg, 1,830 mg, 1,835 mg, 1,840 mg, 1,845 mg, 1,850 mg, 1,855 mg, 1,860 mg, 1,865 mg, 1,870 mg, 1,875 mg, 1,880 mg, 1,885 mg, 1,890 mg, 1,895 mg, 1,900 mg, 1,905 mg, 1,910 mg, 1,915 mg, 1,920 mg, 1,925 mg, 1,930 mg, 1,935 mg, 1,940 mg, 1,945 mg, 1,950 mg, 1,955 mg, 1,960 mg, 1,965 mg, 1,970 mg, 1,975 mg, 1,980 mg, 1,985 mg, 1,990 mg, 1,995 mg, 2,000 mg, 2,005 mg, 2,010 mg, 2,015 mg, 2,020 mg, 2,025 mg, 2,030 mg, 2,035 mg, 2,040 mg, 2,045 mg, 2,050 mg, 2,055 mg, 2,060 mg, 2,065 mg, 2,070 mg, 2,075 mg, 2,080 mg, 2,085 mg, 2,090 mg, 2,095 mg, 2,100 mg, 2,105 mg, 2,110 mg, 2,115 mg, 2,120 mg, 2,125 mg, 2,130 mg, 2,135 mg, 2,140 mg, 2,145 mg, 2,150 mg, 2,155 mg, 2,160 mg, 2,165 mg, 2,170 mg, 2,175 mg, 2,180 mg, 2,185 mg, 2,190 mg, 2,195 mg, 2,200 mg, 2,205 mg, 2,210 mg, 2,215 mg, 2,220 mg, 2,225 mg, 2,230 mg, 2,235 mg, 2,240 mg, 2,245 mg, 2,250 mg, 2,255 mg, 2,260 mg, 2,265 mg, 2,270 mg, 2,275 mg, 2,280 mg, 2,285 mg, 2,290 mg, 2,295 mg, 2,300 mg, 2,305 mg, 2,310 mg, 2,315 mg, 2,320 mg, 2,325 mg, 2,330 mg, 2,335 mg, 2,340 mg, 2,345 mg, 2,350 mg, 2,355 mg, 2,360 mg, 2,365 mg, 2,370 mg, 2,375 mg, 2,380 mg, 2,385 mg, 2,390 mg, 2,395 mg, 2,400 mg, 2,405 mg, 2,410 mg, 2,415 mg, 2,420 mg, 2,425 mg, 2,430 mg, 2,435 mg, 2,440 mg, 2,445 mg, 2,450 mg, 2,455 mg, 2,460 mg, 2,465 mg, 2,470 mg, 2,475 mg, 2,480 mg, 2,485 mg, 2,490 mg, 2,495 mg, or 2,500 mg per dose.

In yet another aspect, the disclosure features a method of reducing the expression of a proinflammatory and/or contractile gene, such as cyclooxygenase-2 (Cox2), in a pregnant subject, such as a pregnant human subject (e.g., in the myometrium of a pregnant human subject) by administering to the subject therapeutically effective amounts of atosiban and a PGF2α receptor antagonist, such as a compound represented by formula (I)

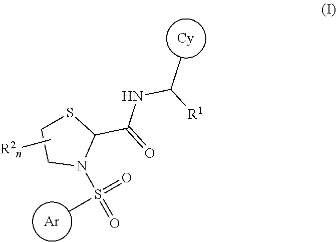

wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof.

In accordance with the foregoing aspect, the compound may be administered to the subject in an amount of from about 250 mg to about 2,500 mg per dose.

For example, the compound may be administered to the subject in an amount of from about 250 mg to about 750 mg per dose, such as an amount of from about 255 mg to about 745 mg, from about 260 mg to about 740 mg, from about 265 mg to about 735 mg, from about 270 mg to about 730 mg, from about 275 mg to about 725 mg, from about 280 mg to about 720 mg, from about 285 mg to about 715 mg, from about 290 mg to about 710 mg, from about 295 mg to about 705 mg, from about 300 mg to about 700 mg, from about 305 mg to about 695 mg, from about 310 mg to about 690 mg, from about 315 mg to about 685 mg, from about 320 mg to about 680 mg, from about 325 mg to about 675 mg, from about 330 mg to about 670 mg, from about 335 mg to about 665 mg, from about 340 mg to about 660 mg, from about 345 mg to about 655 mg, from about 350 mg to about 650 mg, from about 355 mg to about 645 mg, from about 360 mg to about 640 mg, from about 365 mg to about 635 mg, from about 370 mg to about 630 mg, from about 375 mg to about 625 mg, from about 380 mg to about 620 mg, from about 385 mg to about 615 mg, from about 390 mg to about 610 mg, from about 395 mg to about 605 mg, from about 400 mg to about 600 mg, from about 405 mg to about 595 mg, from about 410 mg to about 590 mg, from about 415 mg to about 585 mg, from about 420 mg to about 580 mg, from about 425 mg to about 575 mg, from about 430 mg to about 570 mg, from about 435 mg to about 565 mg, from about 440 mg to about 560 mg, from about 445 mg to about 555 mg, from about 450 mg to about 550 mg, from about 455 mg to about 545 mg, from about 460 mg to about 540 mg, from about 465 mg to about 535 mg, from about 470 mg to about 530 mg, from about 475 mg to about 525 mg, from about 480 mg to about 520 mg, from about 485 mg to about 515 mg, from about 490 mg to about 510 mg, or from about 495 mg to about 505 mg per dose. In some embodiments, the compound is administered to the subject in an amount of about 500 mg per dose.

In some embodiments, the compound is administered to the subject in an amount of from about 250 mg to about 2,000 mg per dose, such as an amount of from about 255 mg to about 1,990 mg, from about 260 mg to about 1,980 mg, from about 265 mg to about 1,970 mg, from about 270 mg to about 1,960 mg, from about 275 mg to about 1,950 mg, from about 280 mg to about 1,940 mg, from about 285 mg to about 1,930 mg, from about 290 mg to about 1,920 mg, from about 295 mg to about 1,910 mg, from about 300 mg to about 1,900 mg, from about 305 mg to about 1,890 mg, from about 310 mg to about 1,880 mg, from about 315 mg to about 1,870 mg, from about 320 mg to about 1,860 mg, from about 325 mg to about 1,850 mg, from about 330 mg to about 1,840 mg, from about 335 mg to about 1,830 mg, from about 340 mg to about 1,820 mg, from about 345 mg to about 1,810 mg, from about 350 mg to about 1,800 mg, from about 355 mg to about 1,790 mg, from about 360 mg to about 1,780 mg, from about 365 mg to about 1,770 mg, from about 370 mg to about 1,760 mg, from about 375 mg to about 1,750 mg, from about 380 mg to about 1,740 mg, from about 385 mg to about 1,730 mg, from about 390 mg to about 1,720 mg, from about 395 mg to about 1,710 mg, from about 400 mg to about 1,700 mg, from about 405 mg to about 1,690 mg, from about 410 mg to about 1,680 mg, from about 415 mg to about 1,670 mg, from about 420 mg to about 1,660 mg, from about 425 mg to about 1,650 mg, from about 430 mg to about 1,640 mg, from about 435 mg to about 1,630 mg, from about 440 mg to about 1,620 mg, from about 445 mg to about 1,610 mg, from about 450 mg to about 1,600 mg, from about 455 mg to about 1,590 mg, from about 460 mg to about 1,580 mg, from about 465 mg to about 1,570 mg, from about 470 mg to about 1,560 mg, from about 475 mg to about 1,550 mg, from about 480 mg to about 1,540 mg, from about 485 mg to about 1,530 mg, from about 490 mg to about 1,520 mg, from about 495 mg to about 1,510 mg, from about 500 mg to about 1,500 mg, from about 505 mg to about 1,495 mg, from about 510 mg to about 1,490 mg, from about 515 mg to about 1,485 mg, from about 520 mg to about 1,480 mg, from about 525 mg to about 1,475 mg, from about 530 mg to about 1,470 mg, from about 535 mg to about 1,465 mg, from about 540 mg to about 1,460 mg, from about 545 mg to about 1,455 mg, from about 550 mg to about 1,450 mg, from about 555 mg to about 1,445 mg, from about 560 mg to about 1,440 mg, from about 565 mg to about 1,435 mg, from about 570 mg to about 1,430 mg, from about 575 mg to about 1,425 mg, from about 580 mg to about 1,420 mg, from about 585 mg to about 1,415 mg, from about 590 mg to about 1,410 mg, from about 595 mg t about 1,405 mg, from about 600 mg to about 1,400 mg, from about 605 mg to about 1,395 mg, from about 610 mg to about 1,390 mg, from about 615 mg to about 1,385 mg, from about 620 mg to about 1,380 mg, from about 625 mg to about 1,375 mg, from about 630 mg to about 1,370 mg, from about 635 mg to about 1,365 mg, from about 640 mg to about 1,360 mg, from about 645 mg to about 1,355 mg, from about 650 mg to about 1,350 mg, from about 655 mg to about 1,345 mg, from about 660 mg to about 1,340 mg, from about 665 mg to about 1,335 mg, from about 670 mg to about 1,330 mg, from about 675 mg to about 1,325 mg, from about 680 mg to about 1,320 mg, from about 685 mg to about 1,315 mg, from about 690 mg to about 1,310 mg, from about 695 mg to about 1,305 mg, from about 700 mg to about 1,300 mg, from about 705 mg to about 1,295 mg, from about 710 mg to about 1,290 mg, from about 715 mg to about 1,285 mg, from about 720 mg to about 1,280 mg, from about 725 mg to about 1,275 mg, from about 730 mg to about 1,270 mg, from about 735 mg to about 1,265 mg, from about 740 mg to about 1,260 mg, from about 745 mg to about 1,255 mg, from about 750 mg to about 1,250 mg, from about 755 mg to about 1,245 mg, from about 760 mg to about 1,240 mg, from about 765 mg to about 1,235 mg, from about 770 mg to about 1,230 mg, from about 775 mg to about 1,225 mg, from about 780 mg to about 1,220 mg, from about 785 mg to about 1,215 mg, from about 790 mg to about 1,210 mg, from about 795 mg to about 1,205 mg, from about 800 mg to about 1,200 mg, from about 805 mg to about 1,195 mg, from about 810 mg to about 1,190 mg, from about 815 mg to about 1,185 mg, from about 820 mg to about 1,180 mg, from about 825 mg to about 1,175 mg, from about 830 mg to about 1,170 mg, from about 835 mg to about 1,165 mg, from about 840 mg to about 1,160 mg, from about 845 mg to about 1,155 mg, from about 850 mg to about 1,150 mg, from about 855 mg to about 1,145 mg, from about 860 mg to about 1,140 mg, from about 865 mg to about 1,135 mg, from about 870 mg to about 1,130 mg, from about 875 mg to about 1,125 mg, from about 880 mg to about 1,120 mg, from about 885 mg to about 1,115 mg, from about 890 mg to about 1,110 mg, from about 895 mg to about 1,105 mg, from about 900 mg to about 1,000 mg, from about 905 mg to about 1,095 mg, from about 910 mg to about 1,090 mg, from about 915 mg to about 1,085 mg, from about 920 mg to about 1,080 mg, from about 925 mg to about 1,075 mg, from about 930 mg to about 1,070 mg, from about 935 mg to about 1,065 mg, from about 940 mg to about 1,060 mg, from about 945 mg to about 1,055 mg, from about 950 mg to about 1,050 mg, from about 955 mg to about 1,045 mg, from about 960 mg to about 1,040 mg, from about 965 mg to about 1,035 mg, from about 970 mg to about 1,030 mg, from about 975 mg to about 1,025 mg, from about 980 mg to about 1,020 mg, from about 985 mg to about 1,015 mg, from about 990 mg to about 1,010 mg, or from about 995 mg to about 1,005 mg per dose. In some embodiments, the compound is administered to the subject in an amount of about 1,000 mg per dose.

In some embodiments, the compound is administered to the subject in an amount of from about 1,500 mg to about 2,500 mg per dose, such as an amount of from about 1,505 mg to about 2,495 mg, from about 1,510 mg to about 2,490 mg, from about 1,515 mg to about 2,485 mg, from about 1,520 mg to about 2,480 mg, from about 1,525 mg to about 2,475 mg, from about 1,530 mg to about 2,470 mg, from about 1,535 mg to about 2,465 mg, from about 1,540 mg to about 2,460 mg, from about 1,545 mg to about 2,455 mg, from about 1,550 mg to about 2,450 mg, from about 1,555 mg to about 2,445 mg, from about 1,560 mg to about 2,440 mg, from about 1,565 mg to about 2,435 mg, from about 1,570 mg to about 2,430 mg, from about 1,575 mg to about 2,425 mg, from about 1,580 mg to about 2,420 mg, from about 1,585 mg to about 2,415 mg, from about 1,590 mg to about 2,410 mg, from about 1,595 mg to about 2,405 mg, from about 1,600 mg to about 2,400 mg, from about 1,605 mg to about 2,395 mg, from about 1,610 mg to about 2,390 mg, from about 1,615 mg to about 2,385 mg, from about 1,620 mg to about 2,380 mg, from about 1,625 mg to about 2,375 mg, from about 1,630 mg to about 2,370 mg, from about 1,635 mg to about 2,365 mg, from about 1,640 mg to about 2,360 mg, from about 1,645 mg to about 2,355 mg, from about 1,650 mg to about 2,350 mg, from about 1,655 mg to about 2,345 mg, from about 1,660 mg to about 2,340 mg, from about 1,665 mg to about 2,335 mg, from about 1,670 mg to about 2,330 mg, from about 1,675 mg to about 2,325 mg, from about 1,680 mg to about 2,320 mg, from about 1,685 mg to about 2,315 mg, from about 1,690 mg to about 2,310 mg, from about 1,695 mg to about 2,305 mg, from about 1,700 mg to about 2,300 mg, from about 1,705 mg to about 2,295 mg, from about 1,710 mg to about 2,290 mg, from about 1,715 mg to about 2,285 mg, from about 1,720 mg to about 2,280 mg, from about 1,725 mg to about 2,275 mg, from about 1,730 mg to about 2,270 mg, from about 1,735 mg to about 2,265 mg, from about 1,740 mg to about 2,260 mg, from about 1,745 mg to about 2,255 mg, from about 1,750 mg to about 2,250 mg, from about 1,755 mg to about 2,245 mg, from about 1,760 mg to about 2,240 mg, from about 1,765 mg to about 2,235 mg, from about 1,770 mg to about 2,230 mg, from about 1,775 mg to about 2,225 mg, from about 1,780 mg to about 2,220 mg, from about 1,785 mg to about 2,215 mg, from about 1,790 mg to about 2,210 mg, from about 1,795 mg to about 2,205 mg, from about 1,800 mg to about 2,200 mg, from about 1,805 mg to about 2,195 mg, from about 1,810 mg to about 2,190 mg, from about 1,815 mg to about 2,185 mg, from about 1,820 mg to about 2,180 mg, from about 1,825 mg to about 2,175 mg, from about 1,830 mg to about 2,170 mg, from about 1,835 mg to about 2,165 mg, from about 1,840 mg to about 2,160 mg, from about 1,845 mg to about 2,155 mg, from about 1,850 mg to about 2,150 mg, from about 1,855 mg to about 2,145 mg, from about 1,860 mg to about 2,140 mg, from about 1,865 mg to about 2,135 mg, from about 1,870 mg to about 2,130 mg, from about 1,875 mg to about 2,125 mg, from about 1,880 mg to about 2,120 mg, from about 1,885 mg to about 2,115 mg, from about 1,890 mg to about 2,110 mg, from about 1,895 mg to about 2,105 mg, from about 1,900 mg to about 2,100 mg, from about 1,905 mg to about 2,095 mg, from about 1,910 mg to about 2,090 mg, from about 1,915 mg to about 2,085 mg, from about 1,920 mg to about 2,080 mg, from about 1,925 mg to about 2,075 mg, from about 1,930 mg to about 2,070 mg, from about 1,935 mg to about 2,065 mg, from about 1,940 mg to about 2,060 mg, from about 1,945 mg to about 2,055 mg, from about 1,950 mg to about 2,050 mg, from about 1,955 mg to about 2,045 mg, from about 1,960 mg to about 2,040 mg, from about 1,965 mg to about 2,035 mg, from about 1,970 mg to about 2,030 mg, from about 1,975 mg to about 2,025 mg, from about 1,980 mg to about 2,020 mg, from about 1,985 mg to about 2,015 mg, from about 1,990 mg to about 2,010 mg, or from about 1,995 mg to about 2,005 mg per dose. In some embodiments, the compound is administered to the subject in an amount of about 2,000 mg per dose.

For example, in some embodiments, the compound is administered to the subject in an amount of about 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, 1,150 mg, 1,155 mg, 1,160 mg, 1,165 mg, 1,170 mg, 1,175 mg, 1,180 mg, 1,185 mg, 1,190 mg, 1,195 mg, 1,200 mg, 1,205 mg, 1,210 mg, 1,215 mg, 1,220 mg, 1,225 mg, 1,230 mg, 1,235 mg, 1,240 mg, 1,245 mg, 1,250 mg, 1,255 mg, 1,260 mg, 1,265 mg, 1,270 mg, 1,275 mg, 1,280 mg, 1,285 mg, 1,290 mg, 1,295 mg, 1,300 mg, 1,305 mg, 1,310 mg, 1,315 mg, 1,320 mg, 1,325 mg, 1,330 mg, 1,335 mg, 1,340 mg, 1,345 mg, 1,350 mg, 1,355 mg, 1,360 mg, 1,365 mg, 1,370 mg, 1,375 mg, 1,380 mg, 1,385 mg, 1,390 mg, 1,395 mg, 1,400 mg, 1,405 mg, 1,410 mg, 1,415 mg, 1,420 mg, 1,425 mg, 1,430 mg, 1,435 mg, 1,440 mg, 1,445 mg, 1,450 mg, 1,455 mg, 1,460 mg, 1,465 mg, 1,470 mg, 1,475 mg, 1,480 mg, 1,485 mg, 1,490 mg, 1,495 mg, 1,500 mg, 1,505 mg, 1,510 mg, 1,515 mg, 1,520 mg, 1,525 mg, 1,530 mg, 1,535 mg, 1,540 mg, 1,545 mg, 1,550 mg, 1,555 mg, 1,560 mg, 1,565 mg, 1,570 mg, 1,575 mg, 1,580 mg, 1,585 mg, 1,590 mg, 1,595 mg, 1,600 mg, 1,605 mg, 1,610 mg, 1,615 mg, 1,620 mg, 1,625 mg, 1,630 mg, 1,635 mg, 1,640 mg, 1,645 mg, 1,650 mg, 1,655 mg, 1,660 mg, 1,665 mg, 1,670 mg, 1,675 mg, 1,680 mg, 1,685 mg, 1,690 mg, 1,695 mg, 1,700 mg, 1,705 mg, 1,710 mg, 1,715 mg, 1,720 mg, 1,725 mg, 1,730 mg, 1,735 mg, 1,740 mg, 1,745 mg, 1,750 mg, 1,755 mg, 1,760 mg, 1,765 mg, 1,770 mg, 1,775 mg, 1,780 mg, 1,785 mg, 1,790 mg, 1,795 mg, 1,800 mg, 1,805 mg, 1,810 mg, 1,815 mg, 1,820 mg, 1,825 mg, 1,830 mg, 1,835 mg, 1,840 mg, 1,845 mg, 1,850 mg, 1,855 mg, 1,860 mg, 1,865 mg, 1,870 mg, 1,875 mg, 1,880 mg, 1,885 mg, 1,890 mg, 1,895 mg, 1,900 mg, 1,905 mg, 1,910 mg, 1,915 mg, 1,920 mg, 1,925 mg, 1,930 mg, 1,935 mg, 1,940 mg, 1,945 mg, 1,950 mg, 1,955 mg, 1,960 mg, 1,965 mg, 1,970 mg, 1,975 mg, 1,980 mg, 1,985 mg, 1,990 mg, 1,995 mg, 2,000 mg, 2,005 mg, 2,010 mg, 2,015 mg, 2,020 mg, 2,025 mg, 2,030 mg, 2,035 mg, 2,040 mg, 2,045 mg, 2,050 mg, 2,055 mg, 2,060 mg, 2,065 mg, 2,070 mg, 2,075 mg, 2,080 mg, 2,085 mg, 2,090 mg, 2,095 mg, 2,100 mg, 2,105 mg, 2,110 mg, 2,115 mg, 2,120 mg, 2,125 mg, 2,130 mg, 2,135 mg, 2,140 mg, 2,145 mg, 2,150 mg, 2,155 mg, 2,160 mg, 2,165 mg, 2,170 mg, 2,175 mg, 2,180 mg, 2,185 mg, 2,190 mg, 2,195 mg, 2,200 mg, 2,205 mg, 2,210 mg, 2,215 mg, 2,220 mg, 2,225 mg, 2,230 mg, 2,235 mg, 2,240 mg, 2,245 mg, 2,250 mg, 2,255 mg, 2,260 mg, 2,265 mg, 2,270 mg, 2,275 mg, 2,280 mg, 2,285 mg, 2,290 mg, 2,295 mg, 2,300 mg, 2,305 mg, 2,310 mg, 2,315 mg, 2,320 mg, 2,325 mg, 2,330 mg, 2,335 mg, 2,340 mg, 2,345 mg, 2,350 mg, 2,355 mg, 2,360 mg, 2,365 mg, 2,370 mg, 2,375 mg, 2,380 mg, 2,385 mg, 2,390 mg, 2,395 mg, 2,400 mg, 2,405 mg, 2,410 mg, 2,415 mg, 2,420 mg, 2,425 mg, 2,430 mg, 2,435 mg, 2,440 mg, 2,445 mg, 2,450 mg, 2,455 mg, 2,460 mg, 2,465 mg, 2,470 mg, 2,475 mg, 2,480 mg, 2,485 mg, 2,490 mg, 2,495 mg, or 2,500 mg per dose.

In some embodiments of any of the above aspects, the ring Ar is selected from substituents (Ia) to (Iy):

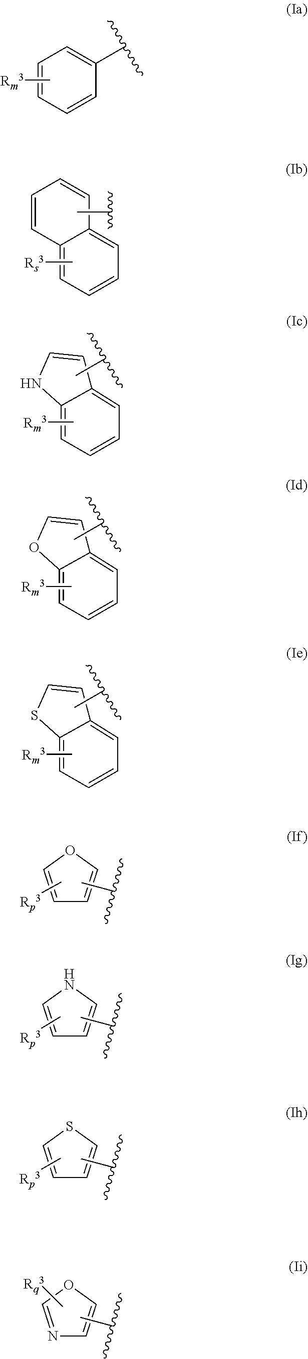

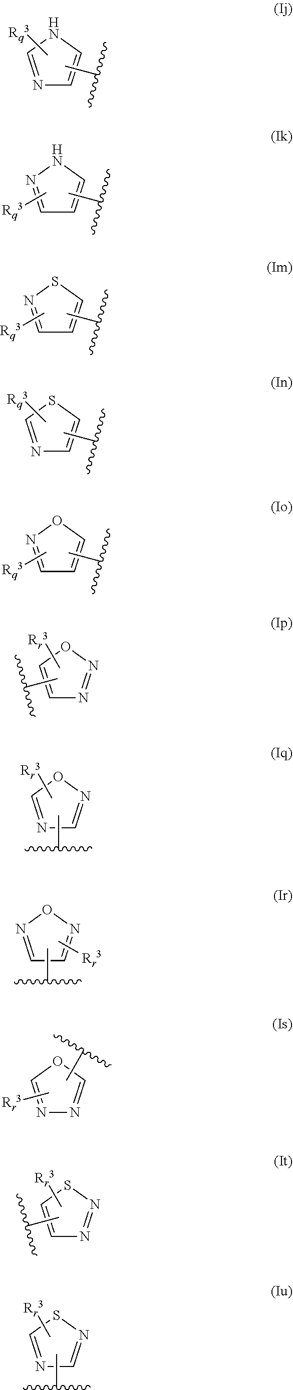

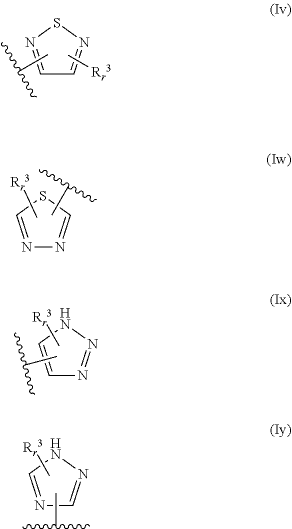

wherein each R³ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each m is independently an integer from 0-5;
each p is independently an integer from 0-3;
each q is independently an integer from 0-2;
each r is independently an integer from 0-1; and
each s is independently an integer from 0-7.

In some embodiments, each R³ is independently selected from substituents (IIa) to (IIy).

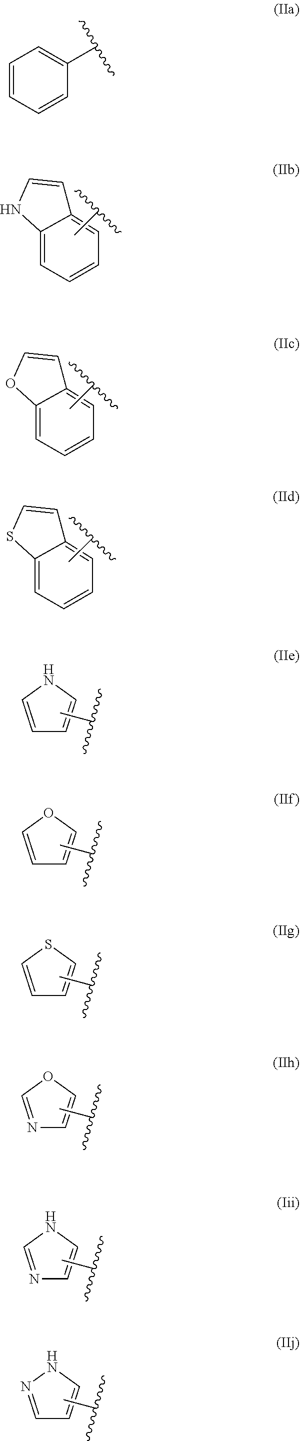

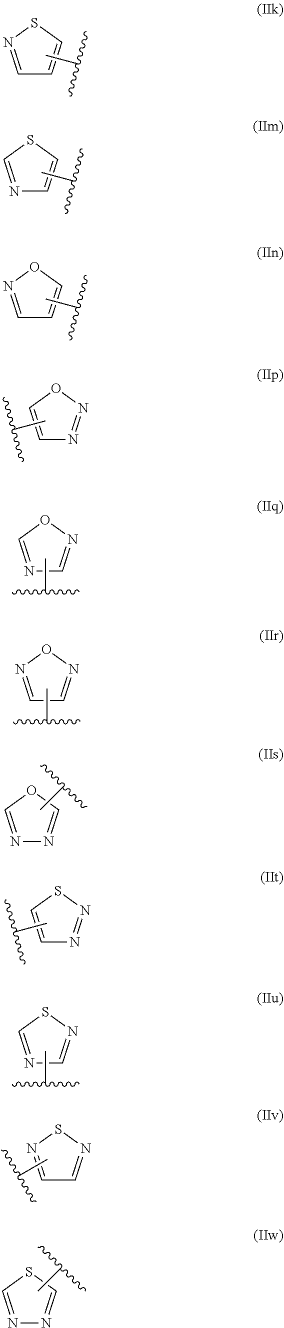

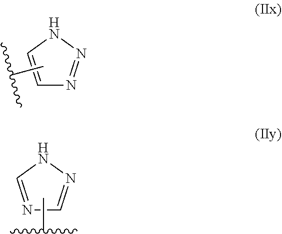

In some embodiments, the ring Ar is a substituent represented by formula (Ia)

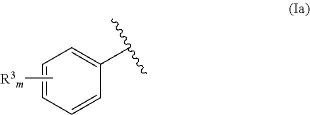

and each R³ is, independently, optionally substituted and optionally fused aryl, optionally substituted and optionally fused heteroaryl, optionally substituted and optionally fused cycloalkyl, or optionally substituted and optionally fused heterocycloalkyl.

In some embodiments, the ring Cy is selected from substituents (IIIa) to (IIIaa):

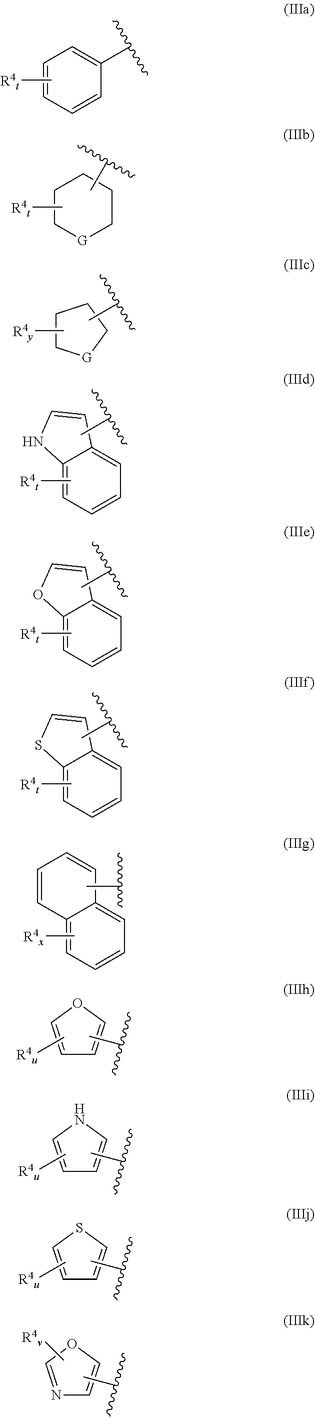

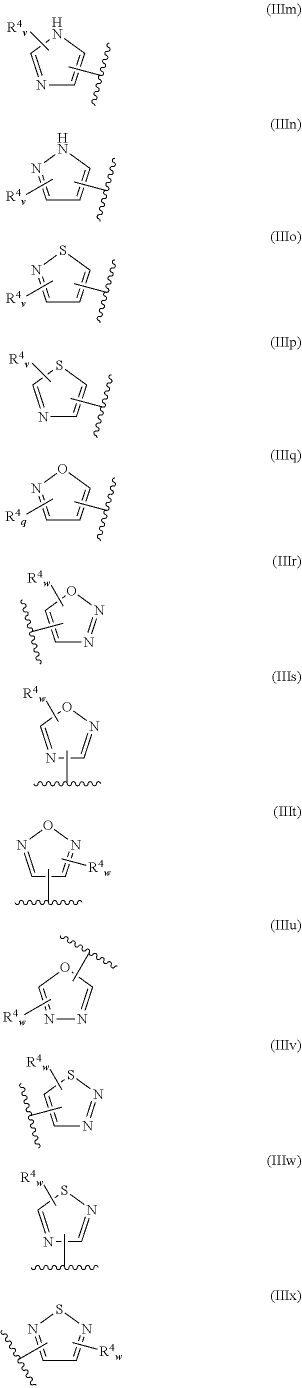

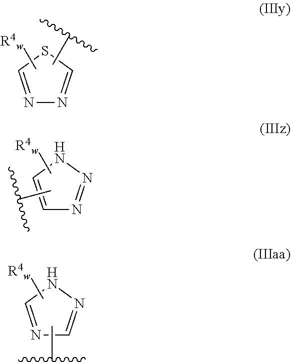

wherein each R⁴ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each G is independently —CH₂—, —CR⁴H—, —NH—, —NR⁴—, —O—, or —S—;
each t is independently an integer from 0-5;
each u is independently an integer from 0-3;
each v is independently an integer from 0-2;
each w is independently an integer from 0-1;
each x is independently an integer from 0-7; and
each y is independently an integer from 0-4.

It is to be understood that the substituent R⁴ may be bound to G in formula (IIIb) or (IIIc). In such instances, G is to be understood as being selected from the group consisting of —CH—, —CR⁴—, and —N—.

In some embodiments, the ring Cy is an optionally substituted aryl group represented by formula (IVa).

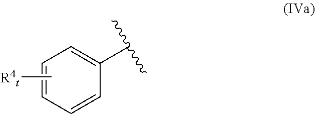

In some embodiments, the ring Cy is a substituted aryl group represented by formula (IVb).

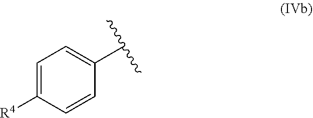

In some embodiments, R¹ is C₁-C₅-alkyl carboxy, C₁-C₅-alkyl acyl, C₁-C₅-alkyl alkoxycarbonyl, C₁-C₅-alkyl acyloxy, substituted C₁-C₅-alkyl carboxy, substituted C₁-C₅-alkyl acyl, substituted C₁-C₅-alkyl alkoxycarbonyl, or substituted C₁-C₅-alkyl acyloxy. In some embodiments, R¹ is optionally substituted C₁-C₅-alkyl acyloxy.

In some embodiments, the compound is represented by formula (V)

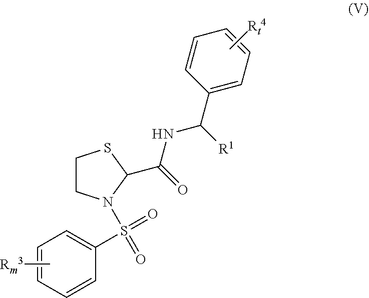

wherein R¹ is C₁-C₅-alkyl carboxy, C₁-C₅-alkyl acyl, C₁-C₅-alkyl alkoxycarbonyl, C₁-C₅-alkyl acyloxy, substituted C₁-C₅-alkyl carboxy, substituted C₁-C₅-alkyl acyl, substituted C₁-C₅-alkyl alkoxycarbonyl, or substituted C₁-C₅-alkyl acyloxy;
each R³ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each R⁴ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
m is an integer from 0-5; and
t is an integer from 0-5,
or a pharmaceutically acceptable salt thereof.

In some embodiments, the compound is represented by formula (Va)

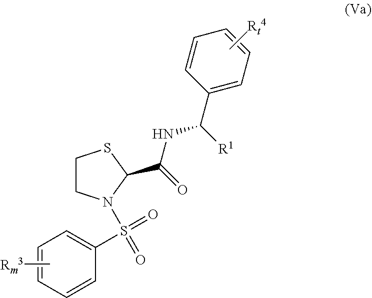

or a pharmaceutically acceptable salt thereof.

In some embodiments, the compound is represented by formula (VI)

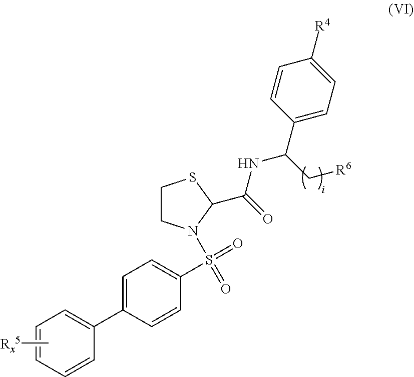

wherein R⁶ is hydroxyl, optionally substituted acyl, optionally substituted alkoxycarbonyl, or optionally substituted acyloxy;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3; and
x is an integer from 0-5,
or a pharmaceutically acceptable salt thereof.

In some embodiments, the compound is represented by formula (VIa)

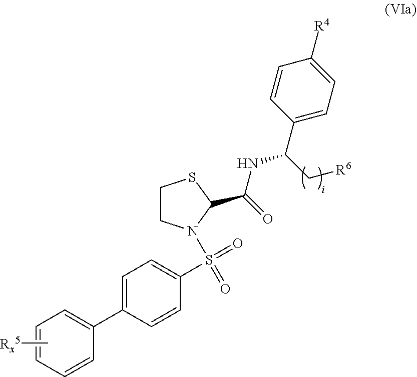

or a pharmaceutically acceptable salt thereof.

In some embodiments, the compound is represented by formula (VII)

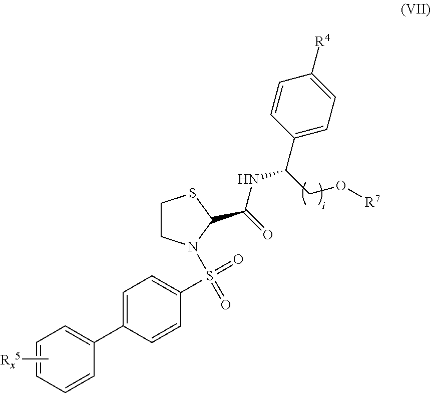

wherein R⁷ is H or optionally substituted aminoacyl;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3; and
x is an integer from 0-5,
or a pharmaceutically acceptable salt thereof.

In some embodiments, the compound is represented by formula (VIII)

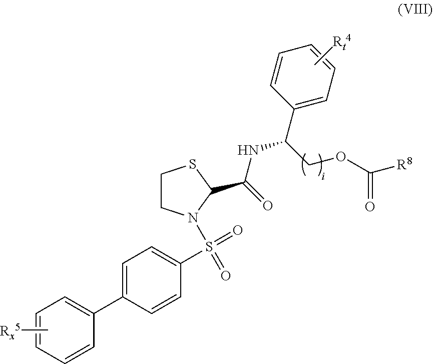

wherein R⁸ is optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl, preferably wherein R⁸ is amino-substituted alkyl, such as 1-amino C₁-C₆ alkyl (e.g., (S)-1-amino C₁-C₆ alkyl or (R)-1-amino C₁-C₆ alkyl, for example, (S)-1-amino-2-methylpropyl, (S)-1-amino-2-methylbutyl, (S)-1-amino-3-methylbutyl, (R)-1-amino-2-methylpropyl, (R)-1-amino-2-methylbutyl, or (R)-1-amino-3-methylbutyl;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3;
t is an integer from 0-5; and
x is an integer from 0-5,
or a pharmaceutically acceptable salt thereof.

In some embodiments, the method includes providing to the subject compound (1).

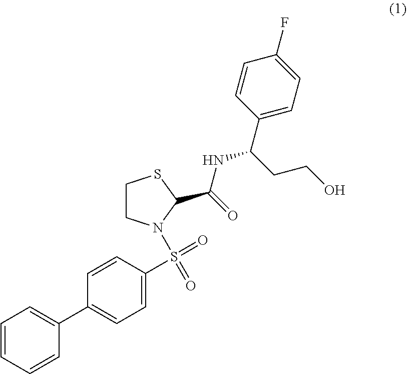

In some embodiments, the method includes administering to the subject compound (2)

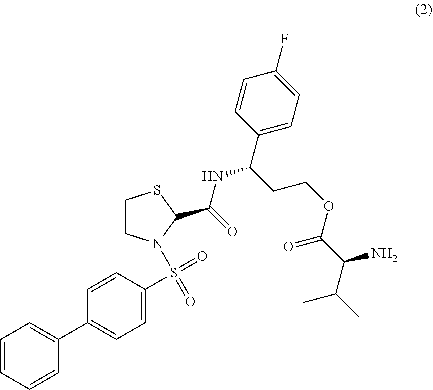

or a pharmaceutically acceptable salt thereof, such as the chloride salt thereof, represented by formula (3).

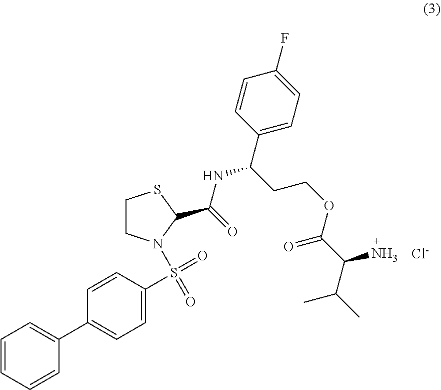

In some embodiments, the compound is in a crystalline state. For example, the compound may be crystalline compound (3), and exhibit:
(a) characteristic X-ray powder diffraction peaks at about 7.0° 2θ, about 8.1° 2θ, about 10.0° 2θ, about 12.0° 2θ, about 13.1° 2θ, about 14.1° 2θ, about 16.4° 2θ, about 18.4° 2θ, about 20.1° 2θ, about 21.0° 2θ, about 23.5° 2θ, and about 29.5° 2θ;
(b) ¹H nuclear magnetic resonance (NMR) peaks centered at about 1.1 ppm, about 3.3 ppm, about 4.9 ppm, about 5.4 ppm, about 7.1 ppm, about 7.7 ppm, about 7.9 ppm, and about 8.0 ppm;
(c) an endotherm at from about 145° C. to about 147° C. as measured by differential scanning calorimetry;
(d) a weight loss of from about 0.2% to about 0.6% when heated from 25° C. to 100° C. as measured by thermogravimetric analysis; and/or
(e) a weight loss of from about 2.5% to about 3.5% when heated from 100° C. to 160° C. as measured by thermogravimetric analysis.

In some embodiments of any of the foregoing aspects of the disclosure, the PGF2α receptor antagonist is administered to the subject in an amount of from about 300 mg to about 2,300 mg per dose, such as an amount of about 300 mg per dose, 350 mg per dose, 400 mg per dose, 450 mg per dose, 500 mg per dose, 550 mg per dose, 600 mg per dose, 650 mg per dose, 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, 1,300 mg per dose, 1,350 mg per dose, 1,400 mg per dose, 1,450 mg per dose, 1,500 mg per dose, 1,550 mg per dose, 1,600 mg per dose, 1,650 mg per dose, 1,700 mg per dose, 1,750 mg per dose, 1,800 mg per dose, 1,850 mg per dose, 1,900 mg per dose, 1,950 mg per dose, 2,000 mg per dose, 2,050 mg per dose, 2,100 mg per dose, 2,150 mg per dose, 2,200 mg per dose, 2,250 mg per dose, or 2,300 mg per dose. The PGF2α receptor antagonist may be administered to the subject in an amount of from about 400 mg to about 2,100 mg per dose, such as an amount of about 400 mg per dose, 450 mg per dose, 500 mg per dose, 550 mg per dose, 600 mg per dose, 650 mg per dose, 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, 1,300 mg per dose, 1,350 mg per dose, 1,400 mg per dose, 1,450 mg per dose, 1,500 mg per dose, 1,550 mg per dose, 1,600 mg per dose, 1,650 mg per dose, 1,700 mg per dose, 1,750 mg per dose, 1,800 mg per dose, 1,850 mg per dose, 1,900 mg per dose, 1,950 mg per dose, 2,000 mg per dose, 2,050 mg per dose, or 2,100 mg per dose. In some embodiments, the PGF2α receptor antagonist is administered to the subject in an amount of from about 450 mg to about 2,050 mg per dose, such as an amount of about 450 mg per dose, 500 mg per dose, 550 mg per dose, 600 mg per dose, 650 mg per dose, 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, 1,300 mg per dose, 1,350 mg per dose, 1,400 mg per dose, 1,450 mg per dose, 1,500 mg per dose, 1,550 mg per dose, 1,600 mg per dose, 1,650 mg per dose, 1,700 mg per dose, 1,750 mg per dose, 1,800 mg per dose, 1,850 mg per dose, 1,900 mg per dose, 1,950 mg per dose, 2,000 mg per dose, or 2,050 mg per dose.

In some embodiments, the PGF2α receptor antagonist is administered to the subject in an amount of from about 100 mg to about 900 mg per dose, such as an amount of about 100 mg per dose, 150 mg per dose, 200 mg per dose, 250 mg per dose, 300 mg per dose, 350 mg per dose, 400 mg per dose, 450 mg per dose, 500 mg per dose, 550 mg per dose, 600 mg per dose, 650 mg per dose, 700 mg per dose, 750 mg per dose, 800 mg per dose, 850 mg per dose, or 900 mg per dose. In some embodiments, the PGF2α receptor antagonist is administered to the subject in an amount of from about 200 mg to about 800 mg per dose, such as an amount of about 200 mg per dose, 250 mg per dose, 300 mg per dose, 350 mg per dose, 400 mg per dose, 450 mg per dose, 500 mg per dose, 550 mg per dose, 600 mg per dose, 650 mg per dose, 700 mg per dose, 750 mg per dose, or 800 mg per dose. In some embodiments, the PGF2α receptor antagonist is administered to the subject in an amount of from about 300 mg to about 700 mg per dose, such as an amount of about 300 mg per dose, 350 mg per dose, 400 mg per dose, 450 mg per dose, 500 mg per dose, 550 mg per dose, 600 mg per dose, 650 mg per dose, or 700 mg per dose. In some embodiments, the PGF2α receptor antagonist is administered to the subject in an amount of from about 400 mg to about 600 mg per dose, such as an amount of about 400 mg per dose, 450 mg per dose, 500 mg per dose, 550 mg per dose, or 600 mg per dose. In some embodiments, the PGF2α receptor antagonist is administered to the subject in an amount of from about 450 mg to about 550 mg per dose, such as an amount of about 450 mg per dose, 500 mg per dose, or 550 mg per dose.

In some embodiments, the PGF2α receptor antagonist is administered to the subject in an amount of from about 600 mg to about 1,400 mg per dose, such as an amount of about 600 mg per dose, 650 mg per dose, 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, 1,300 mg per dose, 1,350 mg per dose, or 1,400 mg per dose. In some embodiments, the PGF2α receptor antagonist is administered to the subject in an amount of from about 700 mg to about 1,300 mg per dose, such as an amount of about 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, or 1,300 mg per dose. In some embodiments, the PGF2α receptor antagonist is administered to the subject in an amount of from about 800 mg to about 1,200 mg per dose, such as an amount of about 800 mg per dose, 850 mg per dose, 900 mg per dose, 950 mg per dose, 1,000 mg per dose, 1,050 mg per dose, 1,100 mg per dose, 1,150 mg per dose, or 1,200 mg per dose. In some embodiments, the PGF2α receptor antagonist is administered to the subject in an amount of from about 900 mg to about 1,100 mg per dose, such as an amount of about 900 mg per dose, 950 mg per dose, 1,000 mg per dose, 1,050 mg per dose, or 1,100 mg per dose. In some embodiments, the PGF2α receptor antagonist is administered to the subject in an amount of from about 950 mg to about 1,050 mg per dose, such as an amount of about 950 mg per dose, 1,000 mg per dose, or 1,050 mg per dose.

In some embodiments, the PGF2α receptor antagonist, such as the compound of any one of formulas (I) through (VIII), for example, compound (1), compound (2), or compound (3), among other PGF2α receptor antagonists described herein, is periodically administered to the subject in one or more doses per 12 hours, 24 hours, 36 hours, 48 hours, 60 hours, 72 hours, 84 hours, 96 hours, week, or month. For instance, the PGF2α receptor antagonist may be administered to the subject in from one to ten doses (e.g., 1, 2, 3, 4, 5, 6, 7, 8, 9, or 10 doses) every 12 hours, one to ten doses (e.g., 1, 2, 3, 4, 5, 6, 7, 8, 9, or 10 doses) every 14 hours, one to ten doses (e.g., 1, 2, 3, 4, 5, 6, 7, 8, 9, or 10 doses) every 16 hours, one to ten doses (e.g., 1, 2, 3, 4, 5, 6, 7, 8, 9, or 10 doses) every 18 hours, one to ten doses (e.g., 1, 2, 3, 4, 5, 6, 7, 8, 9, or 10 doses) every 20 hours, one to ten doses (e.g., 1, 2, 3, 4, 5, 6, 7, 8, 9, or 10 doses) every 22 hours, one to ten doses (e.g., 1, 2, 3, 4, 5, 6, 7, 8, 9, or 10 doses) every 24 hours, one to ten doses (e.g., 1, 2, 3, 4, 5, 6, 7, 8, 9, or 10 doses) every 26 hours, one to ten doses (e.g., 1, 2, 3, 4, 5, 6, 7, 8, 9, or 10 doses) every 28 hours, one to ten doses (e.g., 1, 2, 3, 4, 5, 6, 7, 8, 9, or 10 doses) every 30 hours, one to ten doses (e.g., 1, 2, 3, 4, 6, 7, 8, 9, or 10 doses) every 32 hours, one to ten doses (e.g., 1, 2, 3, 4, 5, 6, 7, 8, 9, or 10 doses) every 34 hours, one to ten doses (e.g., 1, 2, 3, 4, 5, 6, 7, 8, 9, or 10 doses) every 36 hours, one to ten doses (e.g., 1, 2, 3, 4, 5, 6, 7, 8, 9, or 10 doses) every 38 hours, one to ten doses (e.g., 1, 2, 3, 4, 5, 6, 7, 8, 9, or 10 doses) every 40 hours, one to ten doses (e.g., 1, 2, 3, 4, 5, 6, 7, 8, 9, or 10 doses) every 42 hours, one to ten doses (e.g., 1, 2, 3, 4, 5, 6, 7, 8, 9, or 10 doses) every 44 hours, one to ten doses (e.g., 1, 2, 3, 4, 5, 6, 7, 8, 9, or 10 doses) every 46 hours, one to ten doses (e.g., 1, 2, 3, 4, 5, 6, 7, 8, 9, or 10 doses) every 48 hours, one to ten doses (e.g., 1, 2, 3, 4, 5, 6, 7, 8, 9, or 10 doses) every 60 hours, one to ten doses (e.g., 1, 2, 3, 4, 5, 6, 7, 8, 9, or 10 doses) every 72 hours, one to ten doses (e.g., 1, 2, 3, 4, 5, 6, 7, 8, 9, or 10 doses) every 84 hours, one to ten doses (e.g., 1, 2, 3, 4, 5, 6, 7, 8, 9, or 10 doses) every 96 hours, one to ten doses (e.g., 1, 2, 3, 4, 5, 6, 7, 8, 9, or 10 doses) every 108 hours, one to ten doses (e.g., 1, 2, 3, 4, 5, 6, 7, 8, 9, or 10 doses) every 120 hours, one to ten doses (e.g., 1, 2, 3, 4, 5, 6, 7, 8, 9, or 10 doses) every 132 hours, one to ten doses (e.g., 1, 2, 3, 4, 5, 6, 7, 8, 9, or 10 doses) every 144 hours, one to ten doses (e.g., 1, 2, 3, 4, 5, 6, 7, 8, 9, or 10 doses) every 156 hours, or one to ten doses (e.g., 1, 2, 3, 4, 5, 6, 7, 8, 9, or 10 doses) every week.

In some embodiments, the PGF2α receptor antagonist, such as the compound of any one of formulas (I) through (VIII), for example, compound (1), compound (2), or compound (3), among other PGF2α receptor antagonists described herein, is administered to the subject in from one to six doses per day. For example, PGF2α receptor antagonist, such as the compound of any one of formulas (I) through (VIII), for example, compound (1), compound (2), or compound (3), may be administered to the subject once daily. In some embodiments, the PGF2α receptor antagonist is administered to the subject twice daily.

In some embodiments, the PGF2α receptor antagonist is administered to the subject once every 4 to 12 hours, such as once every 4 hours, once every 5 hours, once every 6 hours, once every 7 hours, once every 8 hours, once every 9 hours, once every 10 hours, once every 11 hours, or once every 12 hours.

The PGF2α receptor antagonist, such as the compound of any one of formulas (I) through (VIII), for example, compound (1), compound (2), or compound (3), among other PGF2α receptor antagonists described herein, may be administered to the subject in one or more unit dosage forms that collectively constitute a single dose. For example, the subject may be administered a single dose of the compound of a specified amount, such as a single dose of 250 mg, 500 mg, 750 mg, 1,000 mg, 1,250 mg, 1,500 mg, 1,750 mg, 2,000 mg, or more, by administration of one or more unit dosage forms of the compound to the subject. As a non-limiting example, a single dose of 1,000 mg of the compound may be administered to the subject by way of two individual 500-mg unit dosage forms of the compound. The two 500-mg unit dosage forms collectively constitute a single 1,000-mg dose of the compound if administered to the subject at substantially the same time.

In some embodiments, the PGF2α receptor antagonist, such as the compound of any one of formulas (I) through (VIII), for example, compound (1), compound (2), or compound (3), among other PGF2α receptor antagonists described herein, is administered to the subject in one or more daily doses (e.g., from 1 to 10 doses, such as 1, 2, 3, 4, 5, 6, 7, 8, 9, or 10 doses, or more) totaling from about 250 mg to about 2,500 mg per day, such as an amount of about 250 mg, 300 mg, 350 mg, 400 mg, 450 mg, 500 mg, 550 mg, 600 mg, 650 mg, 700 mg, 750 mg, 800 mg, 850 mg, 900 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,050 mg, 2,100 mg, 2,150 mg, 2,200 mg, 2,250 mg, 2,300 mg, 2,350 mg, 2,400 mg, 2,450 mg, or 2,500 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 300 mg to about 2,000 mg per day, such as an amount of about 300 mg, 350 mg, 400 mg, 450 mg, 500 mg, 550 mg, 600 mg, 650 mg, 700 mg, 750 mg, 800 mg, 850 mg, 900 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, or 2,000 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 400 mg to about 1.600 mg per day, such as an amount of about 400 mg, 450 mg, 500 mg, 550 mg, 600 mg, 650 mg, 700 mg, 750 mg, 800 mg, 850 mg, 900 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, or 1,600 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 700 mg to about 1,300 mg per day, such as an amount of about 700 mg, 750 mg, 800 mg, 850 mg, 900 mg, 1,000 mg, 1,050 mg, 1,100 mg, 1,150 mg, 1,200 mg, 1,250 mg, or 1,300 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 800 mg to about 1,200 mg per day, such as an amount of about 800 mg, 850 mg, 900 mg, 1,000 mg, 1,050 mg, 1,100 mg, 1,150 mg, or 1,200 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 900 mg to about 1,100 mg per day, such as an amount of about 900 mg, 1,000 mg, 1,050 mg, or 1,100 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 950 mg to about 1,050 mg per day, such as an amount of about 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, or 1,050 mg per day.

In some embodiments, the PGF2α receptor antagonist, such as the compound of any one of formulas (I) through (VIII), for example, compound (1), compound (2), or compound (3), among other PGF2α receptor antagonists described herein, is administered to the subject in a single dose per day. For example, the PGF2α receptor antagonist may be administered to the subject in an amount (e.g., a single dose) of from about 250 mg to about 2,500 mg per day, such as an amount of about 250 mg, 300 mg, 350 mg, 400 mg, 450 mg, 500 mg, 550 mg, 600 mg, 650 mg, 700 mg, 750 mg, 800 mg, 850 mg, 900 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,050 mg, 2,100 mg, 2,150 mg, 2,200 mg, 2,250 mg, 2,300 mg, 2,350 mg, 2,400 mg, 2,450 mg, or 2,500 mg per day. In some embodiments, the PGF2α receptor antagonist is administered to the subject in an amount (e.g., a single dose) of about 1,000 mg per day.

In some embodiments, the PGF2α receptor antagonist, such as the compound of any one of formulas (I) through (VIII), for example, compound (1), compound (2), or compound (3), among other PGF2α receptor antagonists described herein, is administered to the subject once daily in an amount of from about 800 mg to about 1,200 mg per dose during a first treatment period. The PGF2α receptor antagonist may subsequently be administered to the subject twice daily in an amount of from about 300 mg to about 700 mg per dose during a second treatment period, i.e., totaling an amount of from about 600 mg to about 1,400 mg per day during the second treatment period.

In some embodiments, the PGF2α receptor antagonist is administered to the subject in an amount of from about 850 mg to about 1,150 mg per dose during the first treatment period, such as an amount of about 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, or 1,150 mg per dose during the first treatment period (i.e., totaling an amount of about 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, or 1,150 mg per day during the first treatment period).

In some embodiments, the PGF2α receptor antagonist is administered to the subject in an amount of from about 900 mg to about 1,100 mg per dose during the first treatment period, such as an amount of about 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, or 1,100 mg per dose during the first treatment period (i.e., totaling an amount of about 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, or 1,100 mg per day during the first treatment period).

In some embodiments, the PGF2α receptor antagonist is administered to the subject in an amount of from about 950 mg to about 1,050 mg per dose during the first treatment period, such as an amount of about 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, or 1,050 mg per dose during the first treatment period (i.e., totaling an amount of about 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, or 1,050 mg per day during the first treatment period). In some embodiments, the PGF2α receptor antagonist is administered to the subject in an amount of about 1,000 mg per dose during the first treatment period.

In some embodiments, the PGF2α receptor antagonist is administered to the subject in an amount of from about 350 mg to about 650 mg per dose during the second treatment period, such as an amount of about 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, or 650 mg per dose during the second treatment period (i.e., totaling an amount of from about 700 mg to about 1,300 mg per day during the second treatment period, such as an amount of about 700 mg, 710 mg, 720 mg, 730 mg, 740 mg, 750 mg, 760 mg, 770 mg, 780 mg, 790 mg, 800 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1,000 mg, 1,010 mg, 1,020 mg, 1,030 mg, 1,040 mg, 1,050 mg, 1,060 mg, 1,070 mg, 1,080 mg, 1,090 mg, 1,100 mg, 1,110 mg, 1,120 mg, 1,130 mg, 1,140 mg, 1,150 mg, 1,160 mg, 1,170 mg, 1,180 mg, 1,190 mg, 1,200 mg, 1,210 mg, 1,220 mg, 1,230 mg, 1,240 mg, 1,250 mg, 1,260 mg, 1,270 mg, 1,280 mg, 1,290 mg, or 1,300 mg per day during the second treatment period).

In some embodiments, the PGF2α receptor antagonist is administered to the subject in an amount of from about 400 mg to about 600 mg per dose during the second treatment period, such as an amount of about 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, or 600 mg per dose during the second treatment period (i.e., totaling an amount of from about 800 mg to about 1,200 mg per day during the second treatment period, such as an amount of about 800 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1,000 mg, 1,010 mg, 1,020 mg, 1,030 mg, 1,040 mg, 1,050 mg, 1,060 mg, 1,070 mg, 1,080 mg, 1,090 mg, 1,100 mg, 1,110 mg, 1,120 mg, 1,130 mg, 1,140 mg, 1,150 mg, 1,160 mg, 1,170 mg, 1,180 mg, 1,190 mg, or 1,200 mg per day during the second treatment period).

In some embodiments, the PGF2α receptor antagonist is administered to the subject in an amount of from about 450 mg to about 550 mg per dose during the second treatment period, such as an amount of about 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, or 550 mg per dose during the second treatment period (i.e., totaling an amount of from about 900 mg to about 1,100 mg per day during the second treatment period, such as an amount of about 900 mg, 910 mg, 920 mg, 930 mg, 940 mg, 950 mg, 960 mg, 970 mg, 980 mg, 990 mg, 1,000 mg, 1,010 mg, 1,020 mg, 1,030 mg, 1,040 mg, 1,050 mg, 1,060 mg, 1,070 mg, 1,080 mg, 1,090 mg, or 1,100 mg per day during the second treatment period). In some embodiments, the PGF2α receptor antagonist is administered to the subject in an amount of about 500 mg per dose during the second treatment period.

In some embodiments, the PGF2α receptor antagonist is administered to the subject once every 10-14 hours during the second treatment period. In some embodiments, the PGF2α receptor antagonist is administered to the subject once every 11-13 hours during the second treatment period. In some embodiments, the PGF2α receptor antagonist is administered to the subject once every 12 hours during the second treatment period.

In some embodiments, the first treatment period has a duration of from 1 to 10 days (e.g., a duration of 1 day, 2 days, 3 days, 4 days, 5 days, 6 days, 7 days, 8 days, 9 days, or 10 days). In some embodiments, the first treatment period has a duration of from 1 to 5 days (e.g., a duration of 1 day, 2 days, 3 days, 4 days, or 5 days). In some embodiments, the first treatment period has a duration of 1 day.

In some embodiments, the second treatment period has a duration of from 1 to 28 days (e.g., a duration of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days). In some embodiments, the second treatment period has a duration of from 4 to 14 days (e.g., a duration of 4 days, 5 days, 6 days, 7 days, 8 days, 9 days, 10 days, 11 days, 12 days, 13 days, or 14 days). In some embodiments, the second treatment period has a duration of from 5 to 10 days (e.g., a duration of 5 days, 6 days, 7 days, 8 days, 9 days, or 10 days). In some embodiments, the second treatment period has a duration of 3 days. In some embodiments, the second treatment period has a duration of 4 days. In some embodiments, the second treatment period has a duration of 5 days. In some embodiments, the second treatment period has a duration of 6 days. In some embodiments, the second treatment period has a duration of 7 days. In some embodiments, the second treatment period has a duration of 8 days. In some embodiments, the second treatment period has a duration of 9 days. In some embodiments, the second treatment period has a duration of 10 days.

In some embodiments, the PGF2α receptor antagonist is administered to the subject until the subject reaches a gestational age of at least about 34 weeks (e.g., a gestational age of from about 34 weeks to about 40 weeks, such as a gestational age of 34 weeks, 35 weeks, 36 weeks, 37 weeks, 38 weeks, 39 weeks, or 40 weeks), or until the subject undergoes delivery. In some embodiments, the PGF2α receptor antagonist is administered to the subject until the subject reaches a gestational age of about 37 weeks.

In some embodiments, the PGF2α receptor antagonist is administered to the subject orally. The PGF2α receptor antagonist may be, for example, formulated a tablet, gel cap, powder, liquid solution, or liquid suspension.

In some embodiments, the atosiban is periodically administered to the subject in one or more doses per 12 hours, 24 hours, 36 hours, 48 hours, 60 hours, 72 hours, 84 hours, 96 hours, week, or month. For instance, the atosiban may be administered to the subject in from one to ten doses (e.g., 1, 2, 3, 4, 5, 6, 7, 8, 9, or 10 doses) every 12 hours, one to ten doses (e.g., 1, 2, 3, 4, 5, 6, 7, 8, 9, or 10 doses) every 14 hours, one to ten doses (e.g., 1, 2, 3, 4, 5, 6, 7, 8, 9, or 10 doses) every 16 hours, one to ten doses (e.g., 1, 2, 3, 4, 5, 6, 7, 8, 9, or 10 doses) every 18 hours, one to ten doses (e.g., 1, 2, 3, 4, 5, 6, 7, 8, 9, or 10 doses) every 20 hours, one to ten doses (e.g., 1, 2, 3, 4, 5, 6, 7, 8, 9, or 10 doses) every 22 hours, one to ten doses (e.g., 1, 2, 3, 4, 5, 6, 7, 8, 9, or 10 doses) every 24 hours, one to ten doses (e.g., 1, 2, 3, 4, 5, 6, 7, 8, 9, or 10 doses) every 26 hours, one to ten doses (e.g., 1, 2, 3, 4, 5, 6, 7, 8, 9, or 10 doses) every 28 hours, one to ten doses (e.g., 1, 2, 3, 4, 5, 6, 7, 8, 9, or 10 doses) every 30 hours, one to ten doses (e.g., 1, 2, 3, 4, 5, 6, 7, 8, 9, or 10 doses) every 32 hours, one to ten doses (e.g., 1, 2, 3, 4, 5, 6, 7, 8, 9, or 10 doses) every 34 hours, one to ten doses (e.g., 1, 2, 3, 4, 5, 6, 7, 8, 9, or 10 doses) every 36 hours, one to ten doses (e.g., 1, 2, 3, 4, 5, 6, 7, 8, 9, or 10 doses) every 38 hours, one to ten doses (e.g., 1, 2, 3, 4, 5, 6, 7, 8, 9, or 10 doses) every hours, one to ten doses (e.g., 1, 2, 3, 4, 5, 6, 7, 8, 9, or 10 doses) every 42 hours, one to ten doses (e.g., 1, 2, 3, 4, 5, 6, 7, 8, 9, or 10 doses) every 44 hours, one to ten doses (e.g., 1, 2, 3, 4, 5, 6, 7, 8, 9, or 10 doses) every 46 hours, one to ten doses (e.g., 1, 2, 3, 4, 5, 6, 7, 8, 9, or 10 doses) every 48 hours, one to ten doses (e.g., 1, 2, 3, 4, 5, 6, 7, 8, 9, or 10 doses) every 60 hours, one to ten doses (e.g., 1, 2, 3, 4, 5, 6, 7, 8, 9, or 10 doses) every 72 hours, one to ten doses (e.g., 1, 2, 3, 4, 5, 6, 7, 8, 9, or 10 doses) every 84 hours, one to ten doses (e.g., 1, 2, 3, 4, 5, 6, 7, 8, 9, or 10 doses) every 96 hours, one to ten doses (e.g., 1, 2, 3, 4, 5, 6, 7, 8, 9, or 10 doses) every 108 hours, one to ten doses (e.g., 1, 2, 3, 4, 5, 6, 7, 8, 9, or 10 doses) every 120 hours, one to ten doses (e.g., 1, 2, 3, 4, 5, 6, 7, 8, 9, or 10 doses) every 132 hours, one to ten doses (e.g., 1, 2, 3, 4, 5, 6, 7, 8, 9, or 10 doses) every 144 hours, one to ten doses (e.g., 1, 2, 3, 4, 5, 6, 7, 8, 9, or 10 doses) every 156 hours, or one to ten doses (e.g., 1, 2, 3, 4, 5, 6, 7, 8, 9, or 10 doses) every week.

In some embodiments, the atosiban is administered to the subject in a single bolus dose of from about 4.0 mg to about 9.5 mg (e.g., a single bolus dose of about 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, or 9.0 mg). In some embodiments, the atosiban is administered to the subject in a single bolus dose of from about 5.0 mg to about 8.5 mg (e.g., a single bolus dose of about 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, or 8.5 mg). In some embodiments, the atosiban is administered to the subject in a single bolus dose of from about 5.5 mg to about 8.0 mg (e.g., a single bolus dose of about 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, or 8.0 mg). In some embodiments, the atosiban is administered to the subject in a single bolus dose of from about 5.75 mg to about 7.75 mg (e.g., a single bolus dose of about 5.75 mg, 6.00 mg, 6.25 mg, 6.50 mg, 6.75 mg, 7.00 mg, 7.25 mg, 7.50 mg, or 7.75 mg). In some embodiments, the atosiban is administered to the subject in a single bolus dose of from about 6.0 mg to about 7.5 mg (e.g., a single bolus dose of about 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, or 7.5 mg). In some embodiments, the atosiban is administered to the subject in a single bolus dose of from about 6.25 mg to about 7.25 mg (e.g., a single bolus dose of about 6.25 mg, 6.50 mg, 6.75 mg, 7.00 mg, or 7.25 mg). In some embodiments, the atosiban is administered to the subject in a single bolus dose of from about 6.5 mg to about 7.0 mg (e.g., a single bolus dose of about 6.50 mg, 6.75 mg, or 7.00. mg). In some embodiments, the atosiban is administered to the subject in a single bolus dose of about 6.75 mg.

In some embodiments, the single bolus dose of atosiban is administered to the subject intravenously.

In some embodiments, following the single bolus dose of atosiban, the atosiban is subsequently administered to the subject in an amount of from about 40 mg to about 70 mg by way of a continuous, intravenous infusion (e.g., an amount of about 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, or 70 mg by way of a continuous, intravenous infusion). In some embodiments, following the single bolus dose of atosiban, the atosiban is subsequently administered to the subject in an amount of from about 45 mg to about 65 mg by way of a continuous, intravenous infusion (e.g., an amount of about 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg by way of a continuous, intravenous infusion). In some embodiments, following the single bolus dose of atosiban, the atosiban is subsequently administered to the subject in an amount of from about 50 mg to about 60 mg by way of a continuous, intravenous infusion (e.g., an amount of about 50 mg, 51 mg, 52 mg, 53 mg, 54 mg, 55 mg, 56 mg, 57 mg, 58 mg, 59 mg, or 60 mg by way of a continuous, intravenous infusion). In some embodiments, following the single bolus dose of atosiban, the atosiban is subsequently administered to the subject in an amount of from about 52 mg to about 56 mg by way of a continuous, intravenous infusion (e.g., an amount of about 52 mg, 53 mg, 54 mg, 55 mg, or 56 mg by way of a continuous, intravenous infusion). In some embodiments, following the single bolus dose of atosiban, the atosiban is subsequently administered to the subject in an amount of about 54 mg by way of a continuous, intravenous infusion.

In some embodiments, the foregoing intravenous infusion is administered continuously over a period of from about 1 hour to about 5 hours, such as a period of about 1.0 hours, 1.25 hours, 1.5 hours, 1.75 hours, 2.0 hours, 2.25 hours, 2.5 hours, 2.75 hours, 3.0 hours, 3.25 hours, 3.5 hours, 3.5 hours, 3.75 hours, 4.0 hours, 4.25 hours, 4.5 hours, 4.75 hours, or 5.0 hours). In some embodiments, the foregoing intravenous infusion is administered continuously over a period of from about 1.5 hours to about 4.5 hours, such as a period of from about 1.5 hours, 1.75 hours, 2.0 hours, 2.25 hours, 2.5 hours, 2.75 hours, 3.0 hours, 3.25 hours, 3.5 hours, 3.5 hours, 3.75 hours, 4.0 hours, 4.25 hours, or 4.5 hours). In some embodiments, the foregoing intravenous infusion is administered continuously over a period of from about 2.0 hours to about 4.0 hours, such as a period of from about 2.0 hours, 2.25 hours, 2.5 hours, 2.75 hours, 3.0 hours, 3.25 hours, 3.5 hours, 3.5 hours, 3.75 hours, or 4.0 hours). In some embodiments, the foregoing intravenous infusion is administered continuously over a period of from about 2.5 hours to about 3.5 hours, such as a period of from about 2.5 hours, 2.75 hours, 3.0 hours, 3.25 hours, or 3.5 hours). In some embodiments, the foregoing intravenous infusion is administered continuously over a period of about 3.0 hours.

In some embodiments, following the foregoing continuous, intravenous infusion, the atosiban is subsequently administered to the subject in an amount of from about 240 mg to about 300 mg by way of a second continuous, intravenous infusion (e.g., an amount of about 240 mg, 245 mg, 250 mg, 255 mg, 260 mg, 265 mg, 270 mg, 275 mg, 280 mg, 285 mg, 290 mg, 295 mg, or 300 mg by way of a second continuous, intravenous infusion). In some embodiments, following the foregoing continuous, intravenous infusion, the atosiban is subsequently administered to the subject in an amount of from about 250 mg to about 290 mg by way of a second continuous, intravenous infusion (e.g., an amount of about 250 mg, 255 mg, 260 mg, 265 mg, 270 mg, 275 mg, 280 mg, 285 mg, or 290 mg by way of a second continuous, intravenous infusion). In some embodiments, following the foregoing continuous, intravenous infusion, the atosiban is subsequently administered to the subject in an amount of from about 250 mg to about 290 mg by way of a second continuous, intravenous infusion (e.g., an amount of about 250 mg, 255 mg, 260 mg, 265 mg, 270 mg, 275 mg, 280 mg, 285 mg, or 290 mg by way of a second continuous, intravenous infusion). In some embodiments, following the foregoing continuous, intravenous infusion, the atosiban is subsequently administered to the subject in an amount of from about 260 mg to about 280 mg by way of a second continuous, intravenous infusion (e.g., an amount of about 260 mg, 265 mg, 270 mg, 275 mg, or 280 mg by way of a second continuous, intravenous infusion). In some embodiments, following the foregoing continuous, intravenous infusion, the atosiban is subsequently administered to the subject in an amount of from about 265 mg to about 275 mg by way of a second continuous, intravenous infusion (e.g., an amount of about 265 mg, 270 mg, or 275 mg by way of a second continuous, intravenous infusion). In some embodiments, following the foregoing continuous, intravenous infusion, the atosiban is subsequently administered to the subject in an amount of about 270 mg by way of a second continuous, intravenous infusion.

In some embodiments, the second intravenous infusion is administered continuously over a period of from about 42 hours to about 48 hours, such as a period of about 42.0 hours, 42.25 hours, 42.5 hours, 42.75 hours, 43.0 hours, 43.25 hours, 43.5 hours, 43.75 hours, 44.0 hours, 44.25 hours, 44.5 hours, 44.75 hours, 45.0 hours, 45.25 hours, 45.5 hours, 45.75 hours, 46.0 hours, 46.25 hours, 46.5 hours, 46.75 hours, 47.0 hours, 47.25 hours, 47.5 hours, 47.75 hours, or 48.0 hours). In some embodiments, the second intravenous infusion is administered continuously over a period of from about 43 hours to about 47 hours, such as a period of about 43.0 hours, 43.25 hours, 43.5 hours, 43.75 hours, 44.0 hours, 44.25 hours, 44.5 hours, 44.75 hours, 45.0 hours, 45.25 hours, 45.5 hours, 45.75 hours, 46.0 hours, 46.25 hours, 46.5 hours, 46.75 hours, or 47.0 hours). In some embodiments, the second intravenous infusion is administered continuously over a period of from about 44 hours to about 46 hours, such as a period of about 44.0 hours, 44.25 hours, 44.5 hours, 44.75 hours, 45.0 hours, 45.25 hours, 45.5 hours, 45.75 hours, or 46.0 hours). In some embodiments, the second intravenous infusion is administered continuously over a period of from about 44.5 hours to about 45.5 hours, such as a period of about 44.5 hours, 44.75 hours, 45.0 hours, 45.25 hours, or 45.5 hours). In some embodiments, the second intravenous infusion is administered continuously over a period of about 44 hours.

In some embodiments, the atosiban is administered to the subject until the subject reaches a gestational age of at least about 34 weeks (e.g., a gestational age of from about 34 weeks to about 40 weeks, such as a gestational age of 34 weeks, 35 weeks, 36 weeks, 37 weeks, 38 weeks, 39 weeks, or 40 weeks), or until the subject undergoes delivery. In some embodiments, the atosiban is administered to the subject until the subject reaches a gestational age of about 37 weeks.

In some embodiments, administration of the PGF2α receptor antagonist and administration of the atosiban commence within about 48 hours of one another. In some embodiments, administration of the PGF2α receptor antagonist and administration of the atosiban commence within about 36 hours of one another. In some embodiments, administration of the PGF2α receptor antagonist and administration of the atosiban commence within about 24 hours of one another. In some embodiments, administration of the PGF2α receptor antagonist and administration of the atosiban commence within about 12 hours of one another. In some embodiments, administration of the PGF2α receptor antagonist and administration of the atosiban commence within about 6 hours of one another. In some embodiments, administration of the PGF2α receptor antagonist and administration of the atosiban commence at substantially the same time.

In some embodiments, the atosiban is formulated as an aqueous solution. The concentration of atosiban in the aqueous solution may be, for example, from about 5.0 mg/ml to about 10 mg mg/ml (e.g., about 5.0 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml, 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9.0 mg/ml, 9.1 mg/ml, 9.2 mg/ml, 9.3 mg/ml, 9.4 mg/ml, 9.5 mg/ml, 9.6 mg/ml, 9.7 mg/ml, 9.8 mg/ml, 9.9 mg/ml, or 10.0 mg/ml). In some embodiments, the concentration of atosiban in the aqueous solution is from about 5.5 mg/ml to about 9.5 mg/ml (e.g., about 5.5 mg/ml, 5.6 mg/ml, 5.7 mg/ml, 5.8 mg/ml, 5.9 mg/ml, 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9.0 mg/ml, 9.1 mg/ml, 9.2 mg/ml, 9.3 mg/ml, 9.4 mg/ml, or 9.5 mg/ml). In some embodiments, the concentration of atosiban in the aqueous solution is from about 6.0 mg/ml to about 9.0 mg/ml (e.g., about 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, or 9.0 mg/ml). In some embodiments, the concentration of atosiban in the aqueous solution is from about 6.5 mg/ml to about 8.5 mg/ml (e.g., about 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, or 8.5 mg/ml). In some embodiments, the concentration of atosiban in the aqueous solution is from about 7.0 mg/ml to about 8.0 mg/ml (e.g., about 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, or 8.0 mg/ml). In some embodiments, the concentration of atosiban in the aqueous solution is about 7.5 mg/ml.

The aqueous solution may further contain one or more pharmaceutically acceptable excipients, such as mannitol. In some embodiments, the concentration of mannitol in the aqueous solution is from about 40 mg/ml to about 60 mg/ml, such as from about 45 mg/ml to about 55 mg/ml (e.g., about 50 mg/ml).

In some embodiments, the aqueous solution has a pH of from about 4.0 to about 5.0 (e.g., a pH of about 4.0, 4.1, 4.2, 4.3, 4.4, 4.5, 4.6, 4.7, 4.8, 4.9, or 5.0). In some embodiments, the aqueous solution has a pH of from about 4.25 to about 4.75 (e.g., a pH of about 4.25, 4.30, 4.35, 4.40, 4.45, 4.50, 4.55, 4.60, 4.65, 4.70, or 4.75). In some embodiments, the aqueous solution has a pH of about 4.5.

In some embodiments, the subject has a singleton or twin pregnancy.

In some embodiments, the subject is undergoing or at risk of undergoing preterm labor. Conditions that may identify the subject as at risk of undergoing preterm labor include (i) the subject having a gestational age of from about 24 weeks to about 36 weeks prior to administration of the atosiban and the PGF2α receptor antagonist (e.g., a gestational age of from about 24 weeks to about 34 weeks, such as a gestational age of from about 28 weeks to about 33 and 6/7 weeks, prior to administration of the atosiban and the PGF2α receptor antagonist), (ii) the subject exhibiting four or more uterine contractions (e.g., each lasting at least 30 seconds) every 30 minutes prior to administration of the atosiban and the PGF2α receptor antagonist, (iii) the subject exhibiting a cervical dilation of from about 1 cm to about 4 cm prior to administration of the atosiban and the PGF2α receptor antagonist, (iv) the subject testing positive for the presence of fetal fibronectin and/or insulin-like growth factor-binding protein-1 (IGFBP-1) in a sample of cervical secretion obtained from the subject prior to administration of the atosiban and the PGF2α receptor antagonist; (v) the subject exhibiting a cervical length of about 25 mm or less prior to administration of the atosiban and the PGF2α receptor antagonist; (vi) the subject exhibiting a progressive cervical dilation prior to administration of the atosiban and the PGF2α receptor antagonist (e.g., a cervical dilation rate of from about 0.5 cm per hour to about 0.7 cm per hour prior to administration of the atosiban and the PGF2α receptor antagonist); and (vii) the subject exhibiting an effacement of at least 50% prior to administration of the atosiban and the PGF2α receptor antagonist. The subject may be one, for example, that exhibits one or more, or all, of these characteristics prior to the first administration of atosiban and the PGF2α receptor antagonist to the subject. In some embodiments, the subject exhibits one or more, or all, of these characteristics up to three hours prior to administration of the atosiban and the PGF2α receptor antagonist.

In some embodiments, the delivery by the subject is delayed by from about four hours to about six weeks following the first administration of the atosiban and the PGF2α receptor antagonist to the subject. For instance, the delivery may be delayed by from about 12 hours to about 28 days, by about 18 hours to about 21 days, by about 24 hours to about 14 days, by about 24 hours to about 14 days, by about 48 hours, or by about 7 days following the first administration of the atosiban and the PGF2α receptor antagonist to the subject. In some embodiments, following administration of the atosiban and the PGF2α receptor antagonist to the subject, the subject undergoes delivery at a gestational stage of at least about 34 weeks, such as a gestational age of about 34 weeks, 35 weeks, 36 weeks, 37 weeks, 38 weeks, 39 weeks, or 40 weeks. In some embodiments, following administration of the atosiban and the PGF2α receptor antagonist to the subject, the subject undergoes delivery at a gestational stage of about 37 weeks. In some embodiments, following administration of the atosiban and the PGF2α receptor antagonist to the subject, the subject undergoes delivery at a gestational stage of from about 36 weeks to about 40 weeks.

In an additional aspect, the disclosure features a kit containing a PGF2α receptor antagonist, such as the compound of any one of formulas (I) through (VIII), for example, compound (1), compound (2), or compound (3), among other PGF2α receptor antagonists described herein, and a package insert instructing a user of the kit to administer the PGF2α receptor antagonist to a pregnant subject (e.g., a pregnant human subject) in accordance with the method of any of the above-described aspects or embodiments of the disclosure. In some embodiments, the PGF2α receptor antagonist is a compound represented by formula (1).

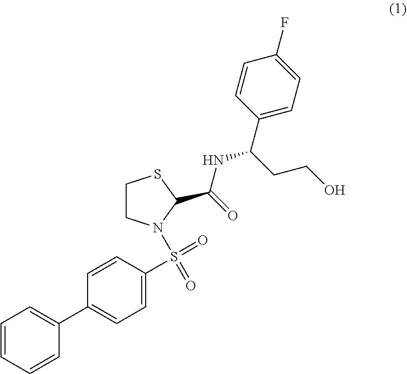

The PGF2α receptor antagonist may be a compound represented by formula (2)

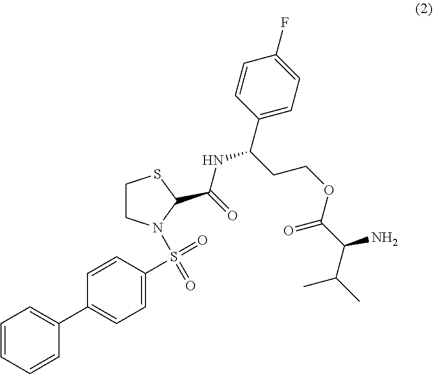

or a pharmaceutically acceptable salt thereof. For example, in some embodiments, the PGF2α receptor antagonist is the chloride salt of compound (2), represented by formula (3), below.

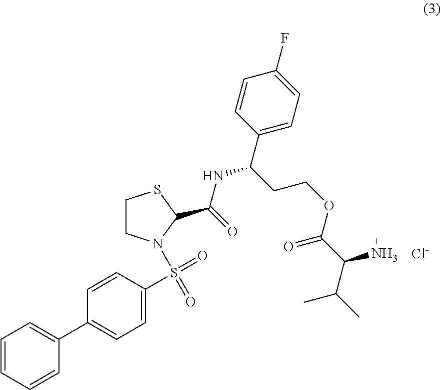

In some embodiments, the kit further contains atosiban, such as in a dosage form of an aqueous solution having a concentration of from about 5.0 mg/ml to about 10.0 mg/ml (e.g., a concentration of about 7.5 mg/ml). The aqueous solution may further contain one or more pharmaceutically acceptable excipients, such as mannitol, for example, at a concentration of from about 40 mg/ml to about 60 mg/ml, such as from about 45 mg/ml to about 55 mg/ml (e.g., about 50 mg/ml).

### Definitions

As used herein, the term “about” refers to a value that is within 10% above or below the value being described. For instance, a value of “about 5 mg” refers to a quantity that is from 4.5 mg to 5.5 mg.

As used herein, the term “affinity” refers to the strength of a binding interaction between two molecules, such as a ligand and a receptor. The term “K”, as used herein, is intended to refer to the inhibition constant of an antagonist for a particular molecule of interest, and is expressed as a molar concentration (M). K values for antagonist-target interactions can be determined, e.g., using methods established in the art. Methods that can be used to determine the K of an antagonist for a molecular target include competitive binding experiments, such as competitive radioligand binding assays, e.g., as described in U.S. Pat. Nos. 8,415,480; 9,447,055; and 9,834,528, the disclosures of each of which are incorporated herein by reference in their entirety. The term “K_{d}”, as used herein, is intended to refer to the dissociation constant, which can be obtained, e.g., from the ratio of the rate constant for the dissociation of the two molecules (k_{d}) to the rate constant for the association of the two molecules (kₐ) and is expressed as a molar concentration (M). K_{d} values for receptor-ligand interactions can be determined, e.g., using methods established in the art. Methods that can be used to determine the K_{d} of a receptor-ligand interaction include surface plasmon resonance, e.g., through the use of a biosensor system such as a BIACORE® system.

As used herein, the terms “benefit” and “response” are used interchangeably in the context of a subject, such as a pregnant human subject undergoing therapy for the treatment or prevention or preterm labor. These terms refers to any clinical improvement in the subject's condition. Exemplary benefits in the context of a subject undergoing, or at risk of undergoing, preterm labor and receiving treatment with atosiban and a prostaglandin receptor F2α (PGF2α) antagonist as described herein (e.g., a 1,3-thiazolidine-2-carboxamide compound described herein, such as (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof, such as (3S)-3-({[(2S)-3-(biphenyl-4-ylsulfonyl)-1,3-thiazolidin-2-yl]carbonyl}-amino)-3-(4-fluorophenyl)propyl L-valinate hydrochloride) include, without limitation, (i) a delay in the onset of delivery by the subject, such as a delay of one or more hours, days, or weeks (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others) following the first administration of atosiban and the PGF2α receptor antagonist to the subject, (ii) a delay in the onset of delivery by the subject such that the subject undergoes delivery at a gestational age of at least about 34 weeks, such as at a gestational age of from about 34 weeks to about 40 weeks (e.g., at a gestational age of about 34 weeks, 35 weeks, 36 weeks, 37 weeks, 38 weeks, 39 weeks, or 40 weeks) following administration of atosiban and the PGF2α receptor antagonist to the subject, (iii) a reduction in vaginal bleeding by the subject following administration of atosiban and the PGF2α receptor antagonist to the subject, (iv) a delay in the onset of amniorrhexis by the subject (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others, following the first administration of atosiban and the PGF2α receptor antagonist to the subject), (v) a reduction in the expression of one or more proinflammatory genes, such as cyclooxygenase-2 (Cox2) by the subject (as assessed, e.g., by observing a decrease in myometrial Cox2 expression) following administration of atosiban and the PGF2α receptor antagonist to the subject, and (vi) a reduction in the frequency of, peak amplitude of, duration of, and/or work done by, uterine contractions in the subject following administration of atosiban and the PGF2α receptor antagonist to the subject.

As used herein, the terms “bolus administration” and “bolus dose” are used interchangeably and refer to administration of a therapeutic agent to a subject such that the entire dose of the therapeutic agent is given to the subject in a single, discrete point in time. Examples of a bolus dose include the administration of an oxytocin receptor antagonist described herein, such as a 6.75-mg dose of atosiban, such that the entire 6.75-mg dose is given to the patient at a single point in time. In contrast, a “continuous administration,” such as a “continuous infusion,” refers to administration of a therapeutic agent (e.g., an oxytocin receptor antagonist described herein, such as atosiban) such that the entire dose of the therapeutic agent is given to the patient at a steady rate over an interval of time. Examples of a continuous administration include the administration of an oxytocin receptor antagonist described herein, such as a 54-mg dose of atosiban, such that the entire 54-mg dose is given to the patient at a steady rate over an interval of time (e.g., over the course of one or more minutes or hours).

As used herein, the term “crystalline” or “crystalline form” means having a physical state that is a regular three-dimensional array of atoms, ions, molecules or molecular assemblies. Crystalline forms have lattice arrays of building blocks called asymmetric units that are arranged according to well-defined symmetries into unit cells that are repeated in three-dimensions. In contrast, the term “amorphous” or “amorphous form” refers to an unorganized (no orderly) structure. The physical state of a therapeutic compound may be determined by exemplary techniques such as x-ray diffraction, polarized light microscopy and/or differential scanning calorimetry.

As used herein, the term “dose” refers to the quantity of a therapeutic agent, such as atosiban and/or PGF2α receptor antagonist described herein, that is administered to a subject for the treatment of a disorder or condition, such as to treat or prevent preterm labor in a pregnant subject (e.g., a pregnant human subject). A therapeutic agent as described herein may be administered in a single dose or in multiple doses. In each case, the therapeutic agent may be administered using one or more unit dosage forms of the therapeutic agent. For instance, a single dose of 1,000 mg of a therapeutic agent may be administered using, e.g., two 500 mg unit dosage forms of the therapeutic agent.

As used herein, the terms “effacement” and “effaced” refer to the preparation of the cervix for delivery, a process that manifests in a progressive thinning of the cervix. Effacement may be expressed as a percentage, such as 10%, 20%, 30%, 40%, 50%, 60%, 70%, 80%, 90%, or 100%. As effacement occurs, the cervix shortens, eventually becoming part of the lower uterine wall. Methods for measuring cervical effacement on the basis of the thickness and dilation of the cervix are known in the art and are described, for example, in U.S. Pat. No. 5,876,357, the disclosure of which is incorporated herein by reference as it pertains to the measurement of cervical effacement.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “gestational age” describes how far along a particular pregnancy is, and is measured from the first day of a pregnant female subject's last menstrual cycle to the current date. As used herein, the term “labor” (which may also be termed birth) relates to the expulsion of the fetus and placenta from the uterus of a pregnant female subject. For a normal human pregnancy, labor generally occurs at a gestational age of approximately 40 weeks. “Preterm labor” as used herein refers to a condition in which labor commences more than three weeks before the full gestation period, which is typically about 40 weeks in human subjects. That is, preterm labor may occur at any stage prior to, e.g., 38 weeks of gestation. Preterm labor typically leads to the occurrence of labor, or physiological changes associated with labor in a pregnant female subject, if not treated. Preterm labor may or may not be associated with vaginal bleeding or rupture of uterine membranes. Preterm labor may also be referred to as premature labor.

As used herein, the term “once daily” refers to administration of a therapeutic agent to a subject in a single dose each day. The time of day at which the therapeutic agent is administered to the subject may be constant or may vary. Similarly, the terms “twice daily,” “three times daily,” and the like refer to administration of a therapeutic agent to a subject in two doses or three doses each day, respectively.

As used herein, the term “progressive cervical dilation” refers to a steady opening of the cervix in a subject, such as a pregnant human subject undergoing or at risk of undergoing preterm labor. A subject is considered to exhibit progressive cervical dilation if the subject's cervix is opening (i.e., dilating) at a constant linear rate, such as a rate of from about 0.5 cm per hour to about 0.7 cm per hour.

As used herein, the term “prostaglandin F2α receptor antagonist” or “PGF2α receptor antagonist” refers to a compound that specifically and/or selectively binds the PGF2α receptor and is capable of inhibiting receptor signaling, for instance, in uterine myocytes. Inhibition of PGF2α receptor signal transduction may manifest, for example, in suppression of phospholipase C activity, which may be observed as a reduction in: (i) phosphatidylinositol-4,5-bisphosphate (PIP₂) cleavage, (ii) diacylglycerol (DAG) production, (iii) inositol-1,4,5-trisphosphate (IP₃) production, and/or (iv) intracellular calcium (Ca²⁺) release by sarcoplasmic reticula. Inhibition of PGF2α receptor signaling may also manifest as a reduction in one or more downstream physiological events associated with calcium mobilization, such as a reduction in uterine muscle contractions and/or reduction in endothelial cell necrosis within the corpus luteum, a progesterone-secreting structure that supports a developing fetus. PGF2α receptor antagonists that may be used in conjunction with the compositions and methods described herein include 1,3-thiazolidine-2-carboxamides, such as (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof (e.g., (3S)-3-({[(2S)-3-(biphenyl-4-ylsulfonyl)-1,3-thiazolidin-2-yl]carbonyl}-amino)-3-(4-fluorophenyl)propyl L-valinate hydrochloride), or another 1,3-thiazolidine-2-carboxamide that gives rise to 3-([1,1′-biphenyl]-4-ylsulfonyl)-N-[1-(4-fluorophenyl)-3-hydroxypropyl]-1,3-thiazolidine-2-carboxamide in vivo, e.g., as described in U.S. Pat. Nos. 8,415,480; 9,447,055; and 9,834,528, the disclosures of each of which are incorporated herein by reference in their entirety.

As used herein, the term “IC₅₀” refers to the concentration of a substance (antagonist) that reduces the efficacy of a reference agonist or the constitutive activity of a biological target by 50%, e.g., as measured in a competitive ligand binding assay. Exemplary competitive ligand binding assays include competitive radioligand binding assays, competitive enzyme-linked immunosorbent assays (ELISA), and fluorescence anisotropy-based assays, among others known in the art.

As used herein in the context of providing or administering two or more therapeutic agents to a subject, the phrase “in combination with” refers to the delivery of two or more therapeutic agents to a subject (e.g., a mammalian subject, such as a pregnant human subject) either (i) concurrently or (ii) at different times such that the later-administered agent is provided to the subject while there is still a detectable concentration of the earlier-administered agent, or a metabolite thereof, in the plasma and/or one or more tissue(s) (e.g., myometrial tissue) of the subject. For example, one therapeutic agent may be administered to a subject in combination with another by administering both agents to the subject concurrently, such as in a single pharmaceutical composition or in separate compositions that are administered to the subject simultaneously (e.g., by different routes of administration). In another example, one therapeutic agent may be administered to a subject in combination with another by first administering to the subject one therapeutic agent and subsequently administering the other therapeutic agent, either by the same or different route of administration, while there is still a detectable quantity of the first agent in the plasma and/or tissue(s) of the subject. After the first administration of each agent (e.g., concurrently or at different times), it is not necessary that the subject receive the remaining agent(s) each and every time the subject receives a dose of the first agent. For instance, two or more agents are said to be administered “in combination with” one another if the subject receives a daily dosage of a first agent and a weekly dosage of the remaining agent(s). The timing of administration of the two or more agents need not coincide.

As used herein, the term “menstrual cycle” refers to a recurring cycle of physiological changes in females, such as human females, that is associated with reproductive fertility. While the cycle length may vary from woman to woman, 28 days is generally taken as representative of the average ovulatory cycle in human females.

As used herein, the term “pharmaceutical composition” means a mixture containing a therapeutic compound to be administered to a patient, such as a mammal, e.g., a human, in order to prevent, treat or control a particular disease or condition affecting the mammal, such as preterm labor or dysmenorrhea.

As used herein, the term “pharmaceutically acceptable” refers to those compounds, materials, compositions and/or dosage forms, which are suitable for contact with the tissues of a patient, such as a mammal (e.g., a human) without excessive toxicity, irritation, allergic response and other problem complications commensurate with a reasonable benefit/risk ratio.

As used herein in the context of administration of a therapeutic agent, the term “periodically” refers to administration of the agent two or more times over the course of a treatment period (e.g., two or more times daily, weekly, monthly, or yearly).

As used herein in the context of therapeutic treatment, the terms “provide” and “providing” refer to the delivery of a therapeutic agent to a subject (e.g., a mammalian subject, such as a human) in need of treatment, such as a subject experiencing or at risk of undergoing preterm labor. A therapeutic agent may be provided to a subject in need thereof, for instance, by direct administration of the therapeutic agent to the subject, or by administration of a prodrug that is converted in vivo to the therapeutic agent upon administration of the prodrug to the subject. Exemplary prodrugs include, without limitation, esters, phosphates, and other chemical functionalities susceptible to hydrolysis upon administration to a subject. Prodrugs include those known in the art, such as those described, for instance, in Vig et al., Adv. Drug Deliv. Rev. 65:1370-1385 (2013), and Huttunen et al., Pharmacol. Rev. 63:750-771 (2011), the disclosures of each of which are incorporated herein by reference.

As used herein, the term “sample” refers to a specimen (e.g., blood, blood component (e.g., serum or plasma), urine, saliva, amniotic fluid, cerebrospinal fluid, tissue (e.g., placental or myometrial), pancreatic fluid, chorionic villus sample, and cells) isolated from a patient.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, proteoglycan, or glycosaminoglycan) that specifically binds to a protein will bind to the protein, e.g., with a K_{D} of less than 100 nM. For example, a ligand that specifically binds to a protein may bind to the protein with a K_{D} of up to 100 nM (e.g., between 1 pM and 100 nM). A ligand that does not exhibit specific binding to a protein or a domain thereof will exhibit a K_{D} of greater than 100 nM (e.g., greater than 200 nM, 300 nM, 400 nM, 500 nM, 600 nm, 700 nM, 800 nM, 900 nM, 1 μM, 100 μM, 500 μM, or 1 mM) for that particular protein or domain thereof. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject’ and “patient” are used interchangeably and refer to an organism, such as a mammal (e.g., a human) that receives therapy for the treatment or prevention of preterm labor as described herein. Patients that may receive therapy, or that are considered to be in need of therapy, for the treatment or prevention of preterm labor include subjects (e.g., pregnant human subjects) that are actively undergoing preterm labor, as well as those at risk of undergoing preterm labor. A variety of clinical indicators can be used to identify a patient as “at risk” of undergoing preterm labor. Examples of patients (e.g., human patients) that are “at risk” of undergoing preterm labor include (i) the subject having a gestational age of from about 24 weeks to about 36 weeks prior to administration of a tocolytic agent, such as atosiban and/or a PGF2α receptor antagonist described herein (e.g., a gestational age of from about 24 weeks to about 34 weeks, such as a gestational age of from about 28 weeks to about 33 and 6/7 weeks, e.g., prior to administration of the atosiban and the PGF2α receptor antagonist), (ii) the subject exhibiting four or more uterine contractions per 30 minutes, e.g., prior to administration of the tocolytic agent(s), (iii) the subject exhibiting a cervical dilation of from about 1 cm to about 4 cm, e.g., prior to administration of the tocolytic agent(s), (iv) the subject testing positive for the presence of fetal fibronectin and/or insulin-like growth factor-binding protein-1 (IGFBP-1) in a sample of cervical secretion obtained from the subject, e.g., prior to administration of the tocolytic agent(s), and (v) the subject exhibiting a cervical length of about 25 mm or less, e.g., prior to administration of the tocolytic agent(s). Pregnant subjects that are “at risk” of undergoing preterm labor may exhibit one or more, or all, of these characteristics, for example, prior to the first administration of atosiban and a PGF2α receptor antagonist in accordance with the compositions and methods described herein.

As used herein, the term “substantially the same time” refers to administration of two or more therapeutic agents to a subject within a few moments of one another or at the exact same time. The term “substantially the same time” is intended to embrace instances in which two or more therapeutic agents are administered to a subject without a significant delay in between administration of each agent, such as instances in which a second or subsequent therapeutic agent is administered to a subject following administration of the first therapeutic agent in the most expeditious manner permitted within the confines of any practical restrictions observed by a health care practitioner. Examples of such practical restrictions include the time required for a practitioner to prepare a patient for an injection, such as an intravenous infusion, following the administration of a first therapeutic agent, which may have been administered orally. For the avoidance of doubt, instances in which a subject is first administered an oral therapeutic agent and is then immediately prepared for, and administered a dose of, an injectable therapeutic agent are considered to be instances of administration of both therapeutic agents at “substantially the same time.”

As used herein in the context of an oxytocin receptor antagonist, such as atosiban, and a PGF2α receptor antagonist, such as a compound of any one of formulas (I) through (VIII) herein, the term “therapeutically effective amounts” refers to quantities of each agent that, when administered in combination with one another, are capable of achieving a beneficial treatment outcome for a subject undergoing or at risk of undergoing preterm labor. For example, “therapeutically effective amounts” of an oxytocin receptor antagonist and a PGF2α receptor antagonist described herein are amounts of each agent that, when administered in combination with one another, are together capable of achieving (i) a delay in the onset of delivery by a pregnant human subject, such as a delay of one or more hours, days, or weeks (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others) following the first administration of each agent to the subject, (ii) a delay in the onset of delivery by the subject such that the subject undergoes delivery at a gestational age of at least about 34 weeks, such as at a gestational age of from about 34 weeks to about 40 weeks (e.g., at a gestational age of about 34 weeks, 35 weeks, 36 weeks, 37 weeks, 38 weeks, 39 weeks, or 40 weeks) following administration of each agent to the subject, (iii) a reduction in vaginal bleeding by the subject following administration of each agent to the subject, (iv) a delay in the onset of amniorrhexis by the subject (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others, following the first administration of each agent to the subject, (v) a reduction in the expression of one or more proinflammatory genes, such as cyclooxygenase-2 (Cox2) by the subject (as assessed, e.g., by observing a decrease in myometrial Cox2 expression) following administration of each agent to the subject, and (vi) a reduction in the frequency of, peak amplitude of, duration of, and/or work done by, uterine contractions in the subject following administration of each agent to the subject.

The two or more agents are said to be administered “in combination with” one another if they are administered at the same time to the subject (e.g., in a single or in separate compositions) or at different times, such that the later-administered agent is provided to the subject while there is still a detectable concentration of the earlier-administered agent, or a metabolite thereof, in the plasma and/or one or more tissue(s) (e.g., myometrial tissue) of the subject. For example, one therapeutic agent may be administered to a subject in combination with another by administering both agents to the subject concurrently, such as in a single pharmaceutical composition or in separate compositions that are administered to the subject simultaneously (e.g., by different routes of administration). In another example, one therapeutic agent may be administered to a subject in combination with another by first administering to the subject one therapeutic agent and subsequently administering the other therapeutic agent, either by the same or different route of administration. After the first administration of each agent (e.g., concurrently or at different times), it is not necessary that the subject receive the remaining agent each and every time the subject receives a dose of the first agent. For instance, the agents are said to be administered “in combination with” one another if the subject receives a daily dosage of a first agent and a weekly dosage of the remaining agent. The timing of administration of the agents need not coincide.

As used herein, the term “tocolytic agent” refers to a substance capable of delaying the onset of labor in a subject (e.g., a mammalian subject, such as a human subject). Tocolytic agents may function to suppress uterine contractility, for instance, by inhibiting the mobilization of intracellular Ca²⁺. Exemplary tocolytic agents are described, for instance, in Haas et al. Int. J. Women's Health. 6:343-349 (2014), the disclosure of which is incorporated herein by reference. Tocolytic agents for use in conjunction with the compositions and methods described herein include, without limitation, oxytocin receptor antagonists, such as atosiban, as well as PGF2α receptor antagonists, such as a PGF2α receptor antagonist described herein (e.g., a 1,3-thiazolidine-2-carboxamide compound described herein, such as (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof, such as (3S)-3-({[(2S)-3-(biphenyl-4-ylsulfonyl)-1,3-thiazolidin-2-yl]carbonyl}-amino)-3-(4-fluorophenyl)propyl L-valinate hydrochloride).

As used herein in the context of preterm labor, the terms “treat” or “treatment” refer to therapeutic treatment, in which the object is to delay the onset of labor by a pregnant subject (e.g., a pregnant human subject). Successful treatment of a pregnant subject with atosiban and a PGF2α receptor antagonist described herein (e.g., a 1,3-thiazolidine-2-carboxamide compound described herein, such as (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof, such as (3S)-3-({[(2S)-3-(biphenyl-4-ylsulfonyl)-1,3-thiazolidin-2-yl]carbonyl}-amino)-3-(4-fluorophenyl)propyl L-valinate hydrochloride) may manifest in a variety of ways. Desired treatment outcomes that may be achieved using the compositions and methods described herein include, without limitation, a delay in the onset of delivery by the subject, such as a delay of one or more hours, days, or weeks (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others) following the first administration of atosiban and the PGF2α receptor antagonist to the subject. Another clinical indicator of successful treatment is the observation of a delay in the onset of delivery by the subject such that the subject undergoes delivery at a gestational age of at least about 34 weeks, such as at a gestational age of from about 34 weeks to about 40 weeks (e.g., at a gestational age of about 34 weeks, 35 weeks, 36 weeks, 37 weeks, 38 weeks, 39 weeks, or 40 weeks) following administration of atosiban and the PGF2α receptor antagonist to the subject. Successful treatment may also be signaled by a reduction in vaginal bleeding by the subject following administration of atosiban and the PGF2α receptor antagonist to the subject, as well as by a delay in the onset of amniorrhexis by the subject (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others, following the first administration of atosiban and the PGF2α receptor antagonist to the subject. In another example, a subject (e.g., a pregnant human subject) may be considered to have undergone successful treatment upon observing a reduction in the expression of one or more proinflammatory genes, such as cyclooxygenase-2 (Cox2) by the subject (as assessed, e.g., by observing a decrease in myometrial Cox2 expression) following administration of atosiban and the PGF2α receptor antagonist to the subject. Additional clinical indicators of successful treatment of preterm labor include a reduction in the frequency of, peak amplitude of, duration of, and/or work done by, uterine contractions in the subject following administration of atosiban and the PGF2α receptor antagonist to the subject.

As used herein, the term “treatment period” refers to a duration of time over which a patient may be administered a therapeutic agent, such as atosiban and a PGF2α receptor antagonist described herein, so as to treat or prevent preterm labor. Treatment periods as described herein may have a duration of several hours, days, or weeks. For instance, a treatment period for administration of a 1,3-thiazolidine-2-carboxamide compound described herein, such as (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof (for example, (3S)-3-({[(2S)-3-(biphenyl-4-ylsulfonyl)-1,3-thiazolidin-2-yl]carbonyl}-amino)-3-(4-fluorophenyl)propyl L-valinate hydrochloride) may last for from about 1 hour weeks to about 16 weeks, or until the subject undergoes delivery. In some embodiments, for example, the atosiban is administered to the subject one or more times every 12 to 72 hours, and the PGF2α receptor antagonist is administered to the subject one or more times (e.g., once or twice) daily.

As used herein, the term “C₁-C₆-alkyl” refers to monovalent alkyl groups having 1 to 6 carbon atoms. This term is exemplified by groups such as methyl, ethyl, n-propyl, isopropyl, n-butyl, isobutyl, tert-butyl, n-hexyl and the like.

As used herein, the term “aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenanthrenyl and the like.

As used herein, the term “C₁-C₆-alkyl aryl” refers to C₁-C₆-alkyl groups having an aryl substituent, including benzyl, phenethyl and the like.

As used herein, the term “heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydrojbenzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

As used herein, the term “C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like. “C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

As used herein, the term “C₂-C₆-alkenyl aryl” refers to C₂-C₆-alkenyl groups having an aryl substituent, including 2-phenylvinyl and the like.

As used herein, the term “C₂-C₆-alkenyl heteroaryl” refers to C₂-C₆-alkenyl groups having a heteroaryl substituent, including 2-(3-pyridinyl)vinyl and the like.

As used herein, the term “C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

As used herein, the term “C₂-C₆-alkynyl aryl” refers to C₂-C₆-alkynyl groups having an aryl substituent, including phenylethynyl and the like.

As used herein, the term “C₂-C₆-alkynyl heteroaryl” refers to C₂-C₆-alkynyl groups having a heteroaryl substituent, including 2-thienylethynyl and the like.

As used herein, the term “C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

As used herein, the term “heterocycloalkyl” refers to a C₃-C₈-cycloalkyl group according to the definition above, in which up to 3 carbon atoms are replaced by heteroatoms chosen from the group consisting of O, S, NR, R being defined as hydrogen or methyl. Preferred heterocycloalkyl include pyrrolidine, piperidine, piperazine, 1-methylpiperazine, morpholine, and the like.

As used herein, the term “C₁-C₆-alkyl cycloalkyl” refers to C₁-C₆-alkyl groups having a cycloalkyl substituent, including cyclohexylmethyl, cyclopentylpropyl, and the like. “C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

As used herein, the term “carboxy” refers to the group —C(O)OH.

As used herein, the term “C₁-C₅-alkyl carboxy” refers to C₁-C₅-alkyl groups having a carboxy substituent, including 2-carboxyethyl and the like.

As used herein, the term “acyl” refers to the group —C(O)R where R includes “C₁-C₆-alkyl”, “aryl”, “heteroaryl”, “C₁-C₆-alkyl aryl” or “C₁-C₆-alkyl heteroaryl”.

As used herein, the term “C₁-C₅-alkyl acyl” refers to C₁-C₅-alkyl groups having an acyl substituent, including 2-acetylethyl and the like.

As used herein, the term “acyloxy” refers to the group —OC(O)R where R includes “C₁-C₆-alkyl”, “aryl”, “heteroaryl”, “C₁-C₆-alkyl aryl” or “C₁-C₆-alkyl heteroaryl”.

As used herein, the term “C₁-C₅-alkyl acyloxy” refers to C₁-C₅-alkyl groups having an acyloxy substituent, including 2-(acetyloxy)ethyl and the like.

As used herein, the term “alkoxy” refers to the group —O—R where R includes “C₁-C₆-alkyl” or “aryl” or “heteroaryl” or “C₁-C₆-alkyl aryl” or “C₁-C₆-alkyl heteroaryl”. Preferred alkoxy groups include by way of example, methoxy, ethoxy, phenoxy and the like.

As used herein, the term “C₁-C₅-alkyl alkoxy” refers to C₁-C₅-alkyl groups having an alkoxy substituent, including 2-ethoxyethyl and the like.

As used herein, the term “alkoxycarbonyl” refers to the group —C(O)OR where R includes H, “C₁-C₆-alkyl” or “aryl” or “heteroaryl” or “C₁-C₆-alkyl aryl” or “C₁-C₆-alkyl heteroaryl”.

As used herein, the term “C₁-C₅-alkyl alkoxycarbonyl” refers to C₁-C₅-alkyl groups having an alkoxycarbonyl substituent, including 2-(benzyloxycarbonyl)ethyl and the like.

As used herein, the term “aminocarbonyl” refers to the group —C(O)NRR′ where each R, R′ includes independently hydrogen or C₁-C₆-alkyl or aryl or heteroaryl or “C₁-C₆-alkyl aryl” or “C₁-C₆-alkyl heteroaryl”.

As used herein, the term “C₁-C₅-alkyl aminocarbonyl” refers to C₁-C₅-alkyl groups having an aminocarbonyl substituent, including 2-(dimethylaminocarbonyl)ethyl and the like.

As used herein, the term “acylamino” refers to the group —NRC(O)R′ where each R, R′ is independently hydrogen or “C₁-C₆-alkyl” or “aryl” or “heteroaryl” or “C₁-C₆-alkyl aryl” or “C₁-C₆-alkyl heteroaryl”.

As used herein, the term “C₁-C₅-alkyl acylamino” refers to C₁-C₅-alkyl groups having an acylamino substituent, including 2-(propionylamino)ethyl and the like.

As used herein, the term “ureido” refers to the group —NRC(O)NR′R″ where each R, R′, R″ is independently hydrogen or “C₁-C₆-alkyl” or “aryl” or “heteroaryl” or “C₁-C₆-alkyl aryl” or “C₁-C₆-alkyl heteroaryl” “cycloalkyl” or “heterocycloalkyl”, and where R′ and R″, together with the nitrogen atom to which they are attached, can optionally form a 3-8-membered heterocycloalkyl ring.

As used herein, the term “C₁-C₅-alkyl ureido” refers to C₁-C₅-alkyl groups having an ureido substituent, including 2-(N′-methylureido)ethyl and the like.

As used herein, the term “amino” refers to the group —NRR′ where each R,R′ is independently hydrogen or “C₁-C₆-alkyl” or “aryl” or “heteroaryl” or “C₁-C₆-alkyl aryl” or “C₁-C₆-alkyl heteroaryl” or “cycloalkyl” or “heterocycloalkyl” and where R and R′, together with the nitrogen atom to which they are attached, can optionally form a 3-8-membered heterocycloalkyl ring.

As used herein, the term “C₁-C₅-alkyl amino” refers to C₁-C₆-alkyl groups having an amino substituent, including 2-(1-pyrrolidinyl)ethyl and the like.

As used herein, the term “ammonium” refers to a positively charged group —N⁺RR′R″, where each R,R′,R″ is independently “C₁-C₆-alkyl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

As used herein, the term “halogen” refers to fluoro, chloro, bromo and iodo atoms.

As used herein, the term “sulfonyloxy” refers to a group —OSO₂—R wherein R is selected from H, “C₁-C₆-alkyl”, “C₁-C₆-alkyl” substituted with halogens, e.g., an —OSO₂—CF₃ group, “aryl”, “heteroaryl”, “C₁-C₆-alkyl aryl” or “C₁-C₆-alkyl heteroaryl”.

As used herein, the term “C₁-C₅-alkyl sulfonyloxy” refers to C₁-C₅-alkyl groups having a sulfonyloxy substituent, including 2-(methylsulfonyloxy)ethyl and the like.

As used herein, the term “sulfonyl” refers to group “—SO₂—R” wherein R is selected from H, “aryl”, “heteroaryl”, “C₁-C₅-alkyl”, “C₁-C₅-alkyl” substituted with halogens, e.g., an —SO₂—CF₃ group, “C₁-C₆-alkyl aryl” or “C₁-C₆-alkyl heteroaryl”.

As used herein, the term “C₁-C₅-alkyl sulfonyl” refers to C₁-C₅-alkyl groups having a sulfonyl substituent, including 2-(methylsulfonyl)ethyl and the like.

As used herein, the term “sulfinyl” refers to a group “—S(O)—R” wherein R is selected from H, “C₁-C₆-alkyl”, “C₁-C₆-alkyl” substituted with halogens, e.g., a —SO—CF₃ group, “aryl”, “heteroaryl”, “C₁-C₆-alkyl aryl” or “C₁-C₆-alkyl heteroaryl”.

As used herein, the term “C₁-C₆-alkyl sulfinyl” refers to C₁-C₆-alkyl groups having a sulfinyl substituent, including 2-(methylsulfinyl)ethyl and the like.

As used herein, the term “sulfanyl” refers to groups —S—R where R includes “C₁-C₆-alkyl” or “aryl” or “hetero-aryl” or “C₁-C₆-alkyl aryl” or “C₁-C₆-alkyl heteroaryl”. Preferred sulfanyl groups include methylsulfanyl, ethylsulfanyl, and the like.

As used herein, the term “C₁-C₅-alkyl sulfanyl” refers to C₁-C₅-alkyl groups having a sulfanyl substituent, including 2-(ethylsulfanyl)ethyl and the like. “Sulfonylamino” refers to a group —NRSO₂—R′ where each R, R′ is independently hydrogen or “C₁-C₆-alkyl” or “aryl” or “heteroaryl” or “C₁-C₆-alkyl aryl” or “C₁-C₆-alkyl heteroaryl”.

As used herein, the term “C₁-C₅-alkyl sulfonylamino” refers to C₁-C₅-alkyl groups having a sulfonylamino substituent, including 2-(ethylsulfonylamino)ethyl and the like.

Unless otherwise constrained by the definition of the individual substituent, the above set out groups, like “alkyl”, “alkenyl”, “alkynyl”, “aryl” and “heteroaryl” etc. groups can optionally be substituted with from 1 to substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “heteroaryl”, “sulfinyl”, “sulfonyl”, “alkoxy”, “sulfanyl”, “halogen”, “carboxy”, trihalomethyl, cyano, hydroxy, mercapto, nitro, and the like. Alternatively said substitution could also comprise situations where neighboring substituents have undergone ring closure, notably when vicinal functional substituents are involved, thus forming, e.g., lactams, lactones, cyclic anhydrides, but also acetals, thioacetals, aminals formed by ring closure for instance in an effort to obtain a protective group.

As used herein, the term “pharmaceutically acceptable salts or complexes” refers to salts or complexes of the below-identified compounds of formulae (I) and (II) that retain the desired biological activity. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. Said compounds can also be administered as pharmaceutically acceptable quaternary salts known by a person skilled in the art, which specifically include the quaternary ammonium salt of the formula —NR,R′,R″ ⁺Z⁻, wherein R, R′, R″ is independently hydrogen, alkyl, or benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, and Z is a counterion, including chloride, bromide, iodide, —O— alkyl, toluenesulfonate, methyl sulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

As used herein, the term “derivative” refers to structural variants of a reference compound, such as variants that differ from the reference compound by the inclusion and/or location of one or more substituents, as well as variants that are isomers of a reference compound, such as structural isomers (e.g., regioisomers) or stereoisomers (e.g., enantiomers or diastereomers).

The structural compositions described herein also include tautomers, its geometrical isomers, enantiomers, diastereomers, and racemic forms, as well as pharmaceutically acceptable salts thereof. Such salts include, e.g., acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

### BRIEF DESCRIPTION OF THE FIGURES

FIG. 1 is a graph showing the mean (plus/minus standard deviation) plasma concentration of compound (2), when administered to pregnant human subjects receiving atosiban infusion therapy, as described in Part A of Example One, below.

FIG. 2 is a graph showing individual ductus arteriosus pulsatility indices of patients administered compound (2) in combination with atosiban, as described in Part A of Example One, below.

FIG. 3 is a graph showing the mean (plus/minus standard deviation) Amniotic Fluid Index of patients administered compound (2) in combination with atosiban, as described in Part A of Example One, below.

FIG. 4 is a graph showing the percentage of subjects that underwent delivery within 48 hours of initiating treatment with atosiban and compound (2) (in Part B of the study described in Example One, below) as compared to the percentage of subjects that underwent delivery within 48 hours of initiating treatment with atosiban and placebo. Categories along the horizontal axis represent, from left to right: (i) the overall percentage of subjects in each treatment cohort that underwent delivery within 48 hours of initiating therapy; (ii) the percentage of subjects in each cohort having a gestational age (“GA”) of from 24 weeks to 30 weeks that underwent delivery within 48 hours of initiating therapy; (iii) the percentage of subjects in each cohort having a gestational age of from 30 weeks to 34 weeks that underwent delivery within 48 hours of initiating therapy; (iv) the percentage of subjects in each cohort having a singleton pregnancy that underwent delivery within 48 hours of initiating therapy; and (v) the percentage of subjects in each cohort having a twin pregnancy that underwent delivery within 48 hours of initiating therapy. Beneath the horizontal axis, an odds ratio (“OR”) is shown that quantifies the association between inclusion of compound (2) in the treatment regimen and the delay in subjects' onset of labor for each cohort. A 90% confidence internal (“CI”) is provided for each odds ratio.

FIG. 5 is a graph showing the percentage of subjects having a singleton pregnancy that underwent delivery within 48 hours of initiating treatment with atosiban and compound (2) (in Part B of the study described in Example One, below) as compared to the percentage of subjects having a singleton pregnancy that underwent delivery within 48 hours of initiating treatment with atosiban and placebo. Categories along the horizontal axis represent, from left to right: (i) the overall percentage of subjects in each treatment cohort having a singleton pregnancy that underwent delivery within 48 hours of initiating therapy; (ii) the percentage of subjects in each cohort having a singleton pregnancy and a gestational age (“GA”) of from 24 weeks to 30 weeks that underwent delivery within 48 hours of initiating therapy; and (iii) the percentage of subjects in each cohort having a singleton pregnancy and a gestational age of from 30 weeks to 34 weeks that underwent delivery within 48 hours of initiating therapy. Beneath the horizontal axis, an odds ratio (“OR”) is shown that quantifies the association between inclusion of compound (2) in the treatment regimen and the delay in subjects' onset of labor for each cohort. A 90% confidence internal (“CI”) is provided for each odds ratio.

FIG. 6 is a graph showing the percentage of subjects having a singleton pregnancy that underwent delivery within 7 days of initiating treatment with atosiban and compound (2) (in Part B of the study described in Example One, below) as compared to the percentage of subjects having a singleton pregnancy that underwent delivery within 7 days of initiating treatment with atosiban and placebo. Categories along the horizontal axis represent, from left to right: (i) the overall percentage of subjects in each treatment cohort having a singleton pregnancy that underwent delivery within 7 days of initiating therapy; (ii) the percentage of subjects in each cohort having a singleton pregnancy and a gestational age (“GA”) of from 24 weeks to 30 weeks that underwent delivery within 7 days of initiating therapy; and (iii) the percentage of subjects in each cohort having a singleton pregnancy and a gestational age of from 30 weeks to 34 weeks that underwent delivery within 7 days of initiating therapy. Beneath the horizontal axis, an odds ratio (“OR”) is shown that quantifies the association between inclusion of compound (2) in the treatment regimen and the delay in subjects' onset of labor for each cohort. A 90% confidence internal (“CI”) is provided for each odds ratio.

FIG. 7 is a graph showing the reduction in uterine contractility experienced by subjects treated with atosiban and compound (2) (in Part B of the study described in Example One, below) as compared to the reduction in uterine contractility experienced by subjects treated with atosiban and placebo. Values along the vertical axis represent the change from baseline in the quantity of uterine contractions per 30-minute interval experienced by subjects in either cohort. Values along the horizontal axis represent the time (in hours) elapsed after initiation of treatment with either (i) atosiban and placebo or (ii) atosiban and compound (2).

FIG. 8 is a graph showing the delivery-delaying effects of atosiban and compound (2) on pregnant human subjects as compared with the effects of atosiban and placebo. Data were obtained from Part B of the clinical trial described in Example One, below. Values along the vertical axis represent the proportion of trial participants that underwent delivery. Values along the horizontal axis represent the time (in days) elapsed between (i) initiation of treatment with either atosiban and placebo (solid curve) or atosiban and compound (2) (dashed curve) and (ii) delivery.

### DETAILED DESCRIPTION

Provided herein are compositions and methods for the treatment and/or prevention of preterm labor in a patient, such as a mammalian patient (e.g., a humane female patient). Using the compositions and methods described herein, a prostaglandin F2α (PGF2α) receptor antagonist may be administered to a patient undergoing or at risk of undergoing premature parturition, so as to slow the onset of delivery, for instance, by a matter of hours, days, or weeks. The PGF2α receptor antagonist may be administered to the patient in combination with an oxytocin receptor antagonist, such as atosiban. The PGF2α receptor antagonist may be, for example, a 1,3-thiazolidine-2-carboxamide compound described herein, such as (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof, such as (3S)-3-({[(2S)-3-(biphenyl-4-ylsulfonyl)-1,3-thiazolidin-2-yl]carbonyl}-amino)-3-(4-fluorophenyl)propyl L-valinate hydrochloride, or another 1,3-thiazolidine-2-carboxamide that gives rise to 3-([1,1′-biphenyl]-4-ylsulfonyl)-N-[1-(4-fluorophenyl)-3-hydroxypropyl]-1,3-thiazolidine-2-carboxamide in vivo.

The compositions and methods of the present disclosure provide a variety of beneficial treatment outcomes. Using the compositions and methods of the disclosure, patients, such as pregnant human female patients, undergoing or at risk of undergoing preterm labor may be treated so as to prolong the term of their pregnancies, imparting the unborn infants with additional time for vital organs and tissue systems to mature. At young gestational ages, such as gestational ages of from about 24 weeks to about 34 weeks, unborn infants are characterized by critical organ networks that have not sufficiently developed. The compositions and methods of the disclosure provide an opportunity to extend pregnancy for a time sufficient to enable unborn infants to undergo lung and neuronal maturation, among other important developments. This maturation, in turn, can effectuate a substantial increase in the likelihood of neonatal survival.

To obtain these advantageous pharmacological benefits, the patient may be administered therapeutically effective amounts of the PGF2α receptor antagonist and atosiban, for example, either concurrently or at different times. The patient may receive multiple, continuous doses of the PGF2α receptor antagonist and/or the atosiban. The same or different dosing schedules may be used for administration of the PGF2α receptor antagonist and the atosiban. For instance, each time the patient receives a dose of one of these agents, the patient may or may not receive a dose of the second agent. The patient may receive the PGF2α receptor antagonist, for example, one or more times per day (such as once or twice daily) and the atosiban may be administered to the subject, for example, one or more times every 4, 6, 8, 10, 12, 14, 16, 18, 22, 24, 36, or 48 hours, or more. The combined administration of atosiban and the PGF2α receptor antagonist may occur one or more times per day, week, or month, as described herein, and may continue, for example, up until the patient undergoes delivery or until a full gestational term has been reached.

The sections that follow provide a description of the atosiban formulations and PGF2α receptor antagonists that may be used in conjunction with the compositions and methods described herein, as well as the dosing amounts and schedules that may be employed for administering these tocolytic agents.

### PGF2α Receptor Antagonists

PGF2α receptor antagonists that may be used in conjunction with the compositions and methods described herein include 1,3-thiazolidine-2-carboxamide compounds, such as those PGF2α receptor antagonists described, for example, in U.S. Pat. Nos. 8,415,480; 9,447,055; and 9,834,528, the disclosures of each of which are incorporated herein by reference in their entirety. Exemplary PGF2α receptor antagonists include, for instance, compounds represented by formula (I)

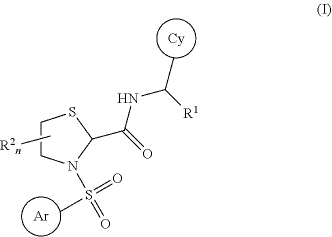

wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof.

In some embodiments, the ring Ar is selected from substituents (Ia) to (Iy):

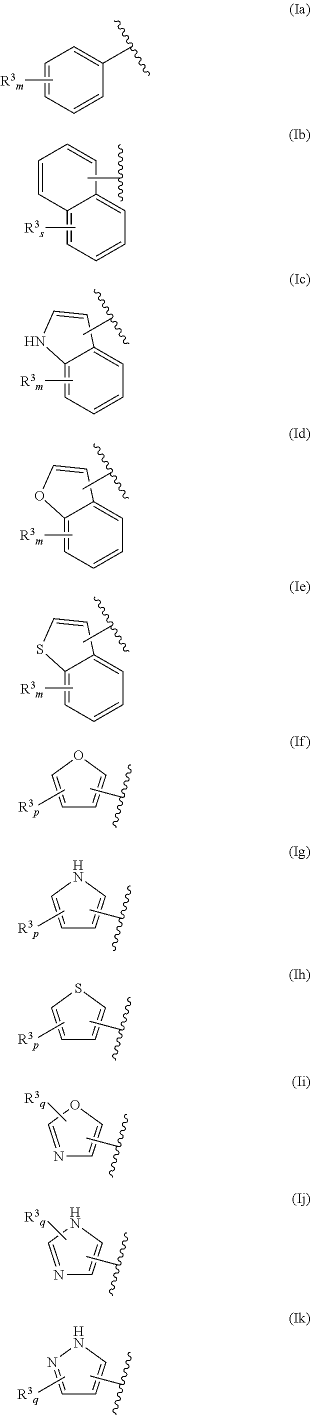

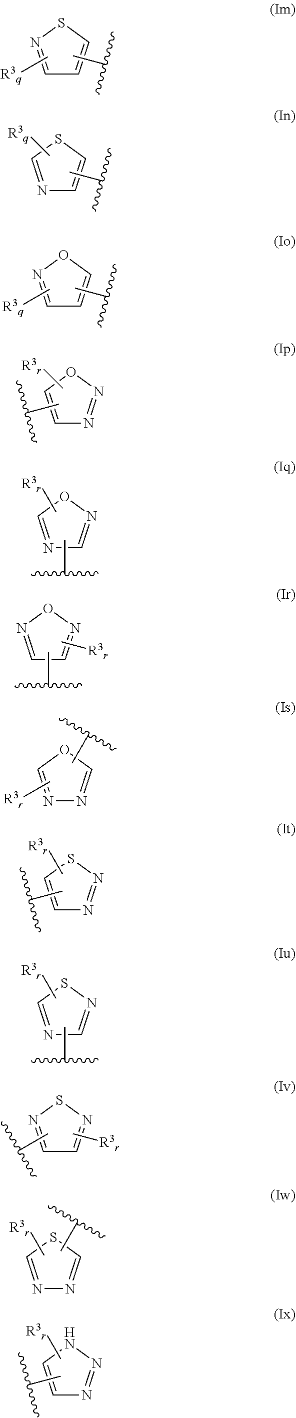

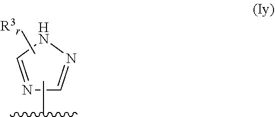

wherein each R³ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each m is independently an integer from 0-5;
each p is independently an integer from 0-3;
each q is independently an integer from 0-2;
each r is independently an integer from 0-1; and
each s is independently an integer from 0-7.

In some embodiments. each R³ is independently selected from substituents (IIa) to (IIy).

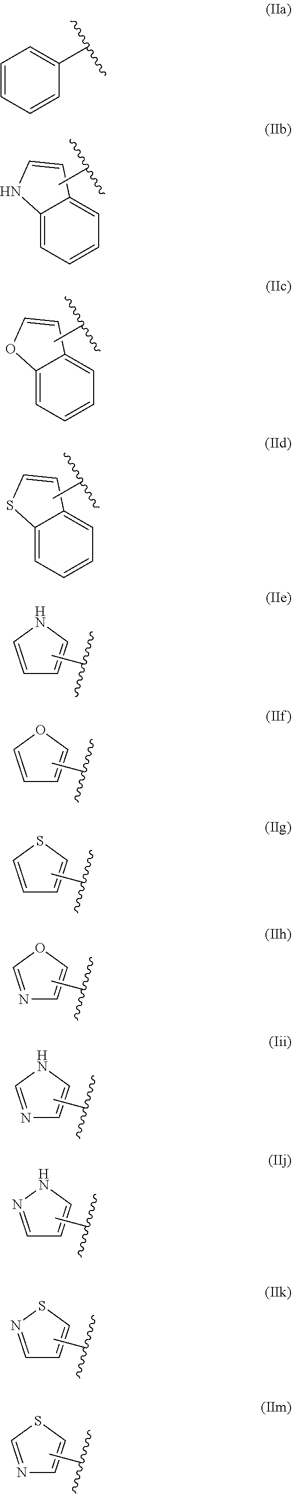

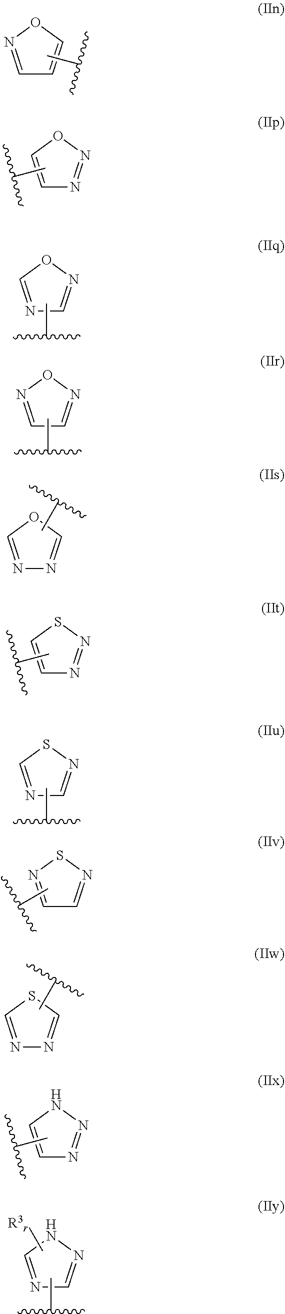

In some embodiments, the ring Ar is a substituent represented by formula (Ia)

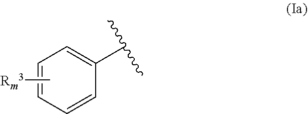

and each R³ is, independently, optionally substituted and optionally fused aryl, optionally substituted and optionally fused heteroaryl, optionally substituted and optionally fused cycloalkyl, or optionally substituted and optionally fused heterocycloalkyl.

In some embodiments, the ring Cy is selected from substituents (IIIa) to (IIIaa):

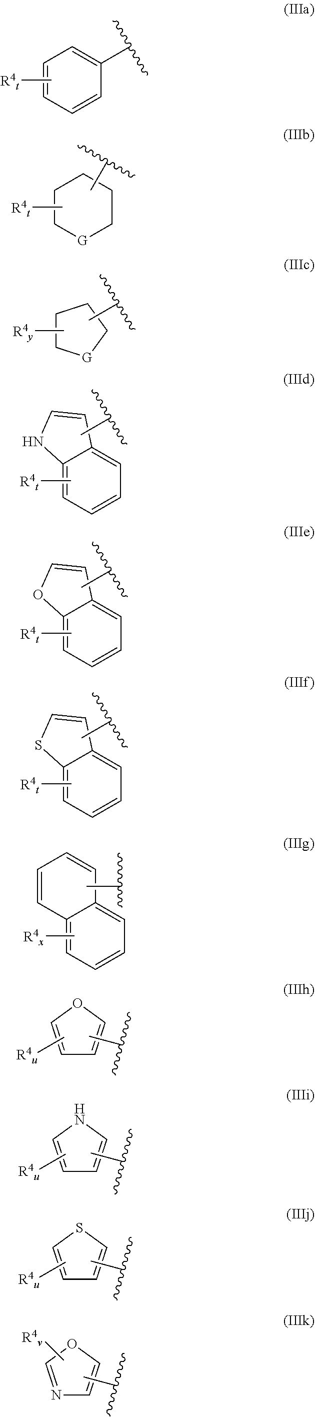

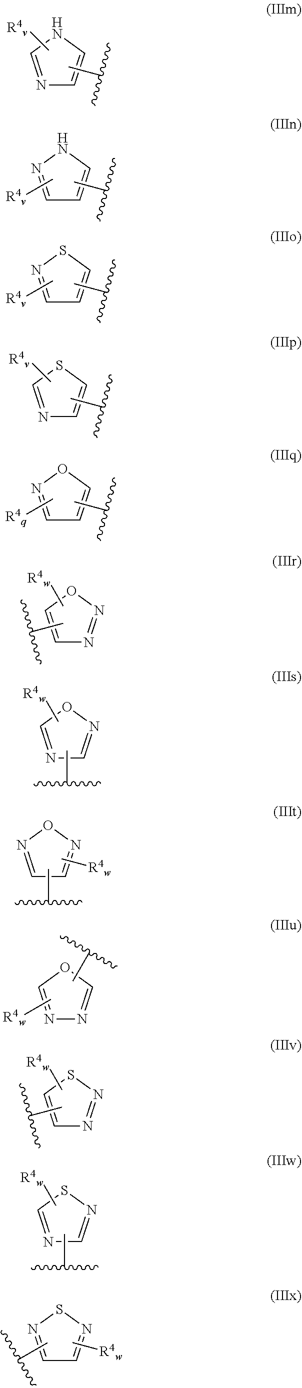

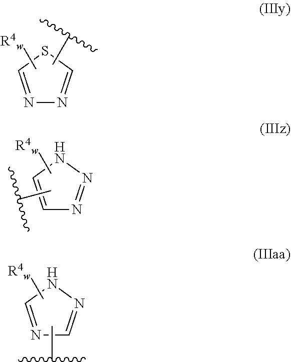

wherein each R⁴ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each G is independently —CH₂—, —CR⁴H—, —NH—, —NR⁴—, —O—, or —S—;
each t is independently an integer from 0-5;
each u is independently an integer from 0-3;
each v is independently an integer from 0-2;
each w is independently an integer from 0-1;
each x is independently an integer from 0-7; and
each y is independently an integer from 0-4.

In some embodiments, the ring Cy is an optionally substituted aryl group represented by formula (IVa).

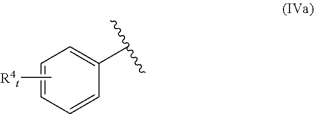

In some embodiments, the ring Cy is a substituted aryl group represented by formula (IVb).

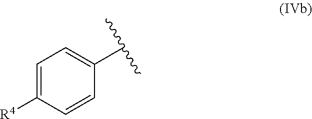

In some embodiments, R¹ is C₁-C₅-alkyl carboxy, C₁-C₅-alkyl acyl, C₁-C₅-alkyl alkoxycarbonyl, C₁-C₅-alkyl acyloxy, substituted C₁-C₅-alkyl carboxy, substituted C₁-C₅-alkyl acyl, substituted C₁-C₅-alkyl alkoxycarbonyl, or substituted C₁-C₅-alkyl acyloxy. In some embodiments, R¹ is optionally substituted C₁-C₅-alkyl acyloxy.

In some embodiments, the compound is represented by formula (V)

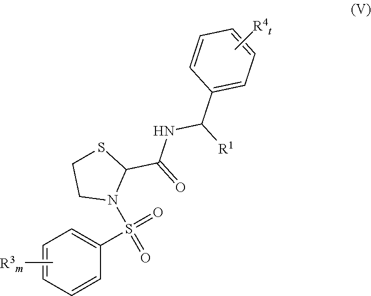

wherein R¹ is C₁-C₅-alkyl carboxy, C₁-C₅-alkyl acyl, C₁-C₅-alkyl alkoxycarbonyl, C₁-C₅-alkyl acyloxy, substituted C₁-C₅-alkyl carboxy, substituted C₁-C₅-alkyl acyl, substituted C₁-C₅-alkyl alkoxycarbonyl, or substituted C₁-C₅-alkyl acyloxy;
each R³ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each R⁴ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
m is an integer from 0-5; and
t is an integer from 0-5,
or a pharmaceutically acceptable salt thereof.

In some embodiments, the compound is represented by formula (Va)

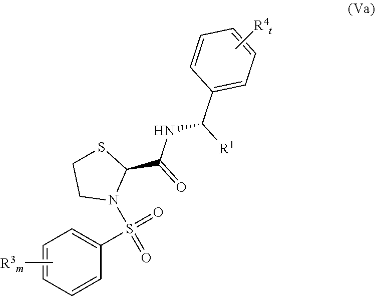

or a pharmaceutically acceptable salt thereof.

In some embodiments, the compound is represented by formula (VI)

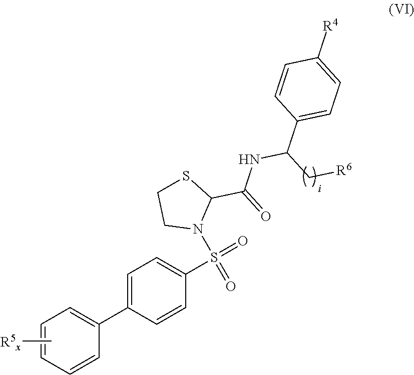

wherein R⁶ is hydroxyl, optionally substituted acyl, optionally substituted alkoxycarbonyl, or optionally substituted acyloxy;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3; and
x is an integer from 0-5,
or a pharmaceutically acceptable salt thereof.

In some embodiments, the compound is represented by formula (VII)

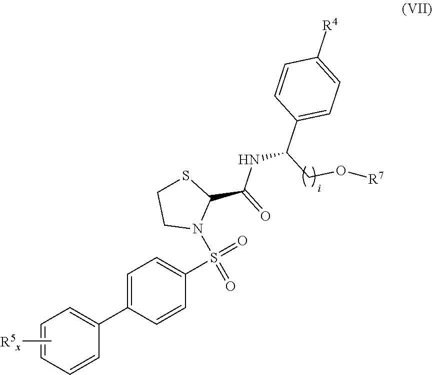

wherein R⁷ is H or optionally substituted aminoacyl;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3; and
x is an integer from 0-5,
or a pharmaceutically acceptable salt thereof.

In some embodiments, the compound is represented by formula (VII)

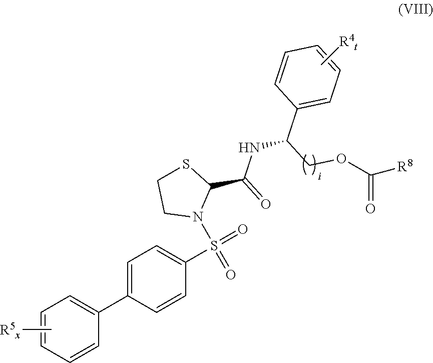

wherein R⁸ is optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl, preferably wherein R⁸ is amino-substituted alkyl, such as 1-amino C₁-C₆ alkyl (e.g., (S)-1-amino C₁-C₆ alkyl or (R)-1-amino C₁-C₆ alkyl, for example, (S)-1-amino-2-methylpropyl, (S)-1-amino-2-methylbutyl, (S)-1-amino-3-methylbutyl, (R)-1-amino-2-methylpropyl, (R)-1-amino-2-methylbutyl, or (R)-1-amino-3-methylbutyl;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3;
t is an integer from 0-5; and
x is an integer from 0-5,
   or a pharmaceutically acceptable salt thereof.

### (3S)-3-({[(2S)-3-(biphenyl-4-ylsulfonyl)-1,3-thiazolidin-2-yl]carbonyl}-amino)-3-(4-fluorophenyl)propyl L-valinate (Compound 2)

Particular PGF2α receptor antagonists that may be administered to a patient (e.g., a pregnant human patient) to treat or prevent preterm labor include (3S)-3-({[(2S)-3-(biphenyl-4-ylsulfonyl)-1,3-thiazolidin-2-yl]carbonyl}-amino)-3-(4-fluorophenyl)propyl L-valinate, represented by formula (2), below) and pharmaceutically acceptable salts thereof. This compound is converted in vivo to 3-([1,1′-biphenyl]-4-ylsulfonyl)-N-[1-(4-fluorophenyl)-3-hydroxypropyl]-1,3-thiazolidine-2-carboxamide (represented by formula (1), below). Compound 1, described in U.S. Pat. No. 8,415,480, is an antagonist of the prostaglandin F receptor, as this compound exhibits an inhibition constant (Ki) of 6 nM for human PGF2α receptor as determined by competitive radioligand binding assays (experimental details of competitive radioligand binding assays useful for the determination of Ki values are described, e.g., in U.S. Pat. No. 8,415,480, Example 51). Following administration to a subject, compound 2 is de-esterified in vivo so as to form compound 1 due to the activity of endogenous esterases, such as those present in the gastrointestinal tract.

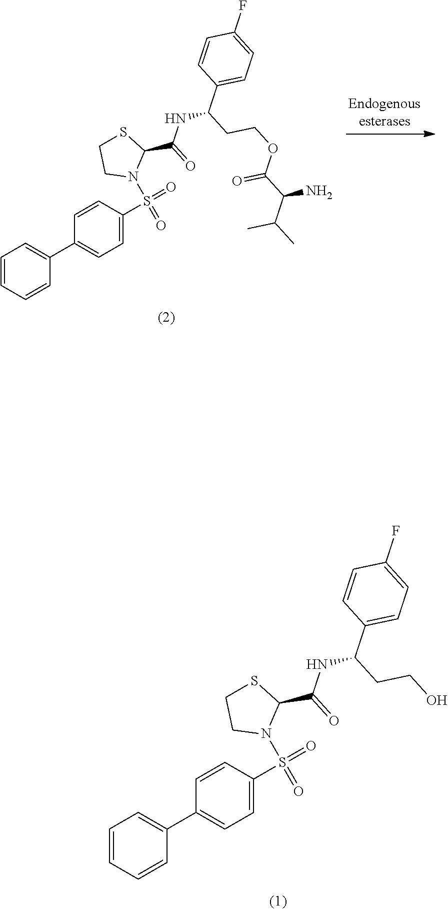 [SMILES: CC(C)[C@H](N)C(=O)OCC[C@H](NC(=O)[C@@H]1SCCN1S(=O)(=O)c1ccc(-c2ccccc2)cc1)c1ccc(F)cc1 ; O=C(N[C@@H](CCO)c1ccc(F)cc1)[C@@H]1SCCN1S(=O)(=O)c1ccc(-c2ccccc2)cc1]

It has been discovered that compound 2 is an inhibitor of the prostaglandin F2α receptor, as compound 2 inhibits the receptor with a Ki of 1 nM. Compound 2 exhibits improvements in several physicochemical characteristics relative to compound 1, including solubility in water as well as in media that simulate the small intestinal contents in the fed (FeSSIF) and fasted (FaSSIF) states. These data are described, e.g., in U.S. Pat. Nos. 9,447,055 and 9,843,528.

In addition to exhibiting enhanced aqueous solubility, compound 2 and salts thereof feature a particularly beneficial absorption mechanism. Compound 2 is de-esterified by ambient esterases in the small intestine and subsequently penetrates the small intestinal epithelium passively. Compound 2 and salts thereof are not substrates for the Pept1 transporter protein, a proton-coupled co-transporter that mediates the absorption of peptidic nutrients. Pept1 is known to mediate the absorption of a variety of valinate esters, as described, for example, in Vig et al., Adv. Drug Deliv. Rev. 65:1370-1385 (2013). Pept1 exhibits broad substrate specificity, as evidenced by the structural diversity of compounds that are transported across the intestinal epithelium by this protein. Despite the presence the valinate ester functionality, compound 2 and salts thereof are not dependent upon this transporter for absorption across the small intestinal epithelium. This is an advantageous property, as compound 2 and salts thereof (for instance, the chloride salt thereof, represented by formula (3)) thus do not compete with natural substrates of Pept1, such as peptidic nutrients, for binding to, and transport by, this protein. Rather, compound 2 and salts thereof are converted in vivo to a form that is readily absorbed in a manner independent of energy and local proton gradient. This beneficial property, coupled with the high aqueous solubility of compound I and salts thereof, fosters a pharmacokinetic profile in which compound 2 and salts thereof readily dissolve in an aqueous environment and are in turn converted into a form capable of transported-independent absorption.

### (3S)-3-({[(2S)-3-(biphenyl-4-ylsulfonyl)-1,3-thiazolidin-2-yl]carbonyl}-amino)-3-(4-fluorophenyl)propyl L-valinate Hydrochloride (Compound 3)

The chloride salt of compound 2, (3S)-3-({[(2S)-3-(biphenyl-4-ylsulfonyl)-1,3-thiazolidin-2-yl]carbonyl}-amino)-3-(4-fluorophenyl)propyl L-valinate hydrochloride, designated as compound 3 herein) exhibits additional advantageous characteristics. For example, this compound is readily crystallized using a several distinct experimental procedures. Compound 3 assumes a single, reproducible crystal form upon crystallization from a variety of media and under different ambient conditions. Moreover, this crystal form of compound 3 exhibits extended stability under ambient conditions and in the presence of elevated relative humidity. Compound 3 exhibits a low hygroscopicity and thus does not demonstrate a propensity to absorb moisture from the local atmosphere. Compound 3 therefore exhibits a resistance to chemical changes induced by the presence of water, such as hydrolysis, as well as a resistance to the incorporation of impurities. For instance, impurities associated with atmospheric water are not readily integrated into the crystalline form of compound 3.

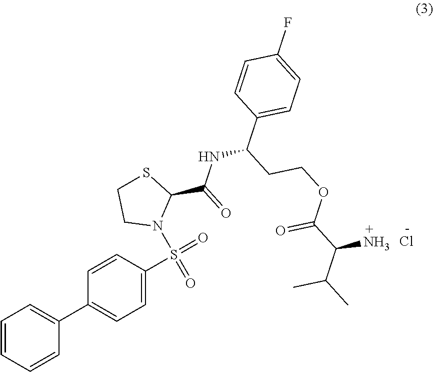 [SMILES: CC(C)[C@H](N)C(=O)OCC[C@H](NC(=O)[C@@H]1SCCN1S(=O)(=O)c1ccc(-c2ccccc2)cc1)c1ccc(F)cc1 ; Cl]

### Additional PGF2α Receptor Antagonists

PGF2α receptor antagonists that may be used in conjunction with the compositions and methods described herein include additional 1,3-thiazolidine-2-carboxamides, such as additional 1,3-thiazolidine-2-carboxamides that give rise to compound (1) in vivo (e.g., by way of de-esterification of a compound of formula (VIII) due to the activity of endogenous esterases in vivo). Examples of additional PGF2α receptor antagonists that may be used in conjunction with the compositions and methods described herein include those compounds set forth in Table 1, below. The syntheses of these compounds are described in U.S. Pat. No. 8,415,480, the disclosure of which is incorporated herein by reference in its entirety.

TABLE 1
Exemplary PGF2α Receptor Antagonists Useful in Combination with Atosiban for the Treatment or
Prevention of Preterm Labor

| | | Reported ¹H NMR and/or ESI |
|---|---|---|
| Ex. | Compound | Spectral Properties |
| 1 | 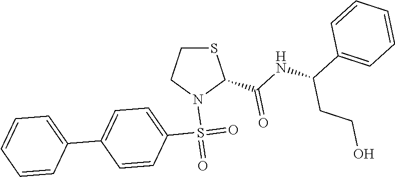 [SMILES: O=C(N[C@@H](CCO)c1ccccc1)[C@H]1SCCN1S(=O)(=O)c1ccc(-c2ccccc2)cc1] | ¹H NMR (300 MHz, CDCl₃); 1.85-2.3 (m, 2H), 2.55-3.15 (m, CH₂S, 2H), 3.65-3.9 (m, CH₂N, CH₂O, 4H), 5.2-5.3 (m, CH, 1H), 5.37 (s, CH, 1H), 7.25-8.0 (m, CH(Ar), 14H); M⁺(ESI⁺): 483.0; M−(ESI−) 481.0 |
| 2 | 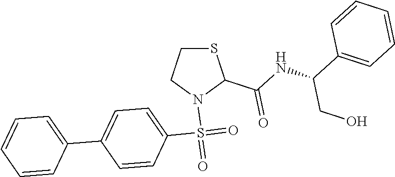 [SMILES: O=C(N[C@@H](CO)c1ccccc1)C1SCCN1S(=O)(=O)c1ccc(-c2ccccc2)cc1] | ¹H NMR (300 MHz, CDCl₃); 2.4-2.9 (m, CH₂S, 2H), 3.5-3.7 (m, CH₂N, 2H), 3.7- 3.9 (m, CH₂O, 2H), 4.9 (m, CH, 1H), 5.2 (s, CH, 1H), 7.1-7.9 (m, CH(Ar), 14H); M⁺(ESI⁺): 469.2; M−(ESI−) 467.1 |
| 3 | 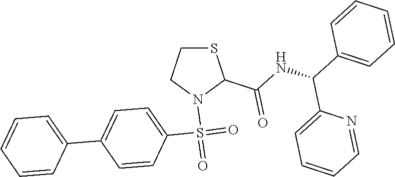 [SMILES: O=C(N[C@H](c1ccccc1)c1ccccn1)C1SCCN1S(=O)(=O)c1ccc(-c2ccccc2)cc1] | ¹H NMR (300 MHz, CDCl₃); 2.5-3.0 (m, CH₂S, 2H), 3.6-4.0 (m, CH₂N, 2H), 5.41 (s, CH, 0.5H), 5.42 (s, CH, 0.5H), 6.07 (m, CH, 1H), 5.2 (s, CH, 1H), 7.1-7.8 (m, CH(Ar), 16H), 7.8-7.9 (m, CH, 1H), 8.5-8.6 (m, CH, 1H); M⁺(ESI⁺): 516.3; M− (ESI−) 514.1. |
| 4 | 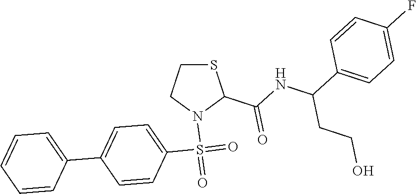 [SMILES: O=C(NC(CCO)c1ccc(F)cc1)C1SCCN1S(=O)(=O)c1ccc(-c2ccccc2)cc1] | ¹H NMR (400 MHz, CDCl₃); 1.8-2.15 (m, CH₂, 2H), 2.5-2.9 (m, CH₂S, 2H), 3.5- 3.8 (m, CH₂N, CH₂O, 4H), 5.15 (m, CH, 1H), 5.25 (s, CH, 1H), 7.1-7.9 (m, CH(Ar), 13H); M⁺(ESI⁺): 501.3. |
| 5 | 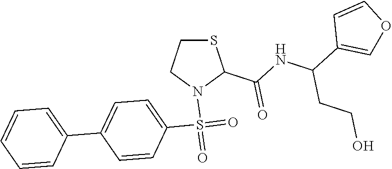 [SMILES: O=C(NC(CCO)c1ccoc1)C1SCCN1S(=O)(=O)c1ccc(-c2ccccc2)cc1] | ¹H NMR (400 MHz, CDCl₃); 1.7-2.2 (m, CH₂, 2H), 2.5-2.9 (m, CH₂S, 2H), 3.6- 3.8 (m, CH₂N, CH₂O, 4H), 5.12 (m, CH, 1H), 5.21 (s, CH, 1H), 6.25-6.35 (d, CH(furyl), 1H), 6.9-7.1 (m, CH(furyl), 1H), 7.3-7.9 (m, CH(Ar), 10H); M⁺(ESI⁺): 473.1; M−(ESI−): 471.1. |
| 6 | 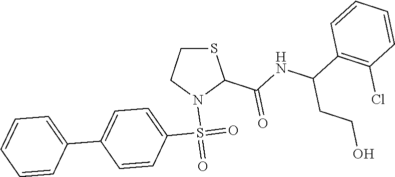 [SMILES: O=C(NC(CCO)c1ccccc1Cl)C1SCCN1S(=O)(=O)c1ccc(-c2ccccc2)cc1] | ¹H NMR (400 MHz, CDCl₃); 1.95-2.15 (m, CH₂, 2H), 2.5-2.9 (m, CH₂S, 2H), 3.6-3.8 (m, CH₂N, CH₂O, 4H), 5.28 (s, CH, 0.5H), 5.29 (s, CH, 0.5H), 5.4-5.5 (m, CH, 1H), 7.1-7.9 (m, CH(Ar), 13H); M⁺(ESI⁺): 517.3. |
| 7 | 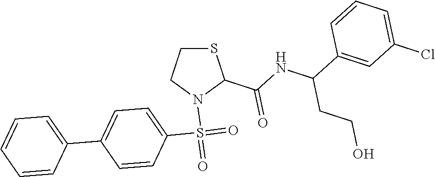 [SMILES: O=C(NC(CCO)c1cccc(Cl)c1)C1SCCN1S(=O)(=O)c1ccc(-c2ccccc2)cc1] | ¹H NMR (400 MHz, CDCl₃); 1.8-2.2 (m, CH₂, 2H), 2.55-3.0 (m, CH₂S, 2H), 3.6- 3.8 (m, CH₂N, CH₂O, 4H), 5.1-5.2 (m, CH, 1H), 5.24 (s, CH, 0.5H), 5.28 (s, CH, 0.5H), 7.2-7.9 (m, CH(Ar), 13H); M⁺(ESI⁺): 517.1; M−(ESI−): 514.8. |
| 8 | 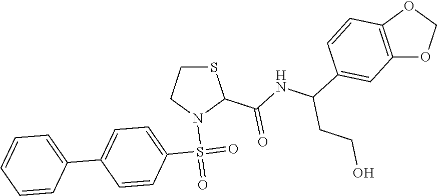 [SMILES: O=C(NC(CCO)c1ccc2c(c1)OCO2)C1SCCN1S(=O)(=O)c1ccc(-c2ccccc2)cc1] | ¹H NMR (400 MHz, CDCl₃); 1.7-2.2 (m, CH₂, 2H), 2.45-3.0 (m, CH₂S, 2H), 3.6- 3.95 (m, CH₂N, CH₂O, 4H), 5.0-5.1 (m, CH, 1H), 5.26 (s, CH, 0.5H), 5.28 (s, CH, 0.5H), 5.87 (s, CH, 1H), 5.89 (s, CH, 1H), 6.8-7.9 (m, CH(Ar), 12H); M⁺(ESI⁺): 527.1; M−(ESI−): 525.0. |
| 9 | 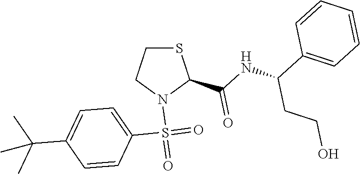 [SMILES: CC(C)(C)c1ccc(S(=O)(=O)N2CCS[C@H]2C(=O)N[C@@H](CCO)c2ccccc2)cc1] | ¹HNMR (400 MHz, CDCl₃); 1.27 (s, CH3, 9H), 1.65-2.15 (m, CH₂, 2H), 2.45-2.95 (m, CH₂S, 2H),3.6- 3.95(m, CH₂N, CH₂O, 4H), 5.1- 5.2(m, CH, 1H), 5.26(s, CH, 1H), 7.3-7.7 (m, CH(Ar), 9H); M⁺(ESI⁺): 463.1; M−(ESI−): 461.6. |
| 10 | 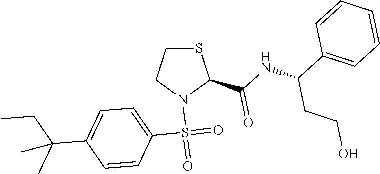 [SMILES: CCC(C)(C)c1ccc(S(=O)(=O)N2CCS[C@H]2C(=O)N[C@@H](CCO)c2ccccc2)cc1] | ¹HNMR (400 MHz, CDCl₃); 0.63(t, J = 7.3 Hz, 3H), 1.29(s, CH₃, 6H), 1.65(q, J = 7.4 Hz, 2H), 1.88 (m, CH₂, 1H), 2.19(m, CH₂, 1H),2.5 (m, CH₂S, 1H), 2.94(dt, J = 12 Hz and 5.6Hz, CH₂S, 1H), 3.65- 3.87(m, CH₂N, CH₂O, 4H), 5.2 (td, J = 6.6 Hz and 3.8 Hz, CH, 1H), 5.32(s, CH, 1H), 7.25- 7.8(m, CH(Ar), 9H); M⁺(ESI⁺): 477.2; M− (ESI−): 475.0. |
| 11 | 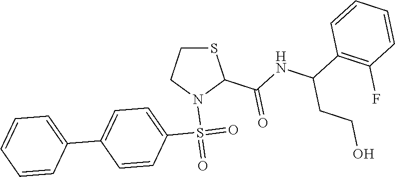 [SMILES: O=C(NC(CCO)c1ccccc1F)C1SCCN1S(=O)(=O)c1ccc(-c2ccccc2)cc1] | ¹H NMR (400 MHz, CDCl₃); 2.24 (m, 3H), 2.25 (m, 1H), 2.95 (m, 1H), 3.63- 3.90 (m, 4H), 5.31 (m, 2H), 6.99-7.86 (m, 13H) .; M⁺(ESI⁺): 501; M−(ESI−): 499. |
| 12 | 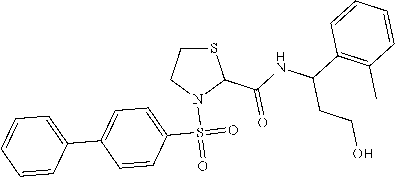 [SMILES: Cc1ccccc1C(CCO)NC(=O)C1SCCN1S(=O)(=O)c1ccc(-c2ccccc2)cc1] | ¹H NMR (300 MHz, CDCl₃); 1.86 (m, 1H), 2.09 (m, 1H), 2.36 (s, 3H), 2.57 (m, 1H), 2.99 (m, 1H), 3.72-3.92 (m, 4H), 5.31-5.40 (m, 2H), 7.19-7.88 (m, 13H); M⁺(ESI⁺): 497; M− (ESI−): 495. |
| 13 | 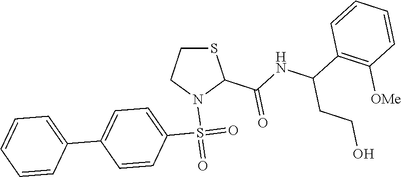 [SMILES: COc1ccccc1C(CCO)NC(=O)C1SCCN1S(=O)(=O)c1ccc(-c2ccccc2)cc1] | ¹H NMR (300 MHz, CDCl₃); 2.00 (m, 2H), 2.57 (m, 1H), 2.87-2.98 (m, 2H), 3.62-3.70 (m, 3H), 3.92 (m, 0.4H), 3.92 (s, 1.8H), 3.98 (s, 1.2H), 4.04 (m, 0.6H), 5.23 (m, 1H), 5.43 (m, 1H), 6.92 (m, 2H), 7.22-7.45 (m, 2H), 7.46 (m, 3H), 7.60 (m, 2H), 7.77 (m, 2H), 7.90 (m, 2H), 8.26 (d, J = 11.7 Hz, 0.6H), 8.47 (d, J = 9.8 Hz, 0.4H); M⁺(ESI⁺): 513; M− (ESI−): 51 1. |
| 14 | 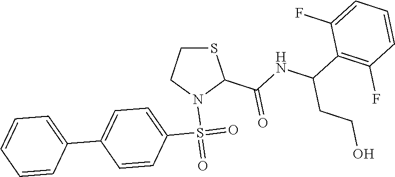 [SMILES: O=C(NC(CCO)c1c(F)cccc1F)C1SCCN1S(=O)(=O)c1ccc(-c2ccccc2)cc1] | ¹H NMR (300 MHz, CDCl₃); 2.01 (m, 2H), 2.69 (m, 1H), 3.00 (m, 1H), 3.69- 3.96 (m, 4H), 4.32 (m, 0.5H), 5.36 (m, 1H), 5.62 (m, 1H), 6.91-7.91 (m, 13H).; M⁺(ESI⁺): 519; M−(ESI−): 517. |
| 15 | 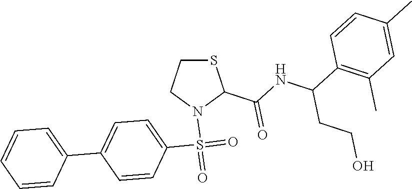 [SMILES: Cc1ccc(C(CCO)NC(=O)C2SCCN2S(=O)(=O)c2ccc(-c3ccccc3)cc2)c(C)c1] | ¹H NMR (300 MHz, CDCl₃); 1.94 (m, 1H), 2.09 (m, 1H), 2.30 (m, 6H), 2.56 (m, 1H), 2.87 (m, 0.5H), 2.99 (m, 0.5H), 3.70 (m, 3H), 391 (m, 1H), 5.31-5.37 (m, 2H), 6.76 (m, 2H), 6.99 (m, 2H), 7.45- 7.93 (m, 9H).; M−(ESI−): 511. M−(ESI−) : 509. |
| 16 | 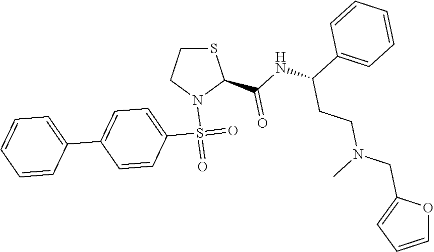 [SMILES: CN(CC[C@H](NC(=O)[C@@H]1SCCN1S(=O)(=O)c1ccc(-c2ccccc2)cc1)c1ccccc1)Cc1ccco1] | ¹H NMR (300 MHz, CDCl₃); ¹H NMR (300 MHz, CDCl₃); 1.85-2.2 (m, 2H), 2.5 (s, CH₃N, 3H), 2.45-3.00 (m, CH₂S, 2H), 2.7-3.0 (m, CH₂N, 2H), 3.6 (m, CH₂N, 2H), 4.2 (m, CH₂N, 2H), 4.7 (m, CH, 1H), 5.3 (s, CH, 1H), 6.37 (m, CH(furyl), 1H), 6.56 (m, CH(furyl), 1H), 7.05-8.0 (m, CH(Ar), 15H), 8.7 (m, NH, 1H); M⁺(ESI⁺): 576.1; M−(ESI−): 573.8. |
| 17 | 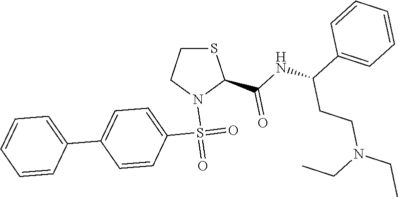 [SMILES: CCN(CC)CC[C@H](NC(=O)[C@@H]1SCCN1S(=O)(=O)c1ccc(-c2ccccc2)cc1)c1ccccc1] | ¹H NMR (400 MHz, DMSO); 1.05 (t, J = 7.3 Hz, 6H), 1.88 (m, CH₂, 2H), 2.79- 2.81 (m, CH₂N, 6H), 3.05 (m, CH₂S, 1H), 3.05-3.50 (broad, H₂O), 3.81 (m, CH₂N, 2H), 4.86 (td, J = 6.6 Hz and 3.8 Hz, CH, 1H), 5.44 (s, CH, 1H), 6.58 (s, CH₂, 2H), 7.24-7.33 (m, CH(Ar), 5H); 7.53 (m, CH(Ar), 3H), 7.76 (m, CH(Ar), 2H), 7.95 (m, CH(Ar), 4H), 8.68 (m, 1H, NH); M⁺(ESI⁺): 538.0; M−(ESI−): 536.0. |
| 18 | 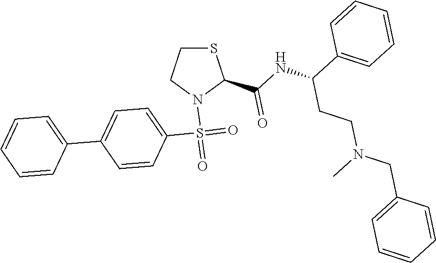 [SMILES: CN(CC[C@H](NC(=O)[C@@H]1SCCN1S(=O)(=O)c1ccc(-c2ccccc2)cc1)c1ccccc1)Cc1ccccc1] | ¹H NMR(300 MHz, CDCl₃); 1.75-2.1 (m, 2H), 2.2(s, CH₃N, 3H), 2.3- 2.9(m, CH₂S, 2H), 3.3-3.5 (m, CH₂N, 2H), 3.6(s, CH₂N, 2H), 3.9(m, CH₂N, 2H), 5.0(m, CH, 1H), 5.3 (s, CH, 1H), 7.0- 8.0(m,CH(Ar), 19H), 8.6(m, NH, 1H); M⁺ (ESI⁺): 586.2; M−(ESI−): 583.8. |
| 19 | 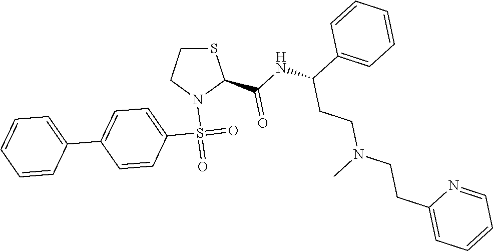 [SMILES: CN(CCc1ccccn1)CC[C@H](NC(=O)[C@@H]1SCCN1S(=O)(=O)c1ccc(-c2ccccc2)cc1)c1ccccc1] | ¹HNMR(400 MHz, DMSO); 1.75(m, CH₂, 6 H), 2.18(s, CH₃, 3H), 2.36(m, CH, 1H), 2.50 (m, CHS, 1H), 2.55- 2.85(m, CH, 3H), 2.95(m, CHS, 1H), 2.37(m, CH₂, 2H), 3.67-3.74 (m, 2H, CH₂N), 4.70(s, CH, 1H), 5.34(s, CH, 1H), 6.47(s, 2H, Hvinyl), 7.14-7.25(m, CH(Ar), 7H); 7.40-7.49(m, CH(Ar), 3H), 7.50-7.70(m,CH(Ar), 3H), 7.83 (m, CH(Ar), 4H), 8.33(m, CH(Ar), 1H), 8.58(m, 1H, NH); M⁺(ESI⁺): 601; M(ESF): 599 |
| 20 | 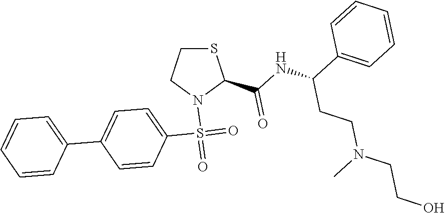 [SMILES: CN(CCO)CC[C@H](NC(=O)[C@@H]1SCCN1S(=O)(=O)c1ccc(-c2ccccc2)cc1)c1ccccc1] | ¹H NMR (300 MHz, CDCl₃); 1.8-2.2 (m, 2H), 2.36(s, CH₃N, 3H), 2.45-3.05 (m, CH₂S, 2H), 2.6- 2.7(m, CH₂N, 4H), 3.7(m, CH₂O, 2H), 3.7- 4.1(m, CH₂N, 2H), 5.1-5.25(m, CH, 1H), 5.48(s, CH, 1H), 7.25- 8.0(m, CH(Ar), 14H), 8.55(m, NH, 1H); M⁺(ESθ: 540.2; M− (ESF): 537.99. |
| 21 | 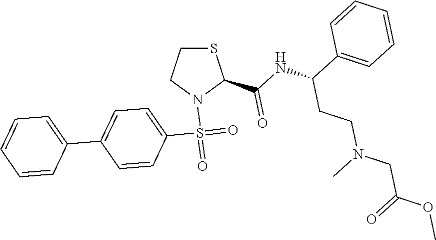 [SMILES: COC(=O)CN(C)CC[C@H](NC(=O)[C@@H]1SCCN1S(=O)(=O)c1ccc(-c2ccccc2)cc1)c1ccccc1] | ¹H NMR (300 MHz, CDCl₃); 1.8-2.2 (m, 2H), 2.4 (s, CH₃N, 3H), 2.5-3.0 (m, CH₂S, 2H), 2.6-2.7 (m, CH₂N, 2H), 3.4 (s, CH₂N, 2H), 3.69 (s, CH₃O, 3H), 3.7- 4.0 (m, CH₂N, 2H), 5.1 (m, CH, 1H), 5.49 (s, CH, 1H), 7.2-8.0 (m, CH(Ar), 14H), 8.6 (m, NH, 1H); M⁺(ESI⁺): 568.2; M−(ESI−): 565.8. |
| 22 | 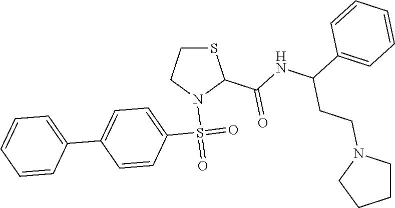 [SMILES: O=C(NC(CCN1CCCC1)c1ccccc1)C1SCCN1S(=O)(=O)c1ccc(-c2ccccc2)cc1] | ¹H NMR (300 MHz, CDCl₃); 2.15-2.4 (m, CH₂, 6H), 2.45-3.05 (m, CH₂S, 2H), 2.5- 3.5 (m, CH₂N, 6H), 3.9-4.25 (m, CH₂N, 2H), 5.2 (m, CH, 1H), 5.5 (s, CH, 1H), 7.2-8.0 (m, CH(Ar), 14H), 8.5 (m, NH, 1H); M⁺(ESI⁺): 536.3; M−(ESI−): 534.2. |
| 23 | 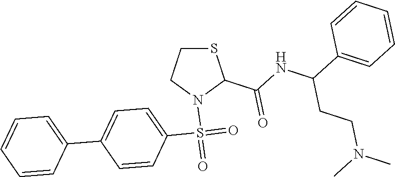 [SMILES: CN(C)CCC(NC(=O)C1SCCN1S(=O)(=O)c1ccc(-c2ccccc2)cc1)c1ccccc1] | ¹H NMR (300 MHz, CDCl₃); 2.2-2.7 (m, CH₂, 2H), 2.45-3.00 (m, CH₂S, 2H), 2.7 (s, CH₃N, 3H), 2.9 (s, CH₃N, 3H), 3.5- 3.9 (m, CH₂N, 2H), 4.0-4.25 (m, CH₂N, 2H), 5.0 (m, CH, 1H), 5.3 (s, CH, 1 H), 7.2-8.0 (m, CH (Ar), 14H), 8.5 (m, NH, 1H); M⁺(ESI⁺): 510.2; M−(ESI−): 508.1. |
| 24 | 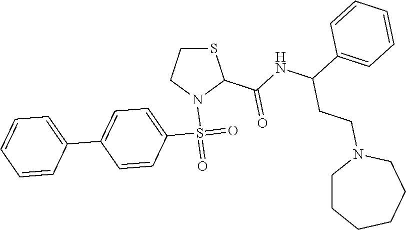 [SMILES: O=C(NC(CCN1CCCCCC1)c1ccccc1)C1SCCN1S(=O)(=O)c1ccc(-c2ccccc2)cc1] | M⁺(ESI⁺): 564.3; M−(ESI−): 562.3. |
| 25 | 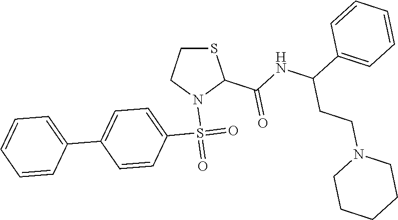 [SMILES: O=C(NC(CCN1CCCCC1)c1ccccc1)C1SCCN1S(=O)(=O)c1ccc(-c2ccccc2)cc1] | M⁺(ESI⁺): 550.3; M−(ESI−): 548.2. |
| 26 | 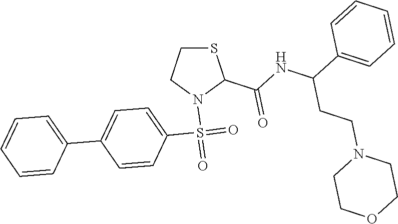 [SMILES: O=C(NC(CCN1CCOCC1)c1ccccc1)C1SCCN1S(=O)(=O)c1ccc(-c2ccccc2)cc1] | M⁺(ESI⁺): 552.3; M−(ESI−): 550.2. |
| 27 | 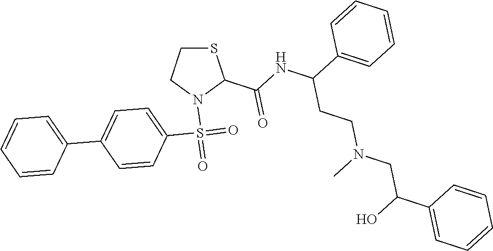 [SMILES: CN(CCC(NC(=O)C1SCCN1S(=O)(=O)c1ccc(-c2ccccc2)cc1)c1ccccc1)CC(O)c1ccccc1] | M⁺(ESI⁺): 616.3; M−(ESI−): 614.9. |
| 28 | 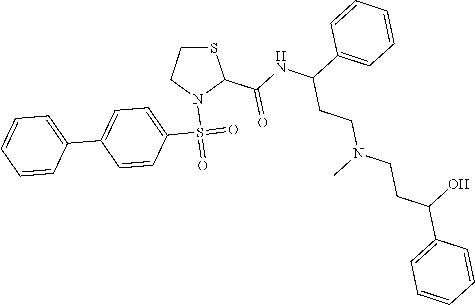 [SMILES: CN(CCC(O)c1ccccc1)CCC(NC(=O)C1SCCN1S(=O)(=O)c1ccc(-c2ccccc2)cc1)c1ccccc1] | M⁺(ESI⁺): 630.4; M−(ESI−): 628.2. |
| 29 | 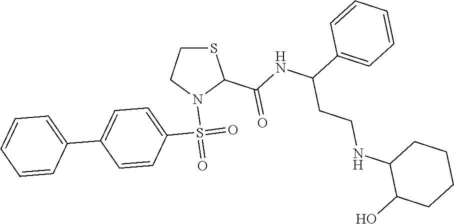 [SMILES: O=C(NC(CCNC1CCCCC1O)c1ccccc1)C1SCCN1S(=O)(=O)c1ccc(-c2ccccc2)cc1] | M⁺(ESI⁺): 580.6; M−(ESI−): 578.8. |
| 30 | 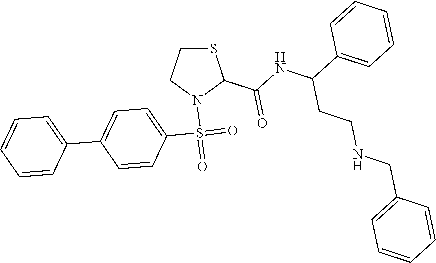 [SMILES: O=C(NC(CCNCc1ccccc1)c1ccccc1)C1SCCN1S(=O)(=O)c1ccc(-c2ccccc2)cc1] | M⁺(ESI⁺): 571.8; M−(ESI−): 569.99. |
| 31 | 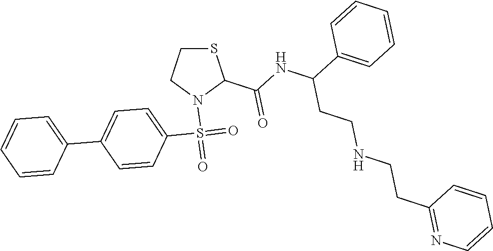 [SMILES: O=C(NC(CCNCCc1ccccn1)c1ccccc1)C1SCCN1S(=O)(=O)c1ccc(-c2ccccc2)cc1] | M⁺(ESI⁺): 586.9; M−(ESI−): 585.3. |
| 32 | 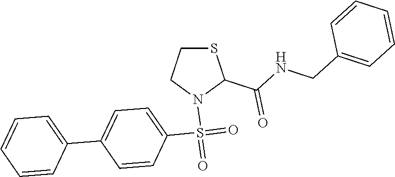 [SMILES: O=C(NCc1ccccc1)C1SCCN1S(=O)(=O)c1ccc(-c2ccccc2)cc1] | ¹H NMR (400 MHz, CDCl₃); 2.6-3.1 (m, CH₂S, 2H), 3.7-4.1 (m, CH₂N, 2H), 4.6- 4.7 (m, NCH₂Ar, 2H), 5.6 (s, CH, 1H), 7.3-8.15 (m, CH(Ar), 14H); M⁺(ESI⁺): 439.1; M−(ESI−): 437.0. |
| 33 | 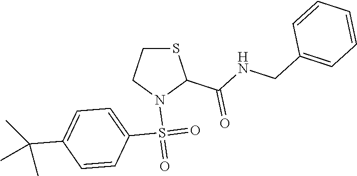 [SMILES: CC(C)(C)c1ccc(S(=O)(=O)N2CCSC2C(=O)NCc2ccccc2)cc1] | ¹H NMR (300 MHz, CDCl₃); 1.1 (s, CH₃, 3H), 2.3-2.8 (m, CH₂S, 2H), 3.5-3.8 (m, CH₂N, 2H), 4.2-4.4 (m, NCH₂Ar, 2H), 5.2 (s, CH, 1H), 7.0-7.65 (m, CH(Ar), 9H); M⁺(ESI⁺): 419.8; M−(ESI−): 417.4. |
| 34 | 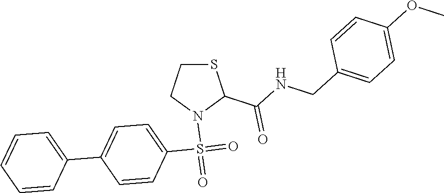 [SMILES: COc1ccc(CNC(=O)C2SCCN2S(=O)(=O)c2ccc(-c3ccccc3)cc2)cc1] | ¹H NMR (400 MHz, CDCl₃); 2.6-3.1 (m, CH₂S, 2H), 3.7-4.1 (m, CH₂N, 2H), 3.8 (s, CH₃O, 3H), 4.6-4.7 (m, NCH₂Ar, 2H), 5.6 (s, CH, 1H), 7.3-8.1 (m, CH(Ar), 13H); M⁺(ESI⁺): 469.1; M−(ESI−): 467.4. |
| 35 | 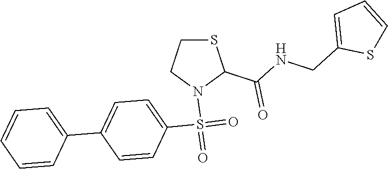 [SMILES: O=C(NCc1cccs1)C1SCCN1S(=O)(=O)c1ccc(-c2ccccc2)cc1] | ¹H NMR (400 MHz, CDCl₃); 2.55-3.1 (m, CH₂S, 2H), 3.65-4.05 (m, CH₂N, 2H), 4.6-4.85 (m, NCH₂Ar, 2H), 5.5 (s, CH, 2H), 6.95-8.05 (m, CH(Ar), 12H); M⁺(ESI⁺): 445.1; M−(ESI−): 443.1. |
| 36 | 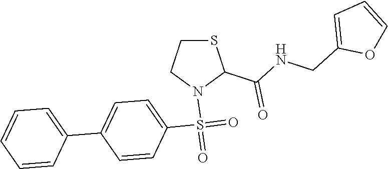 [SMILES: O=C(NCc1ccco1)C1SCCN1S(=O)(=O)c1ccc(-c2ccccc2)cc1] | ¹H NMR (300 MHz, CDCl₃); 2.6-3.1 (m, CH₂S, 2H), 3.8-4.3 (m, CH₂N, 2H), 4.6- 4.75 (m, NCH₂Ar, 2H), 5.7 (s, CH, 2H), 6.6 (m, CH(furyl), 2H), 7.45-8.3 (m, CH(Ar), 10H); M⁺(ESI⁺): 429.5; M−(ESI−): 427.5. |
| 37 | 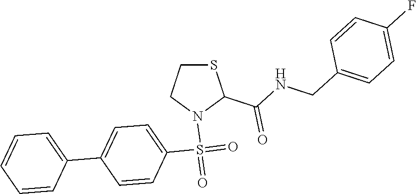 [SMILES: O=C(NCc1ccc(F)cc1)C1SCCN1S(=O)(=O)c1ccc(-c2ccccc2)cc1] | ¹H NMR (400 MHz, CDCl₃); 2.65-3.1 (m, CH₂S, 2H), 3.7-4.1 (m, CH₂N, 2H), 4.6- 4.7 (m, NCH₂Ar, 2H), 5.6 (s, CH, 1H), 6.9-7.9 (m, CH(Ar), 13H); M⁺(ESI⁺): 457.4; M−(ESI−): 455.2. |
| 38 | 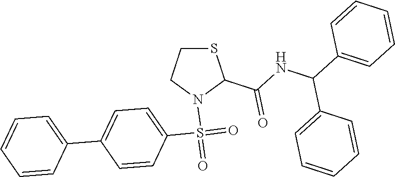 [SMILES: O=C(NC(c1ccccc1)c1ccccc1)C1SCCN1S(=O)(=O)c1ccc(-c2ccccc2)cc1] | ¹H NMR (400 MHz, CDCl₃); 2.3-2.9 (m, CH₂S, 2H), 3.5-3.9 (m, CH₂N, 2H), 5.3 (s, CH, 1H), 6.2 (s x2, NCHAr, 1H), 7.3- 8.15 (m, CH(Ar), 19H); M⁺(ESI⁺): 515.3; M−(ESI−): 513.7. |
| 39 | 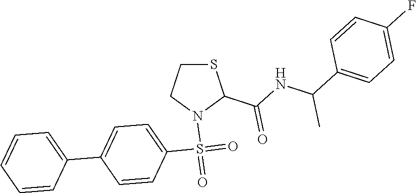 [SMILES: CC(NC(=O)C1SCCN1S(=O)(=O)c1ccc(-c2ccccc2)cc1)c1ccc(F)cc1] | M⁺(ESI⁺): 471.2; M−(ESI−): 469.0. |
| 40 | 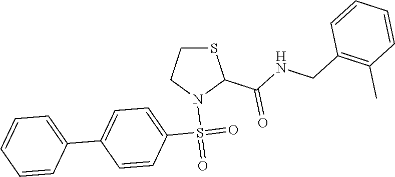 [SMILES: Cc1ccccc1CNC(=O)C1SCCN1S(=O)(=O)c1ccc(-c2ccccc2)cc1] | M⁺(ESI⁺): 453.2; M−(ESI−): 451.0. |
| 41 | 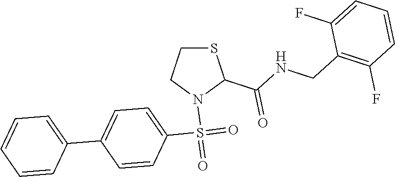 [SMILES: O=C(NCc1c(F)cccc1F)C1SCCN1S(=O)(=O)c1ccc(-c2ccccc2)cc1] | M⁺(ESI⁺): 475.2; M−(ESI−): 473.0. |
| 42 | 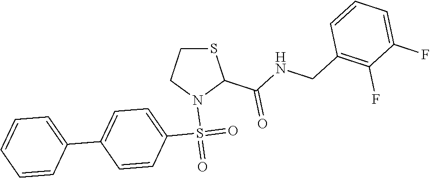 [SMILES: O=C(NCc1cccc(F)c1F)C1SCCN1S(=O)(=O)c1ccc(-c2ccccc2)cc1] | M⁺(ESI⁺): 475.4; M−(ESI−): 472.6. |
| 43 | 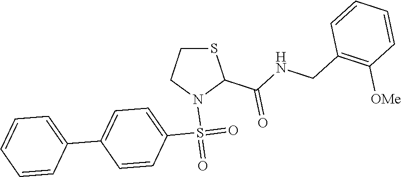 [SMILES: COc1ccccc1CNC(=O)C1SCCN1S(=O)(=O)c1ccc(-c2ccccc2)cc1] | ¹H NMR (400 MHz, CDCl₃); 2.6-3.1 (m, CH₂S, 2H), 3.7-4.1 (m, CH₂N, 2H), 3.8 (s, CH₃O, 3H), 4.6-4.7 (m, NCH₂Ar, 2H), 5.6 (s, CH, 1H), 7.3-8.1 (m, CH(Ar), 13H); M⁺(ESI⁺): 469.2; M−(ESI−): 467.6. |
| 44 | 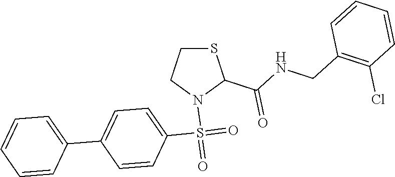 [SMILES: O=C(NCc1ccccc1Cl)C1SCCN1S(=O)(=O)c1ccc(-c2ccccc2)cc1] | M⁺(ESI⁺): 473.3; M−(ESI−): 470.2. |
| 45 | 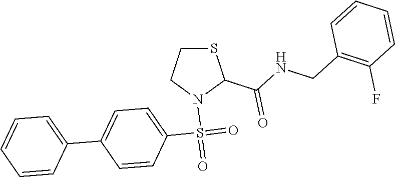 [SMILES: O=C(NCc1ccccc1F)C1SCCN1S(=O)(=O)c1ccc(-c2ccccc2)cc1] | M⁺(ESI⁺): 457.03; M−(ESI−): 455.4. |
| 46 | 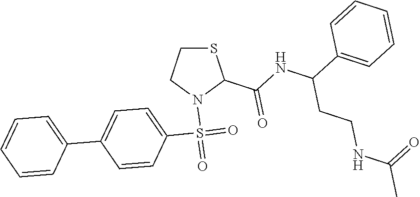 [SMILES: CC(=O)NCCC(NC(=O)C1SCCN1S(=O)(=O)c1ccc(-c2ccccc2)cc1)c1ccccc1] | ¹H NMR (400 MHz, CDCl₃); 1.80-2.10 (m, 6H, CH₃, CH₂S, CH₂), 2.54 (m, 1H, CH₂S), 2.97-3.10 (m, 2H, CH₂), 3.50- 3.90 (m, 3H, NH, CH₂N), 5.0-5.10 (m, 1H, CH), 5.24 (s, 1H, CH), 5.98-6.11 (m, 1H, NH), 7.25-7.89 (m, 14H, CH(Ar)); M⁺(ESI⁺): 523.71; M−(ESI−): 522.05. |
| 47 | 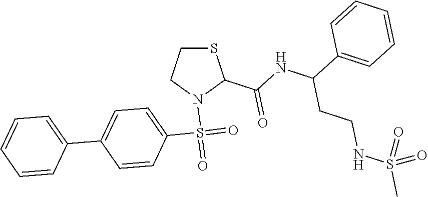 [SMILES: CS(=O)(=O)NCCC(NC(=O)C1SCCN1S(=O)(=O)c1ccc(-c2ccccc2)cc1)c1ccccc1] | ¹H NMR (400 MHz, CDCl₃); 1.80-2.10 (m, 3H, CH₂S, CH₂), 2.88 (s, 3H, CH₃), 3.0-3.50 (m, 3H, CH₂S, CH₂), 3.70-3.85 (m, 2H, CH₂N), 4.45 (m, 1H, NH), 5.13- 5.21 (m, 1H, CH), 5.35 (s, 1H, CH), 6.86-6.94 (m, 1H, NH), 7.35-8.10 (m, 14H, CH(Ar)); M⁺(ESI⁺): 560.21; M−(ESI−): 558.47. |
| 48 | 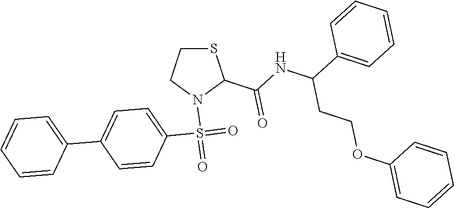 [SMILES: O=C(NC(CCOc1ccccc1)c1ccccc1)C1SCCN1S(=O)(=O)c1ccc(-c2ccccc2)cc1] | ¹H NMR (400 MHz, CDCl₃); 2.22-2.40 (m, 2H, CH₂), 2.41-2.50 (m, 1H, CH₂S), 2.78-2.81 (m, 1H, CH₂S), 3.56-3.93 (m, 4H, CH₂N, CH₂O), 5.21 (m, 1H, CH), 5.31 (s, 1H, CH), 6.79-6.92 (m, 4H, CH(Ar) and NH), 7.20-7.85 (m, 16H, CH(Ar)); M⁺(ESI⁺): 559.39; M− (ESI−): 557.61. |
| 49 | 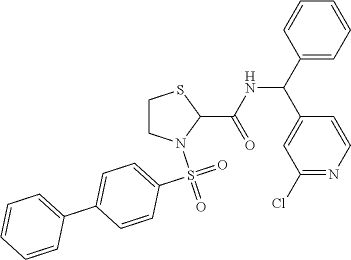 [SMILES: O=C(NC(c1ccccc1)c1ccnc(Cl)c1)C1SCCN1S(=O)(=O)c1ccc(-c2ccccc2)cc1] | ¹H NMR (300 MHz, CDCl₃); 2.58 (m, 1H), 2.93 (m, 1H), 3.77 (m, 2H), 5.28 (s, 0.5H), 5.31 (s, 0.5H), 6.07 (m, 1H), 7.13 (m, 4H), 7.37 (m, 7H), 7.55 (m, 2H), 7.71 (m, 2H), 7.87 (m, 2H), 8.30 (m, 1H). M⁺(ESI⁺): 550; M−(ESI−): 548. |
| 50 | 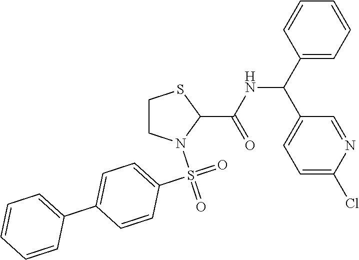 [SMILES: O=C(NC(c1ccccc1)c1ccc(Cl)nc1)C1SCCN1S(=O)(=O)c1ccc(-c2ccccc2)cc1] | ¹H NMR (300 MHz, CDCl₃); 2.60 (m, 1H), 2.94 (m, 1H), 3.72 (m, 1H), 3.86 (m, 1H), 5.31 (s, 0.5H), 5.36 (s, 0.5H), 6.21 (m, 1H), 7.36 (m, 11H), 7.60 (m, 2H), 7.76 (m, 2H), 7.90 (m, 2H), 8.34 (m, 1H). M⁺(ESI⁺): 550; M−(ESI−): 548. |
| 51 | 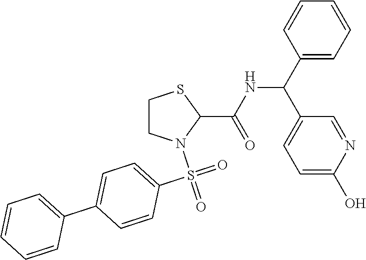 [SMILES: O=C(NC(c1ccccc1)c1ccc(O)nc1)C1SCCN1S(=O)(=O)c1ccc(-c2ccccc2)cc1] | ¹H NMR (300 MHz, DMSO-d₆); 2.58 (m, 1H), 2.97 (m, 1H), 3.69 (m, 2H), 5.41 (m, 1H), 5.65 (m, 1H), 6.18 (m, 1H), 7.20 (m, 10H), 7.60 (m, 2H), 7.77 (m, 4H), 8.73 (m, 1H), 11.37 (br s, 1 H). M⁺(ESI⁺): 532; M−(ESI−): 530. |
| 52 | 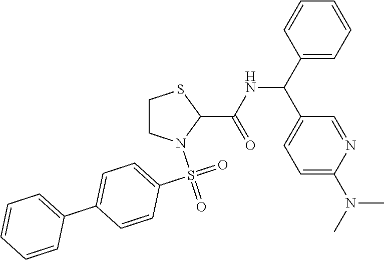 [SMILES: CN(C)c1ccc(C(NC(=O)C2SCCN2S(=O)(=O)c2ccc(-c3ccccc3)cc2)c2ccccc2)cn1] | ¹H NMR (300 MHz, CDCl₃); 2.61 (m, 1H), 2.99 (m, 1H), 3.15 (s, 6H), 3.72 (m, 1H), 3.90 (s, 1H), 5.36 (s, 0.5H), 5.39 (s, 0.5H), 6.10 (m, 1H), 6.56 (m, 1H), 7.13 (m, 1H), 7.28 (m, 6H), 7.46 (m, 3H), 7.60 (m, 2H), 7.73 (m, 2H), 7.91 (m, 2H), 8.02 (m, 0.5H), 8.09 (m, 0.5H). M⁺(ESI⁺): 559; M−(ESI−): 558. |
| 53 | 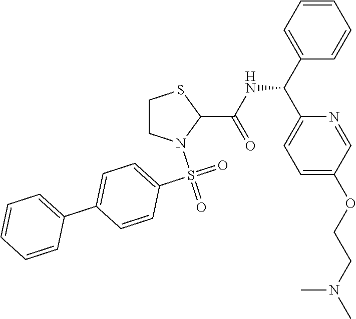 [SMILES: CN(C)CCOc1ccc([C@H](NC(=O)C2SCCN2S(=O)(=O)c2ccc(-c3ccccc3)cc2)c2ccccc2)nc1] | ¹H NMR (300 MHz, CDCl₃); 2.63 (m, 7H), 2.99 (m, 3H), 3.71 (m, 1H), 4.02 (m, 1H), 4.26 (br s, 2H), 5.48 (s, 0.5H), 5.49 (s, 0.5H), 6.02 (s, 0.5H), 6.05 (s, 0.5H), 7.25 (m, 6H), 7.44 (m, 4FI), 7.58 (m, 2FI), 7.70 (m, 2H), 7.93 (m, 2H), 8.33 (m, 2H). M⁺(ESI⁺): 603; M−(ESI−): 601. |
| 54 | 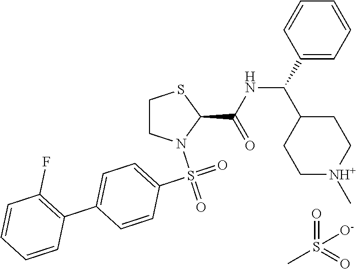 | ¹H NMR (300 MHz, DMSO-c/6); 1.40 (m, 2H), 1.92 (m, 2H), 2.29 (s, 3H), 2.70 (m, 3H), 3.06 (m, 1H), 3.31 (m, 4H), 3.82 (m, 2H), 4.58 (m, 1H), 5.50 (s, 1H), 7.34 (m, 7H), 7.56 (m, 2H), 7.90 (m, 4H), 8.57 (m, 1H), 9.10 (m, 1H). M⁺(ESI⁺): 554; M−(ESI−): 552. |
| 55 | 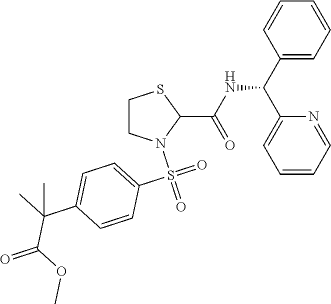 [SMILES: COC(=O)C(C)(C)c1ccc(S(=O)(=O)N2CCSC2C(=O)N[C@H](c2ccccc2)c2ccccn2)cc1] | ¹H NMR (300 MHz, CDCl₃); δ 8.63 (s, 1H); 7.82-7.91 (m, 3H); 7.33-7.51 (m, 9H); 6.2 (s, 1H); 5.65 (s, 1H); 3.88 (m, 2H); 3.66 (s, 3H); 3.10 (m, 1H); 2.65 (m, 1H); 1.6 (s, 6H). M⁺(ESI⁺): 540.1; M−(ESI−): 538.0. |
| 56 | 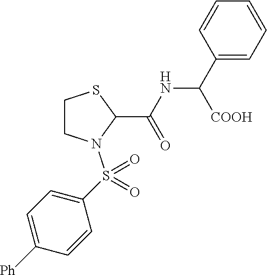 [SMILES: O=C(O)C(NC(=O)C1SCCN1S(=O)(=O)c1ccc(-c2ccccc2)cc1)c1ccccc1] | ¹H NMR (300 MHz, DMSO-d6); 2.68 (m, 1H), 3.08 (m, 1H), 3.84 (m, 2H), 5.30 (m, 1H), 5.76 (s, 0.5H), 5.82 (s, 0.5H), 7.44 (m, 8H), 7.78 (m, 2H), 7.93 (m, 4H), 8.91 (m, 1H), 13.10 (br s, 1H). M⁺(ESI⁺): 483; M−(ESI−): 481. |
| 57 | 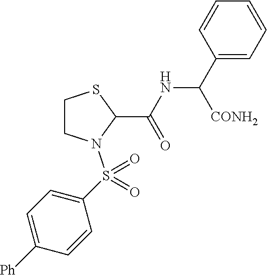 [SMILES: NC(=O)C(NC(=O)C1SCCN1S(=O)(=O)c1ccc(-c2ccccc2)cc1)c1ccccc1] | ¹HNMR (300 MHz, DMSO-d6); 2.64 (m, 1H), 3.04 (m, 1H), 3.78 (m, 2H), 5.34 (m, 1H), 5.84 (m, 1H), 7.29 (m, 4H), 7.47 (m, 4H), 7.76 (m, 2H), 7.90 (m, 4H), 8.77 (m, 1H). M⁺(ESI⁺): 482; M−(ESI−): 480. |
| 58 | 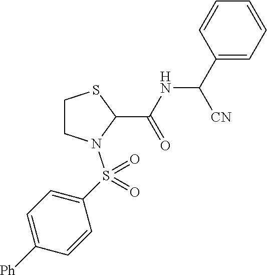 [SMILES: N#CC(NC(=O)C1SCCN1S(=O)(=O)c1ccc(-c2ccccc2)cc1)c1ccccc1] | ¹H NMR (300 MHz, CDCl₃); 2.61 (m, 1H), 2.99 (m, 1H), 3.72 (m, 2H), 5.35 (s, 0.5H), 5.41 (s, 0.5H), 6.06 (m, 0.5H), 6.17 (m, 0.5H), 7.17 (m, 1H), 7.47 (m, 8H), 7.61 (m, 2H), 7.77 (m, 2H), 7.91 (m, 2H). M⁺(ESI⁺): 464; M−(ESI−): 462. |
| 59 | 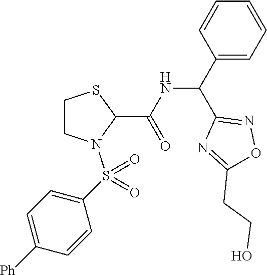 [SMILES: O=C(NC(c1ccccc1)c1noc(CCO)n1)C1SCCN1S(=O)(=O)c1ccc(-c2ccccc2)cc1] | ¹H NMR (300 MHz, CDCl₃); 2.35 (br s, 2H), 2.55 (m, 2H), 3.00 (m, 1H), 3.60- 3.93 (m, 2H), 5.30 (s, 0.5H), 5.40 (s, 0.5H),), 5.64 (m, 1H) 7.05-8.17 (m, 14H, H arom.). M⁺(ESI⁺): 497. M−(ESI−): 495. |
| 60 | 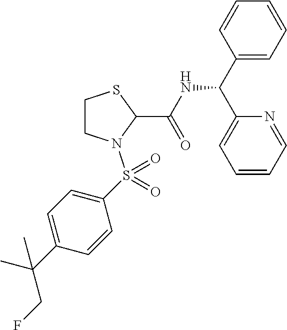 [SMILES: CC(C)(CF)c1ccc(S(=O)(=O)N2CCSC2C(=O)N[C@H](c2ccccc2)c2ccccn2)cc1] | ¹H NMR (300 MHz, CDCl₃); δ 8.07 (m, 2H); 7.82-7.97 (m, 3H); 7.28-7.58 (m, 9H); 6.26 (s, 1H); 3.86 (m, 2H); 3.36 (d, 2H); 3.635 (m, 1H); 2.71 (m, 1H); 1.36 (s, 1H) M⁺(ESI⁺): 512.4. M−(ESI−): 510.3. ¹H-RMN (CH₂Cl2) δ 8.58 (m, 2H); 7.83 (t, Jt = 8.29, 2H); 7.65 (m, 1H); 7.19- 7.40 (m, 9H); 6.10 (s, 1H); 5.46 (s, 1H); 3.95-4.06 (m, 1H); 3.65-3.78 (m, 1H); 2.92-2.99 (m, 3H); 2.55-2.64 (m, 1H); 1.25-1.37 (m, 6H). ¹⁹F-RMN (CH₂Cl₂) δ −138.6. M⁺(ESI⁺): 514.2; M− (ESF): 512.2 |
| 61 | 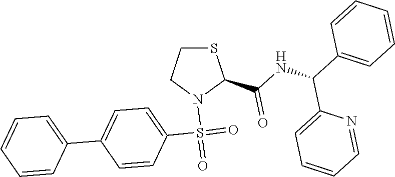 [SMILES: O=C(N[C@H](c1ccccc1)c1ccccn1)[C@@H]1SCCN1S(=O)(=O)c1ccc(-c2ccccc2)cc1] | M⁺(ESI⁺): 516; M−(ESI−): 514. |
| 62 | 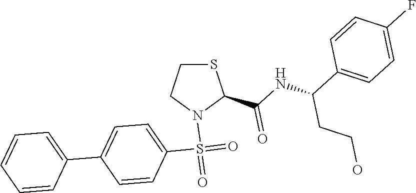 [SMILES: O=C(N[C@@H](CCO)c1ccc(F)cc1)[C@@H]1SCCN1S(=O)(=O)c1ccc(-c2ccccc2)cc1] | M⁺(ESI⁺): 501.6; M−(ESI−): 499.2. |
| 63 | 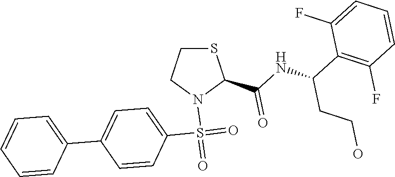 [SMILES: O=C(N[C@@H](CCO)c1c(F)cccc1F)[C@@H]1SCCN1S(=O)(=O)c1ccc(-c2ccccc2)cc1] | M⁺(ESI⁺): 519.9; M−(ESI−): 517.8 |
| 64 | 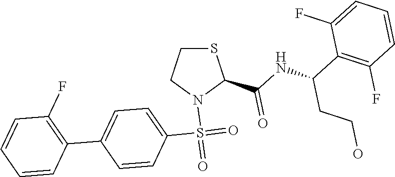 [SMILES: O=C(N[C@@H](CCO)c1c(F)cccc1F)[C@@H]1SCCN1S(=O)(=O)c1ccc(-c2ccccc2F)cc1] | M⁺(ESI⁺): 537.9; M−(ESI−): 535.9. |
| 65 | 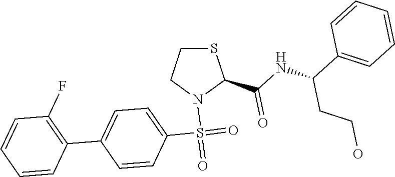 [SMILES: O=C(N[C@@H](CCO)c1ccccc1)[C@@H]1SCCN1S(=O)(=O)c1ccc(-c2ccccc2F)cc1] | M⁺(ESI⁺): 501.9; M−(ESI−): 499.5. |
| 66 | 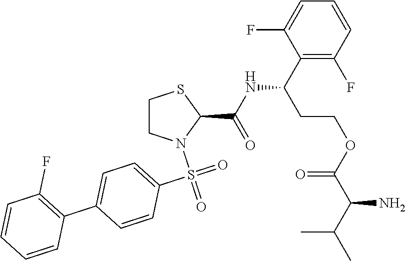 [SMILES: CC(C)[C@H](N)C(=O)OCC[C@H](NC(=O)[C@@H]1SCCN1S(=O)(=O)c1ccc(-c2ccccc2F)cc1)c1c(F)cccc1F] | M⁺(ESI⁺): 636.7; M−(ESI−): 634.3. |
| 67 | 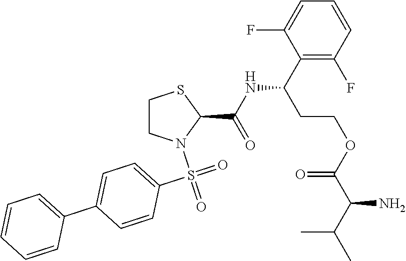 [SMILES: CC(C)[C@H](N)C(=O)OCC[C@H](NC(=O)[C@@H]1SCCN1S(=O)(=O)c1ccc(-c2ccccc2)cc1)c1c(F)cccc1F] | M⁺(ESI⁺): 618.9; M−(ESI−): 616.5. |
| 68 | 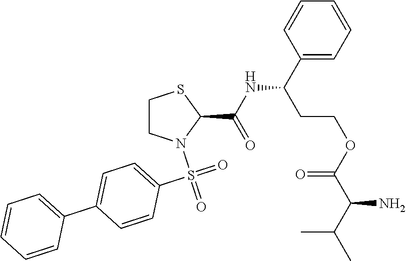 [SMILES: CC(C)[C@H](N)C(=O)OCC[C@H](NC(=O)[C@@H]1SCCN1S(=O)(=O)c1ccc(-c2ccccc2)cc1)c1ccccc1] | M⁺(ESI⁺): 582.9; M−(ESI−): 581.3. |
| 69 | 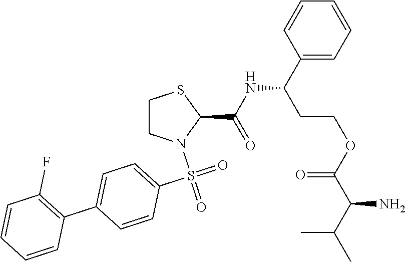 [SMILES: CC(C)[C@H](N)C(=O)OCC[C@H](NC(=O)[C@@H]1SCCN1S(=O)(=O)c1ccc(-c2ccccc2F)cc1)c1ccccc1] | M⁺(ESI⁺): 600.8; M−(ESI−): 598.6. |
| 70 | 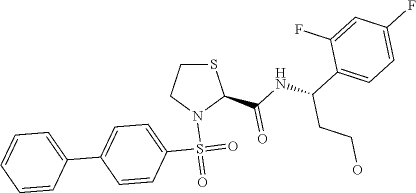 [SMILES: O=C(N[C@@H](CCO)c1ccc(F)cc1F)[C@@H]1SCCN1S(=O)(=O)c1ccc(-c2ccccc2)cc1] | M⁺(ESI⁺): 519.6; M−(ESI−): 517.6. |

### Atosiban

Atosiban, sold under the name TRACTOCILE®, is an oxytocin receptor antagonist that inhibits the release of calcium ions from the sarcoplasmic reticulum into the cytoplasm of uterine myocytes. Uterine contractility is dependent, in part, upon this mobilization of divalent calcium ions. Atosiban may inhibit calcium ion influx in uterine myocytes without altering serum calcium concentrations. Atosiban is represented structurally as compound (4), below.

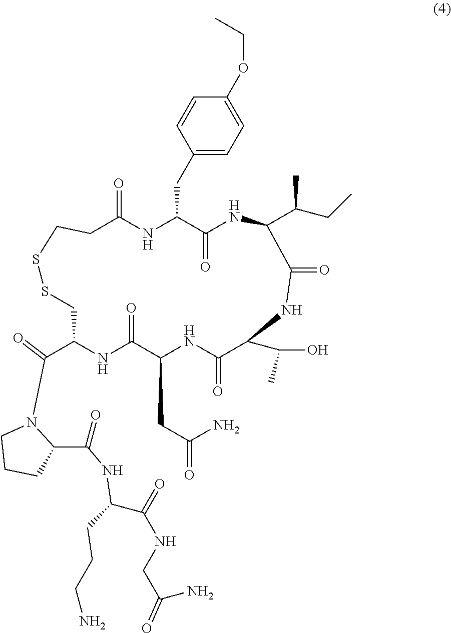 [SMILES: CCOc1ccc(C[C@H]2NC(=O)CCSSC[C@@H](C(=O)N3CCC[C@H]3C(=O)N[C@@H](CCCN)C(=O)NCC(N)=O)NC(=O)[C@H](CC(N)=O)NC(=O)[C@H]([C@@H](C)O)NC(=O)[C@H]([C@@H](C)CC)NC2=O)cc1]

Exemplary methods for the preparation of atosiban are described, for instance, in U.S. Pat. Nos. 4,504,469 and 4,402,942, and may involve solid-phase peptide synthesis as well as solution-phase cyclization, for instance, by disulfide bond formation. It is to be understood that the foregoing compound can be synthesized by alternative methods, for instance, by substituting one of the amide-bond forming agents shown U.S. Pat. No. 4,504,469 or 4,402,942 with another amide-bond forming agent described herein or known in the art.

Oxytocin antagonists useful in conjunction with the compositions and methods described herein include atosiban, as well as salts, derivatives, variants, crystal forms, and formulations thereof, such as a salt, derivative, variant, crystal form, or formulation described in U.S. Pat. No. 4,504,469 or 4,402,942, the disclosures of each of which are incorporated herein by reference in their entirety.

### Methods of Treatment

### Combination Therapy

A PGF2α receptor antagonist, such as a 1,3-thiazolidine-2-carboxamide compound described herein, may be administered to a subject (e.g., a pregnant human subject) in combination with atosiban so as to treat or prevent preterm parturition. The combined administration of these agents, such as in accordance with a dosing schedule described above, provides important clinical benefits. For instance, the compositions and methods described herein enable pregnancy to be prolonged by one or more hours, days, or weeks. Particular examples of clinical benefits engendered by the compositions and methods described herein include without limitation, (i) a delay in the onset of delivery by the subject, such as a delay of one or more hours, days, or weeks (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others) following the first administration of atosiban and the PGF2α receptor antagonist to the subject, (ii) a delay in the onset of delivery by the subject such that the subject undergoes delivery at a gestational age of at least about 34 weeks, such as at a gestational age of from about 34 weeks to about 40 weeks (e.g., at a gestational age of about 34 weeks, 35 weeks, 36 weeks, 37 weeks, 38 weeks, 39 weeks, or 40 weeks) following administration of atosiban and the PGF2α receptor antagonist to the subject, (iii) a reduction in vaginal bleeding by the subject following administration of atosiban and the PGF2α receptor antagonist to the subject, (iv) a delay in the onset of amniorrhexis by the subject (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others, following the first administration of atosiban and the PGF2α receptor antagonist to the subject), (v) a reduction in the expression of one or more proinflammatory genes, such as cyclooxygenase-2 (Cox2) by the subject (as assessed, e.g., by observing a decrease in myometrial Cox2 expression) following administration of atosiban and the PGF2α receptor antagonist to the subject, and (vi) a reduction in the frequency of, peak amplitude of, duration of, and/or work done by, uterine contractions in the subject following administration of atosiban and the PGF2α receptor antagonist to the subject.

### Antenatal Corticosteroids

Additionally, atosiban and a PGF2α receptor antagonist, such as a PGF2α receptor antagonist described herein, may be administered to a patient (e.g., a patient undergoing or at risk of undergoing preterm labor) in conjunction with a corticosteroid. Antenatal corticosteroids, such as betamethasone, dexamethasone, and hydrocortisone, represent a class of therapeutic agents that can be administered to a subject in order to accelerate fetal lung maturation prior to birth. Treatment with antenatal corticosteroids is associated with an overall reduction in neonatal death, respiratory distress syndrome, intraventricular hemorrhage, necrotizing enterocolitis, respiratory support, intensive care admissions, and systemic infections in the first 48 hours of life. Additionally, antenatal corticosteroid therapy is effective in women with premature rupture of membranes (PROM) and pregnancy-related hypertension syndromes. There is evidence to suggest benefit across a wide range of gestational ages, such as from about 26 to about 34 weeks, among others (Miracle et al. J. Perinat. Med. 36:191-196 (2008), the disclosure of which is incorporated herein by reference). Thus, using the compositions and methods described herein, a patient may be administered atosiban and a PGF2α receptor antagonist (such as a compound represented by any one of formulas (I) through (VIII), e.g., compound (1), (2), or (3)) so as to delay the onset of delivery, thereby providing additional time to administer an antenatal corticosteroid in order to accelerate fetal lung development before birth.

### Assessing Patient Response

A variety of methods known in the art and described herein can be used to determine whether a patient (e.g., a patient undergoing or at risk of undergoing preterm labor) is responding favorably to tocolytic treatment. For example, successful treatment with atosiban and a PGF2α receptor antagonist, such as a 1,3-thiazolidine-2-carboxamide compound described herein, may be signaled by:
(a) a delay in the onset of delivery by the subject, such as a delay of one or more hours, days, or weeks (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others) following the first administration of atosiban and the PGF2α receptor antagonist to the subject;
(b) a delay in the onset of delivery by the subject such that the subject undergoes delivery at a gestational age of at least about 34 weeks, such as at a gestational age of from about 34 weeks to about 40 weeks (e.g., at a gestational age of about 34 weeks, 35 weeks, 36 weeks, 37 weeks, 38 weeks, 39 weeks, or 40 weeks) following administration of atosiban and the PGF2α receptor antagonist to the subject;
(c) a reduction in vaginal bleeding by the subject following administration of atosiban and the PGF2α receptor antagonist to the subject;
(d) a delay in the onset of amniorrhexis by the subject (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others, following the first administration of atosiban and the PGF2α receptor antagonist to the subject);
(e) a reduction in the expression of one or more proinflammatory genes, such as cyclooxygenase-2 (Cox2) by the subject (as assessed, e.g., by observing a decrease in myometrial Cox2 expression) following administration of atosiban and the PGF2α receptor antagonist to the subject; and/or
(f) a reduction in the frequency of, peak amplitude of, duration of, and/or work done by, uterine contractions in the subject following administration of atosiban and the PGF2α receptor antagonist to the subject.

### Routes of Administration and Dosing

Using the compositions and methods described herein, atosiban and a PGF2α receptor antagonist may be administered to a subject (e.g., a pregnant human subject) so as to treat or prevent preterm labor. Exemplary dosage amounts and schedules that may be used for administration of atosiban and the PGF2α receptor antagonists of the disclosure are described in the sections that follow.

### Atosiban

In accordance with the compositions and methods of the disclosure, atosiban may be administered to a subject in only one dose or may be periodically administered to the subject in one or more doses, for example, per 12 hours, 24 hours, 36 hours, 48 hours, 60 hours, 72 hours, 84 hours, 96 hours, week, or month. For instance, the atosiban may be administered to the subject in from one to ten doses (e.g., 1, 2, 3, 4, 5, 6, 7, 8, 9, or 10 doses) every 12 hours, one to ten doses (e.g., 1, 2, 3, 4, 5, 6, 7, 8, 9, or 10 doses) every 14 hours, one to ten doses (e.g., 1, 2, 3, 4, 5, 6, 7, 8, 9, or 10 doses) every 16 hours, one to ten doses (e.g., 1, 2, 3, 4, 5, 6, 7, 8, 9, or 10 doses) every 18 hours, one to ten doses (e.g., 1, 2, 3, 4, 5, 6, 7, 8, 9, or 10 doses) every 20 hours, one to ten doses (e.g., 1, 2, 3, 4, 5, 6, 7, 8, 9, or 10 doses) every 22 hours, one to ten doses (e.g., 1, 2, 3, 4, 5, 6, 7, 8, 9, or 10 doses) every 24 hours, one to ten doses (e.g., 1, 2, 3, 4, 5, 6, 7, 8, 9, or 10 doses) every 26 hours, one to ten doses (e.g., 1, 2, 3, 4, 5, 6, 7, 8, 9, or 10 doses) every 28 hours, one to ten doses (e.g., 1, 2, 3, 4, 5, 6, 7, 8, 9, or 10 doses) every 30 hours, one to ten doses (e.g., 1, 2, 3, 4, 5, 6, 7, 8, 9, or 10 doses) every 32 hours, one to ten doses (e.g., 1, 2, 3, 4, 5, 6, 7, 8, 9, or 10 doses) every 34 hours, one to ten doses (e.g., 1, 2, 3, 4, 5, 6, 7, 8, 9, or 10 doses) every 36 hours, one to ten doses (e.g., 1, 2, 3, 4, 5, 6, 7, 8, 9, or 10 doses) every 38 hours, one to ten doses (e.g., 1, 2, 3, 4, 5, 6, 7, 8, 9, or 10 doses) every 40 hours, one to ten doses (e.g., 1, 2, 3, 4, 5, 6, 7, 8, 9, or 10 doses) every 42 hours, one to ten doses (e.g., 1, 2, 3, 4, 5, 6, 7, 8, 9, or 10 doses) every 44 hours, one to ten doses (e.g., 1, 2, 3, 4, 5, 6, 7, 8, 9, or 10 doses) every 46 hours, one to ten doses (e.g., 1, 2, 3, 4, 5, 6, 7, 8, 9, or 10 doses) every 48 hours, one to ten doses (e.g., 1, 2, 3, 4, 5, 6, 7, 8, 9, or 10 doses) every 60 hours, one to ten doses (e.g., 1, 2, 3, 4, 5, 6, 7, 8, 9, or 10 doses) every 72 hours, one to ten doses (e.g., 1, 2, 3, 4, 5, 6, 7, 8, 9, or 10 doses) every 84 hours, one to ten doses (e.g., 1, 2, 3, 4, 5, 6, 7, 8, 9, or 10 doses) every 96 hours, one to ten doses (e.g., 1, 2, 3, 4, 5, 6, 7, 8, 9, or 10 doses) every 108 hours, one to ten doses (e.g., 1, 2, 3, 4, 5, 6, 7, 8, 9, or 10 doses) every 120 hours, one to ten doses (e.g., 1, 2, 3, 4, 5, 6, 7, 8, 9, or 10 doses) every 132 hours, one to ten doses (e.g., 1, 2, 3, 4, 5, 6, 7, 8, 9, or 10 doses) every 144 hours, one to ten doses (e.g., 1, 2, 3, 4, 5, 6, 7, 8, 9, or 10 doses) every 156 hours, or one to ten doses (e.g., 1, 2, 3, 4, 5, 6, 7, 8, 9, or 10 doses) every week.

In some embodiments, the atosiban is administered to the subject in a single bolus dose of from about 4.0 mg to about 9.5 mg (e.g., a single bolus dose of about 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, or 9.0 mg). In some embodiments, the atosiban is administered to the subject in a single bolus dose of from about 5.0 mg to about 8.5 mg (e.g., a single bolus dose of about 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, or 8.5 mg). In some embodiments, the atosiban is administered to the subject in a single bolus dose of from about 5.5 mg to about 8.0 mg (e.g., a single bolus dose of about 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, or 8.0 mg). In some embodiments, the atosiban is administered to the subject in a single bolus dose of from about 5.75 mg to about 7.75 mg (e.g., a single bolus dose of about 5.75 mg, 6.00 mg, 6.25 mg, 6.50 mg, 6.75 mg, 7.00 mg, 7.25 mg, 7.50 mg, or 7.75 mg). In some embodiments, the atosiban is administered to the subject in a single bolus dose of from about 6.0 mg to about 7.5 mg (e.g., a single bolus dose of about 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, or 7.5 mg). In some embodiments, the atosiban is administered to the subject in a single bolus dose of from about 6.25 mg to about 7.25 mg (e.g., a single bolus dose of about 6.25 mg, 6.50 mg, 6.75 mg, 7.00 mg, or 7.25 mg). In some embodiments, the atosiban is administered to the subject in a single bolus dose of from about 6.5 mg to about 7.0 mg (e.g., a single bolus dose of about 6.50 mg, 6.75 mg, or 7.00. mg). In some embodiments, the atosiban is administered to the subject in a single bolus dose of about 6.75 mg.

In some embodiments, the single bolus dose of atosiban is administered to the subject intravenously.

In some embodiments, following the single bolus dose of atosiban, the atosiban is subsequently administered to the subject in an amount of from about 40 mg to about 70 mg by way of a continuous, intravenous infusion (e.g., an amount of about 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, or 70 mg by way of a continuous, intravenous infusion). In some embodiments, following the single bolus dose of atosiban, the atosiban is subsequently administered to the subject in an amount of from about 45 mg to about 65 mg by way of a continuous, intravenous infusion (e.g., an amount of about 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg by way of a continuous, intravenous infusion). In some embodiments, following the single bolus dose of atosiban, the atosiban is subsequently administered to the subject in an amount of from about 50 mg to about 60 mg by way of a continuous, intravenous infusion (e.g., an amount of about 50 mg, 51 mg, 52 mg, 53 mg, 54 mg, 55 mg, 56 mg, 57 mg, 58 mg, 59 mg, or 60 mg by way of a continuous, intravenous infusion). In some embodiments, following the single bolus dose of atosiban, the atosiban is subsequently administered to the subject in an amount of from about 52 mg to about 56 mg by way of a continuous, intravenous infusion (e.g., an amount of about 52 mg, 53 mg, 54 mg, 55 mg, or 56 mg by way of a continuous, intravenous infusion). In some embodiments, following the single bolus dose of atosiban, the atosiban is subsequently administered to the subject in an amount of about 54 mg by way of a continuous, intravenous infusion.

In some embodiments, the foregoing intravenous infusion is administered continuously over a period of from about 1 hour to about 5 hours, such as a period of about 1.0 hours, 1.25 hours, 1.5 hours, 1.75 hours, 2.0 hours, 2.25 hours, 2.5 hours, 2.75 hours, 3.0 hours, 3.25 hours, 3.5 hours, 3.5 hours, 3.75 hours, 4.0 hours, 4.25 hours, 4.5 hours, 4.75 hours, or 5.0 hours). In some embodiments, the foregoing intravenous infusion is administered continuously over a period of from about 1.5 hours to about 4.5 hours, such as a period of from about 1.5 hours, 1.75 hours, 2.0 hours, 2.25 hours, 2.5 hours, 2.75 hours, 3.0 hours, 3.25 hours, 3.5 hours, 3.5 hours, 3.75 hours, 4.0 hours, 4.25 hours, or 4.5 hours). In some embodiments, the foregoing intravenous infusion is administered continuously over a period of from about 2.0 hours to about 4.0 hours, such as a period of from about 2.0 hours, 2.25 hours, 2.5 hours, 2.75 hours, 3.0 hours, 3.25 hours, 3.5 hours, 3.5 hours, 3.75 hours, or 4.0 hours). In some embodiments, the foregoing intravenous infusion is administered continuously over a period of from about 2.5 hours to about 3.5 hours, such as a period of from about 2.5 hours, 2.75 hours, 3.0 hours, 3.25 hours, or 3.5 hours). In some embodiments, the foregoing intravenous infusion is administered continuously over a period of about 3.0 hours.

In some embodiments, following the foregoing continuous, intravenous infusion, the atosiban is subsequently administered to the subject in an amount of from about 240 mg to about 300 mg by way of a second continuous, intravenous infusion (e.g., an amount of about 240 mg, 245 mg, 250 mg, 255 mg, 260 mg, 265 mg, 270 mg, 275 mg, 280 mg, 285 mg, 290 mg, 295 mg, or 300 mg by way of a second continuous, intravenous infusion). In some embodiments, following the foregoing continuous, intravenous infusion, the atosiban is subsequently administered to the subject in an amount of from about 250 mg to about 290 mg by way of a second continuous, intravenous infusion (e.g., an amount of about 250 mg, 255 mg, 260 mg, 265 mg, 270 mg, 275 mg, 280 mg, 285 mg, or 290 mg by way of a second continuous, intravenous infusion). In some embodiments, following the foregoing continuous, intravenous infusion, the atosiban is subsequently administered to the subject in an amount of from about 250 mg to about 290 mg by way of a second continuous, intravenous infusion (e.g., an amount of about 250 mg, 255 mg, 260 mg, 265 mg, 270 mg, 275 mg, 280 mg, 285 mg, or 290 mg by way of a second continuous, intravenous infusion). In some embodiments, following the foregoing continuous, intravenous infusion, the atosiban is subsequently administered to the subject in an amount of from about 260 mg to about 280 mg by way of a second continuous, intravenous infusion (e.g., an amount of about 260 mg, 265 mg, 270 mg, 275 mg, or 280 mg by way of a second continuous, intravenous infusion). In some embodiments, following the foregoing continuous, intravenous infusion, the atosiban is subsequently administered to the subject in an amount of from about 265 mg to about 275 mg by way of a second continuous, intravenous infusion (e.g., an amount of about 265 mg, 270 mg, or 275 mg by way of a second continuous, intravenous infusion). In some embodiments, following the foregoing continuous, intravenous infusion, the atosiban is subsequently administered to the subject in an amount of about 270 mg by way of a second continuous, intravenous infusion.

In some embodiments, the second intravenous infusion is administered continuously over a period of from about 42 hours to about 48 hours, such as a period of about 42.0 hours, 42.25 hours, 42.5 hours, 42.75 hours, 43.0 hours, 43.25 hours, 43.5 hours, 43.75 hours, 44.0 hours, 44.25 hours, 44.5 hours, 44.75 hours, 45.0 hours, 45.25 hours, 45.5 hours, 45.75 hours, 46.0 hours, 46.25 hours, 46.5 hours, 46.75 hours, 47.0 hours, 47.25 hours, 47.5 hours, 47.75 hours, or 48.0 hours). In some embodiments, the second intravenous infusion is administered continuously over a period of from about 43 hours to about 47 hours, such as a period of about 43.0 hours, 43.25 hours, 43.5 hours, 43.75 hours, 44.0 hours, 44.25 hours, 44.5 hours, 44.75 hours, 45.0 hours, 45.25 hours, 45.5 hours, 45.75 hours, 46.0 hours, 46.25 hours, 46.5 hours, 46.75 hours, or 47.0 hours). In some embodiments, the second intravenous infusion is administered continuously over a period of from about 44 hours to about 46 hours, such as a period of about 44.0 hours, 44.25 hours, 44.5 hours, 44.75 hours, 45.0 hours, 45.25 hours, 45.5 hours, 45.75 hours, or 46.0 hours). In some embodiments, the second intravenous infusion is administered continuously over a period of from about 44.5 hours to about 45.5 hours, such as a period of about 44.5 hours, 44.75 hours, 45.0 hours, 45.25 hours, or 45.5 hours). In some embodiments, the second intravenous infusion is administered continuously over a period of about 44 hours.

In some embodiments, the atosiban is administered to the subject until the subject reaches a gestational age of at least about 34 weeks (e.g., a gestational age of from about 34 weeks to about 40 weeks, such as a gestational age of 34 weeks, 35 weeks, 36 weeks, 37 weeks, 38 weeks, 39 weeks, or 40 weeks), or until the subject undergoes delivery. In some embodiments, the atosiban is administered to the subject until the subject reaches a gestational age of about 37 weeks.

In some embodiments, administration of the PGF2α receptor antagonist and administration of the atosiban commence within about 48 hours of one another. In some embodiments, administration of the PGF2α receptor antagonist and administration of the atosiban commence within about 36 hours of one another. In some embodiments, administration of the PGF2α receptor antagonist and administration of the atosiban commence within about 24 hours of one another. In some embodiments, administration of the PGF2α receptor antagonist and administration of the atosiban commence within about 12 hours of one another. In some embodiments, administration of the PGF2α receptor antagonist and administration of the atosiban commence within about 6 hours of one another. In some embodiments, administration of the PGF2α receptor antagonist and administration of the atosiban commence at substantially the same time.

In some embodiments, the atosiban is formulated as an aqueous solution. The concentration of atosiban in the aqueous solution may be, for example, from about 5.0 mg/ml to about 10 mg mg/ml (e.g., about 5.0 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml, 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9.0 mg/ml, 9.1 mg/ml, 9.2 mg/ml, 9.3 mg/ml, 9.4 mg/ml, 9.5 mg/ml, 9.6 mg/ml, 9.7 mg/ml, 9.8 mg/ml, 9.9 mg/ml, or 10.0 mg/ml). In some embodiments, the concentration of atosiban in the aqueous solution is from about 5.5 mg/ml to about 9.5 mg/ml (e.g., about 5.5 mg/ml, 5.6 mg/ml, 5.7 mg/ml, 5.8 mg/ml, 5.9 mg/ml, 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9.0 mg/ml, 9.1 mg/ml, 9.2 mg/ml, 9.3 mg/ml, 9.4 mg/ml, or 9.5 mg/ml). In some embodiments, the concentration of atosiban in the aqueous solution is from about 6.0 mg/ml to about 9.0 mg/ml (e.g., about 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, or 9.0 mg/ml). In some embodiments, the concentration of atosiban in the aqueous solution is from about 6.5 mg/ml to about 8.5 mg/ml (e.g., about 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, or 8.5 mg/ml). In some embodiments, the concentration of atosiban in the aqueous solution is from about 7.0 mg/ml to about 8.0 mg/ml (e.g., about 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, or 8.0 mg/ml). In some embodiments, the concentration of atosiban in the aqueous solution is about 7.5 mg/ml.

The aqueous solution may further contain one or more pharmaceutically acceptable excipients, such as mannitol. In some embodiments, the concentration of mannitol in the aqueous solution is from about 40 mg/ml to about 60 mg/ml, such as from about 45 mg/ml to about 55 mg/ml (e.g., about 50 mg/ml).

In some embodiments, the aqueous solution has a pH of from about 4.0 to about 5.0 (e.g., a pH of about 4.0, 4.1, 4.2, 4.3, 4.4, 4.5, 4.6, 4.7, 4.8, 4.9, or 5.0). In some embodiments, the aqueous solution has a pH of from about 4.25 to about 4.75 (e.g., a pH of about 4.25, 4.30, 4.35, 4.40, 4.45, 4.50, 4.55, 4.60, 4.65, 4.70, or 4.75). In some embodiments, the aqueous solution has a pH of about 4.5.

### PGF2α Receptor Antagonists

A PGF2α receptor antagonist described herein may be administered to the subject in an amount of from about 300 mg to about 2,300 mg per dose, such as an amount of about 300 mg per dose, 350 mg per dose, 400 mg per dose, 450 mg per dose, 500 mg per dose, 550 mg per dose, 600 mg per dose, 650 mg per dose, 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, 1,300 mg per dose, 1,350 mg per dose, 1,400 mg per dose, 1,450 mg per dose, 1,500 mg per dose, 1,550 mg per dose, 1,600 mg per dose, 1,650 mg per dose, 1,700 mg per dose, 1,750 mg per dose, 1,800 mg per dose, 1,850 mg per dose, 1,900 mg per dose, 1,950 mg per dose, 2,000 mg per dose, 2,050 mg per dose, 2,100 mg per dose, 2,150 mg per dose, 2,200 mg per dose, 2,250 mg per dose, or 2,300 mg per dose. The PGF2α receptor antagonist may be administered to the subject in an amount of from about 400 mg to about 2,100 mg per dose, such as an amount of about 400 mg per dose, 450 mg per dose, 500 mg per dose, 550 mg per dose, 600 mg per dose, 650 mg per dose, 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, 1,300 mg per dose, 1,350 mg per dose, 1,400 mg per dose, 1,450 mg per dose, 1,500 mg per dose, 1,550 mg per dose, 1,600 mg per dose, 1,650 mg per dose, 1,700 mg per dose, 1,750 mg per dose, 1,800 mg per dose, 1,850 mg per dose, 1,900 mg per dose, 1,950 mg per dose, 2,000 mg per dose, 2,050 mg per dose, or 2,100 mg per dose. In some embodiments, the PGF2α receptor antagonist is administered to the subject in an amount of from about 450 mg to about 2,050 mg per dose, such as an amount of about 450 mg per dose, 500 mg per dose, 550 mg per dose, 600 mg per dose, 650 mg per dose, 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, 1,300 mg per dose, 1,350 mg per dose, 1,400 mg per dose, 1,450 mg per dose, 1,500 mg per dose, 1,550 mg per dose, 1,600 mg per dose, 1,650 mg per dose, 1,700 mg per dose, 1,750 mg per dose, 1,800 mg per dose, 1,850 mg per dose, 1,900 mg per dose, 1,950 mg per dose, 2,000 mg per dose, or 2,050 mg per dose.

In some embodiments, the PGF2α receptor antagonist is administered to the subject in an amount of from about 100 mg to about 900 mg per dose, such as an amount of about 100 mg per dose, 150 mg per dose, 200 mg per dose, 250 mg per dose, 300 mg per dose, 350 mg per dose, 400 mg per dose, 450 mg per dose, 500 mg per dose, 550 mg per dose, 600 mg per dose, 650 mg per dose, 700 mg per dose, 750 mg per dose, 800 mg per dose, 850 mg per dose, or 900 mg per dose. In some embodiments, the PGF2α receptor antagonist is administered to the subject in an amount of from about 200 mg to about 800 mg per dose, such as an amount of about 200 mg per dose, 250 mg per dose, 300 mg per dose, 350 mg per dose, 400 mg per dose, 450 mg per dose, 500 mg per dose, 550 mg per dose, 600 mg per dose, 650 mg per dose, 700 mg per dose, 750 mg per dose, or 800 mg per dose. In some embodiments, the PGF2α receptor antagonist is administered to the subject in an amount of from about 300 mg to about 700 mg per dose, such as an amount of about 300 mg per dose, 350 mg per dose, 400 mg per dose, 450 mg per dose, 500 mg per dose, 550 mg per dose, 600 mg per dose, 650 mg per dose, or 700 mg per dose. In some embodiments, the PGF2α receptor antagonist is administered to the subject in an amount of from about 400 mg to about 600 mg per dose, such as an amount of about 400 mg per dose, 450 mg per dose, 500 mg per dose, 550 mg per dose, or 600 mg per dose. In some embodiments, the PGF2α receptor antagonist is administered to the subject in an amount of from about 450 mg to about 550 mg per dose, such as an amount of about 450 mg per dose, 500 mg per dose, or 550 mg per dose.

In some embodiments, the PGF2α receptor antagonist is administered to the subject in an amount of from about 600 mg to about 1,400 mg per dose, such as an amount of about 600 mg per dose, 650 mg per dose, 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, 1,300 mg per dose, 1,350 mg per dose, or 1,400 mg per dose. In some embodiments, the PGF2α receptor antagonist is administered to the subject in an amount of from about 700 mg to about 1,300 mg per dose, such as an amount of about 700 mg per dose, 750 mg per dose, 800 mg per dose, 850 mg per dose, 900 mg per dose, 950 mg per dose, 1,000 mg per dose, 1,050 mg per dose, 1,100 mg per dose, 1,150 mg per dose, 1,200 mg per dose, 1,250 mg per dose, or 1,300 mg per dose. In some embodiments, the PGF2α receptor antagonist is administered to the subject in an amount of from about 800 mg to about 1,200 mg per dose, such as an amount of about 800 mg per dose, 850 mg per dose, 900 mg per dose, 950 mg per dose, 1,000 mg per dose, 1,050 mg per dose, 1,100 mg per dose, 1,150 mg per dose, or 1,200 mg per dose. In some embodiments, the PGF2α receptor antagonist is administered to the subject in an amount of from about 900 mg to about 1,100 mg per dose, such as an amount of about 900 mg per dose, 950 mg per dose, 1,000 mg per dose, 1,050 mg per dose, or 1,100 mg per dose. In some embodiments, the PGF2α receptor antagonist is administered to the subject in an amount of from about 950 mg to about 1,050 mg per dose, such as an amount of about 950 mg per dose, 1,000 mg per dose, or 1,050 mg per dose.

In some embodiments, the PGF2α receptor antagonist, such as the compound of any one of formulas (I) through (VIII), for example, compound (1), compound (2), or compound (3), among other PGF2α receptor antagonists described herein, is periodically administered to the subject in one or more doses per 12 hours, 24 hours, 36 hours, 48 hours, 60 hours, 72 hours, 84 hours, 96 hours, week, or month. For instance, the PGF2α receptor antagonist may be administered to the subject in from one to ten doses (e.g., 1, 2, 3, 4, 5, 6, 7, 8, 9, or 10 doses) every 12 hours, one to ten doses (e.g., 1, 2, 3, 4, 5, 6, 7, 8, 9, or 10 doses) every 14 hours, one to ten doses (e.g., 1, 2, 3, 4, 5, 6, 7, 8, 9, or 10 doses) every 16 hours, one to ten doses (e.g., 1, 2, 3, 4, 5, 6, 7, 8, 9, or 10 doses) every 18 hours, one to ten doses (e.g., 1, 2, 3, 4, 5, 6, 7, 8, 9, or 10 doses) every 20 hours, one to ten doses (e.g., 1, 2, 3, 4, 5, 6, 7, 8, 9, or 10 doses) every 22 hours, one to ten doses (e.g., 1, 2, 3, 4, 5, 6, 7, 8, 9, or 10 doses) every 24 hours, one to ten doses (e.g., 1, 2, 3, 4, 5, 6, 7, 8, 9, or 10 doses) every 26 hours, one to ten doses (e.g., 1, 2, 3, 4, 5, 6, 7, 8, 9, or 10 doses) every 28 hours, one to ten doses (e.g., 1, 2, 3, 4, 5, 6, 7, 8, 9, or 10 doses) every 30 hours, one to ten doses (e.g., 1, 2, 3, 4, 5, 6, 7, 8, 9, or 10 doses) every 32 hours, one to ten doses (e.g., 1, 2, 3, 4, 5, 6, 7, 8, 9, or 10 doses) every 34 hours, one to ten doses (e.g., 1, 2, 3, 4, 5, 6, 7, 8, 9, or 10 doses) every 36 hours, one to ten doses (e.g., 1, 2, 3, 4, 5, 6, 7, 8, 9, or 10 doses) every 38 hours, one to ten doses (e.g., 1, 2, 3, 4, 5, 6, 7, 8, 9, or 10 doses) every 40 hours, one to ten doses (e.g., 1, 2, 3, 4, 5, 6, 7, 8, 9, or 10 doses) every 42 hours, one to ten doses (e.g., 1, 2, 3, 4, 5, 6, 7, 8, 9, or 10 doses) every 44 hours, one to ten doses (e.g., 1, 2, 3, 4, 5, 6, 7, 8, 9, or 10 doses) every 46 hours, one to ten doses (e.g., 1, 2, 3, 4, 5, 6, 7, 8, 9, or 10 doses) every 48 hours, one to ten doses (e.g., 1, 2, 3, 4, 5, 6, 7, 8, 9, or 10 doses) every 60 hours, one to ten doses (e.g., 1, 2, 3, 4, 5, 6, 7, 8, 9, or 10 doses) every 72 hours, one to ten doses (e.g., 1, 2, 3, 4, 5, 6, 7, 8, 9, or 10 doses) every 84 hours, one to ten doses (e.g., 1, 2, 3, 4, 5, 6, 7, 8, 9, or 10 doses) every 96 hours, one to ten doses (e.g., 1, 2, 3, 4, 5, 6, 7, 8, 9, or 10 doses) every 108 hours, one to ten doses (e.g., 1, 2, 3, 4, 5, 6, 7, 8, 9, or 10 doses) every 120 hours, one to ten doses (e.g., 1, 2, 3, 4, 5, 6, 7, 8, 9, or 10 doses) every 132 hours, one to ten doses (e.g., 1, 2, 3, 4, 5, 6, 7, 8, 9, or 10 doses) every 144 hours, one to ten doses (e.g., 1, 2, 3, 4, 5, 6, 7, 8, 9, or 10 doses) every 156 hours, or one to ten doses (e.g., 1, 2, 3, 4, 5, 6, 7, 8, 9, or 10 doses) every week.

In some embodiments, the PGF2α receptor antagonist, such as the compound of any one of formulas (I) through (VIII), for example, compound (1), compound (2), or compound (3), among other PGF2α receptor antagonists described herein, is administered to the subject in from one to six doses per day. For example, PGF2α receptor antagonist, such as the compound of any one of formulas (I) through (VIII), for example, compound (1), compound (2), or compound (3), may be administered to the subject once daily. In some embodiments, the PGF2α receptor antagonist is administered to the subject twice daily.

In some embodiments, the PGF2α receptor antagonist is administered to the subject once every 4 to 12 hours, such as once every 4 hours, once every 5 hours, once every 6 hours, once every 7 hours, once every 8 hours, once every 9 hours, once every 10 hours, once every 11 hours, or once every 12 hours.

The PGF2α receptor antagonist, such as the compound of any one of formulas (I) through (VIII), for example, compound (1), compound (2), or compound (3), among other PGF2α receptor antagonists described herein, may be administered to the subject in one or more unit dosage forms that collectively constitute a single dose. For example, the subject may be administered a single dose of the compound of a specified amount, such as a single dose of 250 mg, 500 mg, 750 mg, 1,000 mg, 1,250 mg, 1,500 mg, 1,750 mg, 2,000 mg, or more, by administration of one or more unit dosage forms of the compound to the subject. As a non-limiting example, a single dose of 1,000 mg of the compound may be administered to the subject by way of two individual 500-mg unit dosage forms of the compound. The two 500-mg unit dosage forms collectively constitute a single 1,000-mg dose of the compound if administered to the subject at substantially the same time.

In some embodiments, the PGF2α receptor antagonist, such as the compound of any one of formulas (I) through (VIII), for example, compound (1), compound (2), or compound (3), among other PGF2α receptor antagonists described herein, is administered to the subject in one or more daily doses (e.g., from 1 to 10 doses, such as 1, 2, 3, 4, 5, 6, 7, 8, 9, or 10 doses, or more) totaling from about 250 mg to about 2,500 mg per day, such as an amount of about 250 mg, 300 mg, 350 mg, 400 mg, 450 mg, 500 mg, 550 mg, 600 mg, 650 mg, 700 mg, 750 mg, 800 mg, 850 mg, 900 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,050 mg, 2,100 mg, 2,150 mg, 2,200 mg, 2,250 mg, 2,300 mg, 2,350 mg, 2,400 mg, 2,450 mg, or 2,500 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 300 mg to about 2,000 mg per day, such as an amount of about 300 mg, 350 mg, 400 mg, 450 mg, 500 mg, 550 mg, 600 mg, 650 mg, 700 mg, 750 mg, 800 mg, 850 mg, 900 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, or 2,000 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 400 mg to about 1.600 mg per day, such as an amount of about 400 mg, 450 mg, 500 mg, 550 mg, 600 mg, 650 mg, 700 mg, 750 mg, 800 mg, 850 mg, 900 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, or 1,600 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 700 mg to about 1,300 mg per day, such as an amount of about 700 mg, 750 mg, 800 mg, 850 mg, 900 mg, 1,000 mg, 1,050 mg, 1,100 mg, 1,150 mg, 1,200 mg, 1,250 mg, or 1,300 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 800 mg to about 1,200 mg per day, such as an amount of about 800 mg, 850 mg, 900 mg, 1,000 mg, 1,050 mg, 1,100 mg, 1,150 mg, or 1,200 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 900 mg to about 1,100 mg per day, such as an amount of about 900 mg, 1,000 mg, 1,050 mg, or 1,100 mg per day.

In some embodiments, the PGF2α receptor antagonist is administered to the subject in one or more daily doses (e.g., from 1 to 10 doses, such as 1, 2, 3, 4, 5, 6, 7, 8, 9, or 10 doses, or more) totaling from about 950 mg to about 1,050 mg per day, such as an amount of about 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, or 1,050 mg per day.

In some embodiments, the PGF2α receptor antagonist, such as the compound of any one of formulas (I) through (VIII), for example, compound (1), compound (2), or compound (3), among other PGF2α receptor antagonists described herein, is administered to the subject in a single dose per day. For example, the PGF2α receptor antagonist may be administered to the subject in an amount (e.g., a single dose) of from about 250 mg to about 2,500 mg per day, such as an amount of about 250 mg, 300 mg, 350 mg, 400 mg, 450 mg, 500 mg, 550 mg, 600 mg, 650 mg, 700 mg, 750 mg, 800 mg, 850 mg, 900 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,050 mg, 2,100 mg, 2,150 mg, 2,200 mg, 2,250 mg, 2,300 mg, 2,350 mg, 2,400 mg, 2,450 mg, or 2,500 mg per day. In some embodiments, the PGF2α receptor antagonist is administered to the subject in an amount (e.g., a single dose) of about 1,000 mg per day.

In some embodiments, the PGF2α receptor antagonist, such as the compound of any one of formulas (I) through (VIII), for example, compound (1), compound (2), or compound (3), among other PGF2α receptor antagonists described herein, is administered to the subject once daily in an amount of from about 800 mg to about 1,200 mg per dose during a first treatment period. The PGF2α receptor antagonist may subsequently be administered to the subject twice daily in an amount of from about 300 mg to about 700 mg per dose during a second treatment period, i.e., totaling an amount of from about 600 mg to about 1,400 mg per day during the second treatment period.

In some embodiments, the PGF2α receptor antagonist is administered to the subject in an amount of from about 850 mg to about 1,150 mg per dose during the first treatment period, such as an amount of about 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, or 1,150 mg per dose during the first treatment period (i.e., totaling an amount of about 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, 1,100 mg, 1,105 mg, 1,110 mg, 1,115 mg, 1,120 mg, 1,125 mg, 1,130 mg, 1,135 mg, 1,140 mg, 1,145 mg, or 1,150 mg per day during the first treatment period).

In some embodiments, the PGF2α receptor antagonist is administered to the subject in an amount of from about 900 mg to about 1,100 mg per dose during the first treatment period, such as an amount of about 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, or 1,100 mg per dose during the first treatment period (i.e., totaling an amount of about 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, 1,050 mg, 1,055 mg, 1,060 mg, 1,065 mg, 1,070 mg, 1,075 mg, 1,080 mg, 1,085 mg, 1,090 mg, 1,095 mg, or 1,100 mg per day during the first treatment period).

In some embodiments, the PGF2α receptor antagonist is administered to the subject in an amount of from about 950 mg to about 1,050 mg per dose during the first treatment period, such as an amount of about 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, or 1,050 mg per dose during the first treatment period (i.e., totaling an amount of about 950 mg, 955 mg, 960 mg, 965 mg, 970 mg, 975 mg, 980 mg, 985 mg, 990 mg, 995 mg, 1,000 mg, 1,005 mg, 1,010 mg, 1,015 mg, 1,020 mg, 1,025 mg, 1,030 mg, 1,035 mg, 1,040 mg, 1,045 mg, or 1,050 mg per day during the first treatment period). In some embodiments, the PGF2α receptor antagonist is administered to the subject in an amount of about 1,000 mg per dose during the first treatment period.

In some embodiments, the PGF2α receptor antagonist is administered to the subject in an amount of from about 350 mg to about 650 mg per dose during the second treatment period, such as an amount of about 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, or 650 mg per dose during the second treatment period (i.e., totaling an amount of from about 700 mg to about 1,300 mg per day during the second treatment period, such as an amount of about 700 mg, 710 mg, 720 mg, 730 mg, 740 mg, 750 mg, 760 mg, 770 mg, 780 mg, 790 mg, 800 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1,000 mg, 1,010 mg, 1,020 mg, 1,030 mg, 1,040 mg, 1,050 mg, 1,060 mg, 1,070 mg, 1,080 mg, 1,090 mg, 1,100 mg, 1,110 mg, 1,120 mg, 1,130 mg, 1,140 mg, 1,150 mg, 1,160 mg, 1,170 mg, 1,180 mg, 1,190 mg, 1,200 mg, 1,210 mg, 1,220 mg, 1,230 mg, 1,240 mg, 1,250 mg, 1,260 mg, 1,270 mg, 1,280 mg, 1,290 mg, or 1,300 mg per day during the second treatment period).

In some embodiments, the PGF2α receptor antagonist is administered to the subject in an amount of from about 400 mg to about 600 mg per dose during the second treatment period, such as an amount of about 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, or 600 mg per dose during the second treatment period (i.e., totaling an amount of from about 800 mg to about 1,200 mg per day during the second treatment period, such as an amount of about 800 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1,000 mg, 1,010 mg, 1,020 mg, 1,030 mg, 1,040 mg, 1,050 mg, 1,060 mg, 1,070 mg, 1,080 mg, 1,090 mg, 1,100 mg, 1,110 mg, 1,120 mg, 1,130 mg, 1,140 mg, 1,150 mg, 1,160 mg, 1,170 mg, 1,180 mg, 1,190 mg, or 1,200 mg per day during the second treatment period).

In some embodiments, the PGF2α receptor antagonist is administered to the subject in an amount of from about 450 mg to about 550 mg per dose during the second treatment period, such as an amount of about 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, or 550 mg per dose during the second treatment period (i.e., totaling an amount of from about 900 mg to about 1,100 mg per day during the second treatment period, such as an amount of about 900 mg, 910 mg, 920 mg, 930 mg, 940 mg, 950 mg, 960 mg, 970 mg, 980 mg, 990 mg, 1,000 mg, 1,010 mg, 1,020 mg, 1,030 mg, 1,040 mg, 1,050 mg, 1,060 mg, 1,070 mg, 1,080 mg, 1,090 mg, or 1,100 mg per day during the second treatment period). In some embodiments, the PGF2α receptor antagonist is administered to the subject in an amount of about 500 mg per dose during the second treatment period.

In some embodiments, the PGF2α receptor antagonist is administered to the subject once every 10-14 hours during the second treatment period. In some embodiments, the PGF2α receptor antagonist is administered to the subject once every 11-13 hours during the second treatment period. In some embodiments, the PGF2α receptor antagonist is administered to the subject once every 12 hours during the second treatment period.

In some embodiments, the first treatment period has a duration of from 1 to 10 days (e.g., a duration of 1 day, 2 days, 3 days, 4 days, 5 days, 6 days, 7 days, 8 days, 9 days, or 10 days). In some embodiments, the first treatment period has a duration of from 1 to 5 days (e.g., a duration of 1 day, 2 days, 3 days, 4 days, or 5 days). In some embodiments, the first treatment period has a duration of 1 day.

In some embodiments, the second treatment period has a duration of from 1 to 28 days (e.g., a duration of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days). In some embodiments, the second treatment period has a duration of from 4 to 14 days (e.g., a duration of 4 days, 5 days, 6 days, 7 days, 8 days, 9 days, 10 days, 11 days, 12 days, 13 days, or 14 days). In some embodiments, the second treatment period has a duration of from 5 to 10 days (e.g., a duration of 5 days, 6 days, 7 days, 8 days, 9 days, or 10 days). In some embodiments, the second treatment period has a duration of 3 days. In some embodiments, the second treatment period has a duration of 4 days. In some embodiments, the second treatment period has a duration of 5 days. In some embodiments, the second treatment period has a duration of 6 days. In some embodiments, the second treatment period has a duration of 7 days. In some embodiments, the second treatment period has a duration of 8 days. In some embodiments, the second treatment period has a duration of 9 days. In some embodiments, the second treatment period has a duration of 10 days.

In some embodiments, the PGF2α receptor antagonist is administered to the subject until the subject reaches a gestational age of at least about 34 weeks (e.g., a gestational age of from about 34 weeks to about 40 weeks, such as a gestational age of 34 weeks, 35 weeks, 36 weeks, 37 weeks, 38 weeks, 39 weeks, or 40 weeks), or until the subject undergoes delivery. In some embodiments, the PGF2α receptor antagonist is administered to the subject until the subject reaches a gestational age of about 37 weeks.

In some embodiments, the PGF2α receptor antagonist is administered to the subject orally. The PGF2α receptor antagonist may be, for example, formulated a tablet, gel cap, powder, liquid solution, or liquid suspension.

### Pharmaceutical Compositions

The tocolytic agents (e.g., atosiban and the PGF2α receptor antagonists) suitable for use with the compositions and methods described herein can be formulated into pharmaceutical compositions for administration to a patient, such as a pregnant human patient, in a biologically compatible form suitable for administration in vivo. A pharmaceutical composition containing, for example, atosiban or a PGF2α receptor antagonist, such as a PGF2α receptor antagonist described herein (e.g., (3S)-3-({[(2S)-3-(biphenyl-4-ylsulfonyl)-1,3-thiazolidin-2-yl]carbonyl}-amino)-3-(4-fluorophenyl)propyl L-valinate or a pharmaceutically acceptable salt thereof, such as the chloride salt thereof), may additionally contain a suitable diluent, carrier, or excipient. PGF2α receptor antagonists can be administered to a patient, for example, orally or by intravenous injection. Under ordinary conditions of storage and use, a pharmaceutical composition may contain a preservative, e.g., to prevent the growth of microorganisms. Procedures and ingredients for the selection and preparation of suitable formulations are described, for example, in Remington: The Science and Practice of Pharmacy (2012, 22^{nd} ed.) and in The United States Pharmacopeia: The National Formulary (2015, USP 38 NF 33).

Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a patient in need of treatment.

A pharmaceutical composition may be administered to a patient, e.g., a human patient, alone or in combination with one or more pharmaceutically acceptable carriers, e.g., as described herein, the proportion of which may be determined by the solubility of the compound, the chemical nature of the compound, and/or the chosen route of administration, among other factors.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regards as their invention.

### Example One. Administration of a PGF2α Receptor Antagonist in Combination with Atosiban Prolongs Pregnancy in Human Patients

Introduction

Compound (2) is an orally-active prostaglandin F2α (PGF2α) receptor antagonist designed to control preterm labor by reducing inflammation and uterine contractions without causing the potentially serious vasoconstriction in the fetus (e.g. premature closure of the ductus arteriosus and/or renal impairment) seen with non-specific prostaglandin inhibitors, such as indomethacin and other non-steroidal anti-inflammatory drugs (NSAIDs).

This example reports results of a Phase II clinical trial of compound (2) in pregnant women with threatened spontaneous preterm labor. In part A of the study, compound (2) was administered in open-label fashion to n=9 patients with gestational ages of from 28 to 34 weeks in order to assess pharmacokinetics and safety. In part B of the study, compound (2) was administered to a more expansive set of n=58 subjects in order to assess efficacy, as well as to further evaluate safety.

Part A

Methods

Subjects participating in Part A of the study were diagnosed with preterm labor and were already receiving an atosiban infusion for 48 hours. Compound (2) was administered orally to each subject in an amount of 500 mg twice daily for 7 days, with a loading dose of 1,000 mg on Day 1.

In Part A of the study, fetal cardiac safety was monitored using Doppler ultrasound, and compound (2) pharmacokinetics were assessed on days 1, 2, 3 and 7 of the study. Amniotic Fluid Index was monitored from days 1 to 14. Time to delivery was also assessed.

Results

A total of 9 patients with singleton pregnancies were enrolled in Part A of the study. Baseline characteristics for subjects that participated in Part A are shown in Table 2, below.

TABLE 2
Baseline characteristics of patients administered
compound (2) and atosiban during Part A

| Parameter | Mean (Standard Deviation) |
|---|---|
| Age (years) | 32.4 (6.1) |
| Gestational Age (weeks) | 30.5 (2.1) |
| Number of previous pregnancies | 1.3 (1.5) |
| Cervical length (mm) | 16 (7.7) |
| Cervical dilation (cm) | 1.1 (1.0) |
| Number of contractions per 30 minutes | 2.2 (5.1) |

It was observed that 8 patients did not deliver within 7 days of administration of compound (2). One patient delivered on day 2 after starting compound (2). Mean±SD time to delivery was 41±23 days.

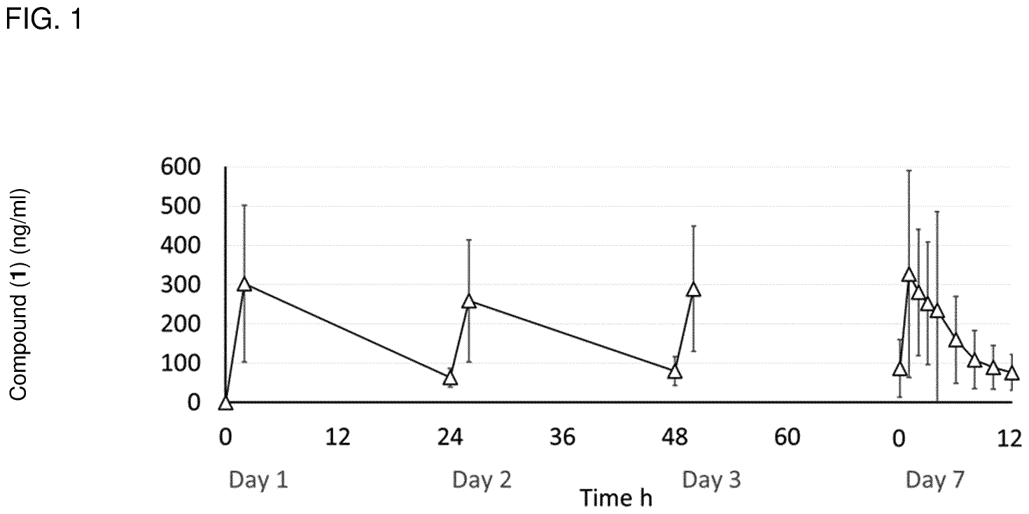

Plasma concentration vs. time profiles for compound (2) (FIG. 1) show that compound (2) was well absorbed from day 1 to day 7. Steady-state concentrations were achieved on day 1. Pharmacokinetic parameters were similar to those observed previously in non-pregnant women.

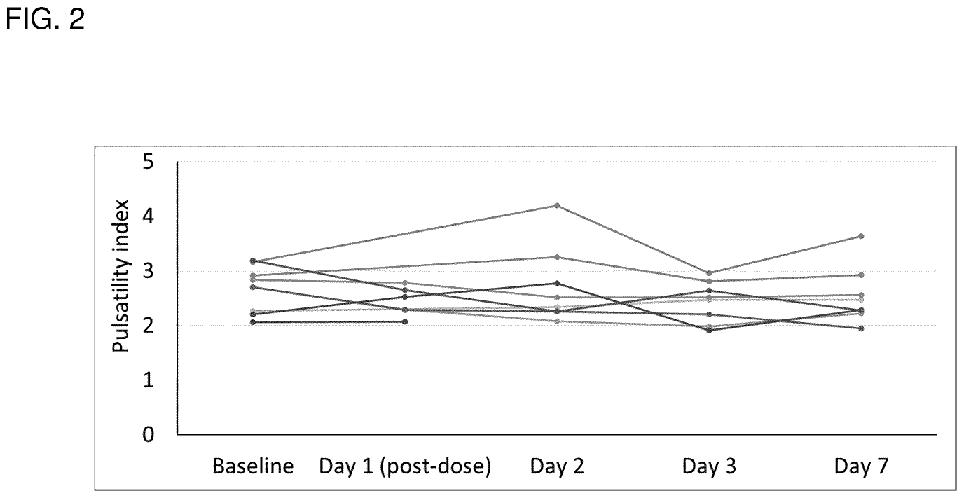

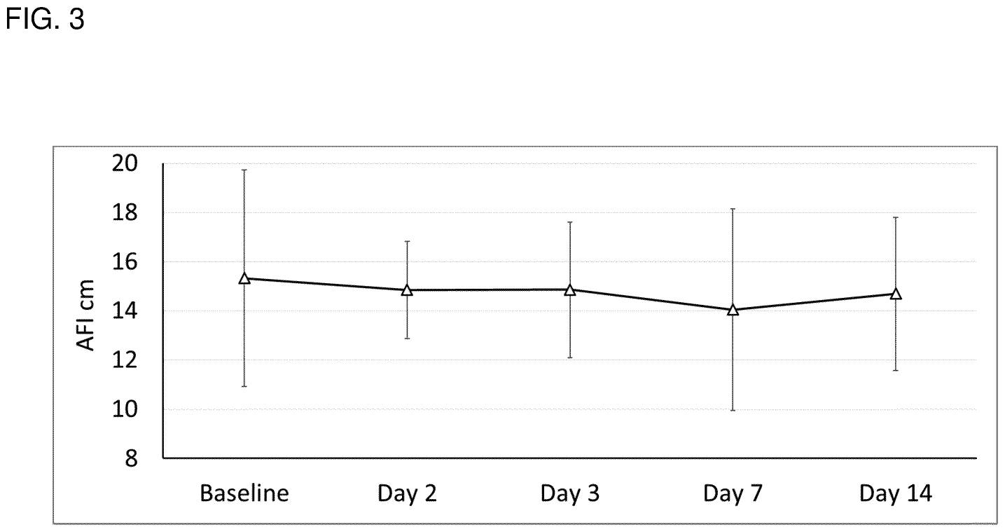

No adverse events were reported for the fetuses. Additionally, there were no clinically significant abnormal findings on the Doppler ultrasound, including no constrictive effect on the ductus arteriosus. The ductus arteriosus Pulsatility Index (FIG. 2) did not decrease over the dosing period. There were also no clinically significant changes in Amniotic Fluid Index (FIG. 3).

Conclusions

Compound (2) was well tolerated after oral administration for 7 days to women with threatened spontaneous PTL receiving atosiban. Compound (2) was also well absorbed, as plasma concentrations and pharmacokinetics were comparable to those observed in non-pregnant women.

Part B

Methods

Having demonstrated the safety of compound (2) in Part A of the study, Part B was designed to evaluate the ability of compound (2), in combination with atosiban, to effectuate a more robust delay in the onset of labor relative to a treatment regimen including placebo and atosiban. A total of n=58 patients were treated with compound (2) and atosiban, and n=55 patients were treated with placebo and atosiban. As in part A, patients underwent a 48-hour infusion with atosiban. On the first day of atosiban treatment, patients were administered either placebo or compound (2) in a loading dose of 1,000 mg. On subsequent days, patients were then administered either placebo or compound (2) twice daily in an amount of 500 mg per dose until delivery.

Participants

Pregnant women aged ≥18 years with singleton or twin pregnancies were eligible for this study if they presented with threatened spontaneous preterm labor between 24 and 33 6/7 weeks of gestation and did not have ruptured membranes. Gestational age was confirmed by a first trimester ultrasound scan or by fertilization date. Threatened spontaneous preterm labor was diagnosed when a subject was found to exhibit the following symptoms: (i) ≥4 uterine contractions per 30 minutes, as measured by electrohysterography, tocodynamometry, or abdominal palpation; (ii) cervical dilatation of 1 to 4 cm inclusive; and (iii) one or more of the following signs of preterm labor: a positive IGFBP-1 or fetal fibronectin test, cervical length ≤25 mm (by transvaginal ultrasound), or progressive cervical change. Subjects were required to begin treatment with atosiban within 24 hours before starting study treatment.

Exclusion criteria included: (i) stillbirth in the current pregnancy or in a previous pregnancy after gestational week 24; (ii) a high risk of stillbirth in the coming days; (iii) estimated ultrasound-based fetal weight outside 2 standard deviations; (iv) oligohydramnios; (v) pathological Doppler ultrasound of the umbilical artery, and (vi) expected iatrogenic delivery in the next 7 days, including, but not limited to, suspected abruptio placenta, signs of chorio-amnionitis, pre-eclampsia, eclampsia, or HELLP-syndrome, or any contraindications for the mother or the fetus to stop labor or prolong pregnancy. Other exclusion criteria included any clinically significant abnormality in the results of the screening safety laboratory tests (including AST, ALT, or total bilirubin greater than two times the upper limit of normal), known positive results from virology tests for HBsAg (not due to vaccination), HCV or HIV 1 or 2, BMI 35 kg/m 2 prior to start of current pregnancy, or had a bariatric surgery, any condition likely to hinder drug absorption, use of cervical cerclage in the current pregnancy or a pessary in situ, current use of anti-hypertensive medication, treatment with tocolytics other than atosiban within specified time before the baseline assessment of uterine contractions, known history of alcohol or drug abuse, and currently participating or have participated in another clinical trial within 30 days prior to screening.

The demographics and baseline characteristics of patients that participated in Part B of the study are shown in Table 3, below.

TABLE 3
Baseline characteristics of patients administered
compound (2) and atosiban during Part B

| | Atosiban + | Atosiban + |
|---|---|---|
| | Placebo | Compound (2) |
| | n=55 | n=58 |
| Mean age -years (SD) | 29.6 (5.1) | 29.7 (5.7) |

Race

| | | |
|---|---|---|
| White - n (%) | 39 (70.9%) | 42 (72.4%) |
| Asian - n (%) | 16 (29.1%) | 14 (24.1%) |
| Mean (SD) gestational age - | 29.7 (3.0) | 30.2 (2.6) |
| weeks | | |
| 24 to 30 weeks - n (%) | 23 (41.8%) | 25 (43.1%) |
| 30 to 34 weeks - n (%) | 32 (58.2%) | 33 (56.9%) |
| Singleton - n (%) | 41 (74.5%) | 42 (72.4%) |
| Twin - n (%) | 14 (25.5% | 16 (27.6%) |
| Mean (SD) contractions/ | 5.79 (2.23) | 6.32 (2.54) |
| 30 mins at screening | | |
| Cervical dilation (cm) | 1.43 (0.79) | 1.21 (0.37) |
| Mean (SD) | | |
| Cervical length of 25 mm or | 53 (96%) | 56 (97%) |
| less - n % | | |
| Progressive cervical change - | 2 (4%) | 2 (3%) |
| n % | | |

Randomization

Eligible women were counselled by their doctor about the study. After informed consent, participants were randomly allocated to receive compound (2) or placebo, 1:1, using a web-based randomization system (IBM Clinical Development Randomization and Dispensing Modules, Durham, NC, USA). Randomization was stratified by gestational age (24 weeks to 29 6/7 weeks and 30 weeks to 33 6/7 weeks) and number of fetuses (singleton or twins). The moment of starting the investigational medicinal product (IMP) was seen as the formal moment of randomization.

Both the compound (2) and placebo granules were manufactured by Aptuit® (Via Fleming, Verona, Italy). The granules were identical in characteristics such as size, thickness, physical properties, and appearance. Treatment kits were supplied by Almac Clinical Services® (Craigavon, UK) and consisted of one box containing a 15 mL bottle with 1666 mg granules (i.e. 500 mg of compound (2)) for oral intake after suspension in water.

The treatment kits list and randomization schedule (in blocks of 4) were generated using Cytel FlexRandomizer® (Cytel, USA) and uploaded in the in the web-based system by an unblinded statistician from Cytel. The trial participants, physicians, care providers and investigators and study management team had no access to the randomization list and allocation concealment was maintained throughout the trial.

Interventions

All participants received intravenous atosiban as standard-of-care therapy according to the approved product label for 48 hours. Treatment with study medication was to be initiated ideally simultaneously or at a maximum within 24 hours after initiating atosiban treatment. The loading dose of compound (2) was 1000 mg, with a maintenance dose of 500 mg twice a day, each 12 hours apart, for 7 days. Women allocated to the control group received the same dosing regimen with placebo. Subjects were instructed to record IMP administration in a patient diary and had to bring both opened and unopened IMP packages to each visit, in order to allow the assessment of compliance with study treatment.

Outcome Measures

Efficacy to delay preterm birth was assessed using the following outcomes:
delivery within 48 hours from start of treatment medication;
delivery within 7 days from start of treatment medication;
delivery before 37 weeks gestational age; and
time to delivery measured from start of treatment medication.

As this study was a proof-of-concept study, a primary efficacy endpoint was not prospectively defined. Other outcomes included the following:
uterine contractions assessed by electrohysterography, tocodynamometry, or abdominal palpation at each hour during the first 6 hours after start of test treatment; and
maternal plasma concentrations and pharmacokinetics of compound (1) (the active metabolite of compound (2)) and fetal cord blood concentrations at the time of delivery.

Infant development is being assessed at 6, 12, and 24 months by the incidence of infants with one or more Ages and Stages Questionnaire® domain score(s) below the cut-off score. Collection of this data is ongoing.

Safety was assessed by way of the following outcomes:
maternal incidence of adverse events (AEs), treatment-emergent adverse events (TEAEs), clinically significant changes in laboratory safety tests, and vital signs from Day 1 until 28 days after birth;
incidence of AEs indicating fetal distress, such as growth retardation and/or changes in fetal heart rate monitoring and/or amniotic fluid index (AFI) or amniotic fluid volume (in twins); and incidence of neonates experiencing adverse events, as well as clinically significant changes in vital signs, Apgar score, weight and head circumference at birth, and a measure of neonatal morbidity from birth until 28 days after birth.

Statistical Analysis

The sample size was estimated using the following assumptions: the rate of delivery within 2 days (48 h), within 7 days (168 h) post-compound (2) administration start, and before 37 weeks gestational age were supposed to be 11.2%, 25.4%, and 28.8% after compound (2) treatment, respectively, as compared to 33%, 40.4%, and 58.5%, respectively, after placebo. Using a two-sided type I error rate of 0.1 and no adjustments for multiplicity, 60 participants per arm would have provided a power of 93%, 55%, and 95% for each endpoint, when analyzed using logistic regression. Power calculations were performed using the LOGISTIC statement within the POWER procedure in SAS version 9.4.

Analyses of the incidence of delivery within 2 days (48 h), within 7 days (168 h) post-compound (2) administration start, and before 37 weeks of gestational age was performed using logistic regression with treatment group as the independent variable. Strata effects (gestational age and number of fetuses) were included within the model. The treatment effect was expressed as an adjusted odds ratio with 90% confidence intervals. Treatment by strata interaction terms were explored. Other covariates explored included the pre-dose uterine contraction rate as well as the possibility of a site-to-site effect. Time to delivery measured from start of IMP administration was analyzed using Cox's proportional-hazards model. In addition, a log-rank test was performed. The analysis was performed using SAS® statistical software Version 9.4. P-values <0.10 were considered to indicate statistical significance.

Results

A total of 122 women consented to the study, of whom 6 did not fulfill the eligibility criteria and one delivered before randomization. After completing screening, 115 women were randomly allocated to the compound (2) (n=58) or placebo (n=57) arms. After randomization, but before the onset of treatment, 2 women in the placebo arm withdrew from the study. The remaining 113 women were included in the full analysis set and safety analysis set.

Baseline maternal characteristics were comparable between the two arms (Table 3). The mean (SD) age was 29.6 (5.4) years and mean weight was 66.6 (11.6) kg. Median gestational age was 31.0 weeks, and 83/113 (73.5%) women had singleton pregnancies versus 30/113 (26.5%) twins.

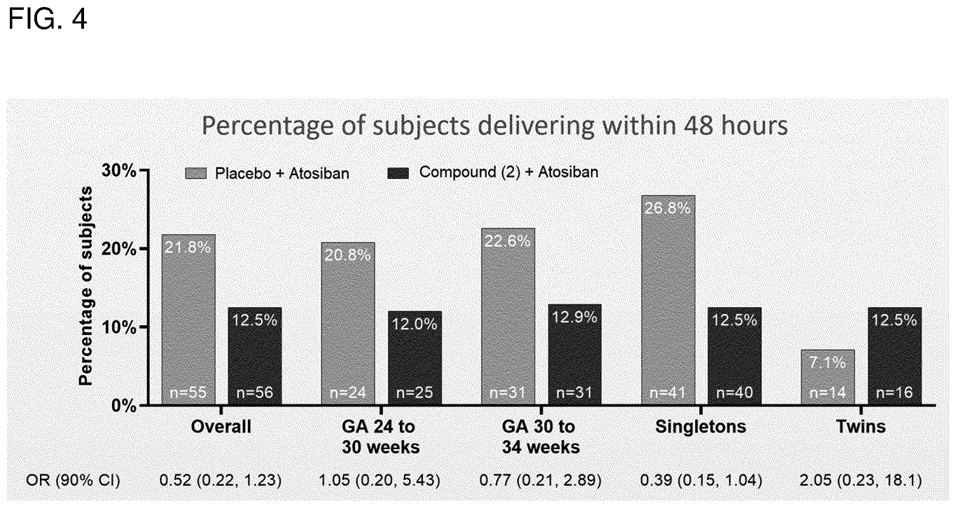

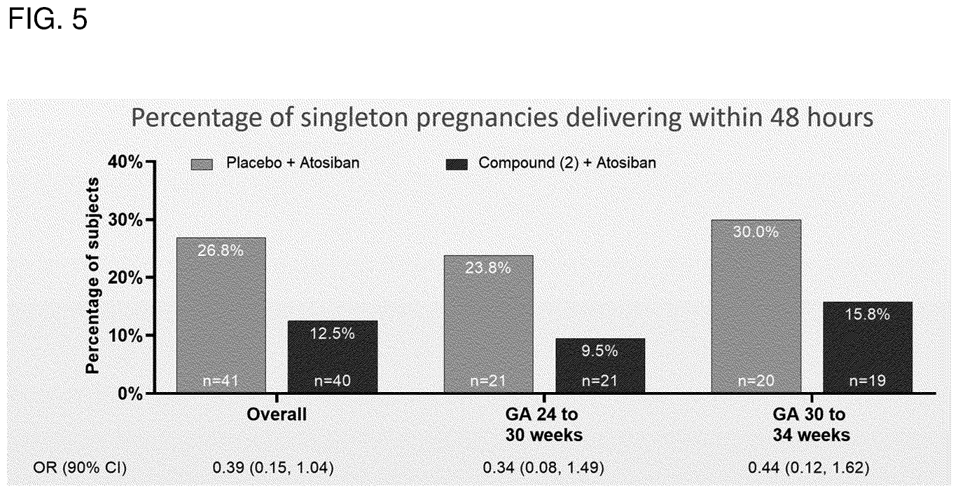

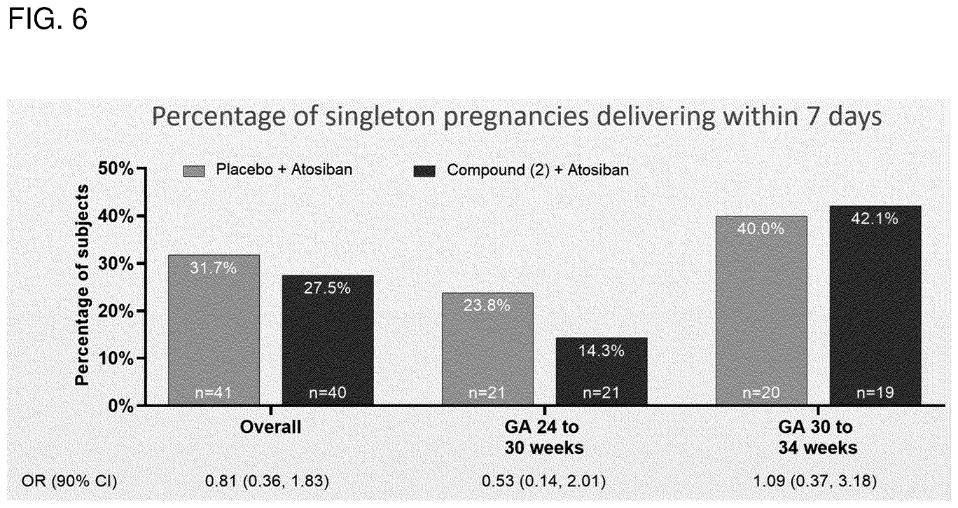

As is shown in FIG. 4, patients that were administered compound (2) and atosiban underwent delivery with a substantially lower frequency within 48 hours of initiating treatment relative to patients that were administered placebo and atosiban. This effect was observed in patients having a gestational age of from 24 to 30 weeks and in those having a gestational age of from 30 to 34 weeks (FIG. 4, categories (ii) and (iii) along the horizontal axis). The ability of compound (2) and atosiban to delay the onset of labor relative to treatment regimens of placebo and atosiban was particularly strong in patients having a singleton pregnancy (FIG. 4, category (iv) along the horizontal axis). FIGS. 5 and 6 further illustrate the labor-delaying effects of compound (2) and atosiban on patients having a singleton pregnancy.

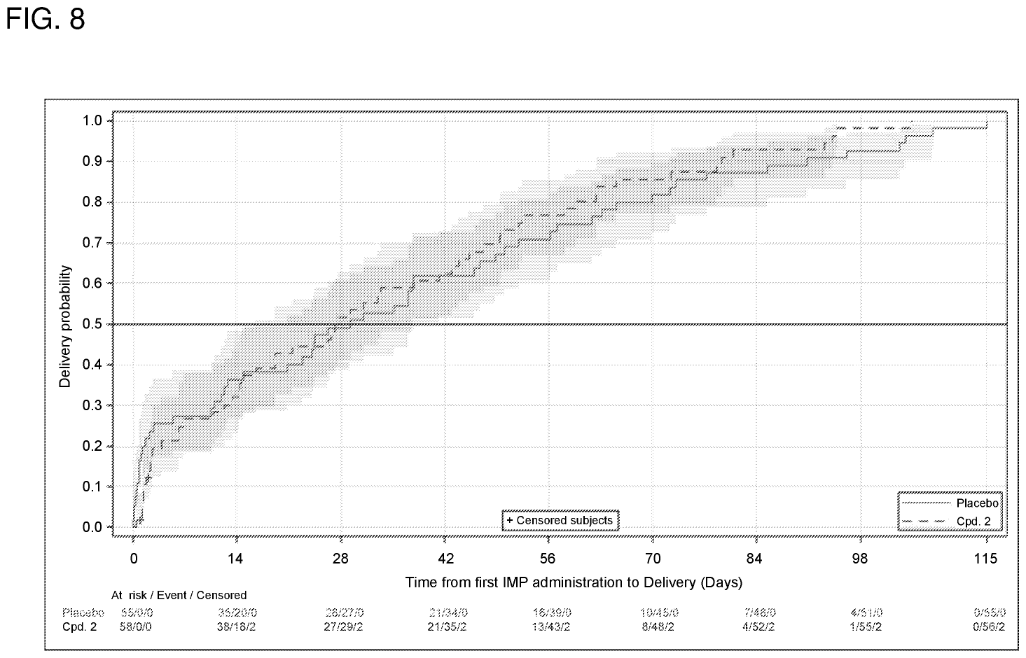

Strikingly, as FIG. 5 shows, only 9.5% of patients having a singleton pregnancy and a gestational age of 24 to 30 weeks underwent delivery within 48 hours of initiating treatment with compound (2) and atosiban, whereas 23.8% of patients having a singleton pregnancy and a gestational age of 24 to 30 weeks underwent delivery within 48 hours of initiating treatment with placebo and atosiban. The pregnancy-prolonging effects of compound (2) are summarized in FIG. 8, which demonstrates the proportion of trial participants that remained undelivered in both the compound (2)+atosiban arm of the study and the placebo+atosiban arm of the study.

The ability of compound (2) and atosiban to prolong pregnancy for 48 hours provides a significant therapeutic benefit, as it provides time for patients to be treated with critical, prenatal medications that improve the likelihood of perinatal survival. These medications include magnesium sulfate, which imparts a neuroprotective effect on the unborn fetus, as well as corticosteroids, such as betamethasone, dexamethasone, and hydrocortisone, which accelerate fetal lung maturation prior to birth. This extension of pregnancy is especially valuable for patients having younger gestational ages, such as gestational ages of from 24 weeks to 30 weeks, as an unborn fetus of this age is in particular need of neuroprotection and lung maturation in order to improve its likelihood of survival. By delaying delivery in a particularly robust manner among patients having young gestational ages, compound (2), when administered in combination with atosiban, provides the benefit of additional time for patients that are vulnerable to preterm labor to be treated with potentially life-saving interventions for the unborn fetus.

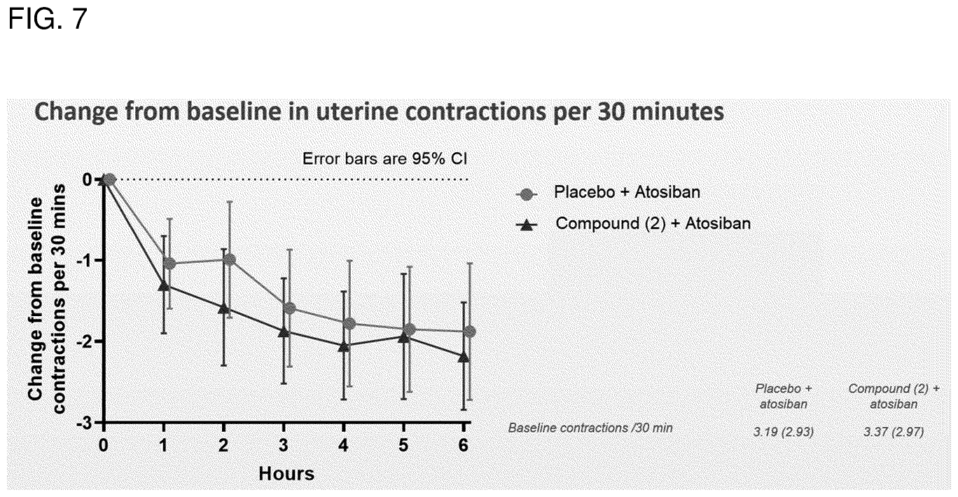

Without being limited by mechanism, one manner in which compound (2) may effectuate this delay in the onset of labor is by reducing uterine contractility. As FIG. 7 shows, compound (2), when administered in combination with atosiban, may effectuate a more sustained reduction in uterine contractions relative to baseline.

A more detailed summary of the efficacy results obtained from this study is shown in Table 4, below.

TABLE 4
Detailed summary of efficacy results from Part B.

| | Atosiban + | Atosiban + | |
|---|---|---|---|
| | Placebo | Compound (2) | OR (90% CI) |
| All women | n = 55 | n = 56 | |
| Delivery ≤48 h - | 12/55 (21.8) | 7/56 (12.5) | 0.52 (0.22, 1.23) |
| n (%) | | | |
| GA 24 to 30 wks | 5/24 (20.8) | 3/25 (12.0) | 1.05 (0.20, 5.43) |
| at BL | | | |
| GA 30 to 34 wks | 7/31 (22.6) | 4/31 (12.9) | 0.77 (0.21, 2.89) |
| at BL | | | |
| Delivery ≤7 | 15/55 (27.3) | 15/56 (26.8) | 1.00 (0.49, 2.04) |
| days - n (%) | | | |
| GA 24 to 30 wks | 6/24 (25.0) | 5/25 (20.0) | 1.07 (0.28, 4.18) |
| at BL | | | |
| GA 30 to 34 wks | 9/31 (29.0) | 10/31 (32.3) | 1.41 (0.52, 3.87) |
| at BL | | | |
| Delivery ≤37 | 33/55 (60.0) | 37/56 (66.1%) | 1.29 (0.63, 2.66) |
| weeks-n (%) | | | |
| Mean time to | 36.8 (33.8) | 33.9 (29.4) | 1.19 (0.86, 1.64)* |
| delivery - days | | | |
| (SD) | | | |
| Singletons only | n = 41 | n = 40 | |
| Delivery ≤48 h - | 11/41 (26.8) | 5/40 (12.5) | 0.39 (0.15, 1.04) |
| n (%) | | | |
| GA 24 to 30 wks | 5/21 (23.8) | 2/21 (9.5) | 0.34 (0.08, 1.49) |
| at BL | | | |
| GA 30 to 34 wks | 6/20 (30.0) | 3/19 (15.8) | 0.44 (0.12, 1.62) |
| at BL | | | |
| Delivery ≤7 | 13/41 (31.7) | 11/40 (27.5) | 0.81 (0.36, 1.83) |
| days - n (%) | | | |
| GA 24 to 30 wks | 5/21 (23.8) | 3/21 (14.3) | 0.53 (0.14, 2.01) |
| at BL | | | |
| GA 30 to 34 wks | 8/20 (40.0) | 8/19 (42.1) | 1.09 (0.37, 3.18) |
| at BL | | | |
| Delivery ≤37 | 20/41 (49) | 21/40 (53) | ND |
| weeks-n (%) | | | |
| Mean time to | 41.9 (37.2) | 40.6 (31.5) | 1.20 (0.82, 1.75)* |
| delivery - days | | | |
| (SD) | | | |
| Twins only | n = 14 | n = 16 | |
| Delivery ≤48 h - | 1/14 (7.1) | 2/16 (12.5) | 2.05 (0.23, 18.1) |
| n (%) | | | |
| Delivery ≤7 | 2/14 (14.3) | 4/16 (25.0) | 1.87 (0.36, 9.79) |
| days - n (%) | | | |
| Delivery ≤37 | 13/14 (93) | 16/16 (100) | ND |
| weeks-n (%) | | | |
| Mean time to | 21.8 (12.5) | 17.1 (12.6) | 1.40 (0.75, 2.64)* |
| delivery - days | | | |
| (SD) | | | |

As Table 4 shows, data were obtained from 56 subjects in the compound (2)+atosiban arm that delivered, and data were obtained from all 55 subjects in the placebo+atosiban arm that delivered. The observed delivery rate within 48 hours after the start of dosing was 7/56 (12.5%) of women in the compound (2) group versus 12/55 (21.8%) in the placebo group (aOR=0.52; 90% CI: 0.22, 1.23). Among women with singleton pregnancies, the observed delivery rate within 48 hours of IMP start in the compound (2) group was 5/40 (12.5%) versus 11/41 (26.8%) in the placebo group (aOR 0.39; 90% CI: 0.15, 1.04). For twin pregnancies, these rates were 2/16 (12.5%) of women receiving compound (2) compared to 1/14 (7.1%) receiving placebo (adjusted OR 2.05; 90% CI: 0.23, 18.1).

Delivery within 7 days (168 h) from IMP start did not differ between the compound (2) (15/58; 26.8%) and placebo (15/55; 27.3%) groups (OR=1.00; 90% CI: 0.49, 2.1). In women with singleton pregnancy between 24 to 30 weeks GA the delivery rate within 7 days in women receiving compound (2) was 14.3% versus 23.8% after placebo (aOR=0.53; 90% CI: 0.14, 2.0).

Significantly, not only was compound (2) found to be more effective than placebo as an add-on to atosiban therapy, compound (2) demonstrated excellent safety. Patients undergoing atosiban infusion that were treated with compound (2) exhibited fewer adverse events than patients treated with placebo. Certain adverse events that were documented in Part B of this study are summarized in Table 5, below.

TABLE 5
Summary of certain adverse events.

| | Atosiban + | Atosiban + |
|---|---|---|
| | Placebo | Compound (2) |
| At least one TEAE | 23 (41.8%) | 24 (41.4%) |
| Serious adverse events (SAEs) | 0 | 0 |
| Severe AEs | 1 (1.8%) | 0 |
| AEs leading to IMP | 0 | 1 (1.7%) |
| discontinuation | | |

Conclusion

Part B of this study demonstrates that compound (2) effectuates a robust delay in the onset of delivery when administered as an add-on to atosiban treatment in patients at risk of undergoing preterm labor. As FIGS. 4-6 show, the pregnancy-extending effects of compound (2) and atosiban are particularly pronounced in patients having singleton pregnancies. The ability of compound (2) and atosiban to delay the onset of labor provides the significant therapeutic benefit of additional time for a patient to receive medications that can enhance the likelihood of survival of the unborn fetus. Importantly, not only was compound (2) observed to achieve a marked reduction in imminent delivery rates, compound (2) was also particularly safe. Taken together, the results of Parts A and B of this study demonstrate that compound (2) can be safely and effectively administered in combination with atosiban so as to delay the onset of labor in patients at risk of undergoing delivery prior to a full gestational term.

### Example Two. Use of Atosiban and a PGF2α Receptor Antagonist for the Treatment or Prevention of Preterm Labor in a Human Patient

Using the compositions and methods described herein, a patient undergoing or at risk of undergoing preterm labor may be administered atosiban in combination with a PGF2α receptor antagonist, such as a compound of any one of formulas (I) through (VIII) described herein (e.g., compound (1), (2), or (3) described herein). A physician of skill in the art may assess the patient for risk of preterm labor, and may determine that the patient is at risk of undergoing preterm labor if the patient has a gestational age of from about 24 weeks to about 36 weeks prior to administration of a tocolytic agent, exhibits four or more uterine contractions per 30 minutes, exhibits a cervical dilation of from about 1 cm to about 4 cm, tests positive for the presence of fetal fibronectin and/or insulin-like growth factor-binding protein-1 (IGFBP-1) in a sample of cervical secretion obtained from the patient, and/or exhibits a cervical length of about 25 mm or less.

Upon determining that the patient is undergoing or at risk of undergoing preterm labor, the patient may be administered a PGF2α receptor antagonist, such as compound (2) or the chloride salt thereof, or another compound that gives rise to compound (1) in vivo, in one or more daily doses. For example, the PGF2α receptor antagonist may be administered to the subject once or twice daily, in a dose of from, e.g., 1,000 mg to 2,000 mg. The subject may be administered atosiban in accordance with a dosing regimen described herein.

Following administration of the atosiban and the PGF2α receptor antagonist, the patient may be assessed for successful treatment or prevention of preterm labor. Successful treatment of a pregnant subject with atosiban and a PGF2α receptor antagonist described herein may be signaled, for instance, by observing a delay in the onset of delivery by the patient. The delay may be a matter of one or more hours, days, or weeks (e.g., a delay of from about 1 hour to about 16 weeks, such as a delay of 1 hour, 6 hours, 12 hours, 18 hours, 24 hours, 48 hours, 72 hours, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks, among others) following the first administration of atosiban and the PGF2α receptor antagonist to the subject. Additionally or alternatively, the physician may determine that the patient has been successfully treated by observing a delay in the onset of delivery such that the subject undergoes delivery at a gestational age of at least about 34 weeks, such as at a gestational age of from about 34 weeks to about 40 weeks, following administration of atosiban and the PGF2α receptor antagonist to the subject. Successful treatment may also be signaled by a reduction in vaginal bleeding, a delay in the onset of amniorrhexis by the patient, and a reduction in the expression of one or more proinflammatory genes, such as cyclooxygenase-2 (Cox2) by the subject (as assessed, e.g., by observing a decrease in myometrial Cox2 expression) following administration of atosiban and the PGF2α receptor antagonist. The patient may also be determined to have been successfully treated upon detecting a decrease in the frequency of, peak amplitude of, duration of, and/or work done by, uterine contractions in the patient.

### Example Three. Use of Atosiban and a PGF2α Receptor Antagonist to Delay Delivery and an Antenatal Corticosteroid to Promote Fetal Lung Maturation Prior to Birth

Using the compositions and methods described herein, a patient undergoing or at risk of undergoing preterm labor may be administered atosiban in combination with a PGF2α receptor antagonist, such as a compound of any one of formulas (I) through (VIII) described herein (e.g., compound (1), (2), or (3) described herein), in order to delay the onset of delivery, as described in Example Two, above. For example, the PGF2α receptor antagonist may be administered to the subject once or twice daily, in a dose of from, e.g., 1,000 mg to 2,000 mg. The subject may be administered atosiban in accordance with a dosing regimen described herein.

In some instances, in addition to administering atosiban and a PGF2α receptor antagonist to the patient, a physician may additionally prescribe and administer an antenatal corticosteroid. Antenatal corticosteroids, such as betamethasone, dexamethasone, and hydrocortisone, can be used to accelerate fetal lung maturation prior to birth. In this way, a patient may be administered atosiban and a PGF2α receptor antagonist (such as a compound represented by any one of formulas (I) through (VIII), e.g., compound (1), (2), or (3)) so as to delay the onset of delivery and provide additional time for administration of an antenatal corticosteroid to promote fetal lung development. Administration of the corticosteroid may reduce the likelihood of neonatal death, respiratory distress syndrome, intraventricular hemorrhage, necrotizing enterocolitis, respiratory support, intensive care admissions, and systemic infections following birth.

### Other Embodiments

All publications, patents, and patent applications mentioned in this specification are incorporated herein by reference to the same extent as if each independent publication or patent application was specifically and individually indicated to be incorporated by reference.

In addition to the methods of treatment described herein, the present disclosure also includes compositions containing one or more of the compounds described herein for use in a method of treatment described herein. The disclosure also includes uses of the compositions and compounds described herein in the manufacture of a medicament for performing a method of treatment described herein.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of delaying the onset of delivery in a pregnant human subject, the method comprising administering to the subject therapeutically effective amounts of atosiban and a compound represented by formula (I) 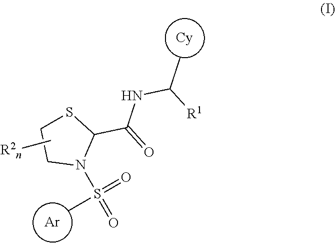
wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof,
wherein the compound is administered to the subject in an amount of from about 250 mg to about 2,500 mg per dose.

2. A method of treating or preventing preterm labor in a pregnant human subject, the method comprising administering to the subject therapeutically effective amounts of atosiban and a compound represented by formula (I) 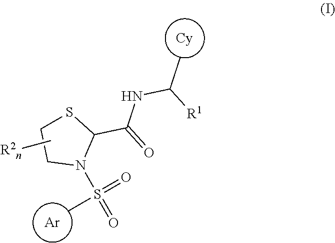
wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof,
wherein the compound is administered to the subject in an amount of from about 250 mg to about 2,500 mg per dose.

3. A method of preventing labor prior to cesarean delivery in a pregnant human subject, the method comprising administering to the subject therapeutically effective amounts of atosiban and a compound represented by formula (I) 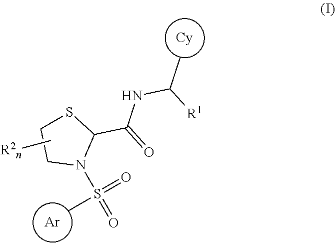
wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅-alkyl sulfinyl, C₁-C₅-alkyl sulfonyl, C₁-C₅-alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₁-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof,
wherein the compound is administered to the subject in an amount of from about 250 mg to about 2,500 mg per dose.

4. The method of any one of claims 1-3, wherein the ring Ar is selected from the group consisting of substituents (Ia) to (Iy): 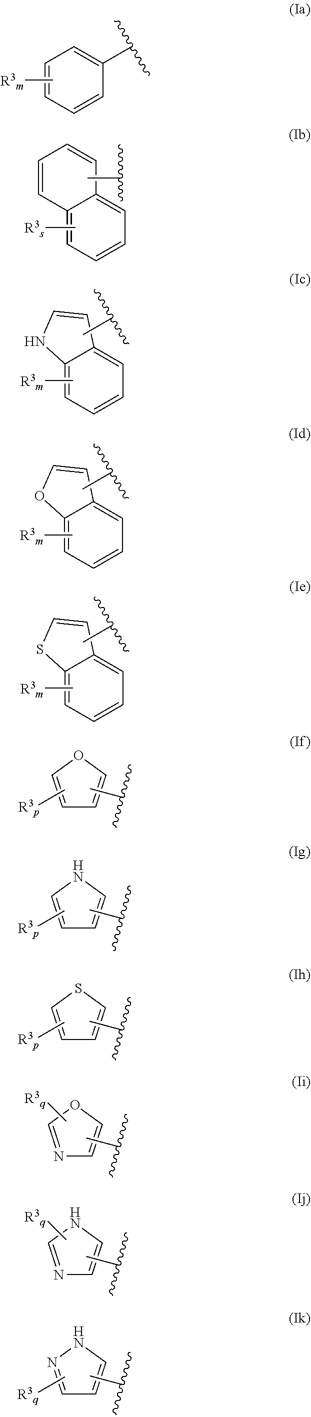 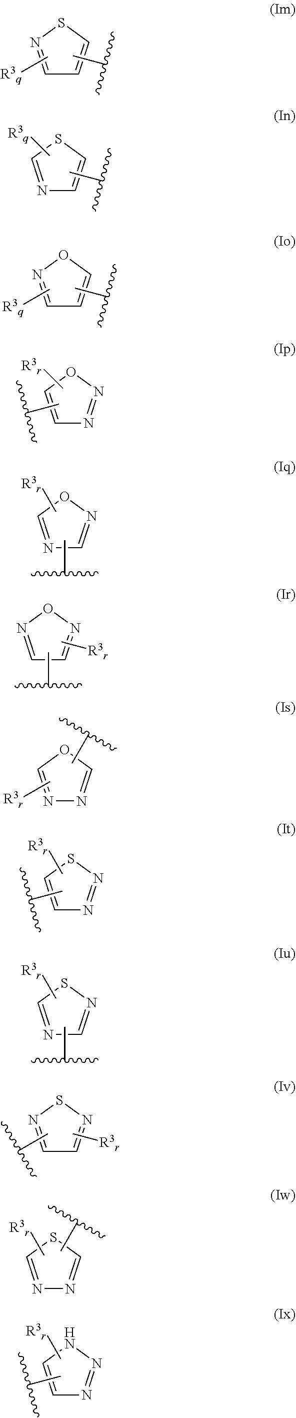 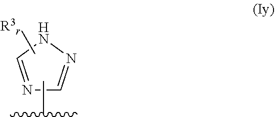
wherein each R³ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each m is independently an integer from 0-5;
each p is independently an integer from 0-3;
each q is independently an integer from 0-2;
each r is independently an integer from 0-1; and
each s is independently an integer from 0-7.

5. The method of claim 4, wherein each R³ is independently selected from the group consisting of substituents (IIa) to (IIy).

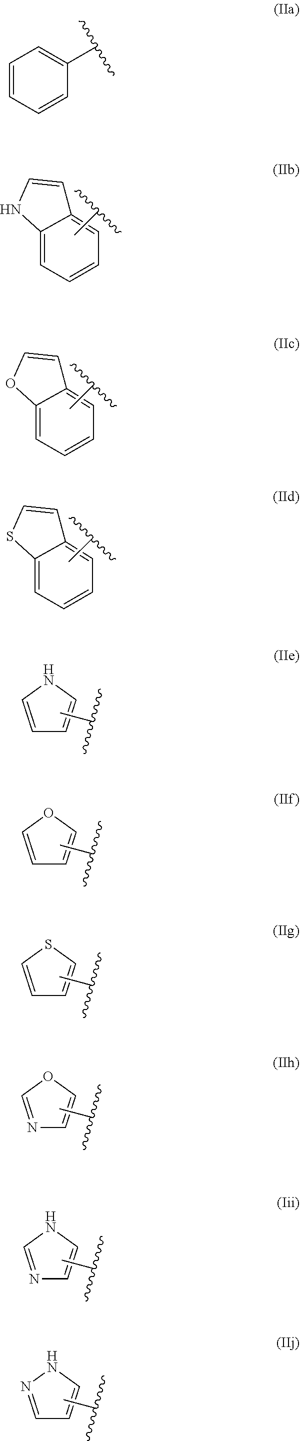

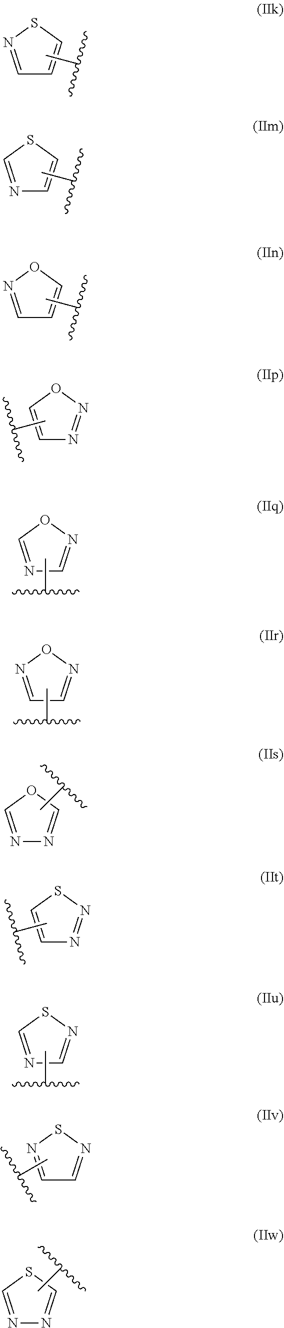

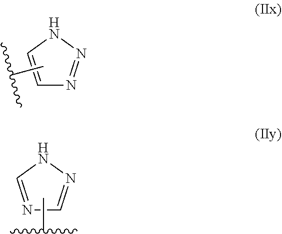

6. The method of claim 4, wherein the ring Ar is a substituent represented by formula (Ia) 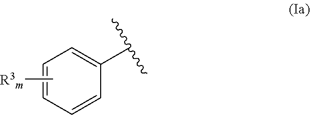
and wherein each R³ is, independently, optionally substituted and optionally fused aryl, optionally substituted and optionally fused heteroaryl, optionally substituted and optionally fused cycloalkyl, or optionally substituted and optionally fused heterocycloalkyl.

7. The method of any one of claims 1-6, wherein the ring Cy is selected from the group consisting of substituents (IIIa) to (IIIaa): 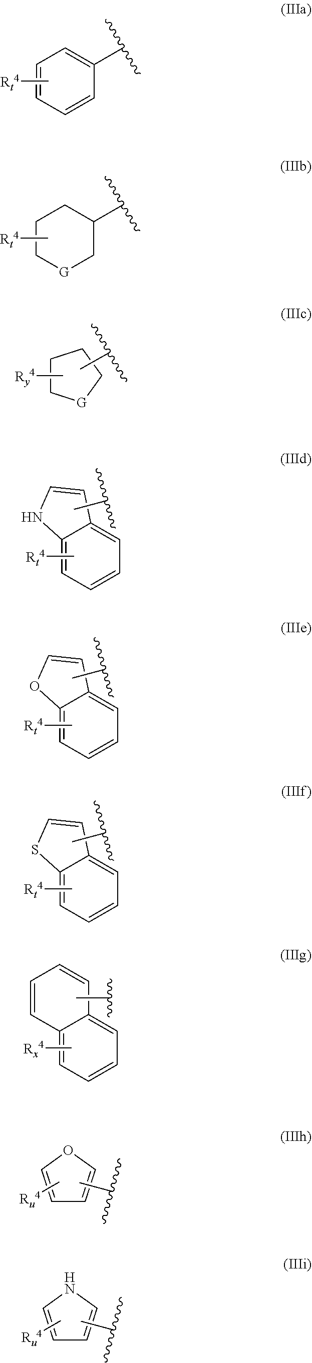 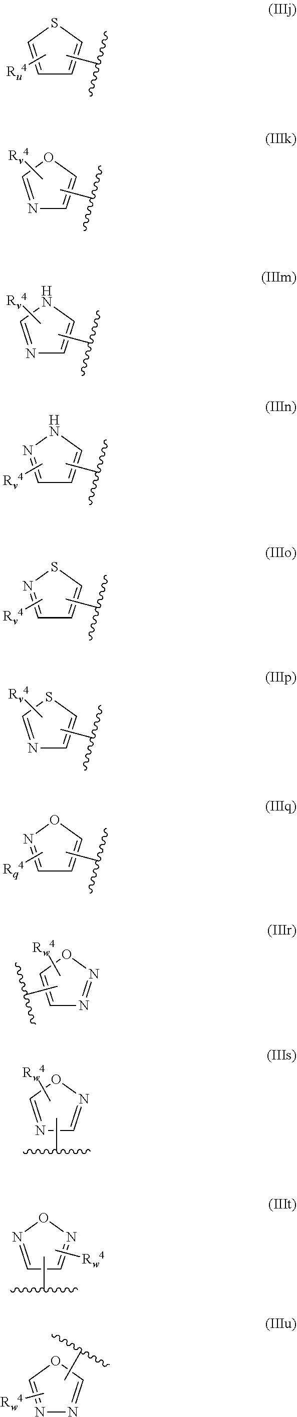 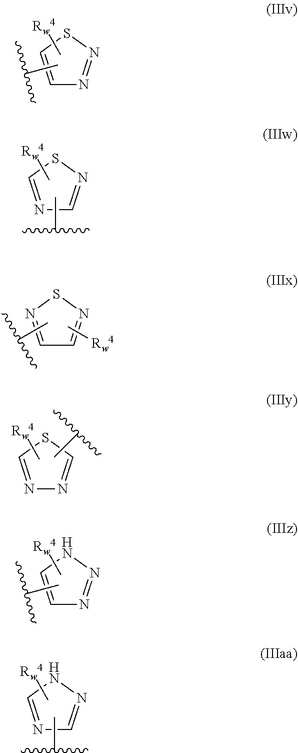
wherein each R⁴ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each G is independently —CH₂—, —CR⁴H—, —NH—, —NR⁴—, —O—, or —S—;
each t is independently an integer from 0-5;
each u is independently an integer from 0-3;
each v is independently an integer from 0-2;
each w is independently an integer from 0-1;
each x is independently an integer from 0-7; and
each y is independently an integer from 0-4.

8. The method of claim 7, wherein the ring Cy is an optionally substituted aryl group represented by formula (IVa).

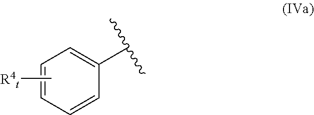

9. The method of claim 8, wherein the ring Cy is a substituted aryl group represented by formula (IVb).

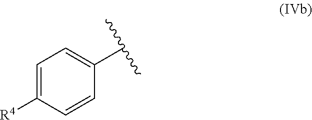

10. The method of any one of claims 1-9, wherein R¹ is C₁-C₅-alkyl carboxy, C₁-C₅-alkyl acyl, C₁-C₅-alkyl alkoxycarbonyl, C₁-C₅-alkyl acyloxy, substituted C₁-C₅-alkyl carboxy, substituted C₁-C₅-alkyl acyl, substituted C₁-C₅-alkyl alkoxycarbonyl, or substituted C₁-C₅-alkyl acyloxy.

11. The method of claim 10, wherein R¹ is optionally substituted C₁-C₅-alkyl acyloxy.

12. The method of any one of claims 1-11, wherein the compound is represented by formula (V) 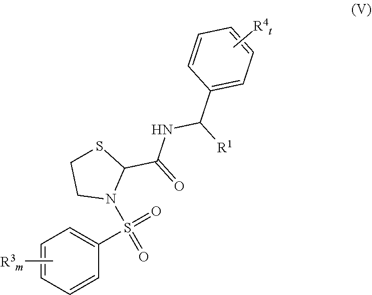
wherein R¹ is C₁-C₅-alkyl carboxy, C₁-C₅-alkyl acyl, C₁-C₅-alkyl alkoxycarbonyl, C₁-C₅-alkyl acyloxy, substituted C₁-C₅-alkyl carboxy, substituted C₁-C₅-alkyl acyl, substituted C₁-C₅-alkyl alkoxycarbonyl, or substituted C₁-C₅-alkyl acyloxy;
each R³ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each R⁴ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
m is an integer from 0-5; and
t is an integer from 0-5,
or a pharmaceutically acceptable salt thereof.

13. The method of claim 12, wherein the compound is represented by formula (Va) 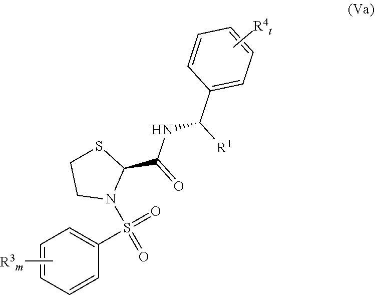
or a pharmaceutically acceptable salt thereof.

14. The method of any one of claims 1-13, wherein the compound is represented by formula (VI) 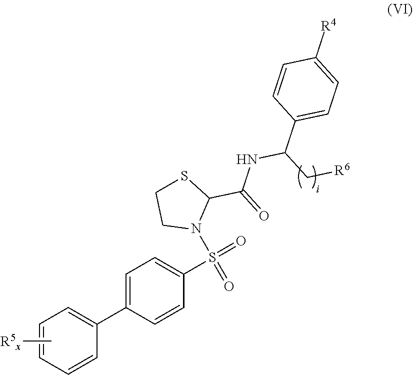
wherein R⁶ is hydroxyl, optionally substituted acyl, optionally substituted alkoxycarbonyl, or optionally substituted acyloxy;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3; and
x is an integer from 0-5,
or a pharmaceutically acceptable salt thereof.

15. The method of claim 14, wherein the compound is represented by formula (VII) 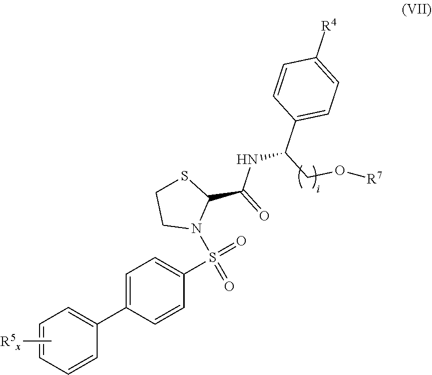
wherein R⁷ is H or optionally substituted aminoacyl;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3; and
x is an integer from 0-5,
or a pharmaceutically acceptable salt thereof.

16. The method of any one of claims 1-15, the method comprising providing to the subject compound (1).

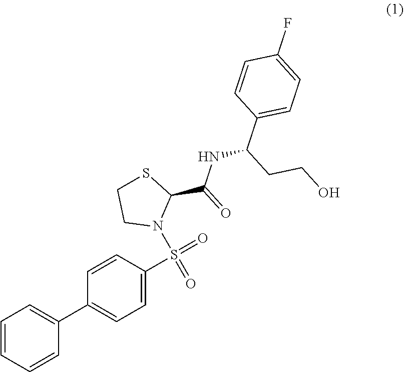 [SMILES: O=C(N[C@@H](CCO)c1ccc(F)cc1)[C@@H]1SCCN1S(=O)(=O)c1ccc(-c2ccccc2)cc1]

17. The method of any one of claims 1-16, the method comprising administering to the subject compound (2) 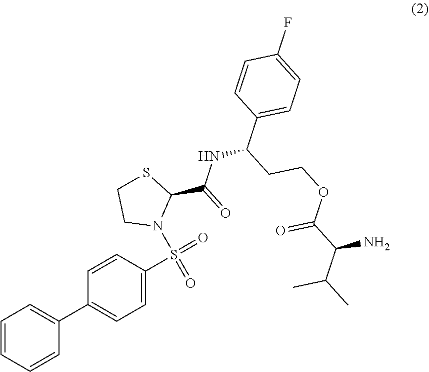 [SMILES: CC(C)[C@H](N)C(=O)OCC[C@H](NC(=O)[C@@H]1SCCN1S(=O)(=O)c1ccc(-c2ccccc2)cc1)c1ccc(F)cc1]
or a pharmaceutically acceptable salt thereof.

18. The method of claim 17, wherein the compound is represented by formula (3).

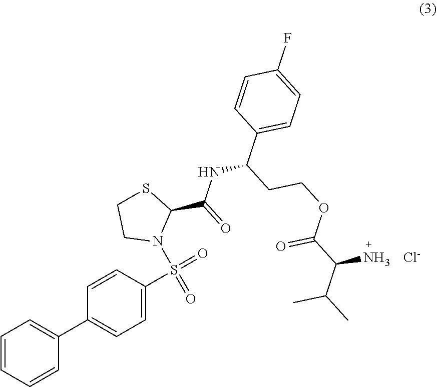 [SMILES: CC(C)[C@H](N)C(=O)OCC[C@H](NC(=O)[C@@H]1SCCN1S(=O)(=O)c1ccc(-c2ccccc2)cc1)c1ccc(F)cc1 ; [Cl-]]

19. The method of claim 18, wherein the compound is in a crystalline state.

20. The method of claim 19, wherein the compound exhibits characteristic X-ray powder diffraction peaks at about 7.0° 2θ, about 8.1° 2θ, about 10.0° 2θ, about 12.0° 2θ, about 13.1° 2θ, about 14.1° 2θ, about 16.4° 2θ, about 18.4° 2θ, about 20.1° 2θ, about 21.0° 2θ, about 23.5° 2θ, and about 29.5° 2θ.

21. The method of claim 19 or 20, wherein the compound exhibits ¹H nuclear magnetic resonance (NMR) peaks centered at about 1.1 ppm, about 3.3 ppm, about 4.9 ppm, about 5.4 ppm, about 7.1 ppm, about 7.7 ppm, about 7.9 ppm, and about 8.0 ppm.

22. The method of any one of claims 19-21, wherein the compound exhibits an endotherm at from about 145° C. to about 147° C. as measured by differential scanning calorimetry.

23. The method of any one of claims 19-22, wherein the compound exhibits a weight loss of from about 0.2% to about 0.6% when heated from 25° C. to 100° C. as measured by thermogravimetric analysis.

24. The method of any one of claims 19-23, wherein the compound exhibits a weight loss of from about 2.5% to about 3.5% when heated from 100° C. to 160° C. as measured by thermogravimetric analysis.

25. The method of any one of claims 1-24, wherein the compound is administered to the subject in an amount of from about 300 mg to about 2,300 mg per dose.

26. The method of claim 25, wherein the compound is administered to the subject in an amount of from about 400 mg to about 2,100 mg per dose.

27. The method of claim 26, wherein the compound is administered to the subject in an amount of from about 450 mg to about 2,050 mg per dose.

28. The method of any one of claims 1-24, wherein the compound is administered to the subject in an amount of from about 600 mg to about 1,400 mg per dose.

29. The method of claim 28, wherein the compound is administered to the subject in an amount of from about 700 mg to about 1,300 mg per dose.

30. The method of claim 29, wherein the compound is administered to the subject in an amount of from about 800 mg to about 1,200 mg per dose.

31. The method of claim 30, wherein the compound is administered to the subject in an amount of from about 900 mg to about 1,100 mg per dose.

32. The method of claim 31, wherein the compound is administered to the subject in an amount of from about 950 mg to about 1,050 mg per dose.

33. The method of claim 32, wherein the compound is administered to the subject in an amount of about 1,000 mg per dose.

34. The method of any one of claims 1-24, wherein the compound is administered to the subject in an amount of from about 100 mg to about 900 mg per dose.

35. The method of claim 34, wherein the compound is administered to the subject in an amount of from about 200 mg to about 800 mg per dose.

36. The method of claim 35, wherein the compound is administered to the subject in an amount of from about 300 mg to about 700 mg per dose.

37. The method of claim 36, wherein the compound is administered to the subject in an amount of from about 400 mg to about 600 mg per dose.

38. The method of claim 37, wherein the compound is administered to the subject in an amount of from about 450 mg to about 550 mg per dose.

39. The method of claim 38, wherein the compound is administered to the subject in an amount of about 500 mg per dose.

40. The method of any one of claims 1-39, wherein the compound is administered to the subject periodically, optionally in one or more doses per 12 hours, 24 hours, 48 hours, or week.

41. The method of claim 40, wherein the compound is administered to the subject in from one to six doses per day.

42. The method of claim 41, wherein one dose of the compound is administered to the subject once daily.

43. The method of claim 41, wherein one dose of the compound is administered to the subject twice daily.

44. The method of claim 41, wherein one dose of the compound is administered to the subject once every 4 to 12 hours.

45. The method of any one of claims 1-44, wherein the compound is administered to the subject in an amount of from about 250 mg to about 2,500 mg per day.

46. The method of claim 45, wherein the compound is administered to the subject in an amount of from about 300 mg to about 2,000 mg per day.

47. The method of claim 46, wherein the compound is administered to the subject in an amount of from about 400 mg to about 1,600 mg per day.

48. The method of claim 47, wherein the compound is administered to the subject in an amount of from about 500 mg to about 1,500 mg per day.

49. The method of claim 48, wherein the compound is administered to the subject in an amount of from about 600 mg to about 1,400 mg per day.

50. The method of claim 49, wherein the compound is administered to the subject in an amount of from about 700 mg to about 1,300 mg per day.

51. The method of claim 50, wherein the compound is administered to the subject in an amount of from about 800 mg to about 1,200 mg per day.

52. The method of claim 51, wherein the compound is administered to the subject in an amount of from about 900 mg to about 1,100 mg per day.

53. The method of claim 52, wherein the compound is administered to the subject in an amount of from about 950 mg to about 1,050 mg per day.

54. The method of claim 51, wherein the compound is administered to the subject in an amount of about 1,000 mg per day.

55. The method of any one of claims 1-54, wherein the compound is administered to the subject once daily in an amount of from about 800 mg to about 1,200 mg per dose during a first treatment period, and wherein the compound is subsequently administered to the subject twice daily in an amount of from about 300 mg to about 700 mg per dose during a second treatment period.

56. The method of claim 55, wherein the compound is administered to the subject in an amount of from about 850 mg to about 1,150 mg per dose during the first treatment period.

57. The method of claim 56, wherein the compound is administered to the subject in an amount of from about 900 mg to about 1,100 mg per dose during the first treatment period.

58. The method of claim 57, wherein the compound is administered to the subject in an amount of from about 950 mg to about 1,050 mg per dose during the first treatment period.

59. The method of claim 58, wherein the compound is administered to the subject in an amount of about 1,000 mg per dose during the first treatment period.

60. The method of any one of claims 55-59, wherein the compound is administered to the subject in an amount of from about 350 mg to about 650 mg per dose during the second treatment period.

61. The method of claim 60, wherein the compound is administered to the subject in an amount of from about 400 mg to about 600 mg per dose during the second treatment period.

62. The method of claim 61, wherein the compound is administered to the subject in an amount of from about 450 mg to about 550 mg per dose during the second treatment period.

63. The method of claim 62, wherein the compound is administered to the subject in an amount of about 500 mg per dose during the second treatment period.

64. The method of any one of claims 55-63, wherein the compound is administered to the subject in one dose every 12 hours during the second treatment period.

65. The method of any one of claim 55-64, wherein the first treatment period has a duration of from one to 10 days.

66. The method of claim 65, wherein the first treatment period has a duration of one day.

67. The method of any one of claims 55-66, wherein the second treatment period has a duration of from one to 28 days,

68. The method of claim 67, wherein the second treatment period has a duration of from four to 14 days.

69. The method of claim 68, wherein the second treatment period has a duration of from five to 10 days.

70. The method of claim 69, wherein the second treatment period has a duration of six days.

71. The method of any one of claims 1-70, wherein the compound is administered to the subject until the subject reaches a gestational age of at least about 34 weeks.

72. The method of claim 71, wherein the compound is administered to the subject until the subject reaches a gestational age of from about 34 weeks to about 40 weeks.

73. The method of claim 72, wherein the compound is administered to the subject until the subject reaches a gestational age of about 37 weeks.

74. The method of any one of claims 1-73, wherein the compound is administered to the subject orally.

75. The method of claim 74, wherein the compound is formulated as a tablet, capsule, gel cap, powder, liquid solution, or liquid suspension.

76. The method of any one of claims 1-75, wherein the atosiban is administered to the subject periodically, optionally in one or more doses per 12 hours, 24 hours, 48 hours, or week.

77. The method of any one of claims 1-76, wherein the atosiban is administered to the subject in a single bolus dose of from about 4.0 mg to about 9.5 mg.

78. The method of claim 77, wherein the atosiban is administered to the subject in a single bolus dose of from about 4.5 mg to about 9.0 mg.

79. The method of claim 78, wherein the atosiban is administered to the subject in a single bolus dose of from about 5.0 mg to about 8.5 mg.

80. The method of claim 79, wherein the atosiban is administered to the subject in a single bolus dose of from about 5.5 mg to about 8.0 mg.

81. The method of claim 80, wherein the atosiban is administered to the subject in a single bolus dose of from about 5.75 mg to about 7.75 mg.

82. The method of claim 81, wherein the atosiban is administered to the subject in a single bolus dose of from about 6.0 mg to about 7.5 mg.

83. The method of claim 82, wherein the atosiban is administered to the subject in a single bolus dose of from about 6.25 mg to about 7.25 mg.

84. The method of claim 83, wherein the atosiban is administered to the subject in a single bolus dose of from about 6.5 mg to about 7.0 mg.

85. The method of claim 84, wherein the atosiban is administered to the subject in a single bolus dose of about 6.75 mg.

86. The method of any one of claims 77-85, wherein the single bolus dose of atosiban is administered to the subject intravenously.

87. The method of any one of claims 77-86, wherein following the single bolus dose of atosiban, the atosiban is subsequently administered to the subject in an amount of from about 40 mg to about 70 mg by way of a continuous, intravenous infusion, optionally over a period of from about 1 hour to about 5 hours.

88. The method of claim 87, wherein following the single bolus dose of atosiban, the atosiban is subsequently administered to the subject in an amount of from about 45 mg to about 65 mg by way of a continuous, intravenous infusion, optionally over a period of from about 1.5 hours to about 4.5 hours.

89. The method of claim 88, wherein following the single bolus dose of atosiban, the atosiban is subsequently administered to the subject in an amount of from about 50 mg to about 60 mg by way of a continuous, intravenous infusion, optionally over a period of from about 2.0 hours to about 4.0 hours.

90. The method of claim 89, wherein following the single bolus dose of atosiban, the atosiban is subsequently administered to the subject in an amount of from about 52 mg to about 56 mg by way of a continuous, intravenous infusion, optionally over a period of from about 2.5 hours to about 3.5 hours.

91. The method of claim 90, wherein following the single bolus dose of atosiban, the atosiban is subsequently administered to the subject in an amount of about 54 mg by way of a continuous, intravenous infusion, optionally over a period of about 3.0 hours.

92. The method of any one of claims 87-91, wherein following the continuous intravenous infusion, the atosiban is subsequently administered to the subject in an amount of from about 240 mg to about 300 mg by way of a second continuous, intravenous infusion, optionally over a period of from about 42 hours to about 48 hours.

93. The method of claim 92, wherein following the continuous intravenous infusion, the atosiban is subsequently administered to the subject in an amount of from about 250 mg to about 290 mg by way of a second continuous, intravenous infusion, optionally over a period of from about 43 hours to about 47 hours.

94. The method of claim 93, wherein following the continuous intravenous infusion, the atosiban is subsequently administered to the subject in an amount of from about 260 mg to about 280 mg by way of a second continuous, intravenous infusion, optionally over a period of from about 44 hours to about 46 hours.

95. The method of claim 94, wherein following the continuous intravenous infusion, the atosiban is subsequently administered to the subject in an amount of from about 265 mg to about 275 mg by way of a second continuous, intravenous infusion, optionally over a period of from about 44.5 hours to about 45.5 hours.

96. The method of claim 95, wherein following the continuous intravenous infusion, the atosiban is subsequently administered to the subject in an amount of about 270 mg by way of a second continuous, intravenous infusion, optionally over a period of about 44 hours.

97. The method of any one of claims 1-96, wherein the atosiban is administered to the subject until the subject reaches a gestational age of at least about 34 weeks.

98. The method of claim 97, wherein the atosiban is administered to the subject until the subject reaches a gestational age of from about 34 weeks to about 40 weeks.

99. The method of claim 98, wherein the atosiban is administered to the subject until the subject reaches a gestational age of about 37 weeks.

100. The method of any one of claims 1-99, wherein administration of the compound and administration of the atosiban commence within about 48 hours of one another.

101. The method of claim 100, wherein administration of the compound and administration of the atosiban commence within about 36 hours of one another.

102. The method of claim 101, wherein administration of the compound and administration of the atosiban commence within about 24 hours of one another.

103. The method of claim 102, wherein administration of the compound and administration of the atosiban commence within about 12 hours of one another.

104. The method of claim 103, wherein administration of the compound and administration of the atosiban commence within about 6 hours of one another.

105. The method of claim 104, wherein administration of the compound and administration of the atosiban commence at substantially the same time.

106. The method of any one of claims 1-105, wherein the atosiban is formulated as an aqueous solution, optionally wherein the concentration of atosiban in the aqueous solution is from about 5.0 mg/ml to about 10.0 mg/ml.

107. The method of claim 106, wherein the concentration of atosiban in the aqueous solution is from about 5.5 mg/ml to about 9.5 mg/ml.

108. The method of claim 107, wherein the concentration of atosiban in the aqueous solution is from about 6.0 mg/ml to about 9.0 mg/ml.

109. The method of claim 108, wherein the concentration of atosiban in the aqueous solution is from about 6.5 mg/ml to about 8.5 mg/ml.

110. The method of claim 109, wherein the concentration of atosiban in the aqueous solution is from about 7.0 mg/ml to about 8.0 mg/ml.

111. The method of claim 110, wherein the concentration of atosiban in the aqueous solution is about 7.5 mg/ml.

112. The method of any one of claims 106-111, wherein the aqueous solution further comprises mannitol.

113. The method of claim 112, wherein the concentration of mannitol in the aqueous solution is from about 40 mg/ml to about 60 mg/ml.

114. The method of claim 113, wherein the concentration of mannitol in the aqueous solution is from about 45 mg/ml to about 55 mg/ml.

115. The method of claim 114, wherein the concentration of mannitol in the aqueous solution is about mg/ml.

116. The method of any one of claims 106-115, wherein the pH of the aqueous solution is from about 4.0 to about 5.0.

117. The method of claim 116, wherein the pH of the aqueous solution is from about 4.25 to about 4.75.

118. The method of claim 117, wherein the pH of the aqueous solution is about 4.5.

119. The method of any one of claims 1-118, wherein the subject is undergoing or at risk of undergoing preterm labor.

120. The method of any one of claims 1-119, wherein the subject has a gestational age of from about 24 weeks to about 36 weeks prior to administration of the atosiban and the compound to the subject.

121. The method of claim 120, wherein the subject has a gestational age of from about 24 weeks to about 34 weeks prior to administration of the atosiban and the compound to the subject, preferably wherein the subject has a gestational age of from about 24 weeks to about 30 weeks prior to administration of the atosiban and the compound to the subject.

122. The method of claim 121, wherein the subject has a gestational age of from about 28 and 0/7 weeks to about 33 and 6/7 weeks prior to administration of the atosiban and the compound to the subject.

123. The method of any one of claims 1-122, wherein the subject has a singleton or twin pregnancy, preferably wherein the subject has a singleton pregnancy.

124. The method of any one of claims 1-123, wherein the subject exhibits four or more uterine contractions per 30 minutes prior to administration of the atosiban and the compound to the subject, optionally wherein the subject exhibits four or more uterine contractions per 30 minutes up to three hours prior to administration of the atosiban and/or the compound to the subject.

125. The method of claim 124, wherein each of the uterine contractions has a duration of at least 30 seconds.

126. The method of any one of claims 1-125, wherein the subject exhibits a cervical dilation of from about 1 cm to about 4 cm prior to administration of the atosiban and the compound to the subject, optionally wherein the subject exhibits a cervical dilation of from about 1 cm to about 4 cm up to three hours prior to administration of the atosiban and/or the compound to the subject.

127. The method of any one of claims 1-126, wherein the subject tests positive for the presence of fetal fibronectin and/or insulin-like growth factor-binding protein-1 (IGFBP-1) in a sample of cervical secretion obtained from the subject prior to administration of the atosiban and the compound to the subject, optionally wherein the subject tests positive for the presence of fetal fibronectin and/or IGFBP-1 in a sample of cervical secretion obtained from the subject up to three hours prior to administration of the atosiban and/or the compound to the subject.

128. The method of any one of claims 1-127, wherein the subject exhibits a cervical length of about 25 mm or less prior to administration of the atosiban and the compound to the subject, optionally wherein the subject exhibits a cervical length of about 25 mm or less up to three hours prior to administration of the atosiban and/or the compound to the subject.

129. The method of any one of claims 1-128, wherein the subject exhibits a progressive cervical dilation prior to administration of the atosiban and the compound to the subject, optionally wherein the subject exhibits a progressive cervical dilation up to three hours prior to administration of the atosiban and/or the compound to the subject.

130. The method of claim 129, wherein the subject exhibits a cervical dilation rate of from about 0.5 cm per hour to about 0.7 cm per hour prior to administration of the atosiban and the compound to the subject, optionally wherein the subject exhibits a cervical dilation rate of from about 0.5 cm per hour to about 0.7 cm per hour up to three hours prior to administration of the atosiban and/or the compound to the subject.

131. The method of any one of claims 1-130, wherein the subject exhibits an effacement of at least 50% prior to administration of the atosiban and the compound to the subject, optionally wherein the subject exhibits an effacement of at least 50% up to three hours prior to administration of the atosiban and/or the compound to the subject.

132. The method of any one of claims 1-131, wherein delivery is delayed by from about four hours to about six weeks following the first administration of the atosiban and/or the compound to the subject.

133. The method of claim 132, wherein delivery is delayed by from about 12 hours to about 28 days following the first administration of the atosiban and/or the compound to the subject.

134. The method of claim 133, wherein delivery is delayed by from about 18 hours to about 21 days following the first administration of the atosiban and/or the compound to the subject.

135. The method of claim 134, wherein delivery is delayed by from about 24 hours to about 14 days following the first administration of the atosiban and/or the compound to the subject.

136. The method of claim 135, wherein delivery is delayed by about 48 hours following the first administration of the atosiban and/or the compound to the subject.

137. The method of claim 136, wherein delivery is delayed by about 7 days following the first administration of the atosiban and/or the compound to the subject.

138. The method of any one of claims 1-137, wherein following administration of the atosiban and the compound to the subject, the subject undergoes delivery at a gestational age of at least about 34 weeks.

139. The method of claim 138, wherein following administration of the atosiban and the compound to the subject, the subject undergoes delivery at a gestational age of at least about 37 weeks.

140. The method of any one of claims 1-139, wherein following administration of the atosiban and the compound to the subject, the subject undergoes delivery at a gestational age of from about 36 weeks to about 40 weeks.

141. A kit comprising a compound represented by formula represented by formula (I) 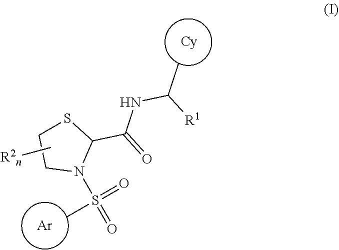
wherein ring Ar is an optionally fused, optionally substituted aryl group or an optionally fused, optionally substituted heteroaryl group;
ring Cy is an optionally fused, optionally substituted aryl group, optionally fused, optionally substituted heteroaryl group, optionally fused, optionally substituted cycloalkyl group, or an optionally fused, optionally substituted heterocycloalkyl group;
R¹ is H, carboxy, acyl, alkoxycarbonyl, C₁-C₅-alkyl carboxy, C₁-C₅-alkyl acyl, C₁-C₅-alkyl alkoxycarbonyl, C₁-C₅-alkyl acyloxy, C₁-C₅-alkyl sulfanyl, C₁-C₅ alkyl sulfinyl, C₁-C₅-alkyl sulfonyl, C₁-C₅ alkyl sulfonyloxy, C₁-C₆-alkyl, C₂-C₆-alkenyl, C₂-C₆-alkynyl, aryl, heteroaryl, C₃-C₈-cycloalkyl, C₁-C₆-alkyl aryl, C₂-C₆-alkyl heteroaryl, C₁-C₆-alkyl cycloalkyl, C₂-C₆-alkenyl aryl, C₂-C₆-alkenyl heteroaryl, C₂-C₆-alkynyl aryl, C₂-C₆-alkynyl heteroaryl, substituted carboxy, substituted acyl, substituted alkoxycarbonyl, substituted C₁-C₅-alkyl carboxy, substituted C₁-C₅-alkyl acyl, substituted C₁-C₅-alkyl alkoxycarbonyl, substituted C₁-C₅-alkyl acyloxy, substituted C₁-C₅-alkyl sulfanyl, substituted C₁-C₅-alkyl sulfinyl, substituted C₁-C₅-alkyl sulfonyl, substituted C₁-C₅-alkyl sulfonyloxy, substituted C₁-C₆-alkyl, substituted C₂-C₆-alkenyl, substituted C₂-C₆-alkynyl, substituted aryl, substituted heteroaryl, substituted C₃-C₈-cycloalkyl, substituted C₁-C₆-alkyl aryl, substituted C₁-C₆-alkyl heteroaryl, substituted C₁-C₆-alkyl cycloalkyl, substituted C₂-C₆-alkenyl aryl, substituted C₂-C₆-alkenyl heteroaryl, substituted C₂-C₆-alkynyl aryl, or substituted C₂-C₆-alkynyl heteroaryl;
each R² is independently C₁-C₆-alkyl, C₂-C₆-alkenyl, C₂-C₆-alkynyl, substituted C₁-C₆-alkyl, substituted C₂-C₆-alkenyl, or substituted C₂-C₆-alkynyl; and
n is an integer from 0 to 2,
or a pharmaceutically acceptable salt thereof,
and wherein the kit further comprises a package insert instructing a user of the kit to administer the compound to a pregnant human subject in accordance with the method of any one of claims 1-140.

142. The kit of claim 141, wherein the compound is represented by formula (V) 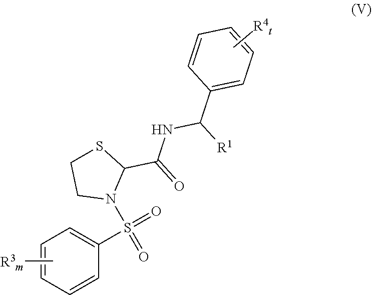
wherein R¹ is C₁-C₅-alkyl carboxy, C₁-C₅-alkyl acyl, C₁-C₅-alkyl alkoxycarbonyl, C₁-C₅-alkyl acyloxy, substituted C₁-C₅-alkyl carboxy, substituted C₁-C₅-alkyl acyl, substituted C₁-C₅-alkyl alkoxycarbonyl, or substituted C₁-C₅-alkyl acyloxy;
each R³ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each R⁴ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
m is an integer from 0-5; and
t is an integer from 0-5,
or a pharmaceutically acceptable salt thereof.

143. The kit of claim 142, wherein the compound is represented by formula (Va) 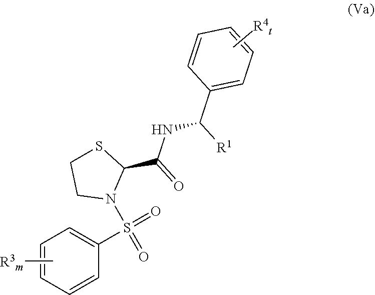
or a pharmaceutically acceptable salt thereof.

144. The kit of any one of claims 141-143, wherein the compound is represented by formula (VI) 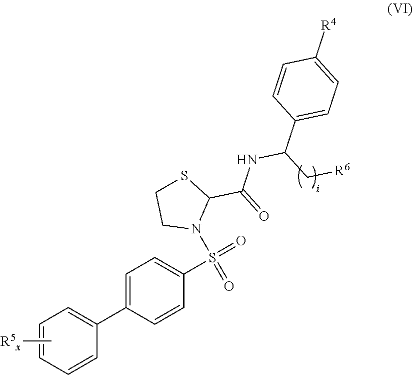
wherein R⁶ is hydroxyl, optionally substituted acyl, optionally substituted alkoxycarbonyl, or optionally substituted acyloxy;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3; and
x is an integer from 0-5,
or a pharmaceutically acceptable salt thereof.

145. The kit of claim 144, wherein the compound is represented by formula (VII) 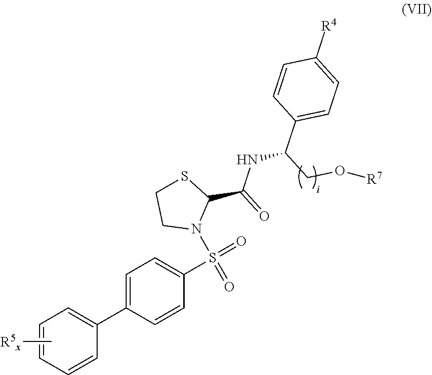
wherein R⁷ is H or optionally substituted aminoacyl;
each R⁵ is independently halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
R⁴ is halogen, haloalkyl, cyano, optionally substituted amino, hydroxyl, thiol, optionally substituted alkoxy, optionally substituted acyloxy, optionally substituted alkoxycarbonyl, carboxy, ureido, alkyl sulfonyl, aryl sulfonyl, heteroaryl sulfonyl, cycloalkyl sulfonyl, heterocycloalkyl sulfonyl, alkyl sulfanyl, aryl sulfanyl, heteroaryl sulfanyl, cycloalkyl sulfanyl, heterocycloalkyl sulfanyl, alkyl sulfinyl, aryl sulfinyl, heteroaryl sulfinyl, cycloalkyl sulfinyl, heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
i is an integer from 0-3; and
x is an integer from 0-5,
or a pharmaceutically acceptable salt thereof.

146. The kit of any one of claims 141-145, wherein the compound is compound (1).

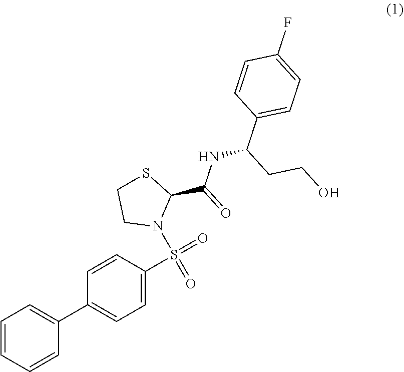 [SMILES: O=C(N[C@@H](CCO)c1ccc(F)cc1)[C@@H]1SCCN1S(=O)(=O)c1ccc(-c2ccccc2)cc1]

147. The kit of any one of claims 141-145, wherein the compound is compound (2) 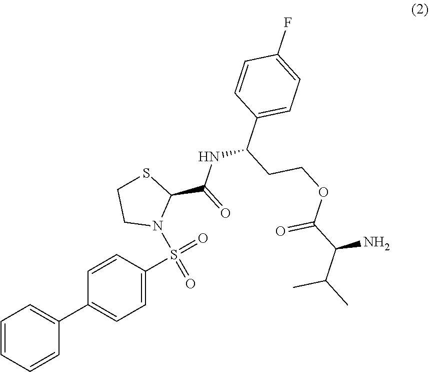 [SMILES: CC(C)[C@H](N)C(=O)OCC[C@H](NC(=O)[C@@H]1SCCN1S(=O)(=O)c1ccc(-c2ccccc2)cc1)c1ccc(F)cc1]
or a pharmaceutically acceptable salt thereof.

148. The kit of claim 147, wherein the compound is represented by formula (3).

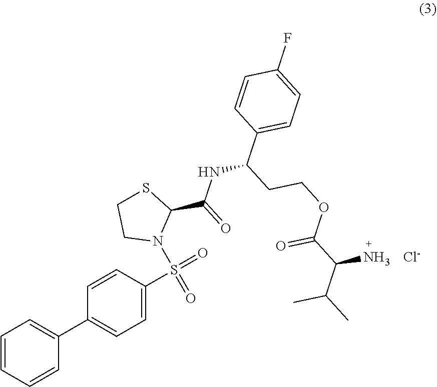 [SMILES: CC(C)[C@H](N)C(=O)OCC[C@H](NC(=O)[C@@H]1SCCN1S(=O)(=O)c1ccc(-c2ccccc2)cc1)c1ccc(F)cc1 ; [Cl-]]

149. The kit of any one of claims 141-148, wherein the compound is formulated for oral administration to the subject.

150. The kit of claim 149, wherein the compound is formulated as a tablet, capsule, gel cap, powder, liquid solution, or liquid suspension.

151. The kit of any one of claims 141-150, wherein the kit further comprises atosiban.

152. The kit of claim 151, wherein the atosiban is formulated for intravenous administration to the subject.

153. The kit of claim 152, wherein the atosiban is formulated as an aqueous solution.
